SEQUENCE LISTING (279 sequences)

SEQ ID NO: 1 - protein, 760 aa, Homo sapiens
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 2 - protein, 760 aa, Macaca fascicularis
MMDQARSAFSNLFGGEPLSYTRFSLARQVDGDNSHVEMKLAVDDEENADNNTKANGTKPKRCGGNICYGTIAVIIFFLIGFMIGYLGYCKGVEPKTECERLAGTESPAREEPEEDFPAAPRLYWDDLKRKLSEKLDTTDFTSTIKLLNENLYVPREAGSQKDENLALYIENQFREFKLSKVWRDQHFVKIQVKDSAQNSVIIVDKNGGLVYLVENPGGYVAYSKAATVTGKLVHANFGTKKDFEDLDSPVNGSIVIVRAGKITFAEKVANAESLNAIGVLIYMDQTKFPIVKADLSFFGHAHLGTGDPYTPGFPSFNHTQFPPSQSSGLPNIPVQTISRAAAEKLFGNMEGDCPSDWKTDSTCKMVTSENKSVKLTVSNVLKETKILNIFGVIKGFVEPDHYVVVGAQRDAWGPGAAKSSVGTALLLKLAQMFSDMVLKDGFQPSRSIIFASWSAGDFGSVGATEWLEGYLSSLHLKAFTYINLDKAVLGTSNFKVSASPLLYTLIEKTMQDVKHPVTGRSLYQDSNWASKVEKLTLDNAAFPFLAYSGIPAVSFCFCEDTDYPYLGTTMDTYKELVERIPELNKVARAAAEVAGQFVIKLTHDTELNLDYERYNSQLLLFLRDLNQYRADVKEMGLSLQWLYSARGDFFRATSRLTTDFRNAEKRDKFVMKKLNDRVMRVEYYFLSPYVSPKESPFRHVFWGSGSHTLSALLESLKLRRQNNSAFNETLFRNQLALATWTIQGAANALSGDVWDIDNEF

SEQ ID NO: 3 - protein, 5 aa, synthetic construct
  REGION 1..5: note = Amino acid sequence of exemplified linker 1
GGGGS

SEQ ID NO: 4 - protein, 6 aa, synthetic construct
  REGION 1..6: note = Amino acid sequence of exemplified linker 2
GGGGGS

SEQ ID NO: 5 - protein, 5 aa, synthetic construct
  REGION 1..5: note = Amino acid sequence of exemplified linker 3
SGGGG

SEQ ID NO: 6 - protein, 6 aa, synthetic construct
  REGION 1..6: note = Amino acid sequence 1 of CDR 1 in the light chain of mouse anti-hTfR antibody No.1
QDVNSA

SEQ ID NO: 7 - protein, 11 aa, synthetic construct
  REGION 1..11: note = Amino acid sequence 2 of CDR 1 in the light chain of mouse anti-hTfR antibody No.1
KASQDVNSAVA

SEQ ID NO: 8 - protein, 6 aa, synthetic construct
  REGION 1..6: note = Amino acid sequence 1 of CDR 2 in the light chain of mouse anti-hTfR antibody No.1
WTSTRH

SEQ ID NO: 9 - protein, 7 aa, synthetic construct
  REGION 1..7: note = Amino acid sequence 2 of CDR 2 in the light chain of mouse anti-hTfR antibody No.1
WTSTRHT

SEQ ID NO: 10 - protein, 9 aa, synthetic construct
  REGION 1..9: note = Amino acid sequence of CDR 3 in the light chain of mouse anti-hTfR antibody No.1
QQHYSTPRT

SEQ ID NO: 11 - protein, 6 aa, synthetic construct
  REGION 1..6: note = Amino acid sequence 1 of CDR 1 in the light chain of mouse anti-hTfR antibody No.2
QSISNN

SEQ ID NO: 12 - protein, 11 aa, synthetic construct
  REGION 1..11: note = Amino acid sequence 2 of CDR 1 in the light chain of mouse anti-hTfR antibody No.2
RASQSISNNLQ

SEQ ID NO: 13 - protein, 6 aa, synthetic construct
  REGION 1..6: note = Amino acid sequence 1 of CDR 2 in the light chain of mouse anti-hTfR antibody No.2
YASQSI

SEQ ID NO: 14 - protein, 7 aa, synthetic construct
  REGION 1..7: note = Amino acid sequence 2 of CDR 2 in the light chain of mouse anti-hTfR antibody No.2
YASQSIS

SEQ ID NO: 15 - protein, 9 aa, synthetic construct
  REGION 1..9: note = Amino acid sequence of CDR 3 in the light chain of mouse anti-hTfR antibody No.2
QQSNSWPRT

SEQ ID NO: 16 - protein, 11 aa, synthetic construct
  REGION 1..11: note = Amino acid sequence 1 of CDR 1 in the light chain of mouse anti-hTfR antibody No.3
QSLVHSNGNTY

SEQ ID NO: 17 - protein, 16 aa, synthetic construct
  REGION 1..16: note = Amino acid sequence 2 of CDR 1 in the light chain of mouse anti-hTfR antibody No.3
RSSQSLVHSNGNTYLH

SEQ ID NO: 18 - protein, 6 aa, synthetic construct
  REGION 1..6: note = Amino acid sequence 1 of CDR 2 in the light chain of mouse anti-hTfR antibody No.3
KVSNRF

SEQ ID NO: 19 - protein, 7 aa, synthetic construct
  REGION 1..7: note = Amino acid sequence 2 of CDR 2 in the light chain of mouse anti-hTfR antibody No.3
KVSNRFS

SEQ ID NO: 20 - protein, 9 aa, synthetic construct
  REGION 1..9: note = Amino acid sequence of CDR 3 in the light chain of mouse anti-hTfR antibody No.3
SQSTHVPWT

SEQ ID NO: 21 - protein, 5 aa, synthetic construct
  REGION 1..5: note = Amino acid sequence 1 of CDR 1 in the light chain of mouse anti-hTfR antibody No.4
SNVNY

SEQ ID NO: 22 - protein, 10 aa, synthetic construct
  REGION 1..10: note = Amino acid sequence 2 of CDR 1 in the light chain of mouse anti-hTfR antibody No.4
SASSNVNYMH

SEQ ID NO: 23 - protein, 6 aa, synthetic construct
  REGION 1..6: note = Amino acid sequence 1 of CDR 2 in the light chain of mouse anti-hTfR antibody No.4
DTSKLA

SEQ ID NO: 24 - protein, 7 aa, synthetic construct
  REGION 1..7: note = Amino acid sequence 2 of CDR 2 in the light chain of mouse anti-hTfR antibody No.4
DTSKLAS

SEQ ID NO: 25 - protein, 9 aa, synthetic construct
  REGION 1..9: note = Amino acid sequence of CDR 3 in the light chain of mouse anti-hTfR antibody No.4
FQGNGNPYT

SEQ ID NO: 26 - protein, 5 aa, synthetic construct
  REGION 1..5: note = Amino acid sequence 1 of CDR 1 in the light chain of mouse anti-hTfR antibody No.5
SSISS

SEQ ID NO: 27 - protein, 10 aa, synthetic construct
  REGION 1..10: note = Amino acid sequence 2 of CDR 1 in the light chain of mouse anti-hTfR antibody No.5
SASSSISSIH

SEQ ID NO: 28 - protein, 6 aa, synthetic construct
  REGION 1..6: note = Amino acid sequence 1 of CDR 2 in the light chain of mouse anti-hTfR antibody No.5
DTSTLA

SEQ ID NO: 29 - protein, 7 aa, synthetic construct
  REGION 1..7: note = Amino acid sequence 2 of CDR 2 in the light chain of mouse anti-hTfR antibody No.5
DTSTLAS

SEQ ID NO: 30 - protein, 8 aa, synthetic construct
  REGION 1..8: note = Amino acid sequence of CDR 3 in the light chain of mouse anti-hTfR antibody No.5
HQRSSYPT

SEQ ID NO: 31 - protein, 6 aa, synthetic construct
  REGION 1..6: note = Amino acid sequence 1 of CDR 1 in the light chain of mouse anti-hTfR antibody No.6
QEISGY

SEQ ID NO: 32 - protein, 11 aa, synthetic construct
  REGION 1..11: note = Amino acid sequence 2 of CDR 1 in the light chain of mouse anti-hTfR antibody No.6
RASQEISGYLS

SEQ ID NO: 33 - protein, 6 aa, synthetic construct
  REGION 1..6: note = Amino acid sequence 1 of CDR 2 in the light chain of mouse anti-hTfR antibody No.6
AASTLD

SEQ ID NO: 34 - protein, 7 aa, synthetic construct
  REGION 1..7: note = Amino acid sequence 2 of CDR 2 in the light chain of mouse anti-hTfR antibody No.6
AASTLDS

SEQ ID NO: 35 - protein, 9 aa, synthetic construct
  REGION 1..9: note = Amino acid sequence of CDR 3 in the light chain of mouse anti-hTfR antibody No.6
LQYSSYPRT

SEQ ID NO: 36 - protein, 5 aa, synthetic construct
  REGION 1..5: note = Amino acid sequence 1 of CDR 1 in the light chain of mouse anti-hTfR antibody No.7
SSVNY

SEQ ID NO: 37 - protein, 10 aa, synthetic construct
  REGION 1..10: note = Amino acid sequence 2 of CDR 1 in the light chain of mouse anti-hTfR antibody No.7
SASSSVNYIH

SEQ ID NO: 38 - protein, 6 aa, synthetic construct
  REGION 1..6: note = Amino acid sequence 1 of CDR 2 in the light chain of mouse anti-hTfR antibody No.7
QTSNLA

SEQ ID NO: 39 - protein, 7 aa, synthetic construct
  REGION 1..7: note = Amino acid sequence 2 of CDR 2 in the light chain of mouse anti-hTfR antibody No.7
QTSNLAS

SEQ ID NO: 40 - protein, 9 aa, synthetic construct
  REGION 1..9: note = Amino acid sequence of CDR 3 in the light chain of mouse anti-hTfR antibody No.7
HQWSSYPWT

SEQ ID NO: 41 - protein, 5 aa, synthetic construct
  REGION 1..5: note = Amino acid sequence 1 of CDR 1 in the light chain of mouse anti-hTfR antibody No.8
SSVSY

SEQ ID NO: 42 - protein, 10 aa, synthetic construct
  REGION 1..10: note = Amino acid sequence 2 of CDR 1 in the light chain of mouse anti-hTfR antibody No.8
SASSSVSYMH

SEQ ID NO: 43 - protein, 6 aa, synthetic construct
  REGION 1..6: note = Amino acid sequence 1 of CDR 2 in the light chain of mouse anti-hTfR antibody No.8
GTSNLA

SEQ ID NO: 44 - protein, 7 aa, synthetic construct
  REGION 1..7: note = Amino acid sequence 2 of CDR 2 in the light chain of mouse anti-hTfR antibody No.8
GTSNLAS

SEQ ID NO: 45 - protein, 9 aa, synthetic construct
  REGION 1..9: note = Amino acid sequence of CDR 3 in the light chain of mouse anti-hTfR antibody No.8
HQWSSYPWT

SEQ ID NO: 46 - protein, 5 aa, synthetic construct
  REGION 1..5: note = Amino acid sequence 1 of CDR 1 in the light chain of mouse anti-hTfR antibody No.9
SSVSL

SEQ ID NO: 47 - protein, 10 aa, synthetic construct
  REGION 1..10: note = Amino acid sequence 2 of CDR 1 in the light chain of mouse anti-hTfR antibody No.9
SASSSVSLMF

SEQ ID NO: 48 - protein, 6 aa, synthetic construct
  REGION 1..6: note = Amino acid sequence 1 of CDR 2 in the light chain of mouse anti-hTfR antibody No.9
FTSYRA

SEQ ID NO: 49 - protein, 7 aa, synthetic construct
  REGION 1..7: note = Amino acid sequence 2 of CDR 2 in the light chain of mouse anti-hTfR antibody No.9
FTSYRAS

SEQ ID NO: 50 - protein, 9 aa, synthetic construct
  REGION 1..9: note = Amino acid sequence of CDR 3 in the light chain of mouse anti-hTfR antibody No.9
QQWTSDPPT

SEQ ID NO: 51 - protein, 6 aa, synthetic construct
  REGION 1..6: note = Amino acid sequence 1 of CDR 1 in the light chain of mouse anti-hTfR antibody No.10
QEISGY

SEQ ID NO: 52 - protein, 11 aa, synthetic construct
  REGION 1..11: note = Amino acid sequence 2 of CDR 1 in the light chain of mouse anti-hTfR antibody No.10
RPSQEISGYLS

SEQ ID NO: 53 - protein, 6 aa, synthetic construct
  REGION 1..6: note = Amino acid sequence 1 of CDR 2 in the light chain of mouse anti-hTfR antibody No.10
AASTLD

SEQ ID NO: 54 - protein, 7 aa, synthetic construct
  REGION 1..7: note = Amino acid sequence 2 of CDR 2 in the light chain of mouse anti-hTfR antibody No.10
AASTLDS

SEQ ID NO: 55 - protein, 9 aa, synthetic construct
  REGION 1..9: note = Amino acid sequence of CDR 3 in the light chain of mouse anti-hTfR antibody No.10
LQYASYPRT

SEQ ID NO: 56 - protein, 6 aa, synthetic construct
  REGION 1..6: note = Amino acid sequence 1 of CDR 1 in the light chain of mouse anti-hTfR antibody No.11
QSIRNY

SEQ ID NO: 57 - protein, 11 aa, synthetic construct
  REGION 1..11: note = Amino acid sequence 2 of CDR 1 in the light chain of mouse anti-hTfR antibody No.11
RASQSIRNYLH

SEQ ID NO: 58 - protein, 6 aa, synthetic construct
  REGION 1..6: note = Amino acid sequence 1 of CDR 2 in the light chain of mouse anti-hTfR antibody No.11
YASQSI

SEQ ID NO: 59 - protein, 7 aa, synthetic construct
  REGION 1..7: note = Amino acid sequence 2 of CDR 2 in the light chain of mouse anti-hTfR antibody No.11
YASQSIS

SEQ ID NO: 60 - protein, 9 aa, synthetic construct
  REGION 1..9: note = Amino acid sequence of CDR 3 in the light chain of mouse anti-hTfR antibody No.11
QQTNSWPYT

SEQ ID NO: 61 - protein, 6 aa, synthetic construct
  REGION 1..6: note = Amino acid sequence 1 of CDR 1 in the light chain of mouse anti-hTfR antibody No.12
HDVKTA

SEQ ID NO: 62 - protein, 11 aa, synthetic construct
  REGION 1..11: note = Amino acid sequence 2 of CDR 1 in the light chain of mouse anti-hTfR antibody No.12
KASHDVKTAVA

SEQ ID NO: 63 - protein, 6 aa, synthetic construct
  REGION 1..6: note = Amino acid sequence 1 of CDR 2 in the light chain of mouse anti-hTfR antibody No.12
WSSTRH

SEQ ID NO: 64 - protein, 7 aa, synthetic construct
  REGION 1..7: note = Amino acid sequence 2 of CDR 2 in the light chain of mouse anti-hTfR antibody No.12
WSSTRHT

SEQ ID NO: 65 - protein, 9 aa, synthetic construct
  REGION 1..9: note = Amino acid sequence of CDR 3 in the light chain of mouse anti-hTfR antibody No.12
QQHFSTPLT

SEQ ID NO: 66 - protein, 6 aa, synthetic construct
  REGION 1..6: note = Amino acid sequence 1 of CDR 1 in the light chain of mouse anti-hTfR antibody No.13
QSIRNY

SEQ ID NO: 67 - protein, 11 aa, synthetic construct
  REGION 1..11: note = Amino acid sequence 2 of CDR 1 in the light chain of mouse anti-hTfR antibody No.13
RASQSIRNYLH

SEQ ID NO: 68 - protein, 6 aa, synthetic construct
  REGION 1..6: note = Amino acid sequence 1 of CDR 2 in the light chain of mouse anti-hTfR antibody No.13
YASQSI

SEQ ID NO: 69 - protein, 7 aa, synthetic construct
  REGION 1..7: note = Amino acid sequence 2 of CDR 2 in the light chain of mouse anti-hTfR antibody No.13
YASQSIS

SEQ ID NO: 70 - protein, 9 aa, synthetic construct
  REGION 1..9: note = Amino acid sequence of CDR 3 in the light chain of mouse anti-hTfR antibody No.13
QQTNSWPYT

SEQ ID NO: 71 - protein, 5 aa, synthetic construct
  REGION 1..5: note = Amino acid sequence 1 of CDR 1 in the light chain of mouse anti-hTfR antibody No.14
SNINS

SEQ ID NO: 72 - protein, 10 aa, synthetic construct
  REGION 1..10: note = Amino acid sequence 2 of CDR 1 in the light chain of mouse anti-hTfR antibody No.14
SASSNINSIH

SEQ ID NO: 73 - protein, 6 aa, synthetic construct
  REGION 1..6: note = Amino acid sequence 1 of CDR 2 in the light chain of mouse anti-hTfR antibody No.14
DTSNLA

SEQ ID NO: 74 - protein, 7 aa, synthetic construct
  REGION 1..7: note = Amino acid sequence 2 of CDR 2 in the light chain of mouse anti-hTfR antibody No.14
DTSNLAS

SEQ ID NO: 75 - protein, 9 aa, synthetic construct
  REGION 1..9: note = Amino acid sequence of CDR 3 in the light chain of mouse anti-hTfR antibody No.14
HQRSGYPFT

SEQ ID NO: 76 - protein, 5 aa, synthetic construct
  REGION 1..5: note = Amino acid sequence 1 of CDR 1 in the heavy chain of mouse anti-hTfR antibody No.1
NYGMS

SEQ ID NO: 77 - protein, 8 aa, synthetic construct
  REGION 1..8: note = Amino acid sequence 2 of CDR 1 in the heavy chain of mouse anti-hTfR antibody No.1
GLTFSNYG

SEQ ID NO: 78 - protein, 8 aa, synthetic construct
  REGION 1..8: note = Amino acid sequence 1 of CDR 2 in the heavy chain of mouse anti-hTfR antibody No.1
INTNGGST

SEQ ID NO: 79 - protein, 17 aa, synthetic construct
  REGION 1..17: note = Amino acid sequence 2 of CDR 2 in the heavy chain of mouse anti-hTfR antibody No.1
NINTNGGSTYYPDSVKG

SEQ ID NO: 80 - protein, 7 aa, synthetic construct
  REGION 1..7: note = Amino acid sequence 1 of CDR 3 in the heavy chain of mouse anti-hTfR antibody No.1
NRYDEDY

SEQ ID NO: 81 - protein, 9 aa, synthetic construct
  REGION 1..9: note = Amino acid sequence 2 of CDR 3 in the heavy chain of mouse anti-hTfR antibody No.1
TNNRYDEDY

SEQ ID NO: 82 - protein, 5 aa, synthetic construct
  REGION 1..5: note = Amino acid sequence 1 of CDR 1in the heavy chain of mouse anti-hTfR antibody No.2
DYVMH

SEQ ID NO: 83 - protein, 8 aa, synthetic construct
  REGION 1..8: note = Amino acid sequence 2 of CDR 1 in the heavy chain of mouse anti-hTfR antibody No.2
GYTFTDYV

SEQ ID NO: 84 - protein, 8 aa, synthetic construct
  REGION 1..8: note = Amino acid sequence 1 of CDR 2 in the heavy chain of mouse anti-hTfR antibody No.2
ISTYYGHG

SEQ ID NO: 85 - protein, 17 aa, synthetic construct
  REGION 1..17: note = Amino acid sequence 2 of CDR 2 in the heavy chain of mouse anti-hTfR antibody No.2
VISTYYGHGSYNQRFKG

SEQ ID NO: 86 - protein, 14 aa, synthetic construct
  REGION 1..14: note = Amino acid sequence 1 of CDR 3 in the heavy chain of mouse anti-hTfR antibody No.2
GGYGSSSLAGNFDV

SEQ ID NO: 87 - protein, 16 aa, synthetic construct
  REGION 1..16: note = Amino acid sequence 2 of CDR 3 in the heavy chain of mouse anti-hTfR antibody No.2
VRGGYGSSSLAGNFDV

SEQ ID NO: 88 - protein, 5 aa, synthetic construct
  REGION 1..5: note = Amino acid sequence 1 of CDR 1 in the heavy chain of mouse anti-hTfR antibody No.3
NYWLG

SEQ ID NO: 89 - protein, 8 aa, synthetic construct
  REGION 1..8: note = Amino acid sequence 2 of CDR 1 in the heavy chain of mouse anti-hTfR antibody No.3
GYSFTNYW

SEQ ID NO: 90 - protein, 8 aa, synthetic construct
  REGION 1..8: note = Amino acid sequence 1 of CDR 2 in the heavy chain of mouse anti-hTfR antibody No.3
IYPGGDYP

SEQ ID NO: 91 - protein, 17 aa, synthetic construct
  REGION 1..17: note = Amino acid sequence 2 of CDR 2 in the heavy chain of mouse anti-hTfR antibody No.3
DIYPGGDYPTYSEKFKV

SEQ ID NO: 92 - protein, 9 aa, synthetic construct
  REGION 1..9: note = Amino acid sequence 1 of CDR 3 in the heavy chain of mouse anti-hTfR antibody No.3
SGNYDEVAY

SEQ ID NO: 93 - protein, 11 aa, synthetic construct
  REGION 1..11: note = Amino acid sequence 2 of CDR 3 in the heavy chain of mouse anti-hTfR antibody No.3
ARSGNYDEVAY

SEQ ID NO: 94 - protein, 5 aa, synthetic construct
  REGION 1..5: note = Amino acid sequence 1 of CDR 1 in the heavy chain of mouse anti-hTfR antibody No.4
NYLIE

SEQ ID NO: 95 - protein, 8 aa, synthetic construct
  REGION 1..8: note = Amino acid sequence 2 of CDR 1 in the heavy chain of mouse anti-hTfR antibody No.4
GYAFTNYL

SEQ ID NO: 96 - protein, 8 aa, synthetic construct
  REGION 1..8: note = Amino acid sequence 1 of CDR 2 in the heavy chain of mouse anti-hTfR antibody No.4
INPGSGGI

SEQ ID NO: 97 - protein, 16 aa, synthetic construct
  REGION 1..16: note = Amino acid sequence 2 of CDR 2 in the heavy chain of mouse anti-hTfR antibody No.4
INPGSGGIIYNEKFTD

SEQ ID NO: 98 - protein, 13 aa, synthetic construct
  REGION 1..13: note = Amino acid sequence 1 of CDR 3 in the heavy chain of mouse anti-hTfR antibody No.4
SNYYGTTYWHFDV

SEQ ID NO: 99 - protein, 15 aa, synthetic construct
  REGION 1..15: note = Amino acid sequence 2 of CDR 3 in the heavy chain of mouse anti-hTfR antibody No.4
ARSNYYGTTYWHFDV

SEQ ID NO: 100 - protein, 5 aa, synthetic construct
  REGION 1..5: note = Amino acid sequence 1 of CDR 1 in the heavy chain of mouse anti-hTfR antibody No.5
NFVIH

SEQ ID NO: 101 - protein, 8 aa, synthetic construct
  REGION 1..8: note = Amino acid sequence 2 of CDR 1 in the heavy chain of mouse anti-hTfR antibody No.5
GYTFTNFV

SEQ ID NO: 102 - protein, 8 aa, synthetic construct
  REGION 1..8: note = Amino acid sequence 1 of CDR 2 in the heavy chain of mouse anti-hTfR antibody No.5
FNPHKNGA

SEQ ID NO: 103 - protein, 16 aa, synthetic construct
  REGION 1..16: note = Amino acid sequence 2 of CDR 2 in the heavy chain of mouse anti-hTfR antibody No.5
FNPHKNGAEYNEKFQV

SEQ ID NO: 104 - protein, 9 aa, synthetic construct
  REGION 1..9: note = Amino acid sequence 1 of CDR 3 in the heavy chain of mouse anti-hTfR antibody No.5
SFYYYSMDY

SEQ ID NO: 105 - protein, 11 aa, synthetic construct
  REGION 1..11: note = Amino acid sequence 2 of CDR 3 in the heavy chain of mouse anti-hTfR antibody No.5
ARSFYYYSMDY

SEQ ID NO: 106 - protein, 5 aa, synthetic construct
  REGION 1..5: note = Amino acid sequence 1 of CDR 1 in the heavy chain of mouse anti-hTfR antibody No.6
TYGVY

SEQ ID NO: 107 - protein, 8 aa, synthetic construct
  REGION 1..8: note = Amino acid sequence 2 of CDR 1 in the heavy chain of mouse anti-hTfR antibody No.6
GFSLSTYG

SEQ ID NO: 108 - protein, 7 aa, synthetic construct
  REGION 1..7: note = Amino acid sequence of CDR 2 in the heavy chain of mouse anti-hTfR antibody No.6
IWGDGST

SEQ ID NO: 109 - protein, 4 aa, synthetic construct
  REGION 1..4: note = Amino acid sequence 1 of CDR 3 in the heavy chain of mouse anti-hTfR antibody No.6
PDDV

SEQ ID NO: 110 - protein, 6 aa, synthetic construct
  REGION 1..6: note = Amino acid sequence 2 of CDR 3 in the heavy chain of mouse anti-hTfR antibody No.6
AKPDDV

SEQ ID NO: 111 - protein, 5 aa, synthetic construct
  REGION 1..5: note = Amino acid sequence 1 of CDR 1 in the heavy chain of mouse anti-hTfR antibody No.7
NYFMS

SEQ ID NO: 112 - protein, 8 aa, synthetic construct
  REGION 1..8: note = Amino acid sequence 2 of CDR 1 in the heavy chain of mouse anti-hTfR antibody No.7
GITFRNYF

SEQ ID NO: 113 - protein, 8 aa, synthetic construct
  REGION 1..8: note = Amino acid sequence 1 of CDR 2 in the heavy chain of mouse anti-hTfR antibody No.7
ISSAGGYT

SEQ ID NO: 114 - protein, 16 aa, synthetic construct
  REGION 1..16: note = Amino acid sequence 2 of CDR 2 in the heavy chain of mouse anti-hTfR antibody No.7
ISSAGGYTYYADSVKG

SEQ ID NO: 115 - protein, 11 aa, synthetic construct
  REGION 1..11: note = Amino acid sequence 1 of CDR 3 in the heavy chain of mouse anti-hTfR antibody No.7
QEVPYPYAMDY

SEQ ID NO: 116 - protein, 13 aa, synthetic construct
  REGION 1..13: note = Amino acid sequence 2 of CDR 3 in the heavy chain of mouse anti-hTfR antibody No.7
ARQEVPYPYAMDY

SEQ ID NO: 117 - protein, 5 aa, synthetic construct
  REGION 1..5: note = Amino acid sequence 1 of CDR 1 in the heavy chain of mouse anti-hTfR antibody No.8
TYGVS

SEQ ID NO: 118 - protein, 8 aa, synthetic construct
  REGION 1..8: note = Amino acid sequence 2 of CDR 1 in the heavy chain of mouse anti-hTfR antibody No.8
GFSLTTYG

SEQ ID NO: 119 - protein, 7 aa, synthetic construct
  REGION 1..7: note = Amino acid sequence of CDR 2 in the heavy chain of mouse anti-hTfR antibody No.8
IWGDGST

SEQ ID NO: 120 - protein, 4 aa, synthetic construct
  REGION 1..4: note = Amino acid sequence 1 of CDR 3 in the heavy chain of mouse anti-hTfR antibody No.8
PDDY

SEQ ID NO: 121 - protein, 6 aa, synthetic construct
  REGION 1..6: note = Amino acid sequence 2 of CDR 3 in the heavy chain of mouse anti-hTfR antibody No.8
AKPDDY

SEQ ID NO: 122 - protein, 5 aa, synthetic construct
  REGION 1..5: note = Amino acid sequence 1 of CDR 1 in the heavy chain of mouse anti-hTfR antibody No.9
NYGVS

SEQ ID NO: 123 - protein, 8 aa, synthetic construct
  REGION 1..8: note = Amino acid sequence 2 of CDR 1 in the heavy chain of mouse anti-hTfR antibody No.9
GYTFTNYG

SEQ ID NO: 124 - protein, 8 aa, synthetic construct
  REGION 1..8: note = Amino acid sequence 1 of CDR 2 in the heavy chain of mouse anti-hTfR antibody No.9
IYTFTGEA

SEQ ID NO: 125 - protein, 16 aa, synthetic construct
  REGION 1..16: note = Amino acid sequence 2 of CDR 2 in the heavy chain of mouse anti-hTfR antibody No.9
IYTFTGEATYIDDFRG

SEQ ID NO: 126 - protein, 8 aa, synthetic construct
  REGION 1..8: note = Amino acid sequence 1 of CDR 3 in the heavy chain of mouse anti-hTfR antibody No.9
RNGAWFED

SEQ ID NO: 127 - protein, 10 aa, synthetic construct
  REGION 1..10: note = Amino acid sequence 2 of CDR 3 in the heavy chain of mouse anti-hTfR antibody No.9
SRRNGAWFED

SEQ ID NO: 128 - protein, 5 aa, synthetic construct
  REGION 1..5: note = Amino acid sequence 1 of CDR 1 in the heavy chain of mouse anti-hTfR antibody No.10
NYAMS

SEQ ID NO: 129 - protein, 8 aa, synthetic construct
  REGION 1..8: note = Amino acid sequence 2 of CDR 1 in the heavy chain of mouse anti-hTfR antibody No.10
GITFRNYA

SEQ ID NO: 130 - protein, 8 aa, synthetic construct
  REGION 1..8: note = Amino acid sequence 1 of CDR 2 in the heavy chain of mouse anti-hTfR antibody No.10
ISSYGGYT

SEQ ID NO: 131 - protein, 16 aa, synthetic construct
  REGION 1..16: note = Amino acid sequence 2 of CDR 2 in the heavy chain of mouse anti-hTfR antibody No.10
ISSYGGYTYYADSVKG

SEQ ID NO: 132 - protein, 11 aa, synthetic construct
  REGION 1..11: note = Amino acid sequence 1 of CDR 3 in the heavy chain of mouse anti-hTfR antibody No.10
QEVPYPYPMDN

SEQ ID NO: 133 - protein, 13 aa, synthetic construct
  REGION 1..13: note = Amino acid sequence 2 of CDR 3 in the heavy chain of mouse anti-hTfR antibody No.10
ARQEVPYPYPMDN

SEQ ID NO: 134 - protein, 5 aa, synthetic construct
  REGION 1..5: note = Amino acid sequence 1 of CDR 1 in the heavy chain of mouse anti-hTfR antibody No.11
IYWIN

SEQ ID NO: 135 - protein, 8 aa, synthetic construct
  REGION 1..8: note = Amino acid sequence 2 of CDR 1 in the heavy chain of mouse anti-hTfR antibody No.11
GYAFSIYW

SEQ ID NO: 136 - protein, 8 aa, synthetic construct
  REGION 1..8: note = Amino acid sequence 1 of CDR 2 in the heavy chain of mouse anti-hTfR antibody No.11
IYPGNGDT

SEQ ID NO: 137 - protein, 16 aa, synthetic construct
  REGION 1..16: note = Amino acid sequence 2 of CDR 2 in the heavy chain of mouse anti-hTfR antibody No.11
IYPGNGDTNYNGKFKG

SEQ ID NO: 138 - protein, 9 aa, synthetic construct
  REGION 1..9: note = Amino acid sequence 1 of CDR 3 in the heavy chain of mouse anti-hTfR antibody No.11
WGDDYAMDF

SEQ ID NO: 139 - protein, 11 aa, synthetic construct
  REGION 1..11: note = Amino acid sequence 2 of CDR 3 in the heavy chain of mouse anti-hTfR antibody No.11
GRWGDDYAMDF

SEQ ID NO: 140 - protein, 5 aa, synthetic construct
  REGION 1..5: note = Amino acid sequence 1 of CDR 1 in the heavy chain of mouse anti-hTfR antibody No.12
NYLIE

SEQ ID NO: 141 - protein, 8 aa, synthetic construct
  REGION 1..8: note = Amino acid sequence 2 of CDR 1 in the heavy chain of mouse anti-hTfR antibody No.12
GYVFINYL

SEQ ID NO: 142 - protein, 8 aa, synthetic construct
  REGION 1..8: note = Amino acid sequence 1 of CDR 2 in the heavy chain of mouse anti-hTfR antibody No.12
IHSGSGGT

SEQ ID NO: 143 - protein, 16 aa, synthetic construct
  REGION 1..16: note = Amino acid sequence 2 of CDR 2 in the heavy chain of mouse anti-hTfR antibody No.12
IHSGSGGTNYNDNFKD

SEQ ID NO: 144 - protein, 10 aa, synthetic construct
  REGION 1..10: note = Amino acid sequence 1 of CDR 3 in the heavy chain of mouse anti-hTfR antibody No.12
RNFGNYYFDY

SEQ ID NO: 145 - protein, 12 aa, synthetic construct
  REGION 1..12: note = Amino acid sequence 2 of CDR 3 in the heavy chain of mouse anti-hTfR antibody No.12
ARRNFGNYYFDY

SEQ ID NO: 146 - protein, 5 aa, synthetic construct
  REGION 1..5: note = Amino acid sequence 1 of CDR 1 in the heavy chain of mouse anti-hTfR antibody No.13
IYWIN

SEQ ID NO: 147 - protein, 8 aa, synthetic construct
  REGION 1..8: note = Amino acid sequence 2 of CDR 1 in the heavy chain of mouse anti-hTfR antibody No.13
GYAFSIYW

SEQ ID NO: 148 - protein, 8 aa, synthetic construct
  REGION 1..8: note = Amino acid sequence 1 of CDR 2 in the heavy chain of mouse anti-hTfR antibody No.13
IYPGNGDT

SEQ ID NO: 149 - protein, 16 aa, synthetic construct
  REGION 1..16: note = Amino acid sequence 2 of CDR 2 in the heavy chain of mouse anti-hTfR antibody No.13
IYPGNGDTNYNGKFKG

SEQ ID NO: 150 - protein, 9 aa, synthetic construct
  REGION 1..9: note = Amino acid sequence 1 of CDR 3 in the heavy chain of mouse anti-hTfR antibody No.13
WGDDYAMDF

SEQ ID NO: 151 - protein, 11 aa, synthetic construct
  REGION 1..11: note = Amino acid sequence 2 of CDR 3 in the heavy chain of mouse anti-hTfR antibody No.13
GRWGDDYAMDF

SEQ ID NO: 152 - protein, 5 aa, synthetic construct
  REGION 1..5: note = Amino acid sequence 1 of CDR 1 in the heavy chain of mouse anti-hTfR antibody No.14
GYVIH

SEQ ID NO: 153 - protein, 8 aa, synthetic construct
  REGION 1..8: note = Amino acid sequence 2 of CDR 1 in the heavy chain of mouse anti-hTfR antibody No.14
GYAFTGYV

SEQ ID NO: 154 - protein, 8 aa, synthetic construct
  REGION 1..8: note = Amino acid sequence 1 of CDR 2 in the heavy chain of mouse anti-hTfR antibody No.14
LNPHKDDS

SEQ ID NO: 155 - protein, 16 aa, synthetic construct
  REGION 1..16: note = Amino acid sequence 2 of CDR 2 in the heavy chain of mouse anti-hTfR antibody No.14
LNPHKDDSEYNEKFRG

SEQ ID NO: 156 - protein, 9 aa, synthetic construct
  REGION 1..9: note = Amino acid sequence 1 of CDR 3 in the heavy chain of mouse anti-hTfR antibody No.14
GYYYYSMDY

SEQ ID NO: 157 - protein, 11 aa, synthetic construct
  REGION 1..11: note = Amino acid sequence 2 of CDR 3 in the heavy chain of mouse anti-hTfR antibody No.14
ARGYYYYSMDY

SEQ ID NO: 158 - protein, 107 aa, synthetic construct
  REGION 1..107: note = Amino acid sequence 1 of the variable region of light chain of humanized anti-hTfR antibody No.1
DIVMTQSPDSLAVSLGERATINCKASQDVNSAVAWYQQKPGQPPKLLIYWTSTRHTGVPDRFSGSGSGTDFTLTISSLQAEDVAVYYCQQHYSTPRTFGGGTKLEIK

SEQ ID NO: 159 - protein, 107 aa, synthetic construct
  REGION 1..107: note = Amino acid sequence 2 of the variable region of light chain of humanized anti-hTfR antibody No.1
DIQMTQSPSSLSASVGDRVTITCKASQDVNSAVAWYQQKPGKAPKLLLYWTSTRHTGVPSRFSGSGSGTDYTLTISSLQPEDFATYYCQQHYSTPRTFGGGTKVEIK

SEQ ID NO: 160 - protein, 107 aa, synthetic construct
  REGION 1..107: note = Amino acid sequence 3 of the variable region of light chain of humanized anti-hTfR antibody No.1
DIQVTQSPSFLSASVGDRVTITCKASQDVNSAVAWYQQKPGKAPKLLIYWTSTRHTGVPSRFSGSGSGTEFTLTISSLQPEDFATYYCQQHYSTPRTFGGGTKVEIK

SEQ ID NO: 161 - protein, 107 aa, synthetic construct
  REGION 1..107: note = Amino acid sequence 4 of the variable region of light chain of humanized anti-hTfR antibody No.1
DIQMTQSPSSLSASVGDRVTITCKASQDVNSAVAWYQQKPGKAPKLLIYWTSTRHTGVPSRFSGSGSGTDFTLTISSLQPEDFATYYCQQHYSTPRTFGGGTKVEIK

SEQ ID NO: 162 - protein, 107 aa, synthetic construct
  REGION 1..107: note = Amino acid sequence 5 of the variable region of light chain of humanized anti-hTfR antibody No.1
DIQMTQSPSSVSASVGDRVTITCKASQDVNSAVAWYQQKPGKAPKLLIYWTSTRHTGVPSRFSGSGSGTDFTLTISSLQPEDFATYYCQQHYSTPRTFGQGTKVEIK

SEQ ID NO: 163 - protein, 107 aa, synthetic construct; identical to SEQ ID NO: 162
  REGION 1..107: note = Amino acid sequence 6 of the variable region of light chain of humanized anti-hTfR antibody No.1

SEQ ID NO: 164 - protein, 214 aa, synthetic construct
  REGION 1..214: note = Amino acid sequence of the light chain of humanized anti-hTfR antibody No.1 containing amino acid sequence 6 as the variable region
DIQVTQSPSFLSASVGDRVTITCKASQDVNSAVAWFQQKPGKAPKLLIYWTSTRHTGVPDRFSGSGSGTVYTLTISSLQPEDFATYYCQQHYSTPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 165 - DNA, 725 bp, synthetic construct
  misc_feature 1..725: note = Nucleotide sequence encoding the amino acid sequence of the light chain of humanized anti-hTfR antibody No.1 containing amino acid sequence 6 as the variable region, synthetic sequence
  source 1..725: mol_type = other DNA
acgcgtgccgccaccatgggctggagctggattctgctgttcctcctgagcgtgacagcaggagtgcacagcgacatccaggtcacacagtcaccaagttttctgagcgcaagcgtgggcgacagggtcactatcacatgcaaggcaagccaggacgtgaactccgcagtggcctggttccagcagaagccagggaaagcacccaagctgctgatctattggacctctacaaggcacaccggtgtcccagatcggttctcaggttccggcagcggaacagtgtatactctgaccatttccagcctgcagcctgaagacttcgctacttactattgccagcagcattactccaccccaagaacatttggcggagggactaaagtggagatcaagaggaccgtggccgctccctccgtcttcatttttccccctagcgacgaacagctgaagagtggcacagcctcagtggtctgtctgctgaacaatttctaccctagggaggctaaagtgcagtggaaggtcgataacgcactgcagtctggaaatagtcaggagtcagtgacagaacaggactccaaagatagcacttattctctgtctagtacactgactctgagcaaggccgattacgaaaagcacaaagtgtatgcttgcgaagtcacccatcaggggctgtcatcaccagtcaccaagtcattcaatagaggcgagtgctaagcggccgc

SEQ ID NO: 166 - protein, 116 aa, synthetic construct
  REGION 1..116: note = Amino acid sequence 1 of the variable region of the heavy chain of humanized anti-hTfR antibody No.1
EVQLLESGGGLVQPGGSLRLSCAASGFTFSNYGMSWVRQAPGKGLEWVSNINTNGGSTYYPDSVKGRFTISRDNSKNTLYLQMNSLRAEDTAVYYCAKNRYDEDYWGQGTLVTVSS

SEQ ID NO: 167 - protein, 116 aa, synthetic construct
  REGION 1..116: note = Amino acid sequence 2 of the variable region of the heavy chain of humanized anti-hTfR antibody No.1
EVQLVESGGGLVQPGGSLRLSCAASGFTFSNYGMSWVRQAPGKGLEWVSNINTNGGSTYYPDSVKGRFTISRDNSKNTLYLQMNSLRAEDTAVYYCAKNRYDEDYWGQGTLVTVSS

SEQ ID NO: 168 - protein, 116 aa, synthetic construct
  REGION 1..116: note = Amino acid sequence 3 of the variable region of the heavy chain of humanized anti-hTfR antibody No.1
EVQLVESGGGLVQPGGSLRLSCSASGFTFSNYGMSWVRQAPGKGLEYVSNINTNGGSTYYPDSVKGRFTISRDNSKNTLYLQMSSLRAEDTAVYYCVKNRYDEDYWGQGTLVTVSS

SEQ ID NO: 169 - protein, 116 aa, synthetic construct
  REGION 1..116: note = Amino acid sequence 4 of the variable region of the heavy chain of humanized anti-hTfR antibody No.1
EVQLVESGGGLVQPGGSLRLSCAASGFTFSNYGMSWVRQAPGKGLEWVANINTNGGSTYYPDSVKGRFTISRDNAKNSLYLQMNSLRAEDTAVYYCARNRYDEDYWGQGTLVTVSS

SEQ ID NO: 170 - protein, 116 aa, synthetic construct
  REGION 1..116: note = Amino acid sequence 5 of the variable region of the heavy chain of humanized anti-hTfR antibody No.1
EVQLVESGGGLVQPGGSLRLSCAASGFTFSNYGMSWVRQAPGKGLVWVSNINTNGGSTYYPDSVKGRFTISRDNAKNTLYLQMNSLRAEDTAVYYCARNRYDEDYWGQGTLVTVSS

SEQ ID NO: 171 - protein, 116 aa, synthetic construct
  REGION 1..116: note = Amino acid sequence 6 of the variable region of the heavy chain of humanized anti-hTfR antibody No.1
EVQLVESGGGLVQPGGSLRLSCAASGLTFSNYGMSWVRQAPGKGLELVANINTNGGSTYYPDSVKGRFTISRDNAKNSLYLQMNSLRAEDTAVYYCTNNRYDEDYWGQGTLVTVSS

SEQ ID NO: 172 - protein, 446 aa, synthetic construct
  REGION 1..446: note = Amino acid sequence of the heavy chain of humanized anti-hTfR antibody No.1 containing amino acid sequence 6 as the variable region
EVQLVESGGGLVQPGGSLRLSCAASGLTFSNYGMSWVRQAPGKGLELVANINTNGGSTYYPDSVKGRFTISRDNAKNSLYLQMNSLRAEDTAVYYCTNNRYDEDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 173 - DNA, 1421 bp, synthetic construct
  misc_feature 1..1421: note = Nucleotide sequence encoding the amino acid sequence of the heavy chain of humanized anti-hTfR antibody No.1 containing amino acid sequence 6 as the variable region, synthetic sequence
  source 1..1421: mol_type = other DNA
acgcgtgccgccaccatgggctggagctggattctgctgttcctcctgagcgtgacagcaggagtgcacagcgaagtgcagctggtcgaatcagggggggggctggtgcagcctggaggcagcctgagactgtcctgcgccgcttctggcttgacctttagcaactacgggatgtcctgggtgcggcaggctcctggcaagggactggagttggtggccaacatcaataccaacggcggaagtacatactatcccgattcagtgaagggccggttcaccatcagcagggacaacgccaagaacagcctgtatctgcagatgaactctctgagggccgaggatacagccgtgtactattgcactaacaaccggtacgacgaggactattggggccagggcaccctggtgacagtgtctagcgcctctaccaagggcccaagcgtgtttcctctggctccatcctctaaatccacctctggcggcacagccgctctgggctgtctggtgaaggattacttcccagagcccgtgacagtgtcttggaacagcggcgccctgacctccggcgtgcacacatttcctgctgtgctgcagagctccggcctgtacagcctgtctagcgtggtgaccgtgccatcctctagcctgggcacccagacatatatctgcaacgtgaatcacaagcccagcaatacaaaggtggataagaaggtggagccaaagtcctgtgacaagacccacacatgccccccttgtcctgctccagagctgctgggaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctgcccgctcctatcgagaagaccatctctaaggccaagggccagcccagagagcctcaggtgtacacactgcctccatcccgggatgagctgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaataagcggccgc

SEQ ID NO: 174 - protein, 107 aa, synthetic construct
  REGION 1..107: note = Amino acid sequence 1 of the variable region of the light chain of humanized anti-hTfR antibody No.2
EIVMTQSPATLSVSPGERATLSCRASQSISNNLQWYQQKPGQAPRLLIYYASQSISGIPARFSGSGSGTEFTLTISSLQSEDFAVYYCQQSNSWPRTFGQGTKVDIK

SEQ ID NO: 175 - protein, 107 aa, synthetic construct
  REGION 1..107: note = Amino acid sequence 2 of the variable region of the light chain of humanized anti-hTfR antibody No.2
EIVMTQSPATLSLSPGERATLSCRASQSISNNLQWYQQKPGQAPRLLIYYASQSISGIPARFSGSGSGTDFTLTISSLEPEDFAVYYCQQSNSWPRTFGGGTKVEIK

SEQ ID NO: 176 - protein, 107 aa, synthetic construct
  REGION 1..107: note = Amino acid sequence 3 of the variable region of the light chain of humanized anti-hTfR antibody No.2
EIVLTQSPDFQSVTPKEKVTITCRASQSISNNLQWYQQKPDQSPKLLIKYASQSISGVPSRFSGSGSGTDFTLTINSLEAEDAATYYCQQSNSWPRTFGQGTKVEIK

SEQ ID NO: 177 - protein, 107 aa, synthetic construct
  REGION 1..107: note = Amino acid sequence 4 of the variable region of the light chain of humanized anti-hTfR antibody No.2
EIVLTQSPATLSLSPGERATLSCRASQSISNNLQWYQQKPGQAPRLLIYYASQSISGIPARFSGSGSGTDFTLTISSLEPEDFAVYYCQQSNSWPRTFGQGTKVEIK

SEQ ID NO: 178 - protein, 107 aa, synthetic construct
  REGION 1..107: note = Amino acid sequence 5 of the variable region of the light chain of humanized anti-hTfR antibody No.2
DIQMTQSPSSLSASVGDRVTITCRASQSISNNLQWYQQKPGKAPKLLIYYASQSISGVPSRFSGSGSGTDFTLTISSLQPEDFATYYCQQSNSWPRTFGGGTKVEIK

SEQ ID NO: 179 - protein, 107 aa, synthetic construct
  REGION 1..107: note = Amino acid sequence 6 of the variable region of the light chain of humanized anti-hTfR antibody No.2
EIVLTQSPDFQSVTPKEKVTITCRASQSISNNLQWYQQKPDQSPKLLIKYASQSISGIPSRFSGSGSGTDFTLTINSLEAEDAATYLCQQSNSWPRTFGQGTKVEIK

SEQ ID NO: 180 - protein, 214 aa, synthetic construct
  REGION 1..214: note = Amino acid sequence of the light chain of humanized anti-hTfR antibody No.2 containing amino acid sequence 6 as the variable region
EIVLTQSPDFQSVTPKEKVTITCRASQSISNNLQWYQQKPDQSPKLLIKYASQSISGIPSRFSGSGSGTDFTLTINSLEAEDAATYLCQQSNSWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 181 - DNA, 725 bp, synthetic construct
  misc_feature 1..725: note = Nucleotide sequence encoding the amino acid sequence of the light chain of humanized anti-hTfR antibody No.2 containing amino acid sequence 6 as the variable region, synthetic sequence
  source 1..725: mol_type = other DNA
acgcgtgccgccaccatgggctggagctggattctgctgttcctcctgagcgtgacagcaggagtgcacagcgaaattgtgctgacccagtctcccgatttccagtccgtgacccccaaggagaaagtcaccatcacatgcagagcatcacagtccattagcaacaatctgcagtggtaccagcagaagccagaccagagccccaagctgctgatcaaatatgcctctcagagtatttcaggcataccttctaggttctccggtagcggctctggaaccgactttactctgaccatcaacagtctggaggctgaagatgccgctacatacttgtgccagcagagtaattcatggcctaggacctttggccaggggacaaaggtggagatcaaaaggactgtggcagccccaagtgtcttcatttttcccccttcagacgaacagctgaagagcggcacagcatctgtggtctgtctgctgaacaatttctacccacgggaggctaaggtgcagtggaaagtcgataacgcactgcagtccggaaatagccaggagtctgtgactgaacaggacagtaaggattcaacctattccctgtccagcacactgactctgagcaaagccgattacgagaagcacaaagtgtatgcttgcgaagtcacacatcaggggctgtctagtcccgtgactaagtcttttaataggggtgaatgttaagcggccgc

SEQ ID NO: 182 - protein, 120 aa, synthetic construct
  REGION 1..120: note = Amino acid sequence 1 of the variable region of the heavy chain of humanized anti-hTfR antibody No.2
QVQLVQSGAEVKKPGASVKVSCKASGYTFTDYVMHWVRQAPGQGLEWMGVISTYYGHGSYNQRFKGRVTMTRDTSTSTVYMELSSLRSEDTAVYYCARGGYGSSSLAGNFDVWGQGTLVT

SEQ ID NO: 183 - protein, 120 aa, synthetic construct
  REGION 1..120: note = Amino acid sequence 2 of the variable region of the heavy chain of humanized anti-hTfR antibody No.2
QVQLVQSGAEVKKPGASVKVSCKASGYTFTDYVMHWVRQAPGQRLEWMGVISTYYGHGSYNQRFKGRVTITRDTSASTAYMELSSLRSEDTAVYYCARGGYGSSSLAGNFDVWGQGTLVT

SEQ ID NO: 184 - protein, 120 aa, synthetic construct
  REGION 1..120: note = Amino acid sequence 3 of the variable region of the heavy chain of humanized anti-hTfR antibody No.2
QVQLVQSGAEVKKPGASVKVSCKASGYTFTDYVMHWVRQAPGQGLEWMGVISTYYGHGSYNQRFKGRVTMTTDTSTSAAYMELRSLRSDDTAVYYCASGGYGSSSLAGNFDVWGQGTLVT

SEQ ID NO: 185 - protein, 120 aa, synthetic construct
  REGION 1..120: note = Amino acid sequence 4 of the variable region of the heavy chain of humanized anti-hTfR antibody No.2
QVQLVQSGAEVKKPGASVKVSCKASGYTFTDYVMHWVRQAPGQGLEWMGVISTYYGHGSYNQRFKGRVTMTRDTSISTAYMELSRLRSDDTAVYYCARGGYGSSSLAGNFDVWGQGTTVT

SEQ ID NO: 186 - protein, 120 aa, synthetic construct
  REGION 1..120: note = Amino acid sequence 5 of the variable region of the heavy chain of humanized anti-hTfR antibody No.2
QVQLVQSGAEVKKPGSSVKVSCKASGGTFSDYVMHWVRQAPGQGLEWMGVISTYYGHGSYNQRFKGRVTITADKSTSTAYMELSSLRSEDTAVYYCARGGYGSSSLAGNFDVWGQGTVVT

SEQ ID NO: 187 - protein, 120 aa, synthetic construct
  REGION 1..120: note = Amino acid sequence 6 of the variable region of the heavy chain of humanized anti-hTfR antibody No.2
QVQLVQSGAEVKKPGASVKVSCKASGYTFTDYVMHWVRQAPGQGLEWIGVISTYYGHGSYNQRFKGRATMTVDKSISTAYMELVRLRSDDTAVYYCVRGGYGSSSLAGNFDVWGQGTTVT

SEQ ID NO: 188 - protein, 453 aa, synthetic construct
  REGION 1..453: note = Amino acid sequence of the heavy chain of humanized anti-hTfR antibody No.2 containing amino acid sequence 6 as the variable region
QVQLVQSGAEVKKPGASVKVSCKASGYTFTDYVMHWVRQAPGQGLEWIGVISTYYGHGSYNQRFKGRATMTVDKSISTAYMELVRLRSDDTAVYYCVRGGYGSSSLAGNFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 189 - DNA, 1442 bp, synthetic construct
  misc_feature 1..1442: note = Nucleotide sequence encoding the amino acid sequence of the heavy chain of humanized anti-hTfR antibody No.2 containing amino acid sequence 6 as the variable region, synthetic sequence
  source 1..1442: mol_type = other DNA
acgcgtgccgccaccatgggctggagctggattctgctgttcctcctgagcgtgacagcaggagtgcacagccaggtgcagctggtccagtcaggagccgaagtgaaaaagcccggagcctcagtcaaagtgtcttgtaaagcatcaggttatacatttacagactacgtcatgcactgggtgaggcaggcacctggacagggtctggaatggatcggcgtgatctccacttactatggccatggaagctacaaccagagattcaagggcagggcgacaatgactgtagacaaatcaatttccactgcttatatggagctggtaaggctgcggtccgacgataccgctgtgtactattgcgtacgaggaggatacggctccagctctctggctggtaatttcgatgtgtgggggcagggtaccacagtcaccgtgagttcagcaagcacaaagggcccatctgtgtttccactggccccctccagcaaaagcacctctgggggtacagccgctctgggatgtctggtgaaggattatttcccagagccagtcaccgtgtcctggaacagcggagccctgacatctggagtccacacttttccagctgtgctgcagtctagtgggctgtactccctgtcatccgtggtcactgtccccagctctagtctgggtacccagacatatatctgcaacgtgaatcacaagccatctaataccaaagtcgacaagaaagtggaacccaagtcctgtgataaaactcatacctgccccccttgtcctgcaccagagctgctgggaggaccatccgtgttcctgtttccacccaagcctaaagacaccctgatgattagccgaactcccgaagtcacctgcgtggtcgtggacgtgtctcacgaggaccctgaagtcaagtttaactggtacgtggatggcgtcgaggtgcataatgctaagacaaaaccccgagaggaacagtacaacagtacatatcgtgtcgtgtcagtgctgaccgtcctgcatcaggactggctgaacgggaaggaatataagtgcaaagtgtccaataaggcactgcccgcccctatcgagaaaaccattagcaaggccaaaggacagcctagggaaccacaggtgtacacactgcctccatcccgggacgagctgactaagaaccaggtcagcctgacctgtctggtgaaaggcttctatccttcagatatcgctgtggagtgggaaagtaatggacagccagagaacaattacaagactaccccccctgtgctggactctgatgggagtttctttctgtattctaagctgaccgtggataaaagtcggtggcagcagggtaatgtctttagttgttcagtgatgcacgaagcactgcacaaccactacacccagaaatcactgtcactgtcaccagggaaataagcggccgc

SEQ ID NO: 190 - protein, 112 aa, synthetic construct
  REGION 1..112: note = Amino acid sequence 1 of the variable region of the light chain of humanized anti-hTfR antibody No.3
DVVMTQSPLSLPVTLGQPASISCRSSQSLVHSNGNTYLHWFQQRPGQSPRRLIYKVSNRFSGVPDRFSGSGSGTDFTLKISRVEAEDVGVYYCSQSTHVPWTFGGGTKVEIK

SEQ ID NO: 191 - protein, 112 aa, synthetic construct
  REGION 1..112: note = Amino acid sequence 2 of the variable region of the light chain of humanized anti-hTfR antibody No.3
DIVMTQTPLSLSVTPGQPASISCRSSQSLVHSNGNTYLHWYLQKPGQSPQLLIYKVSNRFSGVPDRFSGSGSGTDFTLKISRVEAEDVGVYYCSQSTHVPWTFGQGTKVEIK

SEQ ID NO: 192 - protein, 112 aa, synthetic construct
  REGION 1..112: note = Amino acid sequence 3 of the variable region of the light chain of humanized anti-hTfR antibody No.3
DIVMTQTPLSSPVTLGQPASISCRSSQSLVHSNGNTYLHWLQQRPGQPPRLLIYKVSNRFSGVPDRFSGSGAGTDFTLKISRVEAEDVGVYYCSQSTHVPWTFGQGTKLEIK

SEQ ID NO: 193 - protein, 112 aa, synthetic construct
  REGION 1..112: note = Amino acid sequence 4 of the variable region of the light chain of humanized anti-hTfR antibody No.3
DIVMTQSPLSLPVTPGEPASISCRSSQSLVHSNGNTYLHWYLQKPGQSPQLLIYKVSNRFSGVPDRFSGSGSGTDFTLKISRVEAEDVGVYYCSQSTHVPWTFGQGTKVEIK

SEQ ID NO: 194 - protein, 112 aa, synthetic construct
  REGION 1..112: note = Amino acid sequence 5 of the variable region of the light chain of humanized anti-hTfR antibody No.3
DIVMTQTPLSLPVTPGEPASISCRSSQSLVHSNGNTYLHWYLQKPGQSPQLLIYKVSNRFSGVPDRFSGSGSGTDFTLKISRVEAEDVGVYYCSQSTHVPWTFGQGTRLEIK

SEQ ID NO: 195 - protein, 112 aa, synthetic construct
  REGION 1..112: note = Amino acid sequence 6 of the variable region of the light chain of humanized anti-hTfR antibody No.3
DIVMTQTPLSLSVTPGQPASISCRSSQSLVHSNGNTYLHWYLQKPGQSPQLLIYKVSNRFSGVPDRFSGSGSGTDFTLKISRVEAEDVGVFFCSQSTHVPWTFGQGTKVEIK

SEQ ID NO: 196 - protein, 219 aa, synthetic construct
  REGION 1..219: note = Amino acid sequence of the light chain of humanized anti-hTfR antibody No.3 containing amino acid sequence 2 as the variable region
DIVMTQTPLSLSVTPGQPASISCRSSQSLVHSNGNTYLHWYLQKPGQSPQLLIYKVSNRFSGVPDRFSGSGSGTDFTLKISRVEAEDVGVYYCSQSTHVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 197 - DNA, 740 bp, synthetic construct
  misc_feature 1..740: note = Nucleotide sequence encoding the amino acid sequence of the light chain of humanized anti-hTfR antibody No.3 containing amino acid sequence 2 as the variable region, synthetic sequence
  source 1..740: mol_type = other DNA
acgcgtgccgccaccatgggctggagctggattctgctgttcctcctgagcgtgacagcaggagtgcacagcgacatcgtgatgacccagactcccctgagcctgagcgtgacacctggccagcctgccagcatcagctgcagaagctctcagagcctggtgcacagcaacggcaacacctacctgcactggtatctgcagaagcccggccagagccctcagctgctgatctacaaggtgtccaacagattcagcggcgtgcccgacagattctccggcagcggctctggcaccgacttcaccctgaagatttccagagtggaagccgaggacgtgggcgtgtactactgcagccagagcacccacgtgccctggacattcggccagggcaccaaggtggaaatcaagagaaccgtggccgctcccagcgtgttcatcttcccacctagcgacgagcagctgaagtccggcacagcctctgtcgtgtgcctgctgaacaacttctacccccgcgaggccaaggtgcagtggaaggtggacaacgccctgcagagcggcaacagccaggaaagcgtgaccgagcaggactccaaggacagcacctacagcctgagcagcaccctgaccctgagcaaggccgactacgagaagcacaaggtgtacgcctgcgaagtgacccaccagggcctgtctagccccgtgaccaagagcttcaacagaggcgagtgctaagcggccgc

SEQ ID NO: 198 - protein, 219 aa, synthetic construct
  REGION 1..219: note = Amino acid sequence of the light chain of humanized anti-hTfR antibody No.3 containing amino acid sequence 4 as the variable region
DIVMTQSPLSLPVTPGEPASISCRSSQSLVHSNGNTYLHWYLQKPGQSPQLLIYKVSNRFSGVPDRFSGSGSGTDFTLKISRVEAEDVGVYYCSQSTHVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 199 - DNA, 740 bp, synthetic construct
  misc_feature 1..740: note = Nucleotide sequence encoding the amino acid sequence of the light chain of humanized anti-hTfR antibody No.3 containing amino acid sequence 4 as the variable region, synthetic sequence
  source 1..740: mol_type = other DNA
acgcgtgccgccaccatgggctggagctggattctgctgttcctcctgagcgtgacagcaggagtgcacagcgacatcgtgatgacccagagccccctgagcctgcctgtgacacctggcgagcctgccagcatcagctgcagatctagccagagcctggtgcacagcaacggcaacacctacctgcactggtatctgcagaagcccggccagagccctcagctgctgatctacaaggtgtccaacagattcagcggcgtgcccgacagattctccggcagcggctctggcaccgacttcaccctgaagatctccagagtggaagccgaggacgtgggcgtgtactactgcagccagagcacccacgtgccctggacattcggccagggcaccaaggtggaaatcaagagaaccgtggccgctcccagcgtgttcatcttcccacctagcgacgagcagctgaagtccggcacagcctctgtcgtgtgcctgctgaacaacttctacccccgcgaggccaaggtgcagtggaaggtggacaacgccctgcagagcggcaacagccaggaaagcgtgaccgagcaggactccaaggacagcacctacagcctgagcagcaccctgaccctgagcaaggccgactacgagaagcacaaggtgtacgcctgcgaagtgacccaccagggcctgtctagccccgtgaccaagagcttcaacagaggcgagtgctaagcggccgc

SEQ ID NO: 200 - protein, 219 aa, synthetic construct
  REGION 1..219: note = Amino acid sequence of the light chain of humanized anti-hTfR antibody No.3 containing amino acid sequence 5 as the variable region
DIVMTQTPLSLPVTPGEPASISCRSSQSLVHSNGNTYLHWYLQKPGQSPQLLIYKVSNRFSGVPDRFSGSGSGTDFTLKISRVEAEDVGVYYCSQSTHVPWTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 201 - DNA, 740 bp, synthetic construct
  misc_feature 1..740: note = Nucleotide sequence encoding the amino acid sequence of the light chain of humanized anti-hTfR antibody No.3 containing amino acid sequence 5 as the variable region, synthetic sequence
  source 1..740: mol_type = other DNA
acgcgtgccgccaccatgggctggagctggattctgctgttcctcctgagcgtgacagcaggagtgcacagcgacatcgtgatgacccagacacccctgagcctgcctgtgacacctggcgagcctgccagcatcagctgcagatctagccagagcctggtgcacagcaacggcaacacctacctgcactggtatctgcagaagcccggccagagccctcagctgctgatctacaaggtgtccaacagattcagcggcgtgcccgacagattctccggcagcggctctggcaccgacttcaccctgaagatctccagagtggaagccgaggacgtgggcgtgtactactgcagccagagcacccacgtgccctggacattcggccagggcaccaggctggaaatcaagagaaccgtggccgctcccagcgtgttcatcttcccacctagcgacgagcagctgaagtccggcacagcctctgtcgtgtgcctgctgaacaacttctacccccgcgaggccaaggtgcagtggaaggtggacaacgccctgcagagcggcaacagccaggaaagcgtgaccgagcaggactccaaggacagcacctacagcctgagcagcaccctgaccctgagcaaggccgactacgagaagcacaaggtgtacgcctgcgaagtgacccaccagggcctgtctagccccgtgaccaagagcttcaacagaggcgagtgctaagcggccgc

SEQ ID NO: 202 - protein, 219 aa, synthetic construct
  REGION 1..219: note = Amino acid sequence of the light chain of humanized anti-hTfR antibody No.3 containing amino acid sequence 6 as the variable region
DIVMTQTPLSLSVTPGQPASISCRSSQSLVHSNGNTYLHWYLQKPGQSPQLLIYKVSNRFSGVPDRFSGSGSGTDFTLKISRVEAEDVGVFFCSQSTHVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 203 - DNA, 740 bp, synthetic construct
  misc_feature 1..740: note = Nucleotide sequence encoding the amino acid sequence of the light chain of humanized anti-hTfR antibody No.3 containing amino acid sequence 6 as the variable region, synthetic sequence
  source 1..740: mol_type = other DNA
acgcgtgccgccaccatgggctggagctggattctgctgttcctcctgagcgtgacagcaggagtgcacagcgacatcgtgatgacccagactcccctgagcctgagcgtgacacctggccagcctgccagcatcagctgcagatccagccagagcctggtgcacagcaacggcaacacctacctgcactggtatctgcagaagcccggccagagccctcagctgctgatctacaaggtgtccaacagattcagcggcgtgcccgacagattctccggcagcggctctggcaccgacttcaccctgaagatttccagagtggaagccgaggacgtgggcgtgttcttctgcagccagagcacccacgtgccctggacattcggccagggcaccaaggtggaaatcaagagaaccgtggccgctcccagcgtgttcatcttcccacctagcgacgagcagctgaagtccggcacagcctctgtcgtgtgcctgctgaacaacttctacccccgcgaggccaaggtgcagtggaaggtggacaacgccctgcagagcggcaacagccaggaaagcgtgaccgagcaggactccaaggacagcacctacagcctgagcagcaccctgaccctgagcaaggccgactacgagaagcacaaggtgtacgcctgcgaagtgacccaccagggcctgtctagccccgtgaccaagagcttcaacagaggcgagtgctaagcggccgc

SEQ ID NO: 204 - protein, 118 aa, synthetic construct
  REGION 1..118: note = Amino acid sequence 1 of the variable region of the heavy chain of humanized anti-hTfR antibody No.3
QVQLVQSGAEVKKPGASVKVSCKASGYTFTNYWLGWVRQAPGQGLEWMGDIYPGGDYPTYSEKFKVRVTMTRDTSTSTVYMELSSLRSEDTAVYYCARSGNYDEVAYWGQGTLVTVSS

SEQ ID NO: 205 - protein, 118 aa, synthetic construct
  REGION 1..118: note = Amino acid sequence 2 of the variable region of the heavy chain of humanized anti-hTfR antibody No.3
EVQLVQSGAEVKKPGESLKISCKGSGYSFTNYWLGWVRQMPGKGLEWMGDIYPGGDYPTYSEKFKVQVTISADKSISTAYLQWSSLKASDTAMYYCARSGNYDEVAYWGQGTLVTVSS

SEQ ID NO: 206 - protein, 118 aa, synthetic construct
  REGION 1..118: note = Amino acid sequence 3 of the variable region of the heavy chain of humanized anti-hTfR antibody No.3
QVQLVQSGAEVKKPGASVKVSCKASGYTFTNYWLGWVRQAPGQGLEWMGDIYPGGDYPTYSEKFKVRVTMTRDTSISTAYMELSRLRSDDTAVYYCARSGNYDEVAYWGQGTTVTVSS

SEQ ID NO: 207 - protein, 118 aa, synthetic construct
  REGION 1..118: note = Amino acid sequence 4 of the variable region of the heavy chain of humanized anti-hTfR antibody No.3
QVQLVQSGAEVKKPGASVKVSCKASGYTFTNYWLGWVRQAPGQRLEWMGDIYPGGDYPTYSEKFKVRVTITRDTSASTAYMELSSLRSEDTAVYYCARSGNYDEVAYWGQGTLVTVSS

SEQ ID NO: 208 - protein, 118 aa, synthetic construct
  REGION 1..118: note = Amino acid sequence 5 of the variable region of the heavy chain of humanized anti-hTfR antibody No.3
QVQLVQSGAEVKKPGSSVKVSCKASGGTFSNYWLGWVRQAPGQGLEWMGDIYPGGDYPTYSEKFKVRVTITADKSTSTAYMELSSLRSEDTAVYYCARSGNYDEVAYWGQGTVVTVSS

SEQ ID NO: 209 - protein, 118 aa, synthetic construct
  REGION 1..118: note = Amino acid sequence 6 of the variable region of the heavy chain of humanized anti-hTfR antibody No.3
EVQLVQSGAEVKKPGESLKISCKGSGYSFMNYWLGWVRQMPGKGLEWIGDIYPGGDYPTYSEKFKVKAIISADTSISTVYLQLSSLKASDTAMYFCARSGNYDEVAYWGQGTLVTVSS

SEQ ID NO: 210 - protein, 448 aa, synthetic construct
  REGION 1..448: note = Amino acid sequence of the heavy chain of humanized anti-hTfR antibody No.3 containing amino acid sequence 2 as the variable region
EVQLVQSGAEVKKPGESLKISCKGSGYSFTNYWLGWVRQMPGKGLEWMGDIYPGGDYPTYSEKFKVQVTISADKSISTAYLQWSSLKASDTAMYYCARSGNYDEVAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 211 - DNA, 1427 bp, synthetic construct
  misc_feature 1..1427: note = Nucleotide sequence encoding the amino acid sequence of the heavy chain of humanized anti-hTfR antibody No.3 containing amino acid sequence 2 as the variable region, synthetic sequence
  source 1..1427: mol_type = other DNA
acgcgtgccgccaccatgggctggagctggattctgctgttcctcctgagcgtgacagcaggagtgcacagcgaggtgcaactagtgcagtctggagcagaggtgaaaaagcccggggagtctctgaagatttcctgtaagggttctggatacagctttaccaactactggctgggatgggtgcgccagatgcccgggaaaggcctggagtggatgggggacatctaccccggcggagactaccctacatacagcgagaagttcaaggtccaggtcaccatctcagccgacaagtccatcagcaccgcctacctgcagtggagcagcctgaaggcctcggacaccgccatgtattactgtgcgagatcaggcaattacgacgaagtggcctactggggccaaggaaccctggtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacgtgcccaccgtgcccagcacctgaactcctgggaggtccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaataagcggccgc

SEQ ID NO: 212 - protein, 445 aa, synthetic construct
  REGION 1..445: note = Amino acid sequence of the heavy chain (IgG4) of humanized anti-hTfR antibody No.3 containing amino acid sequence 2 as the variable region
EVQLVQSGAEVKKPGESLKISCKGSGYSFTNYWLGWVRQMPGKGLEWMGDIYPGGDYPTYSEKFKVQVTISADKSISTAYLQWSSLKASDTAMYYCARSGNYDEVAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 213 - DNA, 1418 bp, synthetic construct
  misc_feature 1..1418: note = Nucleotide sequence encoding the amino acid sequence of the heavy chain (IgG4) of humanized anti-hTfR antibody No.3 containing amino acid sequence 2 as the variable region, synthetic sequence
  source 1..1418: mol_type = other DNA
acgcgtgccgccaccatgggctggagctggattctgctgttcctcctgagcgtgacagcaggagtgcacagcgaggtgcaactagtgcagtctggagcagaggtgaaaaagcccggggagtctctgaagatttcctgtaagggttctggatacagctttaccaactactggctgggatgggtgcgccagatgcccgggaaaggcctggagtggatgggggacatctaccccggcggagactaccctacatacagcgagaagttcaaggtccaggtcaccatctcagccgacaagtccatcagcaccgcctacctgcagtggagcagcctgaaggcctcggacaccgccatgtattactgtgcgagatcaggcaattacgacgaagtggcctactggggccaaggaaccctggtcaccgtctcctcagctagcaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggtccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagagcctctccctgtctccgggtaaataagcggccgc

SEQ ID NO: 214 - DNA, 43 bp, synthetic construct
  misc_feature 1..43: note = Primer hTfR5', synthetic sequence
  source 1..43: mol_type = other DNA
ccgacgcgtcgccaccatgatggatcaagctagatcagcattc

SEQ ID NO: 215 - DNA, 59 bp, synthetic construct
  misc_feature 1..59: note = Primer hTfR3', synthetic sequence
  source 1..59: mol_type = other DNA
ataatgcggccgcttaatgatgatgatgatgatgaaactcattgtcaatgtcccaaacg

SEQ ID NO: 216 - DNA, 20 bp, synthetic construct
  misc_feature 1..20: note = Primer Hyg-Sfi5', synthetic sequence
  source 1..20: mol_type = other DNA
gaggccgcctcggcctctga

SEQ ID NO: 217 - DNA, 29 bp, synthetic construct
  misc_feature 1..29: note = Primer Hyg-BstX3', synthetic sequence
  source 1..29: mol_type = other DNA
aaccatcgtgatgggtgctattcctttgc

SEQ ID NO: 218 - protein, 108 aa, synthetic construct
  REGION 1..108: note = Amino acid sequence of the variable region of the light chain of anti-hTfR antibody No.1
DIVLTQSSKFMSTSVGDRVSITCKASQDVNSAVAWFQQKPGQSPKLLIYWTSTRHTGVPDRFTGSGSGTVYTLTISSVQAEDLALYYCQQHYSTPRTFGGGTKLEIKR

SEQ ID NO: 219 - protein, 116 aa, synthetic construct
  REGION 1..116: note = Amino acid sequence of the variable region of the heavy chain of anti-hTfR antibody No.1
EVQLQESGGGLVQPGGSLKLSCAASGLTFSNYGMSWIRQTPDKRLELVANINTNGGSTYYPDSVKGRFTISRDNAKNTLYLQMNSLKSEDTAIYYCTNNRYDEDYWGQGTSVTVSS

SEQ ID NO: 220 - protein, 108 aa, synthetic construct
  REGION 1..108: note = Amino acid sequence of the variable region of the light chain of anti-hTfR antibody No.2
DIVLTQSPATLSVTPGDSVSLSCRASQSISNNLQWYQQKSHESPRLLIKYASQSISGIPSRFSGSGSGTDFTLSINSVETEDFGMYFCQQSNSWPRTFGAGTKLELKR

SEQ ID NO: 221 - protein, 123 aa, synthetic construct
  REGION 1..123: note = Amino acid sequence of the variable region of the heavy chain of anti-hTfR antibody No.2
EVQLQESGAELVRPGVSVKISCKGSGYTFTDYVMHWVRQSHAKSLEWIGVISTYYGHGSYNQRFKGKATMTVDKSSSTAYLELVRLTSEDSAIYYCVRGGYGSSSLAGNFDVWGAGTTVTVSS

SEQ ID NO: 222 - protein, 113 aa, synthetic construct
  REGION 1..113: note = Amino acid sequence of the variable region of the light chain of anti-hTfR antibody No.3
DIVMTQTTLSLPVSLGDQASISCRSSQSLVHSNGNTYLHWYLQKPGQSPKLLIYKVSNRFSGVPDRFSGSGSGTDFTLKISRVEAEDLGVFFCSQSTHVPWTFGGGTKLEIKR

SEQ ID NO: 223 - protein, 118 aa, synthetic construct
  REGION 1..118: note = Amino acid sequence of the variable region of the heavy chain of anti-hTfR antibody No.3
EVQLQESGAELVRPGTSVKISCKASGYTFMNYWLGWVKQRPGHGLEWIGDIYPGGDYPTYSEKFKVKAILTADTSSSSVYLHLSSLTSEDSAVYFCARSGNYDEVAYWGQGTLVTVST

SEQ ID NO: 224 - protein, 106 aa, synthetic construct
  REGION 1..106: note = Amino acid sequence of the variable region of the light chain of anti-hTfR antibody No.4
DIVLTQTPAIMSASPGEKVTMTCSASSNVNYMHWYQQKSSTSPKLWIYDTSKLASGVPGRFSGSGSGNSYSLTISSMEAEDVATYYCFQGNGNPYTFGGGTKLEIK

SEQ ID NO: 225 - protein, 122 aa, synthetic construct
  REGION 1..122: note = Amino acid sequence of the variable region of the heavy chain of anti-hTfR antibody No.4
EVQLQESGAELVRPGTSVRVFCKASGYAFTNYLIEWVKQRPGQGLEWIGLINPGSGGIIYNEKFTDRATLTADKSSSTAYMQLSSLTSDDSAVYFCARSNYYGTTYWHFDVWGAGTTVTVSS

SEQ ID NO: 226 - protein, 105 aa, synthetic construct
  REGION 1..105: note = Amino acid sequence of the variable region of the light chain of anti-hTfR antibody No.5
DIVMTQTPAIMSASPGEKVTMTCSASSSISSIHWYQQKPGTSPKRWIYDTSTLASGVPVRFSGSGSGTSYSLTISSMEAEDAATYYCHQRSSYPTFGGGTKLEIK

SEQ ID NO: 227 - protein, 118 aa, synthetic construct
  REGION 1..118: note = Amino acid sequence of the variable region of the heavy chain of anti-hTfR antibody No.5
EVQLQESGPELVKPGASVKMSCKASGYTFTNFVIHWVKQRPGQGLEWIGFFNPHKNGAEYNEKFQVKATLTSDKSSTTAYMELNSLTSEDSAVYYCARSFYYYSMDYWGQGTSVTVSS

SEQ ID NO: 228 - protein, 107 aa, synthetic construct
  REGION 1..107: note = Amino acid sequence of the variable region of the light chain of anti-hTfR antibody No.6
DIVLTQTPSSLSASLGERVSLTCRASQEISGYLSWLQQKPDGTIKRLIYAASTLDSGVPKRFSGSRSGSDYSLTISSLESEDFADYYCLQYSSYPRTFGGGTKLEIK

SEQ ID NO: 229 - protein, 112 aa, synthetic construct
  REGION 1..112: note = Amino acid sequence of the variable region of the heavy chain of anti-hTfR antibody No.6
EVQLQESGPGLVAPSQSLSITCSVSGFSLSTYGVYWVRQPPGKGLEWLGIIWGDGSTNYHSALISRLTISKDNSKSQVFLKLNSLQTADTATYYCAKPDDVWGAGTTVTVSS

SEQ ID NO: 230 - protein, 106 aa, synthetic construct
  REGION 1..106: note = Amino acid sequence of the variable region of the light chain of anti-hTfR antibody No.7
DIVLTQSPVIMSASLGEEITLTCSASSSVNYIHWYQQKSGTSPKVLIYQTSNLASGVPSRFSGSRSGTFYSLKISSVEAEDAADYYCHQWSSYPWTFGGGTKLEIK

SEQ ID NO: 231 - protein, 120 aa, synthetic construct
  REGION 1..120: note = Amino acid sequence of the variable region of the heavy chain of anti-hTfR antibody No.7
EVQLQESGGALVKPGGSLKVSCEASGITFRNYFMSWVRQTPEKRLEWVATISSAGGYTYYADSVKGRFTISRDNAGNTLYLQMSSLRSEDTAMYFCARQEVPYPYAMDYWGQGTSVIVSS

SEQ ID NO: 232 - protein, 106 aa, synthetic construct
  REGION 1..106: note = Amino acid sequence of the variable region of the light chain of anti-hTfR antibody No.8
DIVLTQSPAIMSASLGEEITLTCSASSSVSYMHWYQQKSGTSPKVLIYGTSNLASEVPSRFSGSGSGTFYSLTISSVEAEDAADYYCHQWSSYPWTFGGGTKLEIK

SEQ ID NO: 233 - protein, 112 aa, synthetic construct
  REGION 1..112: note = Amino acid sequence of the variable region of the heavy chain of anti-hTfR antibody No.8
EVQLQESGPGLVAPSQSLSITCTVSGFSLTTYGVSWIRQPPGKGLEWLGIIWGDGSTNYRSALISRLSISKDNSKNQVFLKLNSLQTDDTATYYCAKPDDYWGQGTSVTVSS

SEQ ID NO: 234 - protein, 106 aa, synthetic construct
  REGION 1..106: note = Amino acid sequence of the variable region of the light chain of anti-hTfR antibody No.9
DIVLTQSPALMSASPGETVTMTCSASSSVSLMFWYQQKPRSSPKPWIYFTSYRASGVPIRFSGSGSGTSFSLTISNLEAEDAATYYCQQWTSDPPTFGSGTKLEIE

SEQ ID NO: 235 - protein, 117 aa, synthetic construct
  REGION 1..117: note = Amino acid sequence of the variable region of the heavy chain of anti-hTfR antibody No.9
EVQLQESGPELKKPGETVKISCKASGYTFTNYGVSWVKQAPGKDLKWMGWIYTFTGEATYIDDFRGRFAFSLETSANTAYLQINNLKNEDTATYFCSRRNGAWFEDWGQGTLVTVSA

SEQ ID NO: 236 - protein, 107 aa, synthetic construct
  REGION 1..107: note = Amino acid sequence of the variable region of the light chain of anti-hTfR antibody No.10
DIVMTQTPSSLSASLGERVSLTCRPSQEISGYLSWLQQKPDGTIKRLIYAASTLDSGVPKRFSGSRSGSDYSLTISSLESEDFADYYCLQYASYPRTFGGGTKLEIK

SEQ ID NO: 237 - protein, 120 aa, synthetic construct
  REGION 1..120: note = Amino acid sequence of the variable region of the heavy chain of anti-hTfR antibody No.10
EVQLQESGGGLVKPGGSLKLSCEASGITFRNYAMSWVRQTPEKRLEWVATISSYGGYTYYADSVKGRFTVSRDNAGNTLYLQMSSLRSEDTAMYHCARQEVPYPYPMDNWGQGTSVIVSS

SEQ ID NO: 238 - protein, 107 aa, synthetic construct
  REGION 1..107: note = Amino acid sequence of the variable region of the light chain of anti-hTfR antibody No.11
DIVLTQSPATLSVTPGDRVSLSCRASQSIRNYLHWYQQKSHESPRLLIKYASQSISGIPSRFSGSGSGTDFTLSINSVETEDFGMYFCQQTNSWPYTFGGGTKLEIK

SEQ ID NO: 239 - protein, 118 aa, synthetic construct
  REGION 1..118: note = Amino acid sequence of the variable region of the heavy chain of anti-hTfR antibody No.11
EVQLQESGAELVRPGSSVKISCKASGYAFSIYWINWLKQRPGQGLEWIGQIYPGNGDTNYNGKFKGKATLTADKSSNTVYMRLSSLTSEDSAVYFCGRWGDDYAMDFWGQGTSVTVSS

SEQ ID NO: 240 - protein, 107 aa, synthetic construct
  REGION 1..107: note = Amino acid sequence of the variable region of the light chain of anti-hTfR antibody No.12
DIVLTQSHKFMSTVIGDRVSITCKASHDVKTAVAWYQQTPGQSPKLLTYWSSTRHTGVPDRFTGSGSGTDYTLTISSVQAEDLALYYCQQHFSTPLTFGAGTKLELK

SEQ ID NO: 241 - protein, 119 aa, synthetic construct
  REGION 1..119: note = Amino acid sequence of the variable region of the heavy chain of anti-hTfR antibody No.12
EVQLQESGAELARPGTSVKVSCKAAGYVFINYLIEWVKQRPGQGLEWIGVIHSGSGGTNYNDNFKDKATLTRDVSSSTAYMQLSSLTSDDSAVYFCARRNFGNYYFDYWGQGTTLTVSS

SEQ ID NO: 242 - protein, 107 aa, synthetic construct
  REGION 1..107: note = Amino acid sequence of the variable region of the light chain of anti-hTfR antibody No.13
DIVITQTPATLSVTPGDRVSLSCRASQSIRNYLHWYQQKSHESPRLLIKYASQSISGIPSRFSGSGSGTDFTLTINSVETEDFGMYFCQQTNSWPYTFGGGTKLEIK

SEQ ID NO: 243 - protein, 118 aa, synthetic construct; identical to SEQ ID NO: 239
  REGION 1..118: note = Amino acid sequence of the variable region of the heavy chain of anti-hTfR antibody No.13

SEQ ID NO: 244 - protein, 106 aa, synthetic construct
  REGION 1..106: note = Amino acid sequence of the variable region of the light chain of anti-hTfR antibody No.14
DIVLTQTPVIMSAYPGEKVTLTCSASSNINSIHWYQQKPGTSPKRWIYDTSNLASGVPARFSGSGSGTSYSLTISSMEAEDAASYYCHQRSGYPFTFGSGTKLEIK

SEQ ID NO: 245 - protein, 118 aa, synthetic construct
  REGION 1..118: note = Amino acid sequence of the variable region of the heavy chain of anti-hTfR antibody No.14
EVQLQESGPELLKPGASVKMSCKASGYAFTGYVIHWVKQKPGQGLEWIGFLNPHKDDSEYNEKFRGKATLTSDKSSSTAYMDLSSLTSEDSAVYYCARGYYYYSMDYWGQGTSVTVSS

SEQ ID NO: 246 - protein, 525 aa, Homo sapiens
SETQANSTTDALNVLLIIVDDLRPSLGCYGDKLVRSPNIDQLASHSLLFQNAFAQQAVCAPSRVSFLTGRRPDTTRLYDFNSYWRVHAGNFSTIPQYFKENGYVTMSVGKVFHPGISSNHTDDSPYSWSFPPYHPSSEKYENTKTCRGPDGELHANLLCPVDVLDVPEGTLPDKQSTEQAIQLLEKMKTSASPFFLAVGYHKPHIPFRYPKEFQKLYPLENITLAPDPEVPDGLPPVAYNPWMDIRQREDVQALNISVPYGPIPVDFQRKIRQSYFASVSYLDTQVGRLLSALDDLQLANSTIIAFTSDHGWALGEHGEWAKYSNFDVATHVPLIFYVPGRTASLPEAGEKLFPYLDPFDSASQLMEPGRQSMDLVELVSLFPTLAGLAGLQVPPRCPVPSFHVELCREGKNLLKHFRFRDLEEDPYLPGNPRELIAYSQYPRPSDIPQWNSDKPSLKDIKIMGYSIRTIDYRYTVWVGFNPDEFLANFSDIHAGELYFVDSDPLQDHNMYNDSQGGDLFQLLMP

SEQ ID NO: 247 - protein, 973 aa, synthetic construct
  REGION 1..973: note = Amino acid sequence of the fusion protein of heavy chain of anti-hTfR antibody No.1 (humanized 6) and hI2S
EVQLVESGGGLVQPGGSLRLSCAASGLTFSNYGMSWVRQAPGKGLELVANINTNGGSTYYPDSVKGRFTISRDNAKNSLYLQMNSLRAEDTAVYYCTNNRYDEDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSSETQANSTTDALNVLLIIVDDLRPSLGCYGDKLVRSPNIDQLASHSLLFQNAFAQQAVCAPSRVSFLTGRRPDTTRLYDFNSYWRVHAGNFSTIPQYFKENGYVTMSVGKVFHPGISSNHTDDSPYSWSFPPYHPSSEKYENTKTCRGPDGELHANLLCPVDVLDVPEGTLPDKQSTEQAIQLLEKMKTSASPFFLAVGYHKPHIPFRYPKEFQKLYPLENITLAPDPEVPDGLPPVAYNPWMDIRQREDVQALNISVPYGPIPVDFQRKIRQSYFASVSYLDTQVGRLLSALDDLQLANSTIIAFTSDHGWALGEHGEWAKYSNFDVATHVPLIFYVPGRTASLPEAGEKLFPYLDPFDSASQLMEPGRQSMDLVELVSLFPTLAGLAGLQVPPRCPVPSFHVELCREGKNLLKHFRFRDLEEDPYLPGNPRELIAYSQYPRPSDIPQWNSDKPSLKDIKIMGYSIRTIDYRYTVWVGFNPDEFLANFSDIHAGELYFVDSDPLQDHNMYNDSQGGDLFQLLMP

SEQ ID NO: 248 - DNA, 3002 bp, synthetic construct
  misc_feature 1..3002: note = Nucleotide sequence encoding the amino acid sequence of fusion protein of heavy chain of anti-hTfR antibody No.1 (humanized 6) and hI2S, synthetic sequence
  source 1..3002: mol_type = other DNA
acgcgtgccgccaccatgggctggagctggattctgctgttcctcctgagcgtgacagcaggagtgcacagcgaagtgcagctggtcgaatcagggggggggctggtgcagcctggaggcagcctgagactgtcctgcgccgcttctggcttgacctttagcaactacgggatgtcctgggtgcggcaggctcctggcaagggactggagttggtggccaacatcaataccaacggcggaagtacatactatcccgattcagtgaagggccggttcaccatcagcagggacaacgccaagaacagcctgtatctgcagatgaactctctgagggccgaggatacagccgtgtactattgcactaacaaccggtacgacgaggactattggggccagggcaccctggtgacagtgtctagcgcctctaccaagggcccaagcgtgtttcctctggctccatcctctaaatccacctctggcggcacagccgctctgggctgtctggtgaaggattacttcccagagcccgtgacagtgtcttggaacagcggcgccctgacctccggcgtgcacacatttcctgctgtgctgcagagctccggcctgtacagcctgtctagcgtggtgaccgtgccatcctctagcctgggcacccagacatatatctgcaacgtgaatcacaagcccagcaatacaaaggtggataagaaggtggagccaaagtcctgtgacaagacccacacatgccccccttgtcctgctccagagctgctgggaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctgcccgctcctatcgagaagaccatctctaaggccaagggccagcccagagagcctcaggtgtacacactgcctccatcccgggatgagctgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaaggatcttccgaaacgcaggccaactcgaccacagatgctctgaacgttcttctcatcatcgtggatgacctgcgcccctccctgggctgttatggggataagctggtgaggtccccaaatattgaccaactggcatcccacagcctcctcttccagaatgcctttgcgcagcaagcagtgtgcgccccgagccgcgtttctttcctcactggcaggagacctgacaccacccgcctgtacgacttcaactcctactggagggtgcacgctggaaacttctccaccatcccccagtacttcaaggagaatggctatgtgaccatgtcggtgggaaaagtctttcaccctgggatatcttctaaccataccgatgattctccgtatagctggtcttttccaccttatcatccttcctctgagaagtatgaaaacactaagacatgtcgagggccagatggagaactccatgccaacctgctttgccctgtggatgtgctggatgttcccgagggcaccttgcctgacaaacagagcactgagcaagccatacagttgttggaaaagatgaaaacgtcagccagtcctttcttcctggccgttgggtatcataagccacacatccccttcagataccccaaggaatttcagaagttgtatcccttggagaacatcaccctggcccccgatcccgaggtccctgatggcctaccccctgtggcctacaacccctggatggacatcaggcaacgggaagacgtccaagccttaaacatcagtgtgccgtatggtccaattcctgtggactttcagcggaaaatccgccagagctactttgcctctgtgtcatatttggatacacaggtcggccgcctcttgagtgctttggacgatcttcagctggccaacagcaccatcattgcatttacctcggatcatgggtgggctctaggtgaacatggagaatgggccaaatacagcaattttgatgttgctacccatgttcccctgatattctatgttcctggaaggacggcttcacttccggaggcaggcgagaagcttttcccttacctcgacccttttgattccgcctcacagttgatggagccaggcaggcaatccatggaccttgtggaacttgtgtctctttttcccacgctggctggacttgcaggactgcaggttccacctcgctgccccgttccttcatttcacgttgagctgtgcagagaaggcaagaaccttctgaagcattttcgattccgtgacttggaagaagatccgtacctccctggtaatccccgtgaactgattgcctatagccagtatccccggccttcagacatccctcagtggaattctgacaagccgagtttaaaagatataaagatcatgggctattccatacgcaccatagactataggtatactgtgtgggttggcttcaatcctgatgaatttctagctaacttttctgacatccatgcaggggaactgtattttgtggattctgacccattgcaggatcacaatatgtataatgattcccaaggtggagaccttttccagttgttgatgccttaagcggccgc

SEQ ID NO: 249 - protein, 980 aa, synthetic construct
  REGION 1..980: note = Amino acid sequence of the fusion protein of heavy chain of anti-hTfR antibody No.2 (humanized 6) and hI2S
QVQLVQSGAEVKKPGASVKVSCKASGYTFTDYVMHWVRQAPGQGLEWIGVISTYYGHGSYNQRFKGRATMTVDKSISTAYMELVRLRSDDTAVYYCVRGGYGSSSLAGNFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSSETQANSTTDALNVLLIIVDDLRPSLGCYGDKLVRSPNIDQLASHSLLFQNAFAQQAVCAPSRVSFLTGRRPDTTRLYDFNSYWRVHAGNFSTIPQYFKENGYVTMSVGKVFHPGISSNHTDDSPYSWSFPPYHPSSEKYENTKTCRGPDGELHANLLCPVDVLDVPEGTLPDKQSTEQAIQLLEKMKTSASPFFLAVGYHKPHIPFRYPKEFQKLYPLENITLAPDPEVPDGLPPVAYNPWMDIRQREDVQALNISVPYGPIPVDFQRKIRQSYFASVSYLDTQVGRLLSALDDLQLANSTIIAFTSDHGWALGEHGEWAKYSNFDVATHVPLIFYVPGRTASLPEAGEKLFPYLDPFDSASQLMEPGRQSMDLVELVSLFPTLAGLAGLQVPPRCPVPSFHVELCREGKNLLKHFRFRDLEEDPYLPGNPRELIAYSQYPRPSDIPQWNSDKPSLKDIKIMGYSIRTIDYRYTVWVGFNPDEFLANFSDIHAGELYFVDSDPLQDHNMYNDSQGGDLFQLLMP

SEQ ID NO: 250 - DNA, 3023 bp, synthetic construct
  misc_feature 1..3023: note = Nucleotide sequence encoding the amino acid sequence of fusion protein of heavy chain of anti-hTfR antibody No.2 (humanized 6) and hI2S, synthetic sequence
  source 1..3023: mol_type = other DNA
acgcgtgccgccaccatgggctggagctggattctgctgttcctcctgagcgtgacagcaggagtgcacagccaggtgcagctggtccagtcaggagccgaagtgaaaaagcccggagcctcagtcaaagtgtcttgtaaagcatcaggttatacatttacagactacgtcatgcactgggtgaggcaggcacctggacagggtctggaatggatcggcgtgatctccacttactatggccatggaagctacaaccagagattcaagggcagggcgacaatgactgtagacaaatcaatttccactgcttatatggagctggtaaggctgcggtccgacgataccgctgtgtactattgcgtacgaggaggatacggctccagctctctggctggtaatttcgatgtgtgggggcagggtaccacagtcaccgtgagttcagcaagcacaaagggcccatctgtgtttccactggccccctccagcaaaagcacctctgggggtacagccgctctgggatgtctggtgaaggattatttcccagagccagtcaccgtgtcctggaacagcggagccctgacatctggagtccacacttttccagctgtgctgcagtctagtgggctgtactccctgtcatccgtggtcactgtccccagctctagtctgggtacccagacatatatctgcaacgtgaatcacaagccatctaataccaaagtcgacaagaaagtggaacccaagtcctgtgataaaactcatacctgccccccttgtcctgcaccagagctgctgggaggaccatccgtgttcctgtttccacccaagcctaaagacaccctgatgattagccgaactcccgaagtcacctgcgtggtcgtggacgtgtctcacgaggaccctgaagtcaagtttaactggtacgtggatggcgtcgaggtgcataatgctaagacaaaaccccgagaggaacagtacaacagtacatatcgtgtcgtgtcagtgctgaccgtcctgcatcaggactggctgaacgggaaggaatataagtgcaaagtgtccaataaggcactgcccgcccctatcgagaaaaccattagcaaggccaaaggacagcctagggaaccacaggtgtacacactgcctccatcccgggacgagctgactaagaaccaggtcagcctgacctgtctggtgaaaggcttctatccttcagatatcgctgtggagtgggaaagtaatggacagccagagaacaattacaagactaccccccctgtgctggactctgatgggagtttctttctgtattctaagctgaccgtggataaaagtcggtggcagcagggtaatgtctttagttgttcagtgatgcacgaagcactgcacaaccactacacccagaaatcactgtcactgtcaccagggaaaggatcttccgaaacgcaggccaactcgaccacagatgctctgaacgttcttctcatcatcgtggatgacctgcgcccctccctgggctgttatggggataagctggtgaggtccccaaatattgaccaactggcatcccacagcctcctcttccagaatgcctttgcgcagcaagcagtgtgcgccccgagccgcgtttctttcctcactggcaggagacctgacaccacccgcctgtacgacttcaactcctactggagggtgcacgctggaaacttctccaccatcccccagtacttcaaggagaatggctatgtgaccatgtcggtgggaaaagtctttcaccctgggatatcttctaaccataccgatgattctccgtatagctggtcttttccaccttatcatccttcctctgagaagtatgaaaacactaagacatgtcgagggccagatggagaactccatgccaacctgctttgccctgtggatgtgctggatgttcccgagggcaccttgcctgacaaacagagcactgagcaagccatacagttgttggaaaagatgaaaacgtcagccagtcctttcttcctggccgttgggtatcataagccacacatccccttcagataccccaaggaatttcagaagttgtatcccttggagaacatcaccctggcccccgatcccgaggtccctgatggcctaccccctgtggcctacaacccctggatggacatcaggcaacgggaagacgtccaagccttaaacatcagtgtgccgtatggtccaattcctgtggactttcagcggaaaatccgccagagctactttgcctctgtgtcatatttggatacacaggtcggccgcctcttgagtgctttggacgatcttcagctggccaacagcaccatcattgcatttacctcggatcatgggtgggctctaggtgaacatggagaatgggccaaatacagcaattttgatgttgctacccatgttcccctgatattctatgttcctggaaggacggcttcacttccggaggcaggcgagaagcttttcccttacctcgacccttttgattccgcctcacagttgatggagccaggcaggcaatccatggaccttgtggaacttgtgtctctttttcccacgctggctggacttgcaggactgcaggttccacctcgctgccccgttccttcatttcacgttgagctgtgcagagaaggcaagaaccttctgaagcattttcgattccgtgacttggaagaagatccgtacctccctggtaatccccgtgaactgattgcctatagccagtatccccggccttcagacatccctcagtggaattctgacaagccgagtttaaaagatataaagatcatgggctattccatacgcaccatagactataggtatactgtgtgggttggcttcaatcctgatgaatttctagctaacttttctgacatccatgcaggggaactgtattttgtggattctgacccattgcaggatcacaatatgtataatgattcccaaggtggagaccttttccagttgttgatgccttaagcggccgc

SEQ ID NO: 251 - protein, 975 aa, synthetic construct
  REGION 1..975: note = Amino acid sequence of fusion protein of heavy chain of anti-hTfR antibody No.3 (humanized 2) and hI2S, synthetic sequence
EVQLVQSGAEVKKPGESLKISCKGSGYSFTNYWLGWVRQMPGKGLEWMGDIYPGGDYPTYSEKFKVQVTISADKSISTAYLQWSSLKASDTAMYYCARSGNYDEVAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSSETQANSTTDALNVLLIIVDDLRPSLGCYGDKLVRSPNIDQLASHSLLFQNAFAQQAVCAPSRVSFLTGRRPDTTRLYDFNSYWRVHAGNFSTIPQYFKENGYVTMSVGKVFHPGISSNHTDDSPYSWSFPPYHPSSEKYENTKTCRGPDGELHANLLCPVDVLDVPEGTLPDKQSTEQAIQLLEKMKTSASPFFLAVGYHKPHIPFRYPKEFQKLYPLENITLAPDPEVPDGLPPVAYNPWMDIRQREDVQALNISVPYGPIPVDFQRKIRQSYFASVSYLDTQVGRLLSALDDLQLANSTIIAFTSDHGWALGEHGEWAKYSNFDVATHVPLIFYVPGRTASLPEAGEKLFPYLDPFDSASQLMEPGRQSMDLVELVSLFPTLAGLAGLQVPPRCPVPSFHVELCREGKNLLKHFRFRDLEEDPYLPGNPRELIAYSQYPRPSDIPQWNSDKPSLKDIKIMGYSIRTIDYRYTVWVGFNPDEFLANFSDIHAGELYFVDSDPLQDHNMYNDSQGGDLFQLLMP

SEQ ID NO: 252 - DNA, 3011 bp, synthetic construct
  misc_feature 1..3011: note = Nucleotide sequence encoding the amino acid sequence of fusion protein of heavy chain of anti-hTfR antibody No.3 (humanized 2) and hI2S, synthetic sequence
  source 1..3011: mol_type = other DNA
acgcgtgccgccaccatgggctggagctggattctgctgttcctcctgagcgtgacagcaggagtgcacagcgaggtgcaactagtgcagtctggagcagaggtgaaaaagcccggggagtctctgaagatttcctgtaagggttctggatacagctttaccaactactggctgggatgggtgcgccagatgcccgggaaaggcctggagtggatgggggacatctaccccggcggagactaccctacatacagcgagaagttcaaggtccaggtcaccatctcagccgacaagtccatcagcaccgcctacctgcagtggagcagcctgaaggcctcggacaccgccatgtattactgtgcgagatcaggcaattacgacgaagtggcctactggggccaaggaaccctggtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacgtgcccaccgtgcccagcacctgaactcctgggaggtccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggatcttccgaaacgcaggccaactcgaccacagatgctctgaacgttcttctcatcatcgtggatgacctgcgcccctccctgggctgttatggggataagctggtgaggtccccaaatattgaccaactggcatcccacagcctcctcttccagaatgcctttgcgcagcaagcagtgtgcgccccgagccgcgtttctttcctcactggcaggagacctgacaccacccgcctgtacgacttcaactcctactggagggtgcacgctggaaacttctccaccatcccccagtacttcaaggagaatggctatgtgaccatgtcggtgggaaaagtctttcaccctgggatatcttctaaccataccgatgattctccgtatagctggtcttttccaccttatcatccttcctctgagaagtatgaaaacactaagacatgtcgagggccagatggagaactccatgccaacctgctttgccctgtggatgtgctggatgttcccgagggcaccttgcctgacaaacagagcactgagcaagccatacagttgttggaaaagatgaaaacgtcagccagtcctttcttcctggccgttgggtatcataagccacacatccccttcagataccccaaggaatttcagaagttgtatcccttggagaacatcaccctggcccccgatcccgaggtccctgatggcctaccccctgtggcctacaacccctggatggacatcaggcaacgggaagacgtccaagccttaaacatcagtgtgccgtatggtccaattcctgtggactttcagcggaaaatccgccagagctactttgcctctgtgtcatatttggatacacaggtcggccgcctcttgagtgctttggacgatcttcagctggccaacagcaccatcattgcatttacctcggatcatgggtgggctctaggtgaacatggagaatgggccaaatacagcaattttgatgttgctacccatgttcccctgatattctatgttcctggaaggacggcttcacttccggaggcaggcgagaagcttttcccttacctcgacccttttgattccgcctcacagttgatggagccaggcaggcaatccatggaccttgtggaacttgtgtctctttttcccacgctggctggacttgcaggactgcaggttccacctcgctgccccgttccttcatttcacgttgagctgtgcagagaaggcaagaaccttctgaagcattttcgattccgtgacttggaagaagatccgtacctccctggtaatccccgtgaactgattgcctatagccagtatccccggccttcagacatccctcagtggaattctgacaagccgagtttaaaagatataaagatcatgggctattccatacgcaccatagactataggtatactgtgtgggttggcttcaatcctgatgaatttctagctaacttttctgacatccatgcaggggaactgtattttgtggattctgacccattgcaggatcacaatatgtataatgattcccaaggtggagaccttttccagttgttgatgccttaataagcggccgc

SEQ ID NO: 253 - DNA, 4460 bp, synthetic construct
  misc_feature 1..4460: note = Nucleotide sequence of the DNA formed of a cDNA encoding chimeric hTfR and a loxP-flanked neomycin resistance gene placed on the cDNA's 3' side, synthetic sequence
  source 1..4460: mol_type = other DNA
gtttatcctcccttgtagcagctgagaatgatggatcaagccagatcagcattctctaacttgtttggtggggaaccattgtcatacacccggtttagccttgctcggcaagtagatggagataacagtcatgtggagatgaaactggctgcagatgaagaagaaaatgccgacaataacatgaaggctagtgtcagaaaacccaagaggtttaatggaagactctgctttgcagctattgcactagtcattttcttcttgattggattcatgagtggctacctgggctattgtaaaggggtagaaccaaaaactgagtgtgagagactggcaggaaccgagtctccagtgagggaggagccaggagaggacttccctgcagcacgtcgcttatattgggatgacctgaagagaaagttgtcggagaaactggacagcacagacttcaccggcaccatcaagctgctgaatgaaaattcatatgtccctcgtgaggctggatctcaaaaagatgaaaatcttgcgttgtatgttgaaaatcaatttcgtgaatttaaactcagcaaagtctggcgtgatcaacattttgttaagattcaggtcaaagacagcgctcaaaactcggtgatcatagttgataagaacggtagacttgtttacctggtggagaatcctgggggttatgtggcgtatagtaaggctgcaacagttactggtaaactggtccatgctaattttggtactaaaaaagattttgaggatttatacactcctgtgaatggatctatagtgattgtcagagcagggaaaatcacctttgcagaaaaggttgcaaatgctgaaagcttaaatgcaattggtgtgttgatatacatggaccagactaaatttcccattgttaacgcagaactttcattctttggacatgctcatctggggacaggtgacccttacacacctggattcccttccttcaatcacactcagtttccaccatctcggtcatcaggattgcctaatatacctgtccagacaatctccagagctgctgcagaaaagctgtttgggaatatggaaggagactgtccctctgactggaaaacagactctacatgtaggatggtaacctcagaaagcaagaatgtgaagctcactgtgagcaatgtgctgaaagagataaaaattcttaacatctttggagttattaaaggctttgtagaaccagatcactatgttgtagttggggcccagagagatgcatggggccctggagctgcaaaatccggtgtaggcacagctctcctattgaaacttgcccagatgttctcagatatggtcttaaaagatgggtttcagcccagcagaagcattatctttgccagttggagtgctggagactttggatcggttggtgccactgaatggctagagggatacctttcgtccctgcatttaaaggctttcacttatattaatctggataaagcggttcttggtaccagcaacttcaaggtttctgccagcccactgttgtatacgcttattgagaaaacaatgcaaaatgtgaagcatccggttactgggcaatttctatatcaggacagcaactgggccagcaaagttgagaaactcactttagacaatgctgctttccctttccttgcatattctggaatcccagcagtttctttctgtttttgcgaggacacagattatccttatttgggtaccaccatggacacctataaggaactgattgagaggattcctgagttgaacaaagtggcacgagcagctgcagaggtcgctggtcagttcgtgattaaactaacccatgatgttgaattgaacctggactatgagaggtacaacagccaactgctttcatttgtgagggatctgaaccaatacagagcagacataaaggaaatgggcctgagtttacagtggctgtattctgctcgtggagacttcttccgtgctacttccagactaacaacagatttcgggaatgctgagaaaacagacagatttgtcatgaagaaactcaatgatcgtgtcatgagagtggagtatcacttcctctctccctacgtatctccaaaagagtctcctttccgacatgtcttctggggctccggctctcacacgctgccagctttactggagaacttgaaactgcgtaaacaaaataacggtgcttttaatgaaacgctgttcagaaaccagttggctctagctacttggactattcagggagctgcaaatgccctctctggtgacgtttgggacattgacaatgagttttaacgtggctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgatcctctagttaagcttcccagcggccgctatcgaattccgatcatattcaataacccttaatataacttcgtataatgtatgctatacgaagttattaggtctgaagaggagtttacgtccagccaagctaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagccaatatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatccgctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatccctcgatcgagatccggaacccttaatataacttcgtataatgtatgctatacgaagttattaggtccctcgaagaggttcactagttctagagcatttaaatacgtgctagc

SEQ ID NO: 254 - DNA, 5003 bp, synthetic construct
  misc_feature 1..5003: note = Nucleotide sequence of the 5'-arm of targeting vector
  source 1..5003: mol_type = other DNA
aaagacattcccagggtccctaagggctccatctgggagcgcgctatctgtagtcctgttgtggtttaagataccactctctttgttatttgtaaatttaccacaggctgggcagtaaagtacagataggaagaaaagagtgtgaacgtttgcagttttaacagaactccttttatttgctatgcattgcaacaaccctctaaaagcaataatggcgataaacatgcaagttaatctacaactttatcaccctagtaaatcattggttcctatttcccttccataggctgaacatagtgagcactggtagctctttgcttggcttaagcgctgcttgttaaaatctaaaaaaaaaaaaaaaaaagatttgcagttttgctttaaagtttttcctttaaagcatctctgagctgcaaaaactcgtctttaggatatggttttggcggagacctaagcctgcaagtaggaatattttaaagagtaaaggctgatactttcgaacttgcaccgcagggttgggtgtcggtcaaggttaggcggagggcgacaggccaactccaaagacacggcgggggaggggtgcggtggagaaagcgagccgcagccaatcgcacgcgctctcccgacacctgccgccttggcgccttttctaggctactccgcgcacgcactggctgcgcgcgcagttcgcctccagcggtcttgggggagcacctcggtaggtgtacgtgcggaaggaagtgacgtagatccagagggccggccggggggtggggccgagctataagctttgggtgggaggcagcgctgccttcagaaggcgtgcggagcgcgggctgctgcattgcggactgtagaggcgcttcctagtgagtgactcccttgtcagcggcacggcccatcgtggtcctcgcgtggcgggcggaccagagcgagacgccagggcctgggtggtgcgggcggggaggcggaggggtgtcgcggagtccggggctgaggagcgcgggttgcaggtgcagcgcggtgggtgtggggagccgctgtaccctgcgcccctcgggtcctccgggccttcgcaggccagtgctaggccgcgggttcgagagtcaccacgctgaggcgcaggcttgttccgccgggagcacgtggtggcggctggaggaagtcgccccagggaacggctgtcggggtacgtgggtgaccttggggcccctcgcaggagggcgtcacagctgaaaaggacaaagctgttttctattcggttactagtgtcacggacatttagaggggcggggggagcttccaataactgcacgttggaacttcggcaccacctggtcggtttttttgccagtctctccctcttggcccagcgtgtggaatctcatttttctagggcagaataggtctgaacgctgcaggtaatactaagaacgtctctagcatctcctaagatgggagaacgtagaaatacgacctctttgtacgagctcttttagaactagctgtagagaaccagcgtgcaccctggtgttggacagctctctaaaatggtgtttgagggtaagaaaactgcatttgcaaatttttcagttagcacactttgtcccgagcgtaaaatgaaatgatctccttatacttaggcagaagactggactggattgctgttcagtttctgtgctatttttttaaaataggatttaagtgggaaatagttgtgttacagaaattctcggctattctgtgctatttttttttaaataggatttaagtgggaaatagttgtgttacagaaattctcggctacctgatacttttattctaagattagatgagttggctctgagctgtgaaatatgacctctttgacaaagacatttaagctgattcaggatgttatctacaaagaaaacgggatttagcttgtgtgggtccacttgcatttattttctgttaaggattataataaactgctttatacaggaatccaatatcagctgttttttatatagagagcataatactttttactttgagagaggatgttgtcaaggaatggtggctgtgaacctggcctgtgttgactggttagatctgtctgcctaaccccaccctaaggctaagtagttatatgcttgtggcaatgtgcttatttataatagggcaagattatgggctaaatttgggttagacaacaatgaaagttaattaaacgaccctcaggccttgggtctactatgtgtaagtgatttccttctctcccagatgagtgctatacaaaataaacttcagtgacctcaagtggttttgaccttttggttgctattcagaaaactatggaaatgaaaacctgctacctatttcctattgccttttcaatttcccaaagaaggtctcccctatgaatccatgggtagccttgaactcagatccacctgcttctgtttggagagtgataagattaaaggcaagtgccaccacacccagcaaagtaggctcttaaaactaaaacctttgcagtcgggcatggtgccacacacctttggtcccagcactggggggtaaggcagaggcaggtggatcactgagtttgaggtcagctagtgctgaagagtaagagcctgtctttaaaacatctcaacagctgggcagtcgtggtccattcctttaatcccagcacttgggaggcagaggcaagtggatttttgagttcgaggccagcctggtctacagagtgagttccaggacagccagggatacacagagaaaccctgtctcaaaaaaccaaaatataaataaataaataaagattgggagagaagtcaaggatctcataggtggtcagggagctacaactgcagtagtaaagaagtaggacttaaaagaacagggccggcactaattttgaggatctagatcgggccctcaattaaggaacttccttttgtgtgaactcagaatttgaaatgaaatgtgcttgtcagaaccattgcatggcttattttttaatgaaaagtctggctagtatctgcttatcttccagcttccagctcaaagttaaggtcatagatcaaaagaactatgtctttatcttagttgtatcttaatttttattagaattgaatggttttcctaatgtttggtaacatcaaaggtgtgtaagtaaaagtgagaaatcaagattaactttctcttggcaaagattgttgacattggtgacatcttggaccaaatgagaattgttttacttttaaatgtcccatcaacagctctcagttaggctgttctatctggtttgtcttgccatgcttgcagagtatagatttgacaatttgaaaattcaaaaagctatataaataggtatgttgctatatgtaagattttaaatgagtcagttaagacttaaagaataactgggtttattttatcttgtcaggttatcactgtgtagaccaggttgaccttgaaaacaaattctctgcttcccaagtgctgggattaaaggcgtgtgtcactagctctgacactggctactttggaactactatggtgttcacaaatgcagagttgagtgttgggattaaatggaaatttcatgtctttttttttaactttcccttctacacagggcttctctgtgtagtcctggctgtccttgtagctctagactaggctgaattcaaactcagatccacccacctaagtgctgagattaaaggcatgtgccaccactgcccagttctgaattgttggggttttttttttgttgttattcttaattttagttcgatgaattaaaatcgaaataacttgtttcttagaaaaataaggtgtaattgggttataaagccaaatttagacattaataccaacagcctgtttaggctcaaaattgttcaatcattttattagtattattattaatcatatcaacttgagacctgtttgggaaagcagaatattttagggatagctatttcagacaagcattaatgtgttagctgtttttttccccctagaatatgattaaaattggctcagggtggggccttctagttctggctctagcgattgggtctgtttctggtgaggtggtagtgataaactgtaacagaagggacaagagattgggcttctgagaacatgtatgatctggtatgtgactttaatcattaaagcatggggattcaaaaatactaatgaataggccttagaactagtcctgagtgttttgtaaaataacagtcttaattctcctagtttctggatttttttctttgtctttggatactaagtttaagcatttatttggacagagtggtgcccactagctgttctattctagtacttgggaggcaagaggcagaacgaacatggccagcttggacaacttaagaaaactgaaaggcttgccatcctagtttttcttttgattaaacacacttatattctaactagttttctctatcctttgggttttgttttgttttttatttgttttgtagctctagctggctggcttcggaattgcctgcctcccaagtgctaggattaaaggcatcacagtcactacccaattgatatcatgattcttaattcaacttctaagaacaaattatcacactctgaatctaacatggaatagcattatccatgttcagatatctttgtctcaaggctcaggtttatcctttgtagctttctttttgctatcccacctctattcagcatccagtcaaggatcactgagttggttatcagtaaattaaacattttaattaatgtctaggagacaggttatggtatagttctcagtgctgggaagttgacatggtaggatctcagttcatggccagccagaactttctggtgagatcctggttcaagcactacagagcttttctagaaaagtaactatatttaggagtaagtttgatataatgacaatcccatcgtaagccttcagtaacctgatgcattggtctctgttttaatatcaggta

SEQ ID NO: 255 - DNA, 2604 bp, synthetic construct
  misc_feature 1..2604: note = Nucleotide sequence of the 3'-arm of targeting vector
  source 1..2604: mol_type = other DNA
gcattctctaacttggtaaggtactttcatctatctgaaaaaatgttcagtaaaaaacaaaacaaaaacatgacttcacccagtgagtacaagatgatacccccaattttttttttttttattatatgtatgtacactgtagctgtcttcagacacaccagaagagggcgtcagatctcatcagatggttgtgagccaccatgtggttgctgggacttgaacactggacctttggaaaagcagttgggtactcttacccactgagctatcacaccagccccaataccccaaattttgaccagccttttttttttttttttttttttttttaaatctgaactaatggtattaaggtgaaacagattgcacaaaagaggtactaggtttttctttgaggcaaggtctttctatattgcctcaggttgaccttaaactccaacttcctttttttccccaagtgctggagattgtagacagatatatacaacaccccaaaacaaatatgtttagtttcgattaagattcattatgtggggctagagggatgacttagaggttaagaacactgcctgctcttccagaggtcctgagttcaattcccagcaaccacgtggtggctcacaaccatctgtaatgggatccgatgccctctttctggtgtgtttgaagacagctacagtgtcctcatacataaaaaaaataaacaaacagatcttttaaaaaaaaaggtacagtgtacttatacattatataaatgaatgattctttaaaaaattaattatgggaaatacttatgaagaatagggtagctttggctgttttggaaacgttatataacaaggtagaactaaaatgtatgccagtaatcccagaggaatcattagccagtcagggctagtctgagcaatgtggcaagataaacccatctctttaaaaaaaaaaaaaagtattataaatagaaatgttataggaaacaggaaatagaaaccctcgaaaggctgaatgaaagagtattagtgggctggagagatggttcagcggttaagagcactatctcctgagttcagttcccagtgaccacatggtggctcacagccatctgtaatgagatctgacgccctcttctggggtgtctgaagacagcgacagtgtactcacataaaataaatacataaagactgttagttagccttcatctaccatttacagaactgggcacagaaaggagttcatcagttataaagggtaactttccatatgaatgtttgtcatattattatgcatatagtataatgaccaaactactgtaatgtcttaatatttgtatctcttttctcttttttaaaaatatcagtttggtggggaaccattgtcatacacccggtttagccttgctcggcaagtagatggagataacagtcatgtggagatgaaactggctgcagatgaagaagaaaatgccgacaataacatgaaggctagtgtcagaaaacccaagaggtttaatggaagactctgctttgcagctattgcactagtcattttcttcttgattggtaagaatgagtggccattcagaaggatttcttatgactaactagttcttagactagctagttcttagactagctagttcttgtttcttttggatgaggagatgctttgtactttaaatggcactggggctccctacctgccggcagattaggtcctgcaagatgggaaacgtttacattatggatgttttattagagatatgcaggacatttggaatagtactaagaaaggcttccagtaagacaaggtgtgcacccatgtcttaaacaggtcactgtaaaatatgacttagttgtggtaatttaaattccattaaactcaggttcataattttctattagattctcatagtttaattaaaagttttcagggataagttaaaaatgagttctgtgagtttagctctaaaacttcctgtttttaggattcatgagtggctacctgggctattgtaagcgtgtagaacaaaaagaggagtgtgtgaaactggctgaaacggaggagacagacaagtcagaaaccatggaaacagaggatgttcctacatcatctcgcttatattgggcagacctcaaaacactgttgtcagagaagttgaactccatagagtttgctgacaccatcaagtaagctcaacttcccaagttcagtcctgatggaaacgtttttgttgggggggtagggagacttgaaaggctttcagagggtcctcctgacaatgtggaactatgctgacaggaaattaggacttacctggagagcctcatagcctcctttttcttcagaatcgttttatcagttgtagtttagtgtgggatgtcagattttcttctgttctaatattccttttaaaaattttttaaaattaaaattactttttatgtattatgagtatagcctgcatatatgaatgaatgtgcattactaattacacttatctcctagtgcctacataagccttatagatggttgtgagccagcatgtgggtgctggaatccaaaatggttcttgcaagaccaaatatgttacatcccagagccattaca

SEQ ID NO: 256 - protein, 166 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 257 - protein, 616 aa, synthetic construct
  REGION 1..616: note = Amino acid sequence of the fusion protein of heavy chain of anti-hTfR antibody No.3 (humanized 2) and hEPO
EVQLVQSGAEVKKPGESLKISCKGSGYSFTNYWLGWVRQMPGKGLEWMGDIYPGGDYPTYSEKFKVQVTISADKSISTAYLQWSSLKASDTAMYYCARSGNYDEVAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 258 - DNA, 1931 bp, synthetic construct
  misc_feature 1..1931: note = Nucleotide sequence encoding the amino acid sequence of fusion protein of heavy chain of anti-hTfR antibody No. 3 (humanized 2) and hEPO, synthetic sequence
  source 1..1931: mol_type = other DNA
acgcgtgccgccaccatgggctggagctggattctgctgttcctcctgagcgtgacagcaggagtgcacagcgaggtgcaactagtgcagtctggagcagaggtgaaaaagcccggggagtctctgaagatttcctgtaagggttctggatacagctttaccaactactggctgggatgggtgcgccagatgcccgggaaaggcctggagtggatgggggacatctaccccggcggagactaccctacatacagcgagaagttcaaggtccaggtcaccatctcagccgacaagtccatcagcaccgcctacctgcagtggagcagcctgaaggcctcggacaccgccatgtattactgtgcgagatcaggcaattacgacgaagtggcctactggggccaaggaaccctggtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacgtgcccaccgtgcccagcacctgaactcctgggaggtccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggatctgccccaccacgcctcatctgtgacagccgagtcctggagaggtacctcttggaggccaaggaggccgagaatatcacgacgggctgtgctgaacactgcagcttgaatgagaatatcactgtcccagacaccaaagttaatttctatgcctggaagaggatggaggtcgggcagcaggccgtagaagtctggcagggcctggccctgctgtcggaagctgtcctgcggggccaggccctgttggtcaactcttcccagccgtgggagcccctgcagctgcatgtggataaagccgtcagtggccttcgcagcctcaccactctgcttcgggctctgggagcccagaaggaagccatctcccctccagatgcggcctcagctgctccactccgaacaatcactgctgacactttccgcaaactcttccgagtctactccaatttcctccggggaaagctgaagctgtacacaggggaggcctgcaggacaggggacagataagcggccgc

SEQ ID NO: 259 - protein, 489 aa, Homo sapiens
RPPNIVLIFADDLGYGDLGCYGHPSSTTPNLDQLAAGGLRFTDFYVPVSLCTPSRAALLTGRLPVRMGMYPGVLVPSSRGGLPLEEVTVAEVLAARGYLTGMAGKWHLGVGPEGAFLPPHQGFHRFLGIPYSHDQGPCQNLTCFPPATPCDGGCDQGLVPIPLLANLSVEAQPPWLPGLEARYMAFAHDLMADAQRQDRPFFLYYASHHTHYPQFSGQSFAERSGRGPFGDSLMELDAAVGTLMTAIGDLGLLEETLVIFTADNGPETMRMSRGGCSGLLRCGKGTTYEGGVREPALAFWPGHIAPGVTHELASSLDLLPTLAALAGAPLPNVTLDGFDLSPLLLGTGKSPRQSLFFYPSYPDEVRGVFAVRTGKYKAHFFTQGSAHSDTTADPACHASSSLTAHEPPLLYDLSKDPGENYNLLGGVAGATPEVLQALKQLQLLKAQLDAAVTFGPSQVARGEDPALQICCHPGCTPRPACCHCPDPHA

SEQ ID NO: 260 - protein, 939 aa, synthetic construct
  REGION 1..939: note = Amino acid sequence of the fusion protein of heavy chain of anti-hTfR antibody No.3 (humanized 2) and hARSA
EVQLVQSGAEVKKPGESLKISCKGSGYSFTNYWLGWVRQMPGKGLEWMGDIYPGGDYPTYSEKFKVQVTISADKSISTAYLQWSSLKASDTAMYYCARSGNYDEVAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSRPPNIVLIFADDLGYGDLGCYGHPSSTTPNLDQLAAGGLRFTDFYVPVSLCTPSRAALLTGRLPVRMGMYPGVLVPSSRGGLPLEEVTVAEVLAARGYLTGMAGKWHLGVGPEGAFLPPHQGFHRFLGIPYSHDQGPCQNLTCFPPATPCDGGCDQGLVPIPLLANLSVEAQPPWLPGLEARYMAFAHDLMADAQRQDRPFFLYYASHHTHYPQFSGQSFAERSGRGPFGDSLMELDAAVGTLMTAIGDLGLLEETLVIFTADNGPETMRMSRGGCSGLLRCGKGTTYEGGVREPALAFWPGHIAPGVTHELASSLDLLPTLAALAGAPLPNVTLDGFDLSPLLLGTGKSPRQSLFFYPSYPDEVRGVFAVRTGKYKAHFFTQGSAHSDTTADPACHASSSLTAHEPPLLYDLSKDPGENYNLLGGVAGATPEVLQALKQLQLLKAQLDAAVTFGPSQVARGEDPALQICCHPGCTPRPACCHCPDPHA

SEQ ID NO: 261 - DNA, 2900 bp, synthetic construct
  misc_feature 1..2900: note = Nucleotide sequence encoding the amino acid sequence of fusion protein of heavy chain of anti-hTfR antibody No.3 (humanized 2) and hARSA, synthetic sequence
  source 1..2900: mol_type = other DNA
acgcgtgccgccaccatgggctggagctggattctgctgttcctcctgagcgtgacagcaggagtgcacagcgaggtgcaactagtgcagtctggagcagaggtgaaaaagcccggggagtctctgaagatttcctgtaagggttctggatacagctttaccaactactggctgggatgggtgcgccagatgcccgggaaaggcctggagtggatgggggacatctaccccggcggagactaccctacatacagcgagaagttcaaggtccaggtcaccatctcagccgacaagtccatcagcaccgcctacctgcagtggagcagcctgaaggcctcggacaccgccatgtattactgtgcgagatcaggcaattacgacgaagtggcctactggggccaaggaaccctggtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacgtgcccaccgtgcccagcacctgaactcctgggaggtccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggatctcgtccgcccaacatcgtgctgatctttgccgacgacctcggctatggggacctgggctgctatgggcaccccagctctaccactcccaacctggaccagctggcggcgggagggctgcggttcacagacttctacgtgcctgtgtctctgtgcacaccctctagggccgccctcctgaccggccggctcccggttcggatgggcatgtaccctggcgtcctggtgcccagctcccgggggggcctgcccctggaggaggtgaccgtggccgaagtcctggctgcccgaggctacctcacaggaatggccggcaagtggcaccttggggtggggcctgagggggccttcctgcccccccatcagggcttccatcgatttctaggcatcccgtactcccacgaccagggcccctgccagaacctgacctgcttcccgccggccactccttgcgacggtggctgtgaccagggcctggtccccatcccactgttggccaacctgtccgtggaggcgcagcccccctggctgcccggactagaggcccgctacatggctttcgcccatgacctcatggccgacgcccagcgccaggatcgccccttcttcctgtactatgcctctcaccacacccactaccctcagttcagtgggcagagctttgcagagcgttcaggccgcgggccatttggggactccctgatggagctggatgcagctgtggggaccctgatgacagccataggggacctggggctgcttgaagagacgctggtcatcttcactgcagacaatggacctgagaccatgcgtatgtcccgaggcggctgctccggtctcttgcggtgtggaaagggaacgacctacgagggcggtgtccgagagcctgccttggccttctggccaggtcatatcgctcccggcgtgacccacgagctggccagctccctggacctgctgcctaccctggcagccctggctggggccccactgcccaatgtcaccttggatggctttgacctcagccccctgctgctgggcacaggcaagagccctcggcagtctctcttcttctacccgtcctacccagacgaggtccgtggggtttttgctgtgcggactggaaagtacaaggctcacttcttcacccagggctctgcccacagtgataccactgcagaccctgcctgccacgcctccagctctctgactgctcatgagcccccgctgctctatgacctgtccaaggaccctggtgagaactacaacctgctggggggtgtggccggggccaccccagaggtgctgcaagccctgaaacagcttcagctgctcaaggcccagttagacgcagctgtgaccttcggccccagccaggtggcccggggcgaggaccccgccctgcaaatctgctgtcatcctggctgcaccccccgcccagcttgctgccattgcccagatccccatgcctaagcggccgc

SEQ ID NO: 262 - protein, 279 aa, Homo sapiens
DPPAPLPLVIWHGMGDSCCNPLSMGAIKKMVEKKIPGIYVLSLEIGKTLMEDVENSFFLNVNSQVTTVCQALAKDPKLQQGYNAMGFSQGGQFLRAVAQRCPSPPMINLISVGGQHQGVFGLPRCPGESSHICDFIRKTLNAGAYSKVVQERLVQAEYWHDPIKEDVYRNHSIFLADINQERGINESYKKNLMALKKFVMVKFLNDSIVDPVDSEWFGFYRSGQAKETIPLQETSLYTQDRLGLKEMDNAGQLVFLATEGDHLQLSEEWFYAHIIPFLG

SEQ ID NO: 263 - protein, 729 aa, synthetic construct
  REGION 1..729: note = Amino acid sequence of the fusion protein of heavy chain of anti-hTfR antibody No.3 (humanized 2) and hPPT-1
EVQLVQSGAEVKKPGESLKISCKGSGYSFTNYWLGWVRQMPGKGLEWMGDIYPGGDYPTYSEKFKVQVTISADKSISTAYLQWSSLKASDTAMYYCARSGNYDEVAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSDPPAPLPLVIWHGMGDSCCNPLSMGAIKKMVEKKIPGIYVLSLEIGKTLMEDVENSFFLNVNSQVTTVCQALAKDPKLQQGYNAMGFSQGGQFLRAVAQRCPSPPMINLISVGGQHQGVFGLPRCPGESSHICDFIRKTLNAGAYSKVVQERLVQAEYWHDPIKEDVYRNHSIFLADINQERGINESYKKNLMALKKFVMVKFLNDSIVDPVDSEWFGFYRSGQAKETIPLQETSLYTQDRLGLKEMDNAGQLVFLATEGDHLQLSEEWFYAHIIPFLG

SEQ ID NO: 264 - DNA, 2270 bp, synthetic construct
  misc_feature 1..2270: note = Nucleotide sequence encoding the amino acid sequence of fusion protein of heavy chain of anti-hTfR antibody No.3 (humanized 2) and hPPT-1, synthetic sequence
  source 1..2270: mol_type = other DNA
acgcgtgccgccaccatgggctggagctggattctgctgttcctcctgagcgtgacagcaggagtgcacagcgaggtgcaactagtgcagtctggagcagaggtgaaaaagcccggggagtctctgaagatttcctgtaagggttctggatacagctttaccaactactggctgggatgggtgcgccagatgcccgggaaaggcctggagtggatgggggacatctaccccggcggagactaccctacatacagcgagaagttcaaggtccaggtcaccatctcagccgacaagtccatcagcaccgcctacctgcagtggagcagcctgaaggcctcggacaccgccatgtattactgtgcgagatcaggcaattacgacgaagtggcctactggggccaaggaaccctggtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacgtgcccaccgtgcccagcacctgaactcctgggaggtccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggatctgacccgccggcgccgctgccgttggtgatctggcatgggatgggagacagctgttgcaatcccttaagcatgggtgctattaaaaaaatggtggagaagaaaatacctggaatttacgtcttatctttagagattgggaagaccctgatggaggacgtggagaacagcttcttcttgaatgtcaattcccaagtaacaacagtgtgtcaggcacttgctaaagatcctaaattgcagcaaggctacaatgctatgggattctcccagggaggccaatttctgagggcagtggctcagagatgcccttcacctcccatgatcaatctgatctcggttgggggacaacatcaaggtgtttttggactccctcgatgcccaggagagagctctcacatctgtgacttcatccgaaaaacactgaatgctggggcgtactccaaagttgttcaggaacgcctcgtgcaagccgaatactggcatgaccccataaaggaggatgtgtatcgcaaccacagcatcttcttggcagatataaatcaggagcggggtatcaatgagtcctacaagaaaaacctgatggccctgaagaagtttgtgatggtgaaattcctcaatgattccattgtggaccctgtagattcggagtggtttggattttacagaagtggccaagccaaggaaaccattcccttacaggagacctccctgtacacacaggaccgcctggggctaaaggaaatggacaatgcaggacagctagtgtttctggctacagaaggggaccatcttcagttgtctgaagaatggttttatgcccacatcataccattccttggataagcggccgc

SEQ ID NO: 265 - protein, 544 aa, Homo sapiens
SYSPEPDQRRTLPPGWVSLGRADPEEELSLTFALRQQNVERLSELVQAVSDPSSPQYGKYLTLENVADLVRPSPLTLHTVQKWLLAAGAQKCHSVITQDFLTCWLSIRQAELLLPGAEFHHYVGGPTETHVVRSPHPYQLPQALAPHVDFVGGLHHFPPTSSLRQRPEPQVTGTVGLHLGVTPSVIRKRYNLTSQDVGSGTSNNSQACAQFLEQYFHDSDLAQFMRLFGGNFAHQASVARVVGQQGRGRAGIEASLDVQYLMSAGANISTWVYSSPGRHEGQEPFLQWLMLLSNESALPHVHTVSYGDDEDSLSSAYIQRVNTELMKAAARGLTLLFASGDSGAGCWSVSGRHQFRPTFPASSPYVTTVGGTSFQEPFLITNEIVDYISGGGFSNVFPRPSYQEEAVTKFLSSSPHLPPSSYFNASGRAYPDVAALSDGYWVVSNRVPIPWVSGTSASTPVFGGILSLINEHRILSGRPPLGFLNPRLYQQHGAGLFDVTRGCHESCLDEEVEGQGFCSGPGWDPVTGWGTPNFPALLKTLLNP

SEQ ID NO: 266 - protein, 994 aa, synthetic construct
  REGION 1..994: note = Amino acid sequence of the fusion protein of heavy chain of anti-hTfR antibody No.3 (humanized 2) and hTPP-1
EVQLVQSGAEVKKPGESLKISCKGSGYSFTNYWLGWVRQMPGKGLEWMGDIYPGGDYPTYSEKFKVQVTISADKSISTAYLQWSSLKASDTAMYYCARSGNYDEVAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSSYSPEPDQRRTLPPGWVSLGRADPEEELSLTFALRQQNVERLSELVQAVSDPSSPQYGKYLTLENVADLVRPSPLTLHTVQKWLLAAGAQKCHSVITQDFLTCWLSIRQAELLLPGAEFHHYVGGPTETHVVRSPHPYQLPQALAPHVDFVGGLHHFPPTSSLRQRPEPQVTGTVGLHLGVTPSVIRKRYNLTSQDVGSGTSNNSQACAQFLEQYFHDSDLAQFMRLFGGNFAHQASVARVVGQQGRGRAGIEASLDVQYLMSAGANISTWVYSSPGRHEGQEPFLQWLMLLSNESALPHVHTVSYGDDEDSLSSAYIQRVNTELMKAAARGLTLLFASGDSGAGCWSVSGRHQFRPTFPASSPYVTTVGGTSFQEPFLITNEIVDYISGGGFSNVFPRPSYQEEAVTKFLSSSPHLPPSSYFNASGRAYPDVAALSDGYWVVSNRVPIPWVSGTSASTPVFGGILSLINEHRILSGRPPLGFLNPRLYQQHGAGLFDVTRGCHESCLDEEVEGQGFCSGPGWDPVTGWGTPNFPALLKTLLNP

SEQ ID NO: 267 - DNA, 3065 bp, synthetic construct
  misc_feature 1..3065: note = Nucleotide sequence encoding the amino acid sequence of fusion protein of heavy chain of anti-hTfR antibody No.3 (humanized 2) and hTPP-1, synthetic sequence
  source 1..3065: mol_type = other DNA
acgcgtgccgccaccatgggctggagctggattctgctgttcctcctgagcgtgacagcaggagtgcacagcgaggtgcaactagtgcagtctggagcagaggtgaaaaagcccggggagtctctgaagatttcctgtaagggttctggatacagctttaccaactactggctgggatgggtgcgccagatgcccgggaaaggcctggagtggatgggggacatctaccccggcggagactaccctacatacagcgagaagttcaaggtccaggtcaccatctcagccgacaagtccatcagcaccgcctacctgcagtggagcagcctgaaggcctcggacaccgccatgtattactgtgcgagatcaggcaattacgacgaagtggcctactggggccaaggaaccctggtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacgtgcccaccgtgcccagcacctgaactcctgggaggtccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggatctagttacagcccggagcccgaccagcggaggacgctgcccccaggctgggtgtccctgggccgtgcggaccctgaggaagagctgagtctcacctttgccctgagacagcagaatgtggaaagactctcggagctggtgcaggctgtgtcagatcccagctctcctcaatacggaaaatacctgaccctagagaatgtggctgatctggtgaggccatccccactgaccctccacacggtgcaaaaatggctcttggcagccggagcccagaagtgccattctgtgatcacacaggactttctgacttgctggctgagcatccgacaagcagagctgctgctccctggggctgagtttcatcactatgtgggaggacctacggaaacccatgttgtaaggtccccacatccctaccagcttccacaggccttggccccccatgtggactttgtggggggactgcaccattttcccccaacatcatccctgaggcaacgtcctgagccgcaggtgacagggactgtaggcctgcatctgggggtaaccccctctgtgatccgtaagcgatacaacttgacctcacaagacgtgggctctggcaccagcaataacagccaagcctgtgcccagttcctggagcagtatttccatgactcagacctggctcagttcatgcgcctcttcggtggcaactttgcacatcaggcatcagtagcccgtgtggttggacaacagggccggggccgggccgggattgaggccagtctagatgtgcagtacctgatgagtgctggtgccaacatctccacctgggtctacagtagccctggccggcatgagggacaggagcccttcctgcagtggctcatgctgctcagtaatgagtcagccctgccacatgtgcatactgtgagctatggagatgatgaggactccctcagcagcgcctacatccagcgggtcaacactgagctcatgaaggctgctgctcggggtctcaccctgctcttcgcctcaggtgacagtggggccgggtgttggtctgtctctggaagacaccagttccgccctaccttccctgcctccagcccctatgtcaccacagtgggaggcacatccttccaggaacctttcctcatcacaaatgaaattgttgactatatcagtggtggtggcttcagcaatgtgttcccacggccttcataccaggaggaagctgtaacgaagttcctgagctctagcccccacctgccaccatccagttacttcaatgccagtggccgtgcctacccagatgtggctgcactttctgatggctactgggtggtcagcaacagagtgcccattccatgggtgtccggaacctcggcctctactccagtgtttgggggtatcctatccttgatcaatgagcacagaatccttagtggccgcccccctcttggctttctcaacccaaggctctaccagcagcatggggcaggactctttgatgtaacccgtggctgccatgagtcctgtctggatgaagaggtagagggccagggtttctgctctggtcctggctgggaccctgtaacaggctggggaacacccaacttcccagctttgctgaagactctactcaacccctaagcggccgc

SEQ ID NO: 268 - protein, 628 aa, Homo sapiens
AEAPHLVHVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRALDYWARPGPFSDPVPYLEVPVPRGPPSPGNP

SEQ ID NO: 269 - protein, 1078 aa, synthetic construct
  REGION 1..1078: note = Amino acid sequence of the fusion protein of heavy chain of anti-hTfR antibody No.3 (humanized 2) and hIDUA
EVQLVQSGAEVKKPGESLKISCKGSGYSFTNYWLGWVRQMPGKGLEWMGDIYPGGDYPTYSEKFKVQVTISADKSISTAYLQWSSLKASDTAMYYCARSGNYDEVAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSAEAPHLVHVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRALDYWARPGPFSDPVPYLEVPVPRGPPSPGNP

SEQ ID NO: 270 - DNA, 3317 bp, synthetic construct
  misc_feature 1..3317: note = Nucleotide sequence encoding the amino acid sequence of fusion protein of heavy chain of anti-hTfR antibody No.3 (humanized 2) and hIDUA, synthetic sequence
  source 1..3317: mol_type = other DNA
acgcgtgccgccaccatgggctggagctggattctgctgttcctcctgagcgtgacagcaggagtgcacagcgaggtgcaactagtgcagtctggagcagaggtgaaaaagcccggggagtctctgaagatttcctgtaagggttctggatacagctttaccaactactggctgggatgggtgcgccagatgcccgggaaaggcctggagtggatgggggacatctaccccggcggagactaccctacatacagcgagaagttcaaggtccaggtcaccatctcagccgacaagtccatcagcaccgcctacctgcagtggagcagcctgaaggcctcggacaccgccatgtattactgtgcgagatcaggcaattacgacgaagtggcctactggggccaaggaaccctggtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacgtgcccaccgtgcccagcacctgaactcctgggaggtccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggatctgccgaggccccgcacctggtgcacgtggacgcggcccgcgcgctgtggcccctgcggcgcttctggaggagcacaggcttctgccccccgctgccacacagccaggctgaccagtacgtcctcagctgggaccagcagctcaacctcgcctatgtgggcgccgtccctcaccgcggcatcaagcaggtccggacccactggctgctggagcttgtcaccaccagggggtccactggacggggcctgagctacaacttcacccacctggacgggtacttggaccttctcagggagaaccagctcctcccagggtttgagctgatgggcagcgcctcgggccacttcactgactttgaggacaagcagcaggtgtttgagtggaaggacttggtctccagcctggccaggagatacatcggtaggtacggactggcgcatgtttccaagtggaacttcgagacgtggaatgagccagaccaccacgactttgacaacgtctccatgaccatgcaaggcttcctgaactactacgatgcctgctcggagggtctgcgcgccgccagccccgccctgcggctgggaggccccggcgactccttccacaccccaccgcgatccccgctgagctggggcctcctgcgccactgccacgacggtaccaacttcttcactggggaggcgggcgtgcggctggactacatctccctccacaggaagggtgcgcgcagctccatctccatcctggagcaggagaaggtcgtcgcgcagcagatccggcagctcttccccaagttcgcggacacccccatttacaacgacgaggcggacccgctggtgggctggtccctgccacagccgtggagggcggacgtgacctacgcggccatggtggtgaaggtcatcgcgcagcatcagaacctgctactggccaacaccacctccgccttcccctacgcgctcctgagcaacgacaatgccttcctgagctaccacccgcaccccttcgcgcagcgcacgctcaccgcgcgcttccaggtcaacaacacccgcccgccgcacgtgcagctgttgcgcaagccggtgctcacggccatggggctgctggcgctgctggatgaggagcagctctgggccgaagtgtcgcaggccgggaccgtcctggacagcaaccacacggtgggcgtcctggccagcgcccaccgcccccagggcccggccgacgcctggcgcgccgcggtgctgatctacgcgagcgacgacacccgcgcccaccccaaccgcagcgtcgcggtgaccctgcggctgcgcggggtgccccccggcccgggcctggtctacgtcacgcgctacctggacaacgggctctgcagccccgacggcgagtggcggcgcctgggccggcccgtcttccccacggcagagcagttccggcgcatgcgcgcggctgaggacccggtggccgcggcgccccgccccttacccgccggtggccgcctgaccctgcgccccgcgctgcggctgccgtcgcttttgctggtgcacgtgtgtgcgcgccccgagaagccgcccgggcaggtcacgcggctccgcgccctgcccctgacccaagggcagctggttctggtctggtcggatgaacacgtgggctccaagtgcctgtggacatacgagatccagttctctcaggacggtaaggcgtacaccccggtcagcaggaagccatcgaccttcaacctctttgtgttcagcccagacacaggtgctgtctctggctcctaccgagttcgagccctggactactgggcccgaccaggccccttctcggaccctgtgccgtacctggaggtccctgtgccaagagggcccccatccccgggcaatccataagcggccgc

SEQ ID NO: 271 - protein, 235 aa, Homo sapiens
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGD

SEQ ID NO: 272 - protein, 685 aa, synthetic construct
  REGION 1..685: note = Amino acid sequence of the fusion protein of heavy chain of anti-hTfR antibody No.3 (humanized 2) and hTNF-alpha receptor
EVQLVQSGAEVKKPGESLKISCKGSGYSFTNYWLGWVRQMPGKGLEWMGDIYPGGDYPTYSEKFKVQVTISADKSISTAYLQWSSLKASDTAMYYCARSGNYDEVAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSLPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGD

SEQ ID NO: 273 - DNA, 2138 bp, synthetic construct
  misc_feature 1..2138: note = Nucleotide sequence encoding the amino acid sequence of fusion protein of heavy chain of anti-hTfR antibody No.3 (humanized 2) and hTNF-alpha receptor, synthetic sequence
  source 1..2138: mol_type = other DNA
acgcgtgccgccaccatgggctggagctggattctgctgttcctcctgagcgtgacagcaggagtgcacagcgaggtgcaactagtgcagtctggagcagaggtgaaaaagcccggggagtctctgaagatttcctgtaagggttctggatacagctttaccaactactggctgggatgggtgcgccagatgcccgggaaaggcctggagtggatgggggacatctaccccggcggagactaccctacatacagcgagaagttcaaggtccaggtcaccatctcagccgacaagtccatcagcaccgcctacctgcagtggagcagcctgaaggcctcggacaccgccatgtattactgtgcgagatcaggcaattacgacgaagtggcctactggggccaaggaaccctggtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacgtgcccaccgtgcccagcacctgaactcctgggaggtccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggatctttgcccgcccaggtggcatttacaccctacgccccggagcccgggagcacatgccggctcagagaatactatgaccagacagctcagatgtgctgcagcaagtgctcgccgggccaacatgcaaaagtcttctgtaccaagacctcggacaccgtgtgtgactcctgtgaggacagcacatacacccagctctggaactgggttcccgagtgcttgagctgtggctcccgctgtagctctgaccaggtggaaactcaagcctgcactcgggaacagaaccgcatctgcacctgcaggcccggctggtactgcgcgctgagcaagcaggaggggtgccggctgtgcgcgccgctgcgcaagtgccgcccgggcttcggcgtggccagaccaggaactgaaacatcagacgtggtgtgcaagccctgtgccccggggacgttctccaacacgacttcatccacggatatttgcaggccccaccaaatctgtaacgtggtggccatccctgggaatgcaagcatggatgcagtctgcacgtccacgtcccccacccggagtatggccccaggggcagtacacttaccccagccagtgtccacacgatcccaacacacgcagccaactccagaacccagcactgctccaagcacctccttcctgctcccaatgggccccagccccccagctgaagggagcactggcgactaagcggccgc

SEQ ID NO: 274 - protein, 482 aa, Homo sapiens
RPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNEL

SEQ ID NO: 275 - protein, 932 aa, synthetic construct
  REGION 1..932: note = Amino acid sequence of the fusion protein of heavy chain of anti-hTfR antibody No.3 (humanized 2) and hSGSH
EVQLVQSGAEVKKPGESLKISCKGSGYSFTNYWLGWVRQMPGKGLEWMGDIYPGGDYPTYSEKFKVQVTISADKSISTAYLQWSSLKASDTAMYYCARSGNYDEVAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSRPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNEL

SEQ ID NO: 276 - DNA, 2879 bp, synthetic construct
  misc_feature 1..2879: note = Nucleotide sequence encoding the amino acid sequence of fusion protein of heavy chain of anti-hTfR antibody No.3 (humanized 2) and hSGSH, synthetic sequence
  source 1..2879: mol_type = other DNA
acgcgtgccgccaccatgggctggagctggattctgctgttcctcctgagcgtgacagcaggagtgcacagcgaggtgcaactagtgcagtctggagcagaggtgaaaaagcccggggagtctctgaagatttcctgtaagggttctggatacagctttaccaactactggctgggatgggtgcgccagatgcccgggaaaggcctggagtggatgggggacatctaccccggcggagactaccctacatacagcgagaagttcaaggtccaggtcaccatctcagccgacaagtccatcagcaccgcctacctgcagtggagcagcctgaaggcctcggacaccgccatgtattactgtgcgagatcaggcaattacgacgaagtggcctactggggccaaggaaccctggtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacgtgcccaccgtgcccagcacctgaactcctgggaggtccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggatctcgtccccggaacgcactgctgctcctcgcggatgacggaggctttgagagtggcgcgtacaacaacagcgccatcgccaccccgcacctggacgccttggcccgccgcagcctcctctttcgcaatgccttcacctcggtcagcagctgctctcccagccgcgccagcctcctcactggcctgccccagcatcagaatgggatgtacgggctgcaccaggacgtgcaccacttcaactccttcgacaaggtgcggagcctgccgctgctgctcagccaagctggtgtgcgcacaggcatcatcgggaagaagcacgtggggccggagaccgtgtacccgtttgactttgcgtacacggaggagaatggctccgtcctccaggtggggcggaacatcactagaattaagctgctcgtccggaaattcctgcagactcaggatgaccggcctttcttcctctacgtcgccttccacgacccccaccgctgtgggcactcccagccccagtacggaaccttctgtgagaagtttggcaacggagagagcggcatgggtcgtatcccagactggaccccccaggcctacgacccactggacgtgctggtgccttacttcgtccccaacaccccggcagcccgagccgacctggccgctcagtacaccaccgtcggccgcatggaccaaggagttggactggtgctccaggagctgcgtgacgccggtgtcctgaacgacacactggtgatcttcacgtccgacaacggtatccccttccccagcggcaggaccaacctgtactggccgggcactgctgaacccttactggtgtcatccccggagcacccaaaacgctggggccaagtcagcgaggcctacgtgagcctcctagacctcacgcccaccatcttggattggttctcgatcccgtaccccagctacgccatctttggctcgaagaccatccacctcactggccggtccctcctgccggcgctggaggccgagcccctctgggccaccgtctttggcagccagagccaccacgaggtcaccatgtcctaccccatgcgctccgtgcagcaccggcacttccgcctcgtgcacaacctcaacttcaagatgccctttcccatcgaccaggacttctacgtctcacccaccttccaggacctcctgaaccgcaccacagctggtcagcccacgggctggtacaaggacctccgtcattactactaccgggcgcgctgggagctctacgaccggagccgggacccccacgagacccagaacctggccaccgacccgcgctttgctcagcttctggagatgcttcgggaccagctggccaagtggcagtgggagacccacgacccctgggtgtgcgcccccgacggcgtcctggaggagaagctctctccccagtgccagcccctccacaatgagctgtaagcggccgc

SEQ ID NO: 277 - protein, 245 aa, synthetic construct
  REGION 1..245: note = Amino acid sequence of anti-hTfR single-chain antibody
EVQLVQSGAEVKKPGESLKISCKGSGYSFTNYWLGWVRQMPGKGLEWMGDIYPGGDYPTYSEKFKVQVTISADKSISTAYLQWSSLKASDTAMYYCARSGNYDEVAYWGQGTLVTVSSGGGGSGGGGSGGGGSDIVMTQTPLSLSVTPGQPASISCRSSQSLVHSNGNTYLHWYLQKPGQSPQLLIYKVSNRFSGVPDRFSGSGSGTDFTLKISRVEAEDVGVYYCSQSTHVPWTFGQGTKVEIK

SEQ ID NO: 278 - protein, 16 aa, synthetic construct
  REGION 1..16: note = Amino acid sequence 2 of CDR 2 in the heavy chain of mouse anti-hTfR antibody No. 6
IIWGDGSTNYHSALIS

SEQ ID NO: 279 - protein, 16 aa, synthetic construct
  REGION 1..16: note = Amino acid sequence 2 of CDR 2 in the heavy chain of mouse anti-hTfR antibody No. 8
IIWGDGSTNYRSALIS

(19) United States
(12) Patent Application Publication
(10) Pub. No.: US 2026/0184781 A1
(43) Pub. Date: Jul. 2, 2026

(54) Anti-Human Transferrin Receptor Antibody Permeating Blood-Brain Barrier

(71) Applicant: JCR Pharmaceuticals Co., Ltd., Hyogo (JP)

(72) Inventors: Hiroyuki Sonoda, Hyogo (JP); Kenichi Takahashi, Hyogo (JP)

(73) Assignee: JCR Pharmaceuticals Co., Ltd., Hyogo (JP)

(21) Appl. No.: 19/422,914

(22) Filed: Dec. 17, 2025

Related U.S. Application Data
(63) Continuation of application No. 17/530,587, filed on Nov. 19, 2021, now abandoned.
Application No. 17/530,587 is a continuation of application No. 15/739,200, now Pat. No. 11,248,045.

(30) Foreign Application Priority Data
Jun. 24, 2015 (JP) 2015-144379

(51) Int. Cl. C07K 16/28; A61K 39/00; A61K 39/395; A61K 45/00; A61P 25/00; C07K 14/475; C07K 14/505; C07K 14/715; C07K 16/18; C07K 16/46; C07K 19/00; C12N 5/10; C12N 9/10; C12N 9/14; C12N 9/16; C12N 9/24; C12N 9/48; C12N 9/64; C12N 15/09; C12N 15/113

(52) U.S. Cl. CPC C07K 16/28; A61K 39/395; A61K 45/00; A61P 25/00; C07K 14/475; C07K 14/505; C07K 14/7151; C07K 16/18; C07K 16/46; C07K 19/00; C12N 5/10; C12N 9/10; C12N 9/14; C12N 9/16; C12N 9/24; C12N 9/2402; C12N 9/48; C12N 9/64; C12N 15/09; C12N 15/113; C12Y 301/02; C12Y 302/01076; C12Y 304/14009; C12Y 310/01001; A61K 2039/505; C07K 2317/24; C07K 2317/92; C07K 2319/30

(57) ABSTRACT

Disclosed are a means to convert compounds having physiological or pharmacological activity and unable to pass through the blood-brain barrier into a form that allows them to pass through the blood-brain barrier, and compounds converted thereby. The means is an anti-human transferrin receptor antibody and the converted compounds are molecular conjugates between physiologically active protein or pharmacologically active low-molecular-weight compounds and an anti-human transferrin receptor antibody.

### SEQUENCE LISTING SUBMISSION VIA PATENT CENTER

The contents of the electronic sequence listing (046124-5460-02.xml; Size: 437,870 bytes; and Date of Creation: Dec. 17, 2025 is herein incorporated by reference in its entirety.

### TECHNICAL FIELD

The present invention relates to an anti-human transferrin receptor antibody to be utilized for conjugation with a compound that needs to exhibit its function in the central nervous system when administered parenterally (a protein or a low-molecular-weight compound and the like) in order to make that compound able to pass through the blood-brain barrier after parenterally administered, and also a method for production thereof, as well as a method of use thereof.

### BACKGROUND ART

Unlike the capillaries in other tissues such as muscles, the capillaries that supply the blood to most of the brain tissues except some areas including the circumventricular organs (pineal gland, pituitary body, area postrema, etc.) differ in that the endothelial cells forming their endothelium are mutually connected by tight intercellular junctions. Passive transfer of substances from the capillaries to the brain is thereby restricted, and although there are some exceptions, substances are unlikely to move into the brain from the blood except such compounds as are lipid-soluble or of low-molecular-weight (not greater than 200 to 500 Dalton) and electrically neutral around the physiological pH. This system, which restricts exchange of substances between the blood and the tissue fluid of the brain through the endothelium of capillaries in the brain, is called the blood-brain barrier or BBB. The blood-brain barrier not only restricts exchange of substances between the blood and the brain but also between the tissue fluid of the central nervous system, including the brain and the spinal chord, and the blood.

Owing to the blood-brain barrier, most of the cells of the central nervous system escape the effects of fluctuating concentrations of substances like hormones and lymphokines in the blood, and their biochemical homeostasis is thus maintained.

The blood-brain barrier, however, imposes a problem when it comes to develop pharmaceutical agents. For mucopolysaccharidosis type I (Hurler syndrome), an inherited metabolic disease caused by α-L-iduronidase deficiency, for example, although an enzyme replacement therapy is carried out by intravenous supplementation with a recombinant α-L-iduronidase as a therapy, the therapy is not effective for the notable abnormality observed in the central nervous system (CNS) in Hurler syndrome because the enzyme cannot pass through the blood-brain barrier.

Development of various methods has been attempted to make those macromolecular substances as proteins or the like, which need to be brought into function in the central nervous system, pass through the blood-brain barrier. In the case of nerve growth factor, for example, while attempts have been made for a method to cause the factor to pass through the blood-brain barrier by allowing liposomes encapsulating the factor to fuse with the cell membrane of endothelial cells in brain capillaries, they have not been reached practical application (Non Patent Literature 1). In the case of α-L-iduronidase, an attempt has been made to enhance the passive transfer of the enzyme through the blood-brain barrier by raising its blood concentration through an increased single dose of the enzyme, and it thus has been demonstrated, using a Hurler syndrome animal model, that the abnormality in the central nervous system (CNS) is ameliorated by that method (Non Patent Literature 2).

Furthermore, circumventing the blood-brain barrier, an attempt has also been made to administer a macromolecular substance directly into the medullary cavity or into the brain. For example, reports have been made about a method in which human α-L-iduronidase was intrathecally administered to a patient with a Hurler syndrome (mucopolysaccharidosis type I) (Patent Literature 1), a method in which human acid sphingomyelinase was administered into the brain ventricles of a patient with Niemann-Pick disease (Patent Literature 2), and a method in which iduronate 2-sulfatase (I2S) was administered into the brain ventricles of Hunter syndrome model animals (Patent Literature 3). While it seems possible by one of such methods to definitely let a pharmaceutical agent act in the central nervous system, they have a problem as being highly invasive.

There have been reported various methods to let a macromolecular substance get into the brain through the blood-brain barrier, in which the macromolecular substance is modified to give it an affinity to membrane proteins existing on the endothelial cells of the brain capillaries. Examples of those membrane proteins which exist on the endothelial cells of the brain capillaries include receptors for compounds such as insulin, transferrin, insulin-like growth factor (IGF-I, IGF-II), LDL, and leptin.

For example, a technique has been reported in which nerve growth factor (NGF) was synthesized in the form of a fusion protein with insulin, and this fusion protein was allowed to pass through the blood-brain barrier via its binding to the insulin receptor (Patent Literatures 4-6). Further, a technique has been reported in which nerve growth factor (NGF) was synthesized in the form of a fusion protein with anti-insulin receptor antibody, and this fusion protein was allowed to pass through the blood-brain barrier via its binding to the insulin receptor (Patent Literatures 4 and 7). Further, a technique has been reported in which nerve growth factor (NGF) was synthesized in the form of a fusion protein with transferrin, and this fusion protein was allowed to pass through the blood-brain barrier via its binding to the transferrin receptor (TfR) (Patent Literature 8). Further, a technique has been reported in which nerve growth factor (NGF) was synthesized in the form of a fusion protein with anti-transferrin receptor antibody (anti-TfR antibody), and this fusion protein is allowed to pass through the blood-brain barrier via its binding to TfR (Patent Literatures 4 and 9).

Looking further into the techniques that utilize an anti-transferrin receptor antibody, there has been reported that in the field of the technique to make a pharmaceutical agent pass through the blood-brain barrier by binding it to an anti-TfR antibody, a single-chain antibody could be used (Non Patent Literature 3). Further, it has been reported that anti-hTfR antibodies exhibiting relatively high dissociation constants with hTfR (low-affinity anti-hTfR antibody) could be favorably used in the technique to make pharmaceutical agents pass through the blood-brain barrier (Patent Literatures 10 and 11, and Non Patent Literature 4). Still further, it has also been reported that an anti-TfR antibodies whose affinity to hTfR varies depending on pH could be employed as a carrier for making pharmaceutical agents pass through the blood-brain barrier (Patent Literature 12, and Non Patent Literature 5).

### CITATION LIST

### Patent Literature

Patent Literature 1: JP2007-504166 A1
Patent Literature 2: JP2009-525963 A1
Patent Literature 3: JP2012-62312 A1
Patent Literature 4: U.S. Pat. No. 5,154,924 B1
Patent Literature 5: JP2011-144178 A1
Patent Literature 6: US2004/0101904 A1
Patent Literature 7: JP2006-511516 A1
Patent Literature 8: JPH06-228199 A1
Patent Literature 9: U.S. Pat. No. 5,977,307 B1
Patent Literature 10: WO 2012/075037
Patent Literature 11: WO 2013/177062
Patent Literature 12: WO 2012/143379

### Non Patent Literature

Non Patent Literature 1: Xie Y. et al., J Control Release. 105. 106-19 (2005)
Non Patent Literature 2: Ou L. et al., Mol Genet Metab. 111. 116-22 (2014)
Non Patent Literature 3: Li J Y. Protein Engineering. 12. 787-96 (1999)
Non Patent Literature 4: Bien-Ly N. et al., J Exp Med. 211. 233-44 (2014)
Non Patent Literature 5: Sada H. PLOS ONE. 9. E96340 (2014)

### SUMMARY OF INVENTION

### Problems to be Solved by the Invention

Against the above background, it is an objective of the present invention to provide an anti-TfR antibody that can be utilized for conjugation with a compound that needs to exhibit its function in the central nervous system when administered parenterally (a protein or a low-molecular-weight compound and the like) in order to make that compound able to pass through the blood-brain barrier, and also a method for production thereof, as well as a method of use thereof.

### Means for Solving the Problems

As a result of intense studies aimed at the above objective, the present inventors have found that anti-human transferrin receptor antibodies (anti-hTfR antibodies) that recognize the extracellular region of hTfR which are obtained by the method for antibody production described in detail in the specification, efficiently passes through the blood-brain barrier when administered to the body, and have completed the present invention thereupon. Thus the present invention provides what follows:
1. An anti-human transferrin receptor antibody, wherein the amino acid sequence of the light chain variable region of the antibody is selected from the group consisting of (1) to (14) below:
(1) an amino acid sequence comprising the amino acid sequence set forth as SEQ ID NO:6 or SEQ ID NO:7 in CDR1, the amino acid sequence set forth as SEQ ID NO:8 or SEQ ID NO:9 or the amino acid sequence Trp-Thr-Ser in CDR2, and the amino acid sequence set forth as SEQ ID NO:10 in CDR3;
(2) an amino acid sequence comprising the amino acid sequence set forth as SEQ ID NO:11 or SEQ ID NO:12 in CDR1, the amino acid sequence set forth as SEQ ID NO:13 or SEQ ID NO:14 or the amino acid sequence Tyr-Ala-Ser in CDR2, and the amino acid sequence set forth as SEQ ID NO:15 in CDR3;
(3) an amino acid sequence comprising the amino acid sequence set forth as SEQ ID NO:16 or SEQ ID NO:17 in CDR1, the amino acid sequence set forth as SEQ ID NO:18 or SEQ ID NO:19 or the amino acid sequence Lys-Val-Ser in CDR2, and the amino acid sequence set forth as SEQ ID NO:20 in CDR3;
(4) an amino acid sequence comprising the amino acid sequence set forth as SEQ ID NO:21 or SEQ ID NO:22 in CDR1, the amino acid sequence set forth as SEQ ID NO:23 or SEQ ID NO:24 or the amino acid sequence Asp-Thr-Ser in CDR2, and the amino acid sequence set forth as SEQ ID NO:25 in CDR3;
(5) an amino acid sequence comprising the amino acid sequence set forth as SEQ ID NO:26 or SEQ ID NO:27 in CDR1, the amino acid sequence set forth as SEQ ID NO:28 or SEQ ID NO:29 or the amino acid sequence Asp-Thr-Ser in CDR2, and the amino acid sequence set forth as SEQ ID NO:30 in CDR3;
(6) an amino acid sequence comprising the amino acid sequence set forth as SEQ ID NO:31 or SEQ ID NO:32 in CDR1, the amino acid sequence set forth as SEQ ID NO:33 or SEQ ID NO:34 or the amino acid sequence Ala-Ala-Ser in CDR2, and the amino acid sequence set forth as SEQ ID NO:35 in CDR3;
(7) an amino acid sequence comprising the amino acid sequence set forth as SEQ ID NO:36 or SEQ ID NO:37 in CDR1, the amino acid sequence set forth as SEQ ID NO:38 or SEQ ID NO:39 or the amino acid sequence Gln-Thr-Ser in CDR2, and the amino acid sequence set forth as SEQ ID NO:40 in CDR3;
(8) an amino acid sequence comprising the amino acid sequence set forth as SEQ ID NO:41 or SEQ ID NO:42 in CDR1, the amino acid sequence set forth as SEQ ID NO:43 or SEQ ID NO:44 or the amino acid sequence Gly-Thr-Ser in CDR2, and the amino acid sequence set forth as SEQ ID NO:45 in CDR3;
(9) an amino acid sequence comprising the amino acid sequence set forth as SEQ ID NO:46 or SEQ ID NO:47 in CDR1, the amino acid sequence set forth as SEQ ID NO:48 or SEQ ID NO:49 or the amino acid sequence Phe-Thr-Ser in CDR2, and the amino acid sequence set forth as SEQ ID NO:50 in CDR3;
(10) an amino acid sequence comprising the amino acid sequence set forth as SEQ ID NO:51 or SEQ ID NO:52 in CDR1, the amino acid sequence set forth as SEQ ID NO:53 or SEQ ID NO:54 or the amino acid sequence Ala-Ala-Ser in CDR2, and the amino acid sequence set forth as SEQ ID NO:55 in CDR3;
(11) an amino acid sequence comprising the amino acid sequence set forth as SEQ ID NO:56 or SEQ ID NO:57 in CDR1, the amino acid sequence set forth as SEQ ID NO:58 or SEQ ID NO:59 or the amino acid sequence Tyr-Ala-Ser in CDR2, and the amino acid sequence set forth as SEQ ID NO:60 in CDR3;
(12) an amino acid sequence comprising the amino acid sequence set forth as SEQ ID NO:61 or SEQ ID NO:62 in CDR1, the amino acid sequence set forth as SEQ ID NO:63 or SEQ ID NO:64 or the amino acid sequence Trp-Ser-Ser in CDR2, and the amino acid sequence set forth as SEQ ID NO:65 in CDR3;
(13) an amino acid sequence comprising the amino acid sequence set forth as SEQ ID NO:66 or SEQ ID NO:67 in CDR1, the amino acid sequence set forth as SEQ ID NO:68 or SEQ ID NO:69 or the amino acid sequence Tyr-Ala-Ser in CDR2, and the amino acid sequence set forth as SEQ ID NO:70 in CDR3; and
(14) an amino acid sequence comprising the amino acid sequence set forth as SEQ ID NO:71 or SEQ ID NO:72 in CDR1, the amino acid sequence set forth as SEQ ID NO:73 or SEQ ID NO:74 or the amino acid sequence Asp-Thr-Ser in CDR2, and the amino acid sequence set forth as SEQ ID NO:75 in CDR3.
2. The anti-human transferrin receptor antibody according to 1 above, wherein the amino acid sequence of the light chain variable region of the antibody is selected from the group consisting of (1) to (14) below:
(1) an amino acid sequence comprising the amino acid sequence set forth as SEQ ID NO:6 in CDR1, the amino acid sequence set forth as SEQ ID NO:8 in CDR2, and the amino acid sequence set forth as SEQ ID NO:10 in CDR3;
(2) an amino acid sequence comprising the amino acid sequence set forth as SEQ ID NO:11 in CDR1, the amino acid sequence set forth as SEQ ID NO:13 in CDR2, and the amino acid sequence set forth as SEQ ID NO:15 in CDR3;
(3) an amino acid sequence comprising the amino acid sequence set forth as SEQ ID NO:16 in CDR1, the amino acid sequence set forth as SEQ ID NO:18 in CDR2, and the amino acid sequence set forth as SEQ ID NO:20 in CDR3;
(4) an amino acid sequence comprising the amino acid sequence set forth as SEQ ID NO:21 in CDR1, the amino acid sequence set forth as SEQ ID NO:23 in CDR2, and the amino acid sequence set forth as SEQ ID NO:25 in CDR3;
(5) an amino acid sequence comprising the amino acid sequence set forth as SEQ ID NO:26 in CDR1, the amino acid sequence set forth as SEQ ID NO:28 in CDR2, and the amino acid sequence set forth as SEQ ID NO:30 in CDR3;
(6) an amino acid sequence comprising the amino acid sequence set forth as SEQ ID NO:31 in CDR1, the amino acid sequence set forth as SEQ ID NO:33 in CDR2, and the amino acid sequence set forth as SEQ ID NO:35 in CDR3;
(7) an amino acid sequence comprising the amino acid sequence set forth as SEQ ID NO:36 in CDR1, the amino acid sequence set forth as SEQ ID NO:38 in CDR2, and the amino acid sequence set forth as SEQ ID NO:40 in CDR3;
(8) an amino acid sequence comprising the amino acid sequence set forth as SEQ ID NO:41 in CDR1, the amino acid sequence set forth as SEQ ID NO:43 in CDR2, and the amino acid sequence set forth as SEQ ID NO:45 in CDR3;
(9) an amino acid sequence comprising the amino acid sequence set forth as SEQ ID NO:46 in CDR1, the amino acid sequence set forth as SEQ ID NO:48 in CDR2, and the amino acid sequence set forth as SEQ ID NO:50 in CDR3;
(10) an amino acid sequence comprising the amino acid sequence set forth as SEQ ID NO:51 in CDR1, the amino acid sequence set forth as SEQ ID NO:53 in CDR2, and the amino acid sequence set forth as SEQ ID NO:55 in CDR3;
(11) an amino acid sequence comprising the amino acid sequence set forth as SEQ ID NO:56 in CDR1, the amino acid sequence set forth as SEQ ID NO:58 in CDR2, and the amino acid sequence set forth as SEQ ID NO:60 in CDR3;
(12) an amino acid sequence comprising the amino acid sequence set forth as SEQ ID NO:61 in CDR1, the amino acid sequence set forth as SEQ ID NO:63 in CDR2, and the amino acid sequence set forth as SEQ ID NO:65 in CDR3;
(13) an amino acid sequence comprising the amino acid sequence set forth as SEQ ID NO:66 in CDR1, the amino acid sequence set forth as SEQ ID NO:68 in CDR2, and the amino acid sequence set forth as SEQ ID NO:70 in CDR3; and
(14) an amino acid sequence comprising the amino acid sequence set forth as SEQ ID NO:71 in CDR1, the amino acid sequence set forth as SEQ ID NO:73 in CDR2, and the amino acid sequence set forth as SEQ ID NO:75 in CDR3.
3. An anti-human transferrin receptor antibody, wherein the amino acid sequences of CDR1, CDR2 and CDR3 in the light chain variable region thereof have a homology not lower than 80% to the amino acid sequences of CDR1, CDR2 and CDR3, respectively, in the light chain according to 1 or 2 above.
4. An anti-human transferrin receptor antibody, wherein the amino acid sequences of CDR1, CDR2 and CDR3 in the light chain variable region thereof have a homology not lower than 90% to the amino acid sequences of CDR1, CDR2 and CDR3, respectively, in the light chain according to 1 or 2 above.
5. An anti-human transferrin receptor antibody, wherein 1 to 5 amino acids are substituted, deleted or added relative to the amino acid sequence that forms at least one of the CDRs in the light chain according to 1 or 2 above.
6. An anti-human transferrin receptor antibody, wherein 1 to 3 amino acids are substituted, deleted or added relative to the amino acid sequence that forms at least one of the CDRs in the light chain according to 1 or 2 above.
7. An anti-human transferrin receptor antibody, wherein the amino acid sequence of the heavy chain variable region of the antibody is selected from the group consisting of (1) to (14) below:
(1) an amino acid sequence comprising the amino acid sequence set forth as SEQ ID NO:76 or SEQ ID NO:77 in CDR1, the amino acid sequence set forth as SEQ ID NO:78 or SEQ ID NO:79 in CDR2, and the amino acid sequence set forth as SEQ ID NO:80 or SEQ ID NO:81 in CDR3;
(2) an amino acid sequence comprising the amino acid sequence set forth as SEQ ID NO:82 or SEQ ID NO:83 in CDR1, the amino acid sequence set forth as SEQ ID NO:84 or SEQ ID NO:85 in CDR2, and the amino acid sequence set forth as SEQ ID NO:86 or SEQ ID NO:87 in CDR3;
(3) an amino acid sequence comprising the amino acid sequence set forth as SEQ ID NO:88 or SEQ ID NO:89 in CDR1, the amino acid sequence set forth as SEQ ID NO:90 or SEQ ID NO:91 in CDR2, and the amino acid sequence set forth as SEQ ID NO:92 or SEQ ID NO:93 in CDR3;
(4) an amino acid sequence comprising the amino acid sequence set forth as SEQ ID NO:94 or SEQ ID NO:95 in CDR1, the amino acid sequence set forth as SEQ ID NO:96 or SEQ ID NO:97 in CDR2, and the amino acid sequence set forth as SEQ ID NO:98 or SEQ ID NO:99 in CDR3;
(5) an amino acid sequence comprising the amino acid sequence set forth as SEQ ID NO:100 or SEQ ID NO:101 in CDR1, the amino acid sequence set forth as SEQ ID NO:102 or SEQ ID NO:103 in CDR2, and the amino acid sequence set forth as SEQ ID NO:104 or SEQ ID NO:105 in CDR3;
(6) an amino acid sequence comprising the amino acid sequence set forth as SEQ ID NO:106 or SEQ ID NO:107 in CDR1, the amino acid sequence set forth as SEQ ID NO:108 or SEQ ID NO:278 in CDR2, and the amino acid sequence set forth as SEQ ID NO:109 or SEQ ID NO:110 in CDR3;
(7) an amino acid sequence comprising the amino acid sequence set forth as SEQ ID NO:111 or SEQ ID NO:112 in CDR1, the amino acid sequence set forth as SEQ ID NO:113 or SEQ ID NO:114 in CDR2, and the amino acid sequence set forth as SEQ ID NO:115 or SEQ ID NO:116 in CDR3;
(8) an amino acid sequence comprising the amino acid sequence set forth as SEQ ID NO:117 or SEQ ID NO:118 in CDR1, the amino acid sequence set forth as SEQ ID NO:119 or SEQ ID NO:279 in CDR2, and the amino acid sequence set forth as SEQ ID NO:120 or SEQ ID NO:121 in CDR3;
(9) an amino acid sequence comprising the amino acid sequence set forth as SEQ ID NO:122 or SEQ ID NO:123 in CDR1, the amino acid sequence set forth as SEQ ID NO:124 or SEQ ID NO:125 in CDR2, and the amino acid sequence set forth as SEQ ID NO:126 or SEQ ID NO:127 in CDR3;
(10) an amino acid sequence comprising the amino acid sequence set forth as SEQ ID NO:128 or SEQ ID NO:129 or CDR1, the amino acid sequence set forth as SEQ ID NO:130 or SEQ ID NO:131 in CDR2, and the amino acid sequence set forth as SEQ ID NO:132 or SEQ ID NO:133 in CDR3;
(11) an amino acid sequence comprising the amino acid sequence set forth as SEQ ID NO:134 or SEQ ID NO:135 in CDR1, the amino acid sequence set forth as SEQ ID NO:136 or SEQ ID NO:137 in CDR2, and the amino acid sequence set forth as SEQ ID NO:138 or SEQ ID NO:139 in CDR3;
(12) an amino acid sequence comprising the amino acid sequence set forth as SEQ ID NO:140 or SEQ ID NO:141 in CDR1, the amino acid sequence set forth as SEQ ID NO:142 or SEQ ID NO:143 in CDR2, and the amino acid sequence set forth as SEQ ID NO:144 or SEQ ID NO:145 in CDR3;
(13) an amino acid sequence comprising the amino acid sequence set forth as SEQ ID NO:146 or SEQ ID NO:147 in CDR1, the amino acid sequence set forth as SEQ ID NO:148 or SEQ ID NO:149 in CDR2, and the amino acid sequence set forth as SEQ ID NO:150 or SEQ ID NO:151 in CDR3; and
(14) an amino acid sequence comprising the amino acid sequence set forth as SEQ ID NO:152 or SEQ ID NO:153 in CDR1, the amino acid sequence set forth as SEQ ID NO:154 or SEQ ID NO:155 in CDR2, and the amino acid sequence set forth as SEQ ID NO:156 or SEQ ID NO:157 in CDR3.
8. The anti-human transferrin receptor antibody according to 7 above, wherein the amino acid sequence of the heavy chain variable region of the antibody is selected from the group consisting of (1) to (14) below:
(1) an amino acid sequence comprising the amino acid sequence set forth as SEQ ID NO:76 in CDR1, the amino acid sequence set forth as SEQ ID NO:78 in CDR2, and the amino acid sequence set forth as SEQ ID NO:80 in CDR3;
(2) an amino acid sequence comprising the amino acid sequence set forth as SEQ ID NO:82 in CDR1, the amino acid sequence set forth as SEQ ID NO:84 in CDR2, and the amino acid sequence set forth as SEQ ID NO:86 in CDR3;
(3) an amino acid sequence comprising the amino acid sequence set forth as SEQ ID NO:88 in CDR1, the amino acid sequence set forth as SEQ ID NO:90 in CDR2, and the amino acid sequence set forth as SEQ ID NO:92 in CDR3;
(4) an amino acid sequence comprising the amino acid sequence set forth as SEQ ID NO:94 in CDR1, the amino acid sequence set forth as SEQ ID NO:96 in CDR2, and the amino acid sequence set forth as SEQ ID NO:98 in CDR3;
(5) an amino acid sequence comprising the amino acid sequence set forth as SEQ ID NO:100 in CDR1, the amino acid sequence set forth as SEQ ID NO:102 in CDR2, and the amino acid sequence set forth as SEQ ID NO:104 in CDR3;
(6) an amino acid sequence comprising the amino acid sequence set forth as SEQ ID NO:106 in CDR1, the amino acid sequence set forth as SEQ ID NO:108 in CDR2, and the amino acid sequence set forth as SEQ ID NO:109 in CDR3;
(7) an amino acid sequence comprising the amino acid sequence set forth as SEQ ID NO:111 in CDR1, the amino acid sequence set forth as SEQ ID NO:113 in CDR2, and the amino acid sequence set forth as SEQ ID NO:115 as CDR3;
(8) an amino acid sequence comprising the amino acid sequence set forth as SEQ ID NO:117 in CDR1, the amino acid sequence set forth as SEQ ID NO:119 in CDR2, and the amino acid sequence set forth as SEQ ID NO:120 in CDR3;
(9) an amino acid sequence comprising the amino acid sequence set forth as SEQ ID NO:122 in CDR1, the amino acid sequence set forth as SEQ ID NO:124 in CDR2, and the amino acid sequence set forth as SEQ ID NO:126 in CDR3;
(10) an amino acid sequence comprising the amino acid sequence set forth as SEQ ID NO:128 in CDR1, the amino acid sequence set forth as SEQ ID NO:130 in CDR2, and the amino acid sequence set forth as SEQ ID NO:132 in CDR3;
(11) an amino acid sequence comprising the amino acid sequence set forth as SEQ ID NO:134 in CDR1, the amino acid sequence set forth as SEQ ID NO:136 in CDR2, and the amino acid sequence set forth as SEQ ID NO:138 in CDR3;
(12) an amino acid sequence comprising the amino acid sequence set forth as SEQ ID NO:140 in CDR1, the amino acid sequence set forth as SEQ ID NO:142 in CDR2, and the amino acid sequence set forth as SEQ ID NO:144 in CDR3;
(13) an amino acid sequence comprising the amino acid sequence set forth as SEQ ID NO:146 in CDR1, the amino acid sequence set forth as SEQ ID NO:148 in CDR2, and the amino acid sequence set forth as SEQ ID NO:150 in CDR3; and
(14) an amino acid sequence comprising the amino acid sequence set forth as SEQ ID NO:152 in CDR1, the amino acid sequence set forth as SEQ ID NO:154 in CDR2, and the amino acid sequence set forth as SEQ ID NO:156 in CDR3.
9. An anti-human transferrin receptor antibody, wherein the amino acid sequence of CDR1, CDR2 and CDR3 in the heavy chain variable region thereof have a homology not lower than 80% to the amino acid sequences of CDR1, CDR2 and CDR3, respectively, in the heavy chain according to 7 or 8 above.
10. An anti-human transferrin receptor antibody, wherein the amino acid sequence of CDR1, CDR2 and CDR3 in the heavy chain variable region thereof have a homology not lower than 90% to the amino acid sequences of CDR1, CDR2 and CDR3, respectively, in the heavy chain according to 7 or 8 above.
11. An anti-human transferrin receptor antibody, wherein 1 to 5 amino acids are substituted, deleted or added relative to the amino acid sequence that forms at least one of the CDRs in the heavy chain according to 7 or 8 above.
12. An anti-human transferrin receptor antibody, wherein 1 to 3 amino acids are substituted, deleted or added relative to the amino acid sequence that forms at least one of the CDRs in the heavy chain according to 7 or 8 above.
13. An anti-human transferrin receptor antibody, wherein the light chain variable region and the heavy chain variable region thereof are selected from the group consisting of (1) to (14) below:
(1) the light chain variable region comprising the amino acid sequence set forth as SEQ ID NO:6 or SEQ ID NO:7 as CDR1, the amino acid sequence set forth as SEQ ID NO:8 or SEQ ID NO:9 or the amino acid sequence Trp-Thr-Ser as CDR2, and the amino acid sequence set forth as SEQ ID NO:10 as CDR3; and the heavy chain variable region comprising the amino acid sequence set forth as SEQ ID NO:76 or SEQ ID NO:77 as CDR1, the amino acid sequence set forth as SEQ ID NO:78 or SEQ ID NO:79 as CDR2, and the amino acid sequence set forth as SEQ ID NO:80 or SEQ ID NO:81 as CDR3;
(2) the light chain variable region comprising the amino acid sequence set forth as SEQ ID NO:11 or SEQ ID NO:12 as CDR1, the amino acid sequence set forth as SEQ ID NO:13 or SEQ ID NO:14 or the amino acid sequence Tyr-Ala-Ser as CDR2, and the amino acid sequence set forth as SEQ ID NO:15 as CDR3; and the heavy chain variable region comprising the amino acid sequence set forth as SEQ ID NO:82 or SEQ ID NO:83 as CDR1, the amino acid sequence set forth as SEQ ID NO:84 or SEQ ID NO:85 as CDR2, and the amino acid sequence set forth as SEQ ID NO:86 or SEQ ID NO:87 as CDR3;
(3) the light chain variable region comprising the amino acid sequence set forth as SEQ ID NO:16 or SEQ ID NO:17 as CDR1, the amino acid sequence set forth as SEQ ID NO:18 or SEQ ID NO:19 or the amino acid sequence Lys-Val-Ser as CDR2, and the amino acid sequence set forth as SEQ ID NO:20 as CDR3; and the heavy chain variable region comprising the amino acid sequence set forth as SEQ ID NO:88 or SEQ ID NO:89 as CDR1, the amino acid sequence set forth as SEQ ID NO:90 or SEQ ID NO:91 as CDR2, and the amino acid sequence set forth as SEQ ID NO:92 or SEQ ID NO:93 as CDR3;
(4) the light chain variable region comprising the amino acid sequence set forth as SEQ ID NO:21 or SEQ ID NO:22 as CDR1, the amino acid sequence set forth as SEQ ID NO:23 or SEQ ID NO:24 or the amino acid sequence Asp-Thr-Ser as CDR2, and the amino acid sequence set forth as SEQ ID NO:25 as CDR3; and the heavy chain variable region comprising the amino acid sequence set forth as SEQ ID NO:94 or SEQ ID NO:95 as CDR1, the amino acid sequence set forth as SEQ ID NO:96 or SEQ ID NO:97 as CDR2, and the amino acid sequence set forth as SEQ ID NO:98 or SEQ ID NO:99 as CDR3;
(5) the light chain variable region comprising the amino acid sequence set forth as SEQ ID NO:26 or SEQ ID NO:27 as CDR1, the amino acid sequence set forth as SEQ ID NO:28 or SEQ ID NO:29 or the amino acid sequence Asp-Thr-Ser as CDR2, and the amino acid sequence set forth as SEQ ID NO:30 as CDR3; and the heavy chain variable region comprising the amino acid sequence set forth as SEQ ID NO:100 or SEQ ID NO:101 as CDR1, the amino acid sequence set forth as SEQ ID NO:102 or SEQ ID NO:103 as CDR2, and the amino acid sequence set forth as SEQ ID NO:104 or SEQ ID NO:105 as CDR3;
(6) the light chain variable region comprising the amino acid sequence set forth as SEQ ID NO:31 or SEQ ID NO:32 as CDR1, the amino acid sequence set forth as SEQ ID NO:33 or SEQ ID NO:34 or the amino acid sequence Ala-Ala-Ser as CDR2, and the amino acid sequence set forth as SEQ ID NO:35 as CDR3; and the heavy chain variable region comprising the amino acid sequence set forth as SEQ ID NO:106 or SEQ ID NO:107 as CDR1, the amino acid sequence set forth as SEQ ID NO:108 or SEQ ID NO:278, as CDR2, and the amino acid sequence set forth as SEQ ID NO:109 or SEQ ID NO:110 as CDR3;
(7) the light chain variable region comprising the amino acid sequence set forth as SEQ ID NO:36 or SEQ ID NO:37 as CDR1, the amino acid sequence set forth as SEQ ID NO:38 or SEQ ID NO:39 or the amino acid sequence Gln-Thr-Ser as CDR2, and the amino acid sequence set forth as SEQ ID NO:40 as CDR3; and the heavy chain variable region comprising the amino acid sequence set forth as SEQ ID NO:111 or SEQ ID NO:112 as CDR1, the amino acid sequence set forth as SEQ ID NO:113 or SEQ ID NO:114 as CDR2, and the amino acid sequence set forth as SEQ ID NO:115 or SEQ ID NO:116 as CDR3;
(8) the light chain variable region comprising the amino acid sequence set forth as SEQ ID NO:41 or SEQ ID NO:42 as CDR1, the amino acid sequence set forth as SEQ ID NO:43 or SEQ ID NO:44 or the amino acid sequence Gly-Thr-Ser as CDR2, and the amino acid sequence set forth as SEQ ID NO:45 as CDR3; and the heavy chain variable region comprising the amino acid sequence set forth as SEQ ID NO:117 or SEQ ID NO:118 as CDR1, the amino acid sequence set forth as SEQ ID NO:119 or SEQ ID NO:279 as CDR2, and the amino acid sequence set forth as SEQ ID NO:120 or SEQ ID NO:121 as CDR3;
(9) the light chain variable region comprising the amino acid sequence set forth as SEQ ID NO:46 or SEQ ID NO:47 as CDR1, the amino acid sequence set forth as SEQ ID NO:48 or SEQ ID NO:49 or the amino acid sequence Phe-Thr-Ser as CDR2, and the amino acid sequence set forth as SEQ ID NO:50 as CDR3; and the heavy chain variable region comprising the amino acid sequence set forth as SEQ ID NO:122 or SEQ ID NO:123 as CDR1, the amino acid sequence set forth as SEQ ID NO:124 or SEQ ID NO:125 as CDR2, and the amino acid sequence set forth as SEQ ID NO:126 or SEQ ID NO:127 as CDR3;
(10) the light chain variable region comprising the amino acid sequence set forth as SEQ ID NO:51 or SEQ ID NO:52 as CDR1, the amino acid sequence set forth as SEQ ID NO:53 or SEQ ID NO:54 or the amino acid sequence Ala-Ala-Ser as CDR2, and the amino acid sequence set forth as SEQ ID NO:55 as CDR3; and the heavy chain variable region comprising the amino acid sequence set forth as SEQ ID NO:128 or SEQ ID NO:129 as CDR1, the amino acid sequence set forth as SEQ ID NO:130 or SEQ ID NO:131 as CDR2, and the amino acid sequence set forth as SEQ ID NO:132 or SEQ ID NO:133 as CDR 3;
(11) the light chain variable region comprising the amino acid sequence set forth as SEQ ID NO:56 or SEQ ID NO:57 as CDR1, the amino acid sequence set forth as SEQ ID NO:58 or SEQ ID NO:59 or the amino acid sequence Tyr-Ala-Ser as CDR2, and the amino acid sequence set forth as SEQ ID NO:60 as CDR3; and the heavy chain variable region comprising the amino acid sequence set forth as SEQ ID NO:134 or SEQ ID NO:135 as CDR1, the amino acid sequence set forth as SEQ ID NO:136 or SEQ ID NO:137 as CDR2, and the amino acid sequence set forth as SEQ ID NO:138 or SEQ ID NO:139 as CDR3;
(12) the light chain variable region comprising the amino acid sequence set forth as SEQ ID NO:61 or SEQ ID NO:62 as CDR1, the amino acid sequence set forth as SEQ ID NO:63 or SEQ ID NO:64 or the amino acid sequence Trp-Ser-Ser as CDR2, and the amino acid sequence set forth as SEQ ID NO:65 as CDR3; and the heavy chain variable region comprising the amino acid sequence set forth as SEQ ID NO:140 or SEQ ID NO:141 as CDR1, the amino acid sequence set forth as SEQ ID NO:142 or SEQ ID NO:143 as CDR2, and the amino acid sequence set forth as SEQ ID NO:144 or SEQ ID NO:145 as CDR3;
(13) the light chain variable region comprising the amino acid sequence set forth as SEQ ID NO:66 or SEQ ID NO:67 as CDR1, the amino acid sequence set forth as SEQ ID NO:68 or SEQ ID NO:69 or the amino acid sequence Tyr-Ala-Ser as CDR2, and the amino acid sequence set forth as SEQ ID NO:70 as CDR3; and the heavy chain variable region comprising the amino acid sequence set forth as SEQ ID NO:146 or SEQ ID NO:147 as CDR1, the amino acid sequence set forth as SEQ ID NO:148 or SEQ ID NO:149 as CDR2, and the amino acid sequence set forth as SEQ ID NO:150 or SEQ ID NO:151 as CDR3; and
(14) the light chain variable region comprising the amino acid sequence set forth as SEQ ID NO:71 or SEQ ID NO:72 as CDR1, the amino acid sequence set forth as SEQ ID NO:73 or SEQ ID NO:74 or the amino acid sequence Asp-Thr-Ser as CDR2, and the amino acid sequence set forth as SEQ ID NO:75 as CDR3; and the heavy chain variable region comprising the amino acid sequence set forth as SEQ ID NO:152 or SEQ ID NO:153 as CDR1, the amino acid sequence set forth as SEQ ID NO:154 or SEQ ID NO:155 as CDR2, and the amino acid sequence set forth as SEQ ID NO:156 or SEQ ID NO:157 as CDR3.
14. The anti-human transferrin receptor antibody according to 13 above, wherein the light chain variable region and the heavy chain variable region thereof are selected from the group consisting of (1) to (14) below:
(1) the light chain variable region comprising the amino acid sequence set forth as SEQ ID NO:6 as CDR1, the amino acid sequence set forth as SEQ ID NO:8 as CDR2, and the amino acid sequence set forth as SEQ ID NO:10 as CDR3; and the heavy chain variable region comprising the amino acid sequence set forth as SEQ ID NO:76 as CDR1, the amino acid sequence set forth as SEQ ID NO:78 as CDR2, and the amino acid sequence set forth as SEQ ID NO:80 as CDR3;
(2) the light chain variable region comprising the amino acid sequence set forth as SEQ ID NO:11 as CDR1, the amino acid sequence set forth as SEQ ID NO:13 as CDR2, and the amino acid sequence set forth as SEQ ID NO:15 as CDR3; and the heavy chain variable region comprising the amino acid sequence set forth as SEQ ID NO:82 as CDR1, the amino acid sequence set forth as SEQ ID NO:84 as CDR2, and the amino acid sequence set forth as SEQ ID NO:86 as CDR3;
(3) the light chain variable region comprising the amino acid sequence set forth as SEQ ID NO:16 as CDR1, the amino acid sequence set forth SEQ ID NO:18 as CDR2, and the amino acid sequence set forth as SEQ ID NO:20 as CDR3; and the heavy chain variable region comprising the amino acid sequence set forth as SEQ ID NO:88 as CDR1, the amino acid sequence set forth as SEQ ID NO:90 as CDR2, and the amino acid sequence set forth as SEQ ID NO:92 as CDR3;
(4) the light chain variable region comprising the amino acid sequence set forth as SEQ ID NO:21 as CDR1, the amino acid sequence set forth as SEQ ID NO:23 as CDR2, and the amino acid sequence set forth as SEQ ID NO:25 as CDR3; and the heavy chain variable region comprising the amino acid sequence set forth as SEQ ID NO:94 as CDR1, the amino acid sequence set forth as SEQ ID NO:96 as CDR2, and the amino acid sequence set forth as SEQ ID NO:98 as CDR3;
(5) the light chain variable region comprising the amino acid sequence set forth as SEQ ID NO:26 as CDR1, the amino acid sequence set forth as SEQ ID NO:28 as CDR2, and the amino acid sequence set forth as SEQ ID NO:30 as CDR3; and the heavy chain variable region comprising the amino acid sequence set forth as SEQ ID NO:100 as CDR1, the amino acid sequence set forth as SEQ ID NO:102 as CDR2, and the amino acid sequence set forth as SEQ ID NO:104 as CDR3;
(6) the light chain variable region comprising the amino acid sequence set forth as SEQ ID NO:31 as CDR1, the amino acid sequence set forth as SEQ ID NO:33 as CDR2, and the amino acid sequence set forth as SEQ ID NO:35 as CDR3; and the heavy chain variable region comprising the amino acid sequence set forth as SEQ ID NO:106 as CDR1, the amino acid sequence set forth as SEQ ID NO:108 as CDR2, and the amino acid sequence set forth as SEQ ID NO:109 as CDR3;
(7) the light chain variable region comprising the amino acid sequence set forth as SEQ ID NO:36 as CDR1, the amino acid sequence set forth as SEQ ID NO:38 as CDR2, and the amino acid sequence set forth as SEQ ID NO:40 as CDR3; and the heavy chain variable region comprising the amino acid sequence set forth as SEQ ID NO:111 as CDR1, the amino acid sequence set forth as SEQ ID NO:113 as CDR2, and the amino acid sequence set forth as SEQ ID NO:115 as CDR3;
(8) the light chain variable region comprising the amino acid sequence set forth as SEQ ID NO:41 as CDR1, the amino acid sequence set forth as SEQ ID NO:43 as CDR2, and the amino acid sequence set forth as SEQ ID NO:45 as CDR3; and the heavy chain variable region comprising the amino acid sequence set forth as SEQ ID NO:117 as CDR1, the amino acid sequence set forth as SEQ ID NO:119 as CDR2, and the amino acid sequence set forth as SEQ ID NO:120 as CDR 3;
(9) the light chain variable region comprising the amino acid sequence set forth as SEQ ID NO:46 as CDR1, the amino acid sequence set forth as SEQ ID NO:48 as CDR2, and the amino acid sequence set forth as SEQ ID NO:50 as CDR3; and the heavy chain variable region comprising the amino acid sequence set forth as SEQ ID NO:122 as CDR1, the amino acid sequence set forth as SEQ ID NO:124 as CDR2, and the amino acid sequence set forth as SEQ ID NO:126 as CDR3;
(10) the light chain variable region comprising the amino acid sequence set forth as SEQ ID NO:51 as CDR1, the amino acid sequence set forth as SEQ ID NO:53 as CDR2, and the amino acid sequence set forth as SEQ ID NO:55 as CDR3; and the heavy chain variable region comprising the amino acid sequence set forth as SEQ ID NO:128 as CDR1, the amino acid sequence set forth as SEQ ID NO:130 as CDR2, and the amino acid sequence set forth as SEQ ID NO:132 as CDR3;
(11) the light chain variable region comprising the amino acid sequence set forth as SEQ ID NO:56 as CDR1, the amino acid sequence set forth as SEQ ID NO:58 as CDR2, and the amino acid sequence set forth as SEQ ID NO:60 as CDR3; and the heavy chain variable region comprising the amino acid sequence set forth as SEQ ID NO:134 as CDR1, the amino acid sequence set forth as SEQ ID NO:136 as CDR2, and the amino acid sequence set forth as SEQ ID NO:138 as CDR3;
(12) the light chain variable region comprising the amino acid sequence set forth as SEQ ID NO:61 as CDR1, the amino acid sequence set forth as SEQ ID NO:63 as CDR2, and the amino acid sequence set forth as SEQ ID NO:65 as CDR3; and the heavy chain variable region comprising the amino acid sequence set forth as SEQ ID NO:140 as CDR1, the amino acid sequence set forth as SEQ ID NO:142 as CDR2, and the amino acid sequence set forth as SEQ ID NO:144 as CDR3;
(13) the light chain variable region comprising the amino acid sequence set forth as SEQ ID NO:66 as CDR1, the amino acid sequence set forth as SEQ ID NO:68 as CDR2, and the amino acid sequence set forth as SEQ ID NO:70 as CDR3; and the heavy chain variable region comprising the amino acid sequence set forth as SEQ ID NO:146 as CDR1, the amino acid sequence set forth as SEQ ID NO:148 as CDR2, and the amino acid sequence set forth as SEQ ID NO:150 as CDR3; and
(14) the light chain variable region comprising the amino acid sequence set forth as SEQ ID NO:71 as CDR1, the amino acid sequence set forth as SEQ ID NO:73 as CDR2, and the amino acid sequence set forth as SEQ ID NO:75 as CDR3; and the heavy chain variable region comprising the amino acid sequence set forth as SEQ ID NO:152 as CDR1, the amino acid sequence set forth as SEQ ID NO:154 as CDR2, and the amino acid sequence set forth as SEQ ID NO:156 as CDR3.
15. An anti-human transferrin receptor antibody, wherein the amino acid sequence of each of CDR1, CDR2 and CDR3 in the light chain and the heavy chain thereof has a homology not lower than 80% to the amino acid sequence of the CDR1, CDR2 and CDR3, of one of the combinations of the light chain and the heavy chain according to 13 or 14 above.
16. An anti-human transferrin receptor antibody, wherein the amino acid sequence of each of CDR1, CDR2 and CDR3 in the light chain and the heavy chain thereof has a homology not lower than 90% to the amino acid sequence of the CDR1, CDR2 and CDR3, of one of the combinations of the light chain and the heavy chain according to 13 or 14 above.
17. An anti-human transferrin receptor antibody, wherein 1 to 5 amino acids are substituted, deleted or added relative to the amino acid sequence that forms at least one CDR in each of the light chain and the heavy chain of one of the combinations of the light chain and the heavy chain according to 13 or 14 above.
18. An anti-human transferrin receptor antibody, wherein 1 to 3 amino acids are substituted, deleted or added relative to the amino acid sequence that forms at least one CDR in each of the light chain and the heavy chain of one of the combinations of the light chain and the heavy chain according to 13 or 14 above.
19. An anti-human transferrin receptor antibody, wherein the light chain variable region of the antibody comprises an amino acid sequence selected from the group consisting of the amino acid sequences set forth as SEQ ID NO:158, SEQ ID NO:159, SEQ ID NO:160, SEQ ID NO:161, SEQ ID NO:162, and SEQ ID NO:163; and wherein the heavy chain variable region of the antibody comprises an amino acid sequence selected from the group consisting of the amino acid sequences set forth as SEQ ID NO:166, SEQ ID NO:167, SEQ ID NO:168, SEQ ID NO:169, SEQ ID NO:170, and SEQ ID NO:171.
20. An anti-human transferrin receptor antibody, wherein the light chain variable region of the antibody comprises an amino acid sequence selected from the group consisting of the amino acid sequences set forth as SEQ ID NO:174, SEQ ID NO:175, SEQ ID NO:176, SEQ ID NO:177, SEQ ID NO:178, and SEQ ID NO:179; and wherein the heavy chain variable region of the antibody comprises an amino acid sequence selected from the group consisting of the amino acid sequences set forth as SEQ ID NO:182, SEQ ID NO:183, SEQ ID NO:184, SEQ ID NO:185, SEQ ID NO:186, and SEQ ID NO:187.
21. An anti-human transferrin receptor antibody, wherein the light chain variable region of the antibody comprises an amino acid sequence selected from the group consisting of the amino acid sequences set forth as SEQ ID NO:190, SEQ ID NO:191, SEQ ID NO:192, SEQ ID NO:193, SEQ ID NO:194, and SEQ ID NO:195, and wherein the heavy chain variable region of the antibody comprises an amino acid sequence selected from the group consisting of the amino acid sequences set forth as SEQ ID NO:204, SEQ ID NO:205, SEQ ID NO:206, SEQ ID NO:207, SEQ ID NO:208, and SEQ ID NO:209.
22. An anti-human transferrin receptor antibody selected from the group consisting of (1) to (6) below:
(1) an antibody, wherein the light chain variable region thereof comprises the amino acid sequence set forth as SEQ ID NO:163; and wherein the heavy chain variable region thereof comprises the amino acid sequence set forth as SEQ ID NO:171,
(2) an antibody, wherein the light chain variable region thereof comprises the amino acid sequence set forth as SEQ ID NO:179; and wherein the heavy chain variable region thereof comprises the amino acid sequence set forth as SEQ ID NO:187,
(3) an antibody, wherein the light chain variable region thereof comprises the amino acid sequence set forth as SEQ ID NO:191; and wherein the heavy chain variable region thereof comprises the amino acid sequence set forth as SEQ ID NO:205,
(4) an antibody, wherein the light chain variable region thereof comprises the amino acid sequence set forth as SEQ ID NO:193; and wherein the heavy chain variable region thereof comprises the amino acid sequence set forth as SEQ ID NO:205,
(5) an antibody, wherein the light chain variable region thereof comprises the amino acid sequence set forth as SEQ ID NO:194; and wherein the heavy chain variable region thereof comprises the amino acid sequence set forth as SEQ ID NO:205, and
(6) an antibody, wherein the light chain variable region thereof comprises the amino acid sequence set forth as SEQ ID NO:195; and wherein the heavy chain variable region thereof comprises the amino acid sequence set forth as SEQ ID NO:205.
23. An anti-human transferrin receptor antibody selected from the group consisting of (1) to (10) below:
(1) an antibody, wherein the light chain thereof comprises the amino acid sequence set forth as SEQ ID NO:164; and wherein the heavy chain thereof comprises the amino acid sequence set forth as SEQ ID NO:172,
(2) an antibody, wherein the light chain thereof comprises the amino acid sequence set forth as SEQ ID NO:180; and wherein the heavy chain thereof comprises the amino acid sequence set forth as SEQ ID NO:188,
(3) an antibody, wherein the light chain thereof comprises the amino acid sequence set forth as SEQ ID NO:196; and wherein the heavy chain thereof comprises the amino acid sequence set forth as SEQ ID NO:210,
(4) an antibody, wherein the light chain thereof comprises the amino acid sequence set forth as SEQ ID NO:198; and wherein the heavy chain thereof comprises the amino acid sequence set forth as SEQ ID NO:210,
(5) an antibody, wherein the light chain thereof comprises the amino acid sequence set forth as SEQ ID NO:200; and wherein the heavy chain thereof comprises the amino acid sequence set forth as SEQ ID NO:210,
(6) an antibody, wherein the light chain thereof comprises the amino acid sequence set forth as SEQ ID NO:202; and wherein the heavy chain thereof comprises the amino acid sequence set forth as SEQ ID NO:210,
(7) an antibody, wherein the light chain thereof comprises the amino acid sequence set forth as SEQ ID NO:196; and wherein the heavy chain thereof comprises the amino acid sequence set forth as SEQ ID NO:212,
(8) an antibody, wherein the light chain thereof comprises the amino acid sequence set forth as SEQ ID NO:198; and wherein the heavy chain thereof comprises the amino acid sequence set forth as SEQ ID NO:212,
(9) an antibody, wherein the light chain thereof comprises the amino acid sequence set forth as SEQ ID NO:200; and wherein the heavy chain thereof comprises the amino acid sequence set forth as SEQ ID NO:212, and
(10) an antibody, wherein the light chain thereof comprises the amino acid sequence set forth as SEQ ID NO:202; and wherein the heavy chain thereof comprises the amino acid sequence set forth as SEQ ID NO:212.
24 An anti-human transferrin receptor antibody having a homology not lower than 80% to the anti-human transferrin receptor antibody according to any one of 19 to 23 above for the amino acid sequence of the light chain variable region and the amino acid sequence of the heavy chain variable region.
25. An anti-human transferrin receptor antibody having a homology not lower than 90% to the anti-human transferrin receptor antibody according to any one of 19 to 23 above for the amino acid sequence of the light chain variable region and the amino acid sequence of the heavy chain variable region.
26. An anti-human transferrin receptor antibody, wherein 1 to 10 amino acids are substituted, deleted or added relative to the amino acid sequence that forms the light chain variable region of the anti-human transferrin receptor antibody according to any one of 19 to 23 above.
27. An anti-human transferrin receptor antibody, wherein 1 to 3 amino acids are substituted, deleted or added relative to the amino acid sequence that forms the light chain variable region of the anti-human transferrin receptor antibody according to any one of 19 to 23 above.
28. An anti-human transferrin receptor antibody, wherein 1 to 10 amino acids are substituted, deleted or added relative to the amino acid sequence that forms the heavy chain variable region of the anti-human transferrin receptor antibody according to any one of 19 to 23 above.
29 An anti-human transferrin receptor antibody, wherein 1 to 3 amino acids are substituted, deleted or added relative to the amino acid sequence that forms the heavy chain variable region of the anti-human transferrin receptor antibody according to any one of 19 to 23 above.
30 An anti-human transferrin receptor antibody, wherein 1 to 10 amino acids are substituted, deleted or added relative to the respective amino acid sequences that form the light chain variable region and the heavy chain variable region of the anti-human transferrin receptor antibody according to any one of 19 to 23 above.
31. An anti-human transferrin receptor antibody, wherein 1 to 3 amino acids are substituted, deleted or added relative to the respective amino acid sequences that form the light chain variable region and the heavy chain variable region of the anti-human transferrin receptor antibody according to any one of 19 to 23 above.
32 The anti-human transferrin receptor antibody according to any one of 1 to 31 above, wherein the antibody has an affinity to both the extracellular region of human transferrin receptor and the extracellular region of monkey transferrin receptor.
33 The anti-human transferrin receptor antibody according to 32 above, wherein the dissociation constant of its complex with the extracellular region of human transferrin receptor is not greater than 1×10⁻⁸ M, and the dissociation constant of its complex with the extracellular region of monkey transferrin receptor is not greater than 5×10⁻⁸ M.
34 The anti-human transferrin receptor antibody according to any one of 1 to 33 above, wherein the antibody is Fab antibody, F(ab′)₂ antibody, or F(ab′) antibody.
35. The anti-human transferrin receptor antibody according to any one of 1 to 33 above, wherein the antibody is a single-chain antibody selected from the group consisting of scFab, scF(ab′), scF(ab′)₂ and scFv.
36. The anti-human transferrin receptor antibody according to 35 above, wherein the light chain and the heavy chain thereof are linked via a linker sequence.
37 The anti-human transferrin receptor antibody according to 35 above, wherein the heavy chain is linked, via a linker sequence, to the light chain on the C-terminal side thereof.
38 The anti-human transferrin receptor antibody according to 35 above, wherein the light chain is linked, via a linker sequence, to the heavy chain on the C-terminal side thereof.
39. The anti-human transferrin receptor antibody according to any one of 36 to 38 above, wherein the linker sequence consists of 8 to 50 amino acid residues.
40. The anti-human transferrin receptor antibody according to 39 above, wherein the linker sequence is selected from the group consisting of the amino acid sequence Gly-Ser, the amino acid sequence Gly-Gly-Ser, the amino acid sequence Gly-Gly-Gly, the amino acid sequences set forth as SEQ ID NO:3, SEQ ID NO:4, and SEQ ID NO:5, and the amino acid sequence consisting of three consecutively linked amino acid sequences each set forth as SEQ ID NO:3.
41. A fusion protein comprising the anti-human transferrin receptor antibody according to any one of 1 to 40 above and the amino acid sequence of a different protein (A) linked to the light chain of the antibody on the C-terminal side or the N-terminal side thereof.
42. A fusion protein comprising an anti-human transferrin receptor antibody and a different protein (A),
wherein the anti-human transferrin receptor antibody is the anti-human transferrin receptor antibody according to any one of 1 to 40 above, and
wherein the different protein (A) is linked to the light chain of the anti-human transferrin receptor antibody on the C-terminal side or the N-terminal side thereof.
43. The fusion protein according to 41 or 42 above, wherein the different protein (A) is linked, via a linker sequence, to the light chain on the C-terminal side or the N-terminal side thereof.
44. The fusion protein according to 43 above, wherein the linker sequence consists of 1 to 50 amino acid residues.
45. The fusion protein according to 44 above, wherein the linker sequence comprises an amino acid sequence selected from the group consisting of a single glycine, a single serine, the amino acid sequence Gly-Ser, the amino acid sequence Gly-Gly-Ser, the amino acid sequence set forth as SEQ ID NO:3, the amino acid sequence set forth as SEQ ID NO:4, the amino acid sequence set forth as SEQ ID NO:5, and the amino acid sequences consisting of 1 to 10 thereof that are consecutively linked.
46. A fusion protein comprising the anti-human transferrin receptor antibody according to any one of 1 to 40 above and the amino acid sequence of a different protein (A) linked to the heavy chain of the antibody on the C-terminal side or the N-terminal side thereof.
47. A fusion protein of an anti-human transferrin receptor antibody and a different protein (A),
wherein the anti-human transferrin receptor antibody is the anti-human transferrin receptor antibody according to any one of 1 to 40 above, and
wherein the different protein (A) is linked to the heavy chain of the anti-human transferrin receptor antibody on the C-terminal side or the N-terminal side thereof.
48. The fusion protein according to 46 or 47 above, wherein the different protein (A) is linked, via a linker sequence, to the heavy chain on the C-terminal side or the N-terminal side thereof.
49. The fusion protein according to 48 above, wherein the linker sequence consists of 1 to 50 amino acid residues.
50. The fusion protein according to 48 above, wherein the linker sequence comprises an amino acid sequence selected from the group consisting of a single glycine, a single serine, the amino acid sequence Gly-Ser, the amino acid sequence Gly-Gly-Ser, the amino acid sequence set forth as SEQ ID NO:3, the amino acid sequence set forth as SEQ ID NO:4, the amino acid sequence set forth as SEQ ID NO:5, and the amino acid sequences consisting of 1 to 10 thereof that are consecutively linked.
51. The fusion protein according to any one of 41 to 50 above, wherein the different protein (A) is a protein originating from human.
52. The fusion protein according to any one of 41 to 51 above, wherein the different protein (A) is selected from the group consisting of nerve growth factor (NGF), lysosomal enzymes, ciliary neurotrophic factor (CNTF), glial cell line-derived neurotrophic factor (GDNF), neurotrophin-3, neurotrophin-4/5, neurotrophin-6, neuregulin-1, erythropoietin, darbepoetin, activin, basic fibroblast growth factor (bFGF), fibroblast growth factor 2 (FGF2), epidermal growth factor (EGF), vascular endothelial growth factor (VEGF), interferon α, interferon β, interferon γ, interleukin 6, granulocyte-macrophage colony stimulating factor (GM-CSF), granulocyte colony stimulating factor (G-CSF), macrophage colony stimulating factor (M-CSF), cytokines, tumor necrosis factor α receptor (TNF-α receptor), PD-1 ligands, enzymes having β-amyloid-degrading activity, anti-β-amyloid antibody, anti-BACE antibody, anti-EGFR antibody, anti-PD-1 antibody, anti-PD-L1 antibody, anti-HER2 antibody, anti-TNF-α antibody, and other antibody medicines.
53. The fusion protein according to any one of 41 to 51 above, wherein the different protein (A) is a lysosomal enzyme, and wherein the lysosomal enzyme is selected from the group consisting of α-L-iduronidase, iduronate 2-sulfatase, glucocerebrosidase, β-galactosidase, GM2 activator protein, β-hexosaminidase A, β-hexosaminidase B, N-acetylglucosamine-1-phosphotransferase, α-mannosidase, β-mannosidase, galactosylceramidase, saposin C, arylsulfatase A, α-L-fucosidase, aspartylglucosaminidase, α-N-acetylgalactosaminidase, acidic sphingomyelinase, α-galactosidase A, β-glucuronidase, heparan N-sulfatase, α-N-acetylglucosaminidase, acetyl CoA: α-glucosaminide N-acetyltransferase, N-Acetylglucosamine-6-sulfate sulfatase, acid ceramidase, amylo-1,6-glucosidase, sialidase, palmitoyl protein thioesterase 1, tripeptidyl-peptidase 1, hyaluronidase 1, CLN1 and CLN2.
54. The fusion protein according to any one of 41 to 51 above, wherein the different protein (A) is iduronate 2-sulfatase.
55. The fusion protein according to 50 above, wherein the different protein (A) is human iduronate 2-sulfatase, and wherein the fusion protein is selected from (1) to (3) below:
(1) the fusion protein, wherein the light chain of the humanized anti-hTfR antibody has the amino acid sequence set forth as SEQ ID NO:164, and wherein the heavy chain of the humanized anti-hTfR antibody is linked, on the C-terminal side thereof and via a linker sequence Gly-Ser, to the human iduronate 2-sulfatase, and the whole linked heavy chain has the amino acid sequence set forth as SEQ ID NO:247.
(2) the fusion protein, wherein the light chain of the humanized anti-hTfR antibody has the amino acid sequence set forth as SEQ ID NO:180, and wherein the heavy chain of the humanized anti-hTfR antibody is linked, on the C-terminal side thereof and via a linker sequence Gly-Ser, to the human iduronate 2-sulfatase, and the whole linked heavy chain has the amino acid sequence set forth as SEQ ID NO:249, and
(3) the fusion protein, wherein the light chain of the humanized anti-hTfR antibody has the amino acid sequence set forth as SEQ ID NO:196, and wherein the heavy chain of the humanized anti-hTfR antibody is linked, on the C-terminal side thereof and via a linker sequence Gly-Ser, to the human iduronate 2-sulfatase, and the whole linked heavy chain has the amino acid sequence set forth as SEQ ID NO:251.
56. The fusion protein according to 50 above, wherein the different protein (A) is human iduronate 2-sulfatase, and wherein the fusion protein is selected from (1) to (3) below:
(1) the fusion protein comprising: the humanized anti-hTfR antibody light chain having the amino acid sequence set forth as SEQ ID NO:164; and the humanized anti-hTfR antibody heavy chain having the amino acid sequence set forth as SEQ ID NO:172 which is linked, on the C-terminal side thereof and via a linker sequence Gly-Ser, to the human iduronate 2-sulfatase set forth as SEQ ID NO:246;
(2) the fusion protein comprising: the humanized anti-hTfR antibody light chain having the amino acid sequence set forth as SEQ ID NO:180; and the humanized anti-hTfR antibody heavy chain having the amino acid sequence set forth as SEQ ID NO:188 which is linked, on the C-terminal side thereof and via a linker sequence Gly-Ser, to the human iduronate 2-sulfatase set forth as SEQ ID NO:246;
(3) the fusion protein comprising: the humanized anti-hTfR antibody light chain having the amino acid sequence set forth as SEQ ID NO:196; and the humanized anti-hTfR antibody heavy chain having the amino acid sequence set forth as SEQ ID NO:210 which is linked, on the C-terminal side thereof and via a linker sequence Gly-Ser, to the human iduronate 2-sulfatase set forth as SEQ ID NO:246.
57. The fusion protein according to 50 above, wherein the different protein (A) is human iduronate 2-sulfatase, and wherein the fusion protein is selected from (1) to (3) below:
(1) the fusion protein comprising: the human iduronate 2-sulfatase linked via a linker sequence Gly-Ser to the heavy chain on the C-terminal side thereof; and the light chain, wherein the amino acid sequence of the former is set forth as SEQ ID NO:247, and the amino acid sequence of the latter is set forth as SEQ ID NO:164,
(2) the fusion protein comprising: the human iduronate 2-sulfatase linked via a linker sequence Gly-Ser to the heavy chain on the C-terminal side thereof; and the light chain, wherein the amino acid sequence of the former is set forth as SEQ ID NO:249, and the amino acid sequence of the latter is set forth as SEQ ID NO:180, and
(3) the fusion protein comprising: the human iduronate 2-sulfatase linked via a linker sequence Gly-Ser to the heavy chain on the C-terminal side thereof; and the light chain, wherein the amino acid sequence of the former is set forth as SEQ ID NO:251, and the amino acid sequence of the latter is set forth as SEQ ID NO:196.
58. A DNA fragment encoding the amino acid sequence of the anti-human transferrin receptor antibody according to any one of 1 to 40 above.
59. A DNA fragment encoding the amino acid sequence of the fusion protein according to any one of 41 to 57 above.
60. An expression vector comprising the DNA fragment according to 58 or 59 above that is incorporated therein.
61. A mammalian cell transformed with the expression vector according to 60 above.
62. An anti-human transferrin receptor antibody-pharmacologically active compound complex, wherein the light chain and/or the heavy chain of the anti-human transferrin receptor antibody according to any one of 1 to 40 above is linked to a low-molecular-weight pharmacologically active compound that needs to be allowed to pass through the blood-brain barrier and exhibit the function thereof in the brain.
63. The anti-human transferrin receptor antibody according to 62 above, wherein the pharmacologically active compound is selected from the group consisting of anticancer drug, therapeutic agent for Alzheimer's disease, therapeutic agent for Parkinson's disease, therapeutic agent for Huntington's disease, therapeutic agent for schizophrenia, antidepressant, therapeutic agent for multiple sclerosis, therapeutic agent for amyotrophic lateral sclerosis, therapeutic agent for tumors of central nervous system including brain tumor, therapeutic agent for lysosomal storage disease accompanied by encephalopathy, therapeutic agent for glycogenosis, therapeutic agent for muscular dystrophy, therapeutic agent for cerebral ischemia, therapeutic agent for prion diseases, therapeutic agent for traumatic central nervous system disorders, therapeutic agent for viral and bacterial central nervous system diseases, pharmaceutical agent used for recovery after brain surgery, pharmaceutical agent used for recovery after spinal surgery, siRNA, antisense DNA, and peptide.
64. Use of the anti-human transferrin receptor antibody according to any one of 1 to 40 above for allowing the different protein (A) or a low-molecular-weight pharmacologically active compound to pass through the blood-brain barrier and exhibit the function thereof in the brain.
65. Use of the anti-human transferrin receptor antibody according to any one of 1 to 40 above for the manufacture of a medicament for parenteral administration for the treatment of a disease condition of the central nervous system, by linking thereto the molecule of a physiologically active protein or a pharmacologically active low-molecular-weight compound for the disease condition.
66. A method for treatment of a disorder of the central nervous system comprising parenterally administering to a patient with the disorder a therapeutically effective amount of the physiologically active protein, or pharmacologically active low-molecular-weight compound, for the disorder, in the form of a conjugate with the molecule of the anti-human transferrin receptor antibody according to any one of 1 to 40 above.
67. Use of the anti-human transferrin receptor antibody according to any one of 54 to 57 above for making human iduronate 2-sulfatase pass through the blood-brain barrier and exhibit the function thereof in the brain.
68. Use of the fusion protein according to any one of 54 to 57 for the production of a pharmaceutical agent for parenteral administration for the treatment of a disease condition of the central nervous system accompanying Hunter syndrome.
69. A method for the treatment of a disease of the central nervous system accompanying Hunter syndrome comprising parenterally administering a therapeutically effective amount of the fusion protein according to any one of 54 to 57 above to a patient with the disease.

### Effects of the Invention

By the present invention, various compounds, such as proteins and low-molecular-weight compounds that, although physiologically or pharmacologically active, have been unusable by parenteral administration because of their no or little ability to pass through the blood-brain barrier, can be provided in the form that allow them to pass through the blood-brain barrier, thus making them new pharmaceutical agents for parenteral administration for the treatment of a disease condition of the central nervous system.

### BRIEF DESCRIPTION OF DRAWINGS

FIG. 1 Substitute photographs for drawings showing the result of the immunohistochemical staining of the anti-hTfR antibody in the cerebral cortex of a cynomolgus monkey after a single intravenous administration of the anti-hTfR antibody. (a) anti-hTfR antibody not administered, (b) anti-hTfR antibody No. 1 administered, (c) anti-hTfR antibody No. 2 administered, (d) anti-hTfR antibody No. 3 administered. The bar at the bottom right in each photograph is a 50-μm gauge.

FIG. 2 A figure showing the result of the immunohistochemical staining of the anti-hTfR antibody in the hippocampus of a cynomolgus monkey after a single intravenous administration of the anti-hTfR antibody. (a) anti-hTfR antibody non-administered, (b) anti-hTfR antibody No. 1 administered, (c) anti-hTfR antibody No. 2 administered, (d) anti-hTfR antibody No. 3 administered. The bar at the bottom right in each photograph is a 50-μm gauge.

FIG. 3 Substitute photographs for drawings showing the result of the immunohistochemical staining of the anti-hTfR antibody in the cerebellum of a cynomolgus monkey after a single intravenous administration of the anti-hTfR antibody. (a) anti-hTfR antibody not administered, (b) anti-hTfR antibody No. 1 administered, (c) anti-hTfR antibody No. 2 administered, (d) anti-hTfR antibody No. 3 administered. The bar at the bottom right in each photograph is a 50-μm gauge.

FIG. 4 A figure showing the amount of a humanized anti-hTfR antibody accumulated in various organs other than the brain of a cynomolgus monkey after a single intravenous administration. The vertical axis indicates the amount of the humanized anti-hTfR antibody (μg/g wet weight) per wet weight of each organ. The white bars represent, from the left, the amount accumulated in each organ of the monkey after administration of humanized anti-hTfR antibody No. 3, humanized anti-hTfR antibody No. 3-2, humanized anti-hTfR antibody No. 3 (IgG4), and humanized anti-hTfR antibody No. 3-2 (IgG4), respectively, and the black bars represent the amount accumulated in respective organs of the monkey after administration of trastuzumab (Herceptin™). “ND” denotes “not detected”.

FIG. 5 Substitute photographs for drawings showing the result of immunohistochemical staining of a humanized anti-hTfR antibody in the cerebral cortex of a cynomolgus monkey after a single intravenous administration. (a) Herceptin administered, (b) humanized anti-hTfR antibody No. 3 administered, (c) humanized anti-hTfR antibody No. 3-2 administered, (d) humanized anti-hTfR antibody No. 3 (IgG4) administered, (e) humanized anti-hTfR antibody No. 3-2 (IgG4) administered. The bar at the bottom right in each photograph is a 20-μm gauge.

FIG. 6 Substitute photographs for drawing showing the result of immunohistochemical staining of a humanized anti-hTfR antibody in the hippocampus of a cynomolgus monkey after a single intravenous administration. (a) Herceptin administered, (b) humanized anti-hTfR antibody No. 3 administered, (c) humanized anti-hTfR antibody No. 3-2 administered, (d) humanized anti-hTfR antibody No. 3 (IgG4) administered, (e) humanized anti-hTfR antibody No. 3-2 (IgG4) administered. The bar at the bottom right at each photograph is a 20-μm gauge.

FIG. 7 A figure showing the result of immunohistochemical staining of humanized anti-hTfR antibody in the cerebellum of a cynomolgus monkey after a single intravenous administration. (a) Herceptin administered, (b) humanized anti-hTfR antibody No. 3 administered, (c) humanized anti-hTfR antibody No. 3-2 administered, (d) humanized anti-hTfR antibody No. 3 (IgG4) administered, (e) humanized anti-hTfR antibody No. 3-2 (IgG4) administered. The bar at the bottom right at each photograph is a 20-μm gauge.

FIG. 8 A figure showing the result of immunohistochemical staining of humanized anti-hTfR antibody in the medulla oblongata of a cynomolgus monkey after a single intravenous administration. (a) Herceptin administered, (b) humanized anti-hTfR antibody No. 3 administered, (c) humanized anti-hTfR antibody No. 3-2 administered, (d) humanized anti-hTfR antibody No. 3 (IgG4) administered, (e) humanized anti-hTfR antibody No. 3-2 (IgG4) administered. The bar at the bottom right at each photograph is a 20-μm gauge.

FIG. 9 A figure showing the amount of I2S-anti-hTfR antibody and rhI2S accumulated in brain tissues of a cynomolgus monkey after a single intravenous administration. The vertical axis indicates the amount of I2S-anti-hTfR antibody and rhI2S per wet weight of the brain tissues (μg/g wet weight). The dotted bars represent the amount of rhI2S accumulated in the brain tissues of the cynomolgus monkey. The hatched bars represent the amount of the I2S-anti-hTfR antibody accumulated in the brain tissues of the cynomolgus monkey. Vertical line segment indicates the standard deviation.

FIG. 10 Graphs showing the amount of the glycosaminoglycan (GAG) in various organs of a 12S gene-knockout mouse (12S-KO mouse) intravenously injected with a humanized anti-hTfR antibody or rhI2S. (a) brain, (b) liver, (c) lung, (d) heart. For each graph, the bars represent, from the left, wild-type mouse (WT), control group (non-administered group), 0.5 mg/kg administered group, 1.0 mg/kg administered group, and 2.0 mg/kg administered group. The vertical axis indicates the amount of GAG of the dry weight of each organ (μg/g dry weight). Vertical line segments indicates the standard deviation, and “**” indicates p<0.01 compared with the control group according to Dunnett's test.

### EMBODIMENTS FOR CARRYING OUT THE INVENTION

In the present invention, the term “antibody” refers mainly to a human antibody, mouse antibody, humanized antibody, as well as a chimeric antibody between human antibody and non-human mammalian antibody, and a chimeric antibody between mouse antibody and non-mouse mammalian antibody, but the meaning of the term is not limited to them insofar as a substance of interest has a property to specifically bind to a certain antigen, and there is no specific limitation as to the animal species of the antibody, either.

In the present invention, the term “human antibody” refers to an antibody whose entire protein is encoded by a gene originating from human. The term “human antibody”, however, also includes an antibody encoded by a gene obtained by introducing a mutation into an original human gene for a purpose of enhancing expression efficiency of the gene, for example, without modifying the original amino acid sequence. The term “human antibody” also includes an antibody which is obtainable through combining two or more genes encoding human antibodies by replacing a certain part of a human antibody with a part of another human antibody. A human antibody includes three complementarity determining regions (abbr. CDRs) in the light chain of the immunoglobulin and three complementarity determining regions (CDRs) in the heavy chain of the immunoglobulin. The three CDRs in the light chain of the immunoglobulin are called, from the N-terminal side, CDR1, CDR2 and CDR3, respectively. The three CDRs in the heavy chain of the immunoglobulin are also called, from the N-terminal side, CDR1, CDR2 and CDR3, respectively. The term “human antibody” also includes a human antibody produced by replacing a CDR of a human antibody with a CDR of another human antibody to modify such properties as the antigen specificity and the affinity of the original human antibodies, etc.

In the present invention, the term “human antibody” also includes an antibody which is produced through modification of the gene of the original human antibody by introducing a mutation, such as substitution, deletion, addition, to the amino acid sequence of the original antibody. When replacing one or more amino acids of the amino acid sequence of the original antibody with other amino acids, the number of amino acid replaced may preferably be 1 to 20, more preferably 1 to 5, and still more preferably 1 to 3. When deleting one or more amino acids of the amino acid sequence of the original antibody, the number of amino acids deleted may preferably be 1 to 20, more preferably 1 to 5, and still more preferably 1 to 3. An antibody produced by a combined mutation of these substitution and deletion of amino acids is also a “human antibody”. In some cases, one or more amino acids, preferably 1 to 20, more preferably 1 to 5, and still more preferably 1 to 3 amino acids may be added inside the amino acid sequence of the original antibody or on its N- or C-terminal side. An antibody produced by a combined mutation of addition, substitution, and deletion of amino acids is also a “human antibody”. The amino acid sequence of such a mutated antibody has a homology of preferably not lower than 80%, more preferably not lower than 90%, still more preferably not lower than 95%, and even more preferably not lower than 98%, to the amino acid sequence of the original antibody. Thus, in the present invention, the term “gene originating from human” includes not only the unmutated gene originating from human but also a gene produced by modifying it.

The homology between the amino acid sequence of an unmutated antibody and the amino acid sequence of an antibody produced by introducing a mutation into it may be readily calculated using well-known homology calculator algorithms. As such algorithms, there are, for example, BLAST (Altschul S F. J Mol. Biol. 215. 403-10 (1990)), a similarity search by Pearson and Lipman (Proc. Natl. Acad. Sci. USA. 85. 2444 (1988)), and the local homology algorithm of Smith and Waterman (Adv. Appl. Math. 2. 482-9 (1981)), and the like.

The term “mouse antibody” refers to an antibody whose entire protein consists of an amino acid sequence which is the same as an antibody encoded by a gene originating from a mouse. Therefore, the term “mouse antibody” also includes an antibody that is encoded by a gene produced by introducing a mutation into the original mouse gene without causing a change in its amino acid sequence but in order, for example, to improve the expression efficiency of the gene. Further, the term “mouse antibody” also includes an antibody produced through combining two or more genes encoding mouse antibodies by replacing a part of a mouse antibody with a part of another mouse antibody. A mouse antibody has three complementarity determining regions (CDRs) in the light chain of the immunoglobulin and three complementarity determining regions (CDRs) in the heavy chain of the immunoglobulin. The three CDRs in the light chain of the immunoglobulin are called, from the N-terminal side, CDR1, CDR2 and CDR3, respectively. The three CDRs in the heavy chain of the immunoglobulin are also called, from the N-terminal side, CDR1, CDR2 and CDR3, respectively. The term “mouse antibody” also includes an antibody produced by replacing a CDR of a mouse antibody with a CDR of another mouse antibody to modify the specificity and affinity of the original mouse antibodies.

In the present invention, the term “mouse antibody” also includes an antibody which is produced through modification of the gene of the original mouse antibody by introducing a mutation, such as substitution, deletion, addition, to the amino acid sequence of the original antibody. When replacing one or more amino acids of the amino acid sequence of the original antibody with other amino acids, the number of amino acid replaced may preferably be 1 to 20, more preferably 1 to 5, and still more preferably 1 to 3. When deleting one or more amino acids of the amino acid sequence of the original antibody, the number of amino acids deleted may preferably be 1 to 20, more preferably 1 to 5, and still more preferably 1 to 3. An antibody produced by a combined mutation of these substitution and deletion of amino acids is also a “mouse antibody”. When adding one or more amino acids, they may be added inside the amino acid sequence of the original antibody or on its N- or C-terminal side, preferably 1 to 20, more preferably 1 to 5, and still more preferably 1 to 3, in number. An antibody produced by a combined mutation of addition, substitution, and deletion of amino acids is also a “mouse antibody”. The amino acid sequence of such a mutated antibody has a homology of preferably not lower than 80%, more preferably not lower than 90%, still more preferably not lower than 95%, and even more preferably not lower than 98%, to the amino acid sequence of the original antibody. Thus, in the present invention, the term “gene originating from mouse” includes not only the unmutated gene originating from mouse but also a gene produced by modifying it.

In the present invention, the term “humanized antibody” refers to an antibody in which part of the amino acid sequence of its variable region (e.g., especially the whole or part of its CDRs) originates from a non-human mammal while the rest originates from human. An example of humanized antibody is an antibody produced by replacing the three complementarity determining regions (CDRs) of the light chain of the immunoglobulin and the three complementarity determining regions (CDRs) of the heavy chain of the immunoglobulin constituting a human antibody, with CDRs from a non-human mammal. As far as it originates from a non-human mammal, there is no particular limitation as to the biological species from which those CDRs originate that are grafted into a proper position of the human antibody, though preferred are mouse, rat, rabbit, horse or non-human primate, more preferred are mouse and rat, and mouse, for example.

In the present invention, the term “chimeric antibody” refers to an antibody produced by connecting fragments of two or more different antibodies originating from two or more different species.

A chimeric antibody between a human antibody and a non-human mammalian antibody is an antibody provided by replacing part of a human antibody with part of a non-human mammalian antibody. As explained below, an antibody is made of an Fc region, a Fab region and a hinge region. A specific example of such chimeric antibodies is a chimeric antibody whose Fc region originates from a human antibody while its Fab region originates from a non-human mammalian antibody. The hinge region either originates from a human antibody or from a non-human mammalian antibody. On the contrary, the term chimeric antibody also includes one whose Fc region originates from a non-human mammalian antibody while its Fab region originates from a human antibody. In such a case also, the hinge region may either originate from a human antibody or from a non-human mammalian antibody. On the contrary, a chimeric antibody is also included as an example whose Fc region originates from a non-human mammalian antibody while its Fab region originates from a human antibody. In this case also, the hinge region may either originate from a human antibody or from a non-human mammalian antibody.

An antibody can be viewed as composed of a variable region and a constant region. Additional examples of chimeric antibodies include an antibody in which the heavy chain constant region (C_{H}) and the light chain constant region (Cₗ) both originate from a human antibody while the heavy chain variable region (V_{H}) and the light chain variable region (V_{L}) both originate from an antibody of a non-human mammal, and conversely, an antibody in which the heavy chain constant region (C_{H}) and the light chain constant region (Cₗ) both originate from an antibody of a non-human mammal, while the heavy chain variable region (V_{H}) and the light chain variable region (V_{L}) both originate from a human antibody. In these, there is no particular limitation as to the biological species of the non-human mammal, as far as it is a non-human mammal, though preferred are mouse, rat, rabbit, horse or non-human primate, and more preferred is mouse.

A chimeric antibody between a mouse antibody and a non-mouse mammalian antibody is an antibody provided by replacing part of a mouse antibody with part of a non-mouse mammalian antibody. Specific examples of such chimeric antibodies include a chimeric antibody whose Fc region originates from a mouse antibody while its Fab region originates from a non-mouse mammalian antibody, and conversely, a chimeric antibody whose Fc region originates from a non-mouse mammal while its Fab region originates from a mouse antibody. In these, there is no particular limitation as to the biological species of the non-mouse mammal, as far as it is a mammal other than mouse, though preferred are rat, rabbit, horse or non-human primate, and more preferred is human.

A chimeric antibody between a human antibody and a mouse antibody is designated in particular “human/mouse chimeric antibody”. Examples of human/mouse chimeric antibodies include a chimeric antibody in which the Fc region originates from a human antibody while the Fab region originates from a mouse antibody, and conversely, a chimeric antibody whose Fc region originates from mouse antibody, while its Fab region originates from a human antibody. A hinge region either originate from a human antibody or a mouse antibody. Additional specific examples of human/mouse chimeric antibodies include those whose heavy chain constant region (C_{H}) and light chain constant region (Cₗ) originate from a human antibody while its heavy chain variable region (V_{H}) and light chain variable region (V_{L}) originate from a mouse antibody, and conversely, those whose heavy chain constant region (C_{H}) and light chain constant region (Cₗ) originate from a mouse antibody while its heavy chain variable region (V_{H}) and light chain variable region (V_{L}) originate from a human antibody.

Originally, an antibody is of the basic structure having four polypeptide chains in total consisting of two immunoglobulin light chains and two immunoglobulin heavy chains. However, in the present invention the term “antibody” refers, besides an antibody having this basic structure, also to:
(1) one consisting of two polypeptide chains: a single immunoglobulin light chain and a single immunoglobulin heavy chain, and also, as explained later,
(2) a single-chain antibody consisting of an immunoglobulin light chain which is linked, on the C-terminal side thereof, to a linker sequence which in turn is linked, on the C-terminal side thereof, to an immunoglobulin heavy chain,
(3) single-chain antibodies consisting of an immunoglobulin heavy chain which is linked, on the C-terminal side thereof, to a linker sequence which in turn is linked, on the C-terminal side thereof, to an immunoglobulin light chain, and
(4) one consisting of a Fab region, i.e., a structure left behind by removal of the Fc region from an antibody having the basic structure, as the original meaning, and one consisting of the Fab region and the whole or part of the hinge region (including Fab, F(ab′), and F(ab′)₂) also are included in the term “antibody” in the present invention.

The term “Fab” refers to a molecule consisting of a single light chain comprising the variable region and the C_{L} region (light chain constant region) and a single heavy chain comprising the variable region and the C_{H}1 region (portion 1 of heavy chain constant region) which are combined by a disulfide bond between their respective cysteine residues. While the heavy chain in a Fab can include part of the hinge region in addition to the variable region and the CHI region (portion 1 of heavy chain constant region), the hinge region in such a case lacks the cysteine residue that otherwise is present in the hinge region and would serve to link two heavy chains of an antibody together. In Fab, the light chain and the heavy chain are connected by a disulfide bond formed between the cysteine residue present in the light chain constant region (C_{L} region) and the cysteine residue located in the heavy chain constant region (C_{H}1 region) or the hinge region. As it lacks the cysteine residue in the hinge region which serves to bind two heavy chains of an antibody, Fab consists of a single light chain and a single heavy chain. The light chain constituting Fab includes a variable region and a C_{L} region. The heavy chain as a component of Fab may either consist of a variable region and a C_{H}1 region or also of part of the hinge region in addition to the variable region and the C_{H}1 region. However, in the letter case, the hinge region is so selected as not to include the cysteine residue that could bind two heavy chains, in order to avoid the formation of a disulfide bond between two heavy chains at their hinge regions. In F(ab′), the heavy chain includes, in addition to a variable region and a C_{H}1 region, the whole or part of a hinge region containing a cysteine residue that could bind two heavy chains. F(ab′)₂ is a molecule consisting of two F(ab′) s bound together through a disulfide bond formed between the cysteine residues present in their respective hinge regions. Further, a polymer such as a dimer and a trimer, which consists of two or more antibodies connected with each other, directly or via a linker, is also included in the term “antibody”. Moreover, in addition to the aforementioned, any molecule that includes part of an immunoglobulin molecule and has a property to specifically bind to the antigen is also included in the term “antibody” in the present invention. Thus, in the present invention, the term “immunoglobulin light chain” includes a molecule that is derived from an original immunoglobulin light chain and having the amino acid sequence of the whole or part of its variable region. Likewise, the term “immunoglobulin heavy chain” includes a molecule that is derived from an original immunoglobulin heavy chain and having the amino acid sequence of the whole or part of its variable region. Therefore, insofar as having the whole or part of the amino acid sequence of the variable region, a molecule is included in the term “immunoglobulin heavy chain”, even if it lacks its Fc region, for example.

In the above, the term “Fc” or “Fc region” refers to a region comprising a fragment consisting of C_{H}2 region (portion 2 of the heavy chain constant region), and C_{H}3 region (portion 3 of the heavy chain constant region) in the antibody molecule.

Furthermore, in the present invention, the term “antibody” also includes:
(5) scFab, scF(ab′), and scF(ab′)₂, which are single-chain antibodies produced by binding the light chain to the heavy chain that form, respectively, the Fab, F(ab′) and F(ab′)₂ mentioned in (4) above, via a linker sequence. Such scFab, scF(ab′) and scF(ab′)₂ may be a molecule in which either the light chain is linked, on the C-terminal side thereof, to a linker sequence, which in turn is linked, on the C-terminal side thereof, to the heavy chain, or the heavy chain is linked, on the C-terminal side thereof, to a linker sequence, which in turn is linked, on the C-terminal side thereof, to the light chain. Furthermore, a scFv, which is a single-chain antibody provided by binding the light chain variable region to the heavy chain variable region, via a linker sequence between them, is also included in the term “antibody” in the present invention. Such scFv may be a molecule in which either the light chain variable region is linked, on the C-terminal side thereof, to a linker sequence, which in turn is linked, on the C-terminal side thereof, to the heavy chain variable region, or the heavy chain variable region is linked, on the C-terminal side thereof, to a linker sequence, which in turn is linked, on the C-terminal side thereof, to the light chain variable region.

Furthermore, in addition to a full-length antibody and those described in (1) to (5) above, the term “antibody” in the present specification includes, any form of antigen-binding fragment which lacks part of the full-length antibody (antibody fragment), a broader concept which includes (4) and (5) above.

The term “antigen-binding fragment” refers to an antibody fragment that retains at least part of the specific binding activity to its antigen. In addition to those described above in (4) and (5), examples of binding fragments include Fab, Fab′, F(ab′)₂, variable region (Fv); a single-chain antibody (scFv) produced by linking the heavy chain variable region (V_{H}) and the light chain variable region (V_{L}), via a proper linker between them; a diabody, which is a dimer of a polypeptide that comprises a heavy chain variable region (V_{H}) and a light chain variable region (V_{L}); a minibody, which is a dimer of a molecule in which the heavy chain (H chain) of a scFv is linked to part of the constant region (C_{H}3), and other low-molecular-antibodies. However, as far as it has an antigen-binding ability, the term is not limited to these molecules. Such binding fragments include not only those produced by treating a full-length molecule of an antibody protein with a proper enzyme but also those produced by proper host cells using a genetically engineered antibody gene.

In the present invention, the term “single-chain antibody” refers to a protein in which an amino acid sequence comprising the whole or part of an immunoglobulin light chain variable region linked, on the C-terminal side thereof, to a linker sequence, which in turn is linked, on the C-terminal side thereof, to the amino acid sequence of the whole or part of an immunoglobulin heavy chain variable region, and having an ability to specifically bind a certain antigen. For example, those described in (2), (3) and (5) are included in “single-chain antibody”. Further, a protein in which an amino acid sequence comprising the whole or part of an immunoglobulin heavy chain variable region is linked, on the C-terminal side thereof, to a linker sequence, which in turn is further linked, on the C-terminal side thereof, to the amino acid sequence of the whole or part of an immunoglobulin light chain variable region, and which has an ability to specifically bind to a certain antigen, is also included in the term “single-chain antibody” in the present invention. In a single-chain antibody in which an immunoglobulin heavy chain is linked, on the C-terminal side thereof and via a linker sequence, to an immunoglobulin light chain, the immunoglobulin heavy chain generally lacks the Fc region. An immunoglobulin light chain variable region has three complementarity determining regions (CDRs) which participate in determining the antigen specificity of an antibody. Likewise, an immunoglobulin heavy chain variable region also has three CDRs. Those CDRs are the primary regions that determine the antigen specificity of an antibody. Therefore, a single-chain antibody preferably contains all the three CDRs of the immunoglobulin heavy chain and all the three CDRs of the immunoglobulin light chain. However, it is also possible to provide a single-chain antibody in which one or more of those CDRs are deleted, insofar as the antigen-specific affinity of the antibody is retained.

In a single-chain antibody, the linker sequence placed between the light chain and the heavy chain of the immunoglobulin is preferably a peptide chain consisting of preferably 2 to 50, more preferably 8 to 50, still more preferably 10 to 30, even more preferably 12 to 18, or 15 to 25, for example 15 or 25 amino acid residues. While there is no particular limitation as to the specific amino acid sequence of such a linker sequence insofar as the anti-hTfR antibody comprising the both chains linked thereby retains the affinity to hTfR, it is preferably made of glycine only, or of glycine and serine: for example the amino acid sequence Gly-Ser, the amino acid sequence Gly-Gly-Ser, the amino acid sequence Gly-Gly-Gly, the amino acid sequence Gly-Gly-Gly-Gly-Ser (SEQ ID NO:3), the amino acid sequence Gly-Gly-Gly-Gly-Gly-Ser (SEQ ID NO:4), the amino acid sequence Ser-Gly-Gly-Gly-Gly-Gly (SEQ ID NO:5), or a sequence which includes 2 to 10 or 2 to 5 repeats of any of those amino acid sequences. For example, in linking the amino acid sequence of the entire immunoglobulin heavy chain variable region, on the C-terminal side thereof and via a linker sequence, to immunoglobulin light chain variable region, the linker sequence is preferably a linker sequence comprising 15 amino acids corresponding to three of the amino acid sequence Gly-Gly-Gly-Gly-Ser (SEQ ID NO:3) consecutively linked.

In the present invention, the term “human transferrin receptor” or “hTfR” refers to a membrane protein having the amino acid sequence set forth as SEQ ID NO:1. The anti-hTfR antibody of the present invention is, in one of its embodiments, that which specifically binds to the region from the cysteine residue at the position 89th from the N-terminal side to the phenylalanine at the C-terminus in the amino acid sequence set forth as SEQ ID NO:1 (i.e., the extracellular region of the hTfR), though it is not limited to this embodiment. Further, in the present invention, the term “monkey transferrin receptor” or “monkey TfR” refers in particular to the membrane protein having the amino acid sequence set forth as SEQ ID NO:2, originating from cynomolgus monkey (Macaca fascicularis). The anti-hTfR antibody of the present invention is, in one of its embodiments, that which binds also to region from the cysteine residue at the position 89th from the N-terminal side to the phenylalanine at the C-terminus in the amino acid sequence set forth as SEQ ID NO:2 (i.e., the extracellular region of the monkey TfR), though it is not limited to this embodiment.

For preparation of an antibody to hTfR, there is known a general method according to which a recombinant human transferrin receptor (rhTfR) is produced using cells which have an introduced expression vector having an incorporated hTfR gene, and then animals such as mice are immunized with this rhTfR. By collecting those cells which produce antibodies to hTfR from the immunized animals and fusing them with myeloma cells, hybridoma cells can be obtained having an ability to produce the antibody.

Further, cells producing an antibody to hTfR can also be obtained by collecting immunocompetent cells from an animal such as mouse, and immunizing them with rhTfR by in vitro immunization. In conducting in vitro immunization, there is no particular limitation as to the animal species from which the immunocompetent cells are derived, though preferred are mouse, rat, rabbit, guinea pig, dog, cat, horse, and primates including human, and more preferred are mouse, rat and human, and still more preferably mouse and human. As mouse immunocompetent cells, spleen cells prepared from mouse spleen may be used, for example. As human immunocompetent cells, such cells can be used as prepared from human peripheral blood, bone marrow, spleen, and the like. By immunizing human immunocompetent cells according to in vitro immunization, a human antibody to hTfR can be obtained.

After immunizing the immunocompetent cells according to in vitro immunization, the cells can be fused with myeloma cells to prepare hybridoma cells having an ability to produce the antibody. Further, it is also possible to extract mRNAs from the immunized cells, synthesize cDNA, perform PCR reaction using the cDNA as a template to amplify a DNA fragment containing the gene encoding the light chain and the heavy chain of the immunoglobulin, and artificially reconstruct the antibody gene using them.

The hybridoma cells freshly obtained above also include such cells that produce antibodies that recognize other proteins than hTfR. Furthermore, not all the hybridoma cells producing an anti-hTfR antibody necessarily produce an anti-hTfR antibody that exhibits high affinities to hTfR.

Likewise, artificially reconstructed antibody genes include such genes as encode antibodies recognizing other proteins than hTfR as antigens. Moreover, not all the genes encoding anti-hTfR antibodies necessarily have desired properties such as encoding an anti-hTfR antibody exhibiting high affinity to hTfR.

Therefore, a selection step is necessary to select hybridoma cells producing an antibody having desired properties (such as high affinity to hTfR) from the hybridoma cells freshly obtained above. Further, in the case where antibody genes are artificially reconstructed, a selection step is necessary to select from the antibody genes a gene encoding an antibody having desired properties (such as high affinities to hTfR). For selecting hybridoma cells that produce antibodies exhibiting high affinities to hTfR (high affinity antibodies), or for selecting genes encoding high affinity antibodies, following methods explained in detail below are effective. Besides, antibodies exhibiting high affinity to hTfR are those whose dissociation constant (K_{D}) with hTfR as measured by the method described in Example 7 is preferably not greater than 1×10⁻⁸ M, more preferably not greater than 1×10⁻⁹ M, still more preferably not greater than 1×10⁻¹⁰ M, and even more preferably not greater than 1×10⁻¹¹ M. For example, those having a dissociation constant of 1×10⁻¹³ M to 1×10⁻⁹ M, or 1×10⁻¹³ M to 1×10⁻¹⁰ M are preferable.

For example, for selecting hybridoma cells which produce high affinity antibodies to anti-hTfR antibody, a method is employed in which recombinant hTfR is added to a plate and held by it, then the culture supernatant of the hybridoma cells is added, and after removing antibody unbound to the recombinant hTfR from the plate, the amount of the antibody held by the plate is measured. According to this method, the higher the affinity to hTfR of the antibody contained in the culture supernatant of the hybridoma cells added to the plate is, the greater the amount of antibody held by the plate becomes. Therefore, by measuring the amount of the antibody held by the plate, it is possible to select those hybridoma cells corresponding to the plates where the antibody is held in the greater amount as cell lines producing an anti-hTfR antibody having the relatively higher affinity to hTfR. It is also possible to isolate the gene encoding the high-affinity antibody by extracting mRNAs from each cell line selected in this manner, synthesizing cDNAs, and amplifying a DNA fragment containing the gene encoding the anti-hTfR antibody by PCR using the cDNA as a template.

In order to select the gene encoding the high-affinity anti-hTfR antibody from the above artificially reconstructed antibody genes, the artificially reconstructed antibody genes are once incorporated into an expression vector, and the expression vector then is introduced into host cells. Although there is no particular limitation as to the cells to be employed as host cells, even whether they are prokaryotic or eukaryotic, insofar as they can express the antibody gene after introduction of an expression vector having the incorporated artificially reconstructed antibody gene, preferred are cells originating mammals such as human, mouse, Chinese hamster, and the like, and particularly preferred are CHO cells originating from Chinese hamster ovary cells, or NS/0 cells originating from mouse myeloma. Further, there is no particular limitation as to an expression vector to be employed for incorporation of the antibody encoding gene and expression of it, and any expression vector may be used as far as it can express the gene when introduced into mammalian cells. The gene incorporated into an expression vector is located downstream of a DNA sequence that can regulate the frequency of transcription of a gene in mammalian cells (gene expression regulatory site). Examples of gene expression regulatory sites that may be employed in the present invention include cytomegalovirus-derived promoter, SV40 early promoter, human elongation factor-1α (EF-1α) promoter, human ubiquitin C promoter.

Mammalian cells having such an introduced expression vector come to express the artificially reconstructed antibody incorporated in the expression vector. In order to select those cells that produce a high-affinity antibody to anti-hTfR antibody from the above obtained cells expressing the artificially reconstructed antibody, a method is employed in which the recombinant hTfR is added to a plate and held by it, then the recombinant hTfR is contacted by the culture supernatant of the cells, and after the removal of antibody unbound to the recombinant hTfR from the plate, the amount of the antibody held by the plate is measured. According to this method, the higher the affinity to hTfR of the antibody contained in the cells culture supernatant is, the greater the amount of antibody held by the plate becomes. Therefore, by measuring the amount of the antibody held by the plate, one can select those cells corresponding to the plate where the antibody is held in the greater amount, as a cell line producing an anti-hTfR antibody having relatively the high-affinity anti-hTfR antibodyR, and eventually can select a gene encoding an anti-hTfR antibody having a high-affinity anti-hTfR antibody to hTfR. Using cell line selected in this manner, one can perform PCR to amplify a DNA fragment containing the gene encoding the anti-hTfR antibody to isolate the gene encoding the high-affinity antibody.

Selection of the gene encoding a high affinity anti-hTfR antibody from the above artificially reconstructed antibody genes can also be carried out by incorporating the artificially reconstructed antibody genes into an expression vector, introducing the expression vector into E. coli cells, culturing the E. coli cells, and selecting the E. coli cells having the desired gene, in the same manner as in the above selection of hybridoma cells, using the culture supernatant of the E. coli cells or an antibody-containing solution prepared by lysing the E. coli cells. E. coli cells thus selected express the gene encoding an anti-hTfR antibody having a relatively high affinity to hTfR. From this cell line, the gene encoding the anti-hTfR antibody having a relatively the high-affinity anti-hTfR antibody to hTfR can be selected. In order to allow the antibody to be secreted into the E. coli culture supernatant, the antibody gene may be incorporated into the expression vector so that a secretion signal sequence is attached on the N-terminal side of the gene.

Another method for selection of the gene encoding a high-affinity anti-hTfR antibody is a method in which the antibody encoded by the above artificially reconstructed antibody gene is expressed and retained on phage particles. For this, the antibody gene is reconstructed as a gene encoding a single-chain antibody. A method for retaining the antibody on the surface of phage particles is disclosed in international publications WO1997/09436 and WO1995/11317, and the like, and thus well known. In order to select phages retaining the high-affinity antibody to anti-hTfR antibody from the phages retaining the antibodies encoded by the artificially reconstructed antibody genes, a method is employed in which a recombinant hTfR is added to a plate and held by it, contacted by the phages, and after removal of the phages unbound to the recombinant hTfR from the plate, the amount of the phages held by the plate is measured. According to this method, the higher the affinity to hTfR of the antibody retained on the phage particles is, the greater the amount of the phage held by the plate becomes. Therefore, by measuring the amount of the phage held by the plate, one can select the phage particles corresponding to the plate where the phages' were held in the greater amount, as the phage particles producing anti-hTfR antibody having a relatively high-affinity anti-hTfR antibody to hTfR, and eventually can select the gene encoding the high-affinity anti-hTfR antibody to hTfR. Using the phage particles thus selected, PCR can be performed to amplify a DNA fragment containing the gene encoding the anti-hTfR antibody and isolate the gene encoding the high-affinity antibody.

It is possible to prepare cDNA or phage DNA from the above cells such as the hybridoma cells producing the high-affinity antibody to anti-hTfR, or from the above phage particles retaining high-affinity antibody to anti-hTfR, and perform PCR or the like using it as a template to amplify and isolate a DNA fragment containing the gene encoding the whole or part of the anti-hTfR antibody light chain, the anti-hTfR antibody heavy chain, or a single-chain antibody. In the same manner, it is also possible to perform PCR or the like to amplify and isolate a DNA fragment containing the gene encoding the whole or part of the light chain variable region of the anti-hTfR antibody, or a DNA fragment containing the gene encoding the whole or part of the heavy chain variable region of the anti-hTfR antibody.

A high-affinity anti-hTfR antibody can be obtained by incorporating the whole or part of the gene encoding the light chain and the heavy chain of this high-affinity anti-hTfR antibody into an expression vector, transforming host cells such as mammalian cells with this expression vector, and culturing the obtained transformant cells. Using the nucleotide sequence of the isolated gene encoding the anti-hTfR antibody, it is also possible to translate the amino acid sequence of the anti-hTfR antibody, and artificially synthesize a DNA fragment encoding the same amino acid sequence. In artificially synthesizing a DNA fragment, the expression level of the anti-hTfR antibody in the host cells can be enhanced by proper selection of the codons.

In order to introduce a mutation such as substitution, deletion, addition and the like into the amino acid sequence of the original anti-hTfR antibody, a mutation may be introduced as desired into the gene encoding the anti-hTfR antibody contained in the isolated DNA fragment. Though the gene encoding the mutated anti-hTfR antibody has a homology preferably not lower than 80%, more preferably not lower than 90%, to the original gene, there is no particular limitation as to the level of homology. By introducing a mutation into the amino acid sequence so as to modify the number or the type of sugar chains bound to the anti-hTfR antibody, it is also possible to enhance the stability of the anti-hTfR antibody in the body.

When introducing a mutation into the gene encoding the whole or part of the light chain variable region of the anti-hTfR antibody, the gene thus mutated has a homology that is preferably not lower than 80%, more preferably not lower than 90%, to the original gene, though there is no particular limitation as to the level of homology. When replacing one or more amino acids of the amino acid sequence of the light chain variable region with other amino acids, the number of amino acids to be replaced is preferably 1 to 10, more preferably 1 to 5, still more preferably 1 to 3, and even more preferably 1 or 2. When deleting one or more amino acids of the amino acid sequence of the light chain variable region, the number of amino acid to be deleted is preferably 1 to 10, more preferably 1 to 5, still more preferably 1 to 3, even more preferably 1 or 2. A combined mutation of these substitution and deletion of amino acids can also be carried out. When adding one or more amino acids to the light chain variable region, they may be added inside, or on the N-terminal side or C-terminal side of, the amino acid sequence of the light chain variable region, and the number of amino acids added is preferably 1 to 10, more preferably 1 to 5, still more preferably 1 to 3, and even more preferably 1 or 2. A combined mutation of these addition, substitution, and deletion of amino acids can also be carried out. The amino acid sequence of the light chain variable region thus mutated has a homology that is preferably not lower than 80%, more preferably not lower than 90%, still more preferably not lower than 95% to the amino acid sequence of the original light chain variable region. In particular, when replacing one or more amino acids of the amino acid sequence of CDR with other amino acids, the number of amino acid replaced is preferably 1 to 5, more preferable 1 to 3, still more preferably 1 or 2. When deleting one or more amino acid of the amino acid sequence of CDR, the number of amino acids to be deleted is preferably 1 to 5, more preferably 1 to 3, still more preferably 1 or 2. A combined mutation of these substitution and deletion of the amino acid can also be carried out. When adding one or more amino acids, they may be added inside, or on the N-terminal side or C-terminal side of, the amino acid sequence, and the number of amino acids added is preferably 1 to 5, more preferably 1 to 3, still more preferably 1 or 2. A combined mutation of these addition, substitution, and deletion of amino acids can also be carried out. The amino acid sequence of respective mutated CDR has a homology that is preferably not lower than 80%, more preferably not lower than 90%, and still more preferably not lower than 95% to the amino acid sequence of the original CDR.

When introducing mutation into the gene encoding the whole or part of the heavy chain variable region of the anti-hTfR antibody, the gene thus mutated has a homology that is preferably not lower than 80%, more preferably not lower than 90%, to the original gene, though there is no particular limitation as to the level of homology. When replacing one or more amino acids of the amino acid sequence of the heavy chain variable region with other amino acids, the number of amino acids to be replaced is preferably 1 to 10, more preferably 1 to 5, still more preferably 1 to 3, and even more preferably 1 or 2. When deleting one or more amino acids of the amino acid sequence of the heavy chain variable region, the number of amino acids to be deleted is preferably 1 to 10, more preferably 1 to 5, still more preferably 1 to 3, even more preferably 1 or 2. A combined mutation of these substitution and deletion of amino acids can also be carried out. When adding one or more amino acid to the heavy chain variable region, they may be added inside, or on the N-terminal side or C-terminal side of, the amino acid sequence of the heavy chain variable region, and the number of amino acids added is preferably 1 to 10, more preferably 1 to 5, still more preferably 1 to 3, and even more preferably 1 or 2. A combined mutation of these addition, substitution, and deletion of amino acids can also be carried out. The amino acid sequence of the heavy chain variable region thus mutated has a homology that is preferably not lower than 80%, more preferably not lower than 90%, still more preferably not lower than 95% to the amino acid sequence of the original heavy chain variable region. In particular, when replacing one or more amino acids of the amino acid sequence of CDR with other amino acids, the number of amino acid replaced is preferably 1 to 5, more preferable 1 to 3, still more preferably 1 or 2. When deleting one or more amino acid of the amino acid sequence of CDR, the number of amino acids to be deleted is preferably 1 to 5, more preferably 1 to 3, still more preferably 1 or 2. A combined mutation of these substitution and deletion of the amino acid can also be carried out. When adding one or more amino acids, they may be added inside, or on the N-terminal side or C-terminal side of, the amino acid sequence, and the number of amino acids added is preferably 1 to 5, more preferably 1 to 3, still more preferably 1 or 2. A combined mutation of these addition, substitution, and deletion of amino acids can also be carried out. The amino acid sequence of respective mutated CDR has a homology that is preferably not lower than 80%, more preferably not lower than 90%, and still more preferably not lower than 95% to the amino acid sequence of the original CDR.

A mutation may be introduced into both the variable regions of the light chain and the heavy chain of the anti-hTfR antibody, by combining the above mutation into the light chain variable region of the anti-hTfR antibody and the above mutation into the heavy chain variable region of the anti-hTfR antibody.

Examples of the above mentioned substitution of one or more amino acids in the amino acid sequence of the light chain and the heavy chain of the anti-hTfR antibody include substitution between acidic amino acids, i.e., aspartic acid and glutamic acid, substitution between amide-type amino acids, i.e., asparagine and glutamine, substitution between basic amino acids, i.e., lysine and arginine, substitution between branched amino acids, i.e., valine, leucine and isoleucine, substitution between aliphatic amino acids, i.e., glycine and alanine, substitution between hydroxyamino acids, i.e., serine and threonine, and substitution between aromatic amino acids, i.e., phenylalanine and tyrosine.

Besides, in the case where a mutation is introduced into the anti-hTfR antibody by adding one or more amino acids to the C-terminus or the N-terminus, if the anti-hTfR antibody and a different protein (A) are fused via the added amino acids, the added amino acids constitutes part of a linker. A detailed explanation will be given later on a linker that is placed between the anti-hTfR antibody and a different protein (A) in the case where the anti-hTfR antibody is fused with the different protein (A).

The anti-hTfR antibody obtained by culturing the cells selected by the above methods and the like to produce an anti-hTfR antibody that has a relatively high-affinity to hTfR, and the anti-hTfR antibody obtained by expression of the gene encoding a high-affinity anti-hTfR antibody, may be modified by introducing a mutation into their amino acid sequences, such as substitution, deletion, addition to give them desired properties. Introduction of a mutation into the amino acid sequence of the anti-hTfR antibody may be performed by introducing a mutation into the gene corresponding to the amino acid sequence.

The affinity of an anti-hTfR antibody to hTfR can be adjusted as desired by introduction of a mutation, such as substitution, deletion, and addition, into the amino acid sequence of a variable region of the antibody. For example, if an antibody has such a high affinity to its antigen that leads to too low a dissociation constant in an aqueous solution, there is a possibility that the antibody could, after administered to the body, fail to dissociate from the antigen, thereby leading to a functional disadvantage. In such a case, a most preferable antibody suitable to a given purpose can be obtained by introducing a mutation into the variable region of the antibody so as to adjust its dissociation constant stepwise to 2 to 5 times, 5 to 10 times, 10 to 100 times, and so on, that of the original antibody. Conversely, the dissociation constant can be adjusted stepwise to 1/2 to 1/5 times, 1/5 to 1/10 times, 1/10 to 1/100 times, and so on, that of the original antibody, by introducing a mutation.

Introduction of a mutation such as substitution, deletion and addition to the amino acid sequence of the anti-hTfR antibody can be performed, for example, either by introducing a mutation into certain positions of the nucleotide sequence of the gene or by random introduction of a mutation, by PCR or the like using the gene encoding the anti-hTfR antibody as a template.

Introduction of a mutation into the amino acid sequence of the anti-hTfR antibody for adjusting the affinity of the antibody to hTfR can be carried out by, for example, incorporating a gene encoding the anti-hTfR antibody as a single-chain antibody into a phagemid, preparing with this phagemid a phage with expressed single-chain antibody on the surface of its capsid, letting the phage multiply while introducing a mutation into the gene encoding the single-chain antibody by application of a mutagen or the like, and selecting, from the multiplied phage, a phage expressing a single-chain antibody having a desired dissociation constant either by the method described above or by purification using an antigen column under a certain condition.

The antibodies having a relatively high-affinity to hTfR obtained by the above-mentioned method of selecting the cells producing a high affinity antibody, are those whose dissociation constant (K_{D}) with hTfR as measured by the method described in Example 7 is preferably not greater than 1×10⁻⁸ M, more preferably not greater than 1×10⁻⁹M, still more preferably not greater than 1×10⁻¹⁰ M, and even more preferably not greater than 1×10⁻¹¹ M. For example, those having a dissociation constant of 1×10⁻¹³ M to 1×10⁻⁹ M, or 1×10⁻¹³ M to 1×10⁻¹⁰ M are preferable. The same also applies if the antibodies are single-chain antibodies. Once an antibody is obtained, it can be modified as desired by, e.g., introducing a mutation to give it a desired property.

Antibody having affinity both to human and monkey TfRs can be obtained by selection of antibodies having affinity to monkey TfR from the antibodies having a relatively high-affinity that have been obtained by the above described method involving selection of the cells producing high affinity antibody. Selection of antibodies having affinity to monkey TfR can be carried out by, for example, ELISA using a recombinant monkey TfR which is prepared utilizing recombinant DNA technologies. In such an ELISA, a recombinant monkey TfR is added to a plate and held by it, and contacted by the anti-hTfR antibody, and, after removal of antibody unbound to the recombinant monkey TfR from the plate, the amount of the antibody held by the plate is measured. The higher the affinity of it to the recombinant monkey hTfR is, the greater the amount of the antibody held by the plate becomes. Consequently, the antibody corresponding to the plate which held the greater amount of antibody can be selected as the antibody having affinity to monkey TfR. Here, the term “monkey” is preferably classified as simians except human, more preferably as Cercopithecidae, still more preferably as macaques, and for example cynomolgus monkey or Rhesus monkey, among which cynomolgus monkey is convenient for use in examination.

An antibody having affinity both to human and monkey hTfRs offers an advantage that it allows pharmacokinetic observation of the antibody administered to the body using a monkey. For example, if a medical drug is being developed utilizing such an anti-hTfR antibody of the present invention, the progress of its development can be remarkably accelerated, for its pharmacokinetic study can be performed using a monkey.

An antibody having a relatively high-affinity to hTfR and having affinity both to human and monkey TfRs, simultaneously, exhibits a dissociation constant with monkey TfR, as measured by the method described in Example 7, that is preferably not greater than 5×10⁻⁸ M, more preferably not greater than 2×10⁻⁸ M, and still more preferably not greater than 1×10⁻⁸M. For example, one which exhibits a dissociation constant of 1×10⁻¹³ M to 2×10⁻⁸M, or 1×10⁻¹³ M to 2×10⁻⁸ M is preferred. The same also applies if the antibody is a single-chain antibody.

If an antibody having a relatively high-affinity to hTfR and obtained by the above method in which those cells producing a high affinity antibody were selected, is an antibody of a non-human animal, it may be converted to a humanized antibody. A humanized antibody is an antibody produced by replacing a proper region of a human antibody with an amino acid sequence of part of the variable region (e.g., the whole or part of the CDRs) of a non-human animal antibody (implant of the sequence into the proper region of a human antibody), while maintaining the specificity to the antigen. Examples of humanized antibodies include an antibody produced by replacing the three complementarity determining regions (CDRs) in the immunoglobulin light chain and the three complementarity determining regions (CDRs) in the immunoglobulin heavy chain, both constituting a human antibody, with CDRs of a non-human mammal. Though there is no particular limitation as to the biological species from which the CDRs to be incorporated into the human antibody are derived so long as it is a non-human mammal, it preferably is a mouse, rat, rabbit, horse, and non-human primate, more preferably a mouse and rat, and still more preferably a mouse.

Methods for preparation of humanized antibody are well known in the art and the most common is a method in which the amino acid sequence of the complementarity determining regions (CDRs) in the variable region of a human antibody is replaced with the CDRs of an antibody of non-human mammal, as devised by Winter et al. (Verhoeyen M. Science. 239, 1534-1536 (1988)). It is also well known that in some cases, corresponding part of an acceptor human antibody needs to be replaced not only with the CDRs of the non-human mammalian antibody but also amino acid sequences occurring in regions outside the CDRs that play a role either in maintaining the structure of the CDRs or in binding to the antigen, in order to reproduce the activity that the donor antibody originally possesses (Queen C. Proc. Natl. Acad. Sci. USA. 86. 10029-10033 (1989)). Here, the regions outside the CDRs are called framework (FR) regions.

Thus, preparation of humanized antibody involves processes of implanting the CDRs (and their neighboring FRs, as the case may be) of non-human mammalian antibody in place of the CDRs (and their neighboring FRs, as the case may be) in the variable region of a human antibody. In such processes, the starting framework region of the variable region of a human antibody can be obtained from a public DNA database and the like which includes germ line antibody genes. For example, germ line DNA sequences, as well as amino acid sequences, of human heavy chain and light chain variable regions can be selected from “VBase” human germline database (available in the Internet, at www.mrc-cpe.cam.ac.uk/vbase). Besides, they can be selected from DNA sequences and amino acid sequences described in published literatures, such as “Kabat E A. Sequences of Proteins of Immunological Interest, 5th Ed., U.S. Department of Health and Human Services, NIH Publication No. 91-3242 (1991)”; “Tomlinson I M. J. fol. Biol. 227. 776-98 (1992)”; and “Cox J P L. Eur. J Immunol. 24:827-836 (1994)”.

As aforementioned, in a humanized antibody, the regions of a non-human mammal antibody to be implanted into the variable regions of the original human antibody generally include CDRs themselves, or CDRs and their neighboring part of FRs. However, such FRs implanted together with CDRs also play a role either in maintaining the structure of the CDRs or in binding to the antigen, thus having a substantial function in determining the complementarity of an antibody, and the term “CDR” in the present invention, therefore, refers to such regions that are, or could be, taken from a non-human mammal antibody and grafted into a humanized antibody, in preparing a humanized antibody. Thus, a region generally considered to be in a FR region is included in a CDR in the present invention as far as it takes part either in maintaining the structure of the CDR or in binding to the antigen, and is thus considered to have a substantial function in determining the complementarity of the antigen.

The anti-hTfR antibody of the present invention, when administered to the body, e.g., by intravenous injection, efficiently binds to hTfR existing on the endothelial cells of the capillaries in the brain. The antibody bound to the hTfR is taken into the brain across the blood-brain barrier by such mechanisms as endocytosis, and transcytosis. Therefore, by binding to the anti-hTfR antibody of the present invention, proteins, low-molecular-weight compounds and the like that need to be brought into function in the brain, can be efficiently delivered into the brain across the blood-brain barrier. Further, the anti-hTfR antibody of the present invention can, after passing through the blood-brain barrier, can reach the cerebral parenchyma, and neuron-like cells in the hippocampus; Purkinje cells and the like of the cerebellum or at least one of them. And it is also expected that it reaches to the neuron-like cells in the striatum of the cerebrum; and the neuron-like cells in the substantia nigra of the mesencephalon. Therefore, it is possible to make one of those proteins, low-molecular-weight compounds and the like, which could act on such tissues or cells, reach the tissues or cells, by binding it to the anti-hTfR antibody of the present invention.

The anti-hTfR antibody of the present invention can be an effective means to make those compounds (proteins, low-molecular-weight compounds and the like) transfer from the blood into the brain and function there, which compounds otherwise cannot pass through the blood-brain barrier when intravenously administered and therefore cannot or can hardly exhibit their physiological or pharmacological functions in the brain. In particular, the anti-hTfR antibody of the present invention can, after passing through the blood-brain barrier, reach the cerebral parenchyma, and neuron-like cells in the hippocampus; Purkinje cells and the like of the cerebellum or at least one of them. And it is also expected that it reaches to the neuron-like cells in the striatum of the cerebrum; as well as to the neuron-like cells in the substantia nigra of the mesencephalon. Therefore, it is possible to make those compounds function or augment their function, in those tissues or cells in the brain by administering those compounds in a combined form with the anti-hTfR antibody molecule, parenterally, e.g., intravenously.

For binding an anti-hTfR antibody to such compounds (proteins, low-molecular-weight compounds and the like), a method is available to bind them together via a non-peptide linker or a peptide linker. As non-peptide linkers, there can be used polyethylene glycol, polypropylene glycol, copolymer of ethylene glycol and propylene glycol, polyoxyethylated polyol, polyvinyl alcohol, polysaccharides, dextran, polyvinyl ether, biodegradable polymer, polymerized lipid, chitins, and hyaluronic acid, or derivatives thereof, or combinations thereof. A peptide linker is a peptide chain consisting of 1 to 50 amino acids linked by peptide bonds or a derivative thereof, whose N-terminus and C-terminus are to be covalently bonded either to an anti-hTfR antibody or a compound such as a protein, a low-molecular-weight compound and the like, respectively, to bind the anti-hTfR antibody to such a compound like a protein or a low-molecular-weight compound.

In particular, a conjugate which is formed by binding the anti-hTfR antibody of the present invention to a desired different protein (A) via PEG as a non-peptide linker, is designated “anti-hTfR antibody-PEG-protein”. An anti-hTfR antibody-PEG-protein can be prepared by first binding the anti-hTfR antibody to PEG to form anti-hTfR antibody-PEG, and then binding the anti-hTfR antibody-PEG to the different protein (A). Alternatively, an anti-hTfR antibody-PEG-protein can be prepared by first binding the different protein (A) to PEG to form “protein-PEG”, and then binding the “protein-PEG” to the anti-hTfR antibody. In order to bind PEG to the anti-hTfR antibody and the different protein (A), a PEG is employed which is modified with such functional groups as carbonate, carbonylimidazole, active ester of carboxylic acid, azlactone, cyclic imide thione, isocyanate, isothiocyanate, imidate, aldehyde or the like. Such functional groups introduced to PEG react mainly with amino groups in the anti-hTfR antibody and a different protein (A) to covalently bind PEG to the hTfR antibody and a different protein (A). Though there is no particular limitation as to the molecular weight and the configuration of PEG employed here, its mean molecular weight (MW) is as follows: preferably MW=500 to 60000, more preferably MW=500 to 20000. For example, such PEG whose mean molecular weight is about 300, about 500, about 1000, about 2000, about 4000, about 10000, about 20000, and the like. PEG is preferably used as a non-peptide linker. The anti-hTfR antibody can be bound to a desired low-molecular-weight compound in the same manner as above.

For example, “anti-hTfR antibody-PEG” can be prepared by mixing the anti-hTfR antibody with a polyethylene glycol having aldehyde groups as functional groups (ALD-PEG-ALD) so that the molar ratio of ALD-PEG-ALD to the antibody is 11, 12.5, 15, 110, 120 and the like, and then adding to the mixture a reducing agent such as NaCNBH₃ to let a reaction take place. Then, by reacting “anti-hTfR antibody-PEG” with a different protein (A) in the presence of a reducing agent such as NaCNBH₃, “anti-hTfR antibody-PEG-protein” is obtained. On the contrary, it is also possible to obtain “anti-hTfR antibody-PEG-protein” by first binding a different protein (A) to ALD-PEG-ALD to prepare “protein-PEG”, and then binding the “protein-PEG” to the anti-hTfR antibody.

The anti-hTfR antibody and a different protein (A) can also be bound together through peptide bonds by linking the anti-hTfR antibody heavy chain or light chain, on the C-terminal side or the N-terminal side thereof, either via a linker sequence or directly, to the N-terminus or the C-terminus of the different protein (A), respectively. Thus the fusion protein between the anti-hTfR antibody and a different protein (A) can be obtained by incorporating into a mammalian expression vector a DNA fragment in which a cDNA encoding the different protein (A) is placed in-frame directly, or via a DNA fragment encoding a linker sequence, on the 3′-end or 5′-end side of a cDNA encoding the heavy chain or light chain of the anti-hTfR antibody, and culturing mammalian cells into which the above expression vector has been introduced. Where the DNA fragment encoding a different protein (A) is linked to the heavy chain, a mammalian expression vector in which a cDNA fragment encoding the anti-hTfR antibody light chain is also introduced into the same host cells, whereas if DNA fragment encoding a different protein (A) is linked to the light chain, a mammalian expression vector in which a cDNA fragment encoding the anti-hTfR antibody heavy chain is also incorporated into the same host cells. In the case where the anti-hTfR antibody is a single-chain antibody, the fusion protein comprising the anti-hTfR antibody and a different protein (A) combined can be obtained by incorporating, into an expression vector (for eukaryotic cells such as mammalian and yeast, or for prokaryotic cells such as E. coli), a DNA fragment which is formed by linking the cDNA encoding a different protein (A), on the 5′-end side or on the 3′-end side thereof, directly or via a DNA fragment encoding a linker sequence, to the cDNA encoding the single-chain anti-hTfR antibody, and allowing the fusion protein be expressed in those cells into which the expression vector has been introduced.

In a fusion protein of the type in which a different protein (A) is linked to the anti-hTfR antibody light chain on the C-terminal side thereof, the anti-human transferrin receptor antibody comprises an amino acid sequence including the whole or part of the light chain variable region and an amino acid sequence including the whole or part of the heavy chain variable region, and the different protein (A) is linked to the light chain of this anti-human transferrin receptor antibody on the C-terminal side thereof. Here, the anti-hTfR antibody light chain and a different protein (A) may be linked together, directly or via a linker.

In a fusion protein of the type in which a different protein (A) is linked to the anti-hTfR antibody heavy chain on the C-terminal side thereof, the anti-human transferrin receptor antibody comprises an amino acid sequence including the whole or part of the light chain variable region and an amino acid sequence including the whole or part of the heavy chain variable region, and the different protein (A) is linked to the heavy chain of this anti-human transferrin receptor antibody on the C-terminal side thereof. Here, the anti-hTfR antibody heavy chain and a different protein (A) may be linked together, directly or via a linker.

In a fusion protein of the type in which a different protein (A) is linked to the anti-hTfR antibody light chain on the N-terminal side thereof, the anti-human transferrin receptor antibody comprises an amino acid sequence including the whole or part of the light chain variable region and an amino acid sequence including the whole or part of the heavy chain variable region, and the different protein (A) is linked to the light chain of this anti-human transferrin receptor antibody on the N-terminal side thereof. Here, the anti-hTfR antibody light chain and a different protein (A) may be linked together, directly or via a linker.

In a fusion protein of the type in which a different protein (A) is linked to the anti-hTfR antibody heavy chain on the N-terminal side thereof, the anti-human transferrin receptor antibody comprises an amino acid sequence including the whole or part of the light chain variable region and an amino acid sequence including the whole or part of the heavy chain variable region, and the different protein (A) is linked to the heavy chain of this anti-human transferrin receptor antibody on the N-terminal side thereof. Here, the anti-hTfR antibody heavy chain and a different protein (A) may be linked together, directly or via a linker.

In the above, the linker sequence placed between the anti-hTfR antibody and a different protein (A) may be a peptide chain consisting preferably of 1 to 50, more preferably of 1 to 17, still more preferably of 1 to 10, even more preferably of 1 to 5 amino acids, and in accordance with the different protein (A) to be linked to the anti-hTfR antibody, the number of amino acids of the linker sequence may be adjusted to 1, 2, 3, 1 to 17, 1 to 10, 10 to 40, 20 to 34, 23 to 31, 25 to 29, 27, etc., as desired. Though there is no particular limitation as to amino acid sequence of the linker sequence insofar as the anti-hTfR antibody linked by it retains the affinity to hTfR and a different protein (A) linked by the linker sequence also exhibits the protein's own physiological activity under a physiological condition, the linker may preferably be composed of glycine and serine. Examples of such linkers include one consisting of a single amino acid either glycine or serine, the amino acid sequence Gly-Ser, the amino acid sequence Gly-Gly-Ser, the amino acid sequence Gly-Gly-Gly-Gly-Ser (SEQ ID NO:3), the amino acid sequence Gly-Gly-Gly-Gly-Gly-Ser (SEQ ID NO:4), the amino acid sequence Ser-Gly-Gly-Gly-Gly-Gly (SEQ ID NO:5), or a sequence which includes 1 to 10 or 2 to 5 of any of those amino acid sequences consecutively linked. They have sequences consisting of 1 to 50, 2 to 17, 2 to 10, 10 to 40, 20 to 34, 23 to 31, 25 to 29, or 27 amino acids. For example, those comprising the amino acid sequence Gly-Ser may preferably be used as linker sequences. Further, a linker sequence comprising 27 amino acids is preferably used that is composed of the amino acid sequence Gly-Ser followed by consecutively linked five copies of the amino acid sequence Gly-Gly-Gly-Gly-Ser (SEQ ID NO:3).

In a fusion protein of the anti-hTfR antibody and a different protein (A), where the anti-hTfR antibody is a single-chain antibody, the amino acid sequence including the whole or part of the immunoglobulin light chain variable region and the amino acid sequence including the whole or part of the immunoglobulin heavy chain variable region are linked, generally via a linker sequence. Insofar as the affinity of the anti-hTfR antibody to hTfR is retained, the amino acid sequence derived from the light chain may be linked, on the C-terminal side thereof, to a linker sequence which in turn being linked, on the C-terminal side thereof, to the amino acid sequence derived from the heavy chain or, conversely, the amino acid sequence derived from the heavy chain may be linked, on the C-terminal side thereof, to a linker sequence which in turn being linked, on the C-terminal side thereof, to the amino acid sequence derived from the light chain.

The linker sequence placed between the light chain and the heavy chain of the immunoglobulin is a peptide chain consisting preferably of 2 to 50, more preferably 8 to 50, still more preferably 10 to 30, even more preferably 12 to 18 or 15 to 25, and for example 15 or 25 amino acids. Though there is no specific limitation as to the linker sequence insofar as the anti-hTfR antibody made of the both chains which are linked via the linker retains the affinity to hTfR and a different protein (A) linked to the antibody also exhibits the protein's own physiological activity under a physiological condition, the linker is preferably composed of glycine, or glycine and serine. Examples of such linkers include the amino acid sequence Gly-Ser, the amino acid sequence Gly-Gly-Ser, the amino acid sequence Gly-Gly-Gly, the amino acid sequence Gly-Gly-Gly-Gly-Ser (SEQ ID NO:3), the amino acid sequence Gly-Gly-Gly-Gly-Gly-Ser (SEQ ID NO:4), the amino acid sequence Ser-Gly-Gly-Gly-Gly-Gly) (SEQ ID NO:5), or a sequence which includes 2 to 10 or 2 to 5 of any of these amino acid sequences consecutively linked. A preferred embodiment of such a linker sequence comprises 15 amino acids consisting of consecutively linked three copies of the amino acid sequence Gly-Gly-Gly-Gly-Ser (SEQ ID NO:3).

In the case where the anti-hTfR antibody is a single-chain antibody, an example of specific embodiments of the fusion protein between the humanized anti-hTfR antibody of the present invention and a different protein (A) is a fusion protein consisting of the different protein (A) which is linked, on the C-terminal side thereof and via a first linker sequence consisting of 27 amino acids composed of the amino acid sequence Gly-Ser followed by consecutively linked five copies of the amino acid sequence Gly-Gly-Gly-Gly-Ser (SEQ ID NO:3), to the single-chain antibody. An example of a preferred embodiment of single-chain antibodies employed here is an antibody having the amino acid sequence set forth as SEQ ID NO:277, which is composed of the amino acid sequence of the anti-hTfR antibody heavy chain variable region set forth as SEQ ID NO:205 that is linked, at the C-terminus thereof and via a first linker sequence consisting of 15 amino acids consisting of consecutively linked three copies of the amino acid sequence Gly-Gly-Gly-Gly-Ser (SEQ ID NO:3), to the anti-hTfR antibody light chain variable region having the amino acid sequence set forth as SEQ ID NO:191.

Where the anti-hTfR antibody is a single-chain antibody, such a fusion protein can be produced by, for example, transforming host cells such as mammalian cells with an expression vector having an incorporated DNA fragment containing a nucleotide sequence encoding the fusion protein, and then culturing the host cells.

Besides, in the present invention, when a peptide chain includes a plurality of linker sequences, each of those linker sequences is designated, from the N-terminal side, the first linker sequence, the second linker sequence, and so on, for convenience.

In the case where the anti-hTfR antibody is Fab, an example of specific embodiments of the fusion protein between a humanized anti-hTfR antibody and a different protein (A) of the present invention is a fusion protein which is composed of the different protein (A) that is fused, on the C-terminal side thereof and via a linker sequence consisting of 27 amino acids composed of Gly-Ser followed by consecutively linked five copies of the amino acid sequence Gly-Gly-Gly-Gly-Ser (SEQ ID NO:3), to a region having the anti-hTfR antibody heavy chain variable region and the C_{H}1 region. Though part of the hinge region may be included in addition to the C_{H}1 region here, the hinge region includes no cysteine residue which would form a disulfide bond between heavy chains.

Though there is no particular limitation as to the different protein (A) to be linked to the anti-hTfR antibody, it is a protein that can exhibit its physiological activity in the body, and in particular, such a protein that needs to get inside the brain and exhibit its function there but, due to its inability to pass through the blood-brain barrier as it is, cannot be expected to function in the brain if simply administered intravenously. Examples of such proteins include lysosomal enzymes such as nerve growth factor (NGF), α-L-iduronidase, iduronate 2-sulfatase, glucocerebrosidase, β-galactosidase, GM2 activator protein, β-hexosaminidase A, β-hexosaminidase B, N-acetylglucosamine-1-phosphotransferase, α-mannosidase, β-mannosidase, galactosylceramidase, saposin C, arylsulfatase A, α-L-fucosidase, aspartylglucosaminidase, α-N-acetylgalactosaminidase, acidic sphingomyelinase, α-galactosidase A, β-glucuronidase, heparan N-sulfatase, α-N-acetylglucosaminidase, acetyl COA:α-glucosaminide N-acetyltransferase, N-Acetylglucosamine-6-sulfate sulfatase, acid ceramidase, amylo-1,6-glucosidase, sialidase, aspartylglucosaminidase (PPT1), tripeptidyl-peptidase 1, hyaluronidase 1, CLN1, and CLN2, and the like.

The nerve growth factor (NGF) linked to the anti-hTfR antibody can be used as a therapeutic agent for dementia in Alzheimer's disease; α-L-iduronidase linked to the anti-hTfR antibody as a therapeutic agent for central nervous system disorders in Hurler syndrome or Hurler-Scheie syndrome; iduronate 2-sulfatase linked to the anti-hTfR antibody as a therapeutic agent for central nervous system disorders in Hunter syndrome; glucocerebrosidase as a therapeutic agent for central nervous system disorders in Gaucher's disease; β-galactosidase as a therapeutic agent for central nervous system disorders in GM1 gangliosidosis Types 1 to 3; GM2 activator protein as a therapeutic agent for central nervous system disorders in GM2-gangliosidosis, AB variant; β-hexosaminidase A as a therapeutic agent for central nervous system disorders in Sandhoff's disease and Tay-Sachs disease; β-hexosaminidase B as a therapeutic agent for central nervous system disorders in Sandhoff's disease; N-acetylglucosamine-1-phosphotransferase as a therapeutic agent for central nervous system disorders in I-cell disease; α-mannosidase as a therapeutic agent for central nervous system disorders in α-mannosidosis; β-mannosidase as a therapeutic agent for central nervous system disorders in β-mannosidosis; galactosylceramidase as a therapeutic agent for central nervous system disorders in Krabbe disease; saposin C as a therapeutic agent for central nervous system disorders in Gaucher's disease-like storage disease; arylsulfatase A as a therapeutic agent for central nervous system disorders in metachromatic white matter degeneration (metachromatic leukodystrophy); α-L-fucosidase as a therapeutic agent for central nervous system disorders in fucosidosis; aspartylglucosaminidase as a therapeutic agent for central nervous system disorders in aspartylglucosaminuria; α-N-acetylgalactosaminidase as a therapeutic agent for central nervous system disorders in Schindler disease and Kawasaki disease; acidic sphingomyelinase as a therapeutic agent for central nervous system disorders in Niemann-Pick disease; α-galactosidase A as a therapeutic agent for central nervous system disorders in Fabry disease; β-glucuronidase as a therapeutic agent for central nervous system disorders in Sly syndrome; heparan N-sulfatase, α-N-acetylglucosaminidase, acetyl CoA:α-glucosaminide N-acetyltransferase and N-Acetylglucosamine-6-sulfate sulfatase as therapeutic agents for central nervous system disorders in Sanfilippo syndrome; acid ceramidase as a therapeutic agent for central nervous system disorders in Farber disease; amylo-1,6-glucosidase as a therapeutic agent for central nervous system disorders in Cori's disease (Forbes-Cori's disease); sialidase as a therapeutic agent for central nervous system disorders in sialidase deficiency; aspartylglucosaminidase as a therapeutic agent for central nervous system disorders in aspartylglucosaminuria; palmitoyl protein thioesterase 1 (PPT-1) as a therapeutic agent for central nervous system disorders in neuronal ceroid lipofuscinosis or Santavuori-Haltia disease; tripeptidyl-peptidase 1 (TPP-1) as a therapeutic agent for central nervous system disorders in neuronal ceroid lipofuscinosis or Jansky-Bielschowsky disease; hyaluronidase 1 as a therapeutic agent for central nervous system disorders in hyaluronidase deficiency; CLN1 and CLN2 as therapeutic agents for central nervous system disorders in Batten disease. In particular, the anti-hTfR antibody of the present invention, after passing through the blood-brain barrier, reaches the brain parenchyma and the hippocampus neuron-like cells of the cerebrum, and Purkinje cells of the cerebellum, and is expected further to reach neuron-like cells of the striatum of the cerebrum and the neuron-like cells of the substantia nigra of the mesencephalon. Therefore, the anti-hTfR antibody can be fused with proteins which need to exhibit their functions in those tissues or cells to strength the pharmacological effects of the proteins. Medical applications of it, however, are not limited thereto.

Further, examples of proteins that can exhibit their pharmacological effects when linked to the anti-hTfR antibody include: lysosomal enzymes, ciliary neurotrophic factor (CNTF), glial cell line derived neurotrophic factor (GDNF), neurotrophin-3, neurotrophin-4/5, neurotrophin-6, neuregulin-1, erythropoietin, darbepoetin, activin, basic fibroblast growth factor (bFGF), fibroblast growth factor 2 (FGF2), epidermal growth factor (EGF), vascular endothelial growth factor (VEGF), interferon α, interferon β, interferon Y, interleukin 6, granulocyte-macrophage colony stimulating factor (GM-CSF), granulocyte colony stimulating factor (G-CSF), macrophage colony stimulating factor (M-CSF), cytokines, tumor necrosis factor α receptor (TNF-α receptor), PD-1 ligands, enzymes having β-amyloid-degrading activity, anti-β-amyloid antibody, anti-BACE antibody, anti-EGFR antibody, anti-PD-1 antibody, anti-PD-L1 antibody, anti-HER2 antibody, anti-TNF-α antibody, and other antibody medicines.

Lysosomal enzymes linked to the anti-hTfR antibody can be used as a therapeutic agent for central nervous system disorders in lysosomal storage diseases; CNTF as a therapeutic agent for amyotrophic lateral sclerosis; GDNF, neurotrophin-3 and neurotrophin-4/5 as therapeutic agents for cerebral ischemia; GDNF as a therapeutic agent for Parkinson's disease; neuregulin-1 as a therapeutic agent for schizophrenia; erythropoietin and darbepoetin as therapeutic agents for cerebral ischemia; bFGF and FGF2 as therapeutic agents for traumatic central nervous system disorders; for recovery after brain surgery and spinal surgery; enzymes having β-amyloid-degrading activity, anti-β-amyloid antibody and anti-BACE antibody as therapeutic agents for Alzheimer's disease; anti-EGFR antibody, anti-PD-1 antibody, anti-PD-L1 antibody, and anti-HER2 antibody as therapeutic agents for tumors of central nervous system including brain tumor; and TNFαR-anti-hTfR antibody as therapeutic agents for a cerebral ischemia and encephalitis.

Possible candidates for a “different protein (A)” to be fused to the anti-hTfR antibody generally include those therapeutic agents for diseases such as neurodegenerative diseases such as Alzheimer's disease, Parkinson's disease, and Huntington's disease; mental disorders such as schizophrenia and depression; multiple sclerosis; amyotrophic lateral sclerosis; tumors of the central nervous system including brain tumor; lysosomal storage diseases accompanied by encephalopathy; glycogenosis; muscular dystrophy; cerebral ischemia; encephalitis; prion diseases; traumatic central nervous system disorders. In addition, therapeutic agent for viral and bacterial central nervous system diseases can also be candidates for a different protein (A) to be fused to the anti-hTfR antibody, in general. Further, pharmaceutical agents that can be used for recovery after brain surgery or spinal surgery can also be candidates for a “different protein (A)” to be fused to the anti-hTfR antibody, in general.

In addition to the above mentioned natural-type (wild-type) proteins, a different protein (A) to be linked to the anti-hTfR antibody may also be one of their analogues in which one or more amino acids of those natural-type (wild-type) proteins are modified, e.g., replaced with other amino acids or deleted, insofar as they fully or partly have the functions of their respective original proteins. When replacing one or more amino acids with other amino acids, the number of amino acids to be replaced is preferably 1 to 10, more preferably 1 to 5, still more preferably 1 to 3. When deleting one or more amino acids, the number of amino acids to be deleted is preferably 1 to 10, more preferably 1 to 5, still more preferably 1 to 3. A combination of such substitution and deletion of amino acids can also be carried out to prepare desired analogues. Further, amino acid sequences produced by adding one or more amino acids inside, or on the N-terminal side or on the C-terminal side of, the amino acid sequence of natural-type (wild-type) proteins or their analogues, are also included in the proteins mentioned above insofar as they fully or partly have the functions of their respective original proteins. The number of amino acids to be added here is preferably 1 to 10, more preferably 1 to 5, still more preferably 1 to 3. It is also possible to prepare desired analogues to the original proteins by combining addition, substitution, and deletion of amino acids.

Besides, in the case where a mutation is introduced into a different protein (A) by adding one or more amino acids on its C-terminus or the N-terminus, if the added amino acids are positioned between the protein and the anti-hTfR antibody when they are fused, the added amino acids constitute part of a linker.

The natural-type human I2S (hI2S) is a lysosomal enzyme composed of 525 amino acids set forth as SEQ ID NO:246. A specific example of fusion proteins of the present invention between the anti-hTfR antibody and a different protein (A) is a type in which the anti-hTfR antibody heavy chain is fused, on the C-terminus thereof and via the amino acid sequence Gly-Ser as a linker sequence, to the natural-type human I2S. Examples of such a type of fusion proteins include:
(1) one whose light chain consists of the amino acid sequence set forth as SEQ ID NO:164, and whose heavy chain linked by peptide pond, on the C-terminal side thereof and via the linker sequence Gly-Ser, to human I2S forms the amino acid sequence set forth as SEQ ID NO:247,
(2) one whose light chain consists of the amino acid sequence set forth as SEQ ID NO:180, and whose heavy chain linked by peptide pond, on the C-terminal side thereof and via the linker sequence Gly-Ser, to human I2S forms the amino acid sequence set forth as SEQ ID NO:249, and
(3) one whose light chain consists of the amino acid sequence set forth as SEQ ID NO:196, and whose heavy chain linked by peptide pond, on the C-terminal side thereof and via the linker sequence Gly-Ser, to human I2S forms the amino acid sequence set forth as SEQ ID NO:251.

In the present invention, though the term “human I2S” or “hI2S” refers, in particular, to the hI2S having the same amino acid sequence as the natural-type hI2S, it also includes those amino acid sequences produced by introducing a mutation, such as substitution, deletion, addition and the like, into the amino acid sequence of the natural-type hI2S, insofar as they have the I2S activity. When replacing one or more of the amino acids of the amino acid sequence of hI2S with other amino acids, the number of amino acids to be replaced is preferably 1 to 10, more preferably 1 to 5, still more preferably 1 to 3, even more preferably 1 to 2. When deleting one or more amino acids of the amino acid sequence of hI2S, the number of amino acids to be deleted is 1 to 10, more preferably 1 to 5, still more preferably 1 to 3, and even more preferably 1 or 2. It is also possible to introduce a combined mutation of such substitution and deletion of amino acids. When adding one or more amino acids to hI2S, they may be added, inside, or on the N-terminal side or the C-terminal side of, the amino acid sequence of hI2S, and the number of amino acids to be added is preferably 1 to 10, more preferably 1 to 5, still more preferably 1 to 3, even more preferably 1 or 2. It is also possible to introduce a combined mutation of such addition, substitution, and deletion of the amino acid. The amino acid sequence of the mutated hI2S has a homology of preferably not lower than 80%, more preferably not lower than 90%, still more preferably not lower than 95% to the amino acid sequence of the original hI2S.

The statement that hI2S has the I2S activity herein means that the hI2S fused to anti-hTfR antibody has an activity not lower than 3% of the activity that the natural-type hI2S intrinsically has. However, the activity is preferably not lower than 10%, more preferably not lower than 20%, still more preferably not lower than 50%, even more preferably not lower than 80% of the activity that the natural-type hI2S intrinsically has. The same also applies if the hI2S fused to the anti-hTfR antibody is mutated.

The fusion protein between the anti-hTfR antibody and human I2S can be produced by, for example, transforming host cells, such as mammalian cells, with an expression vector having an incorporated DNA fragment comprising the nucleotide sequence set forth as SEQ ID NO:250 that encodes the amino acid sequence set forth as SEQ ID NO:249 and an expression vector having an incorporated DNA fragment comprising the nucleotide sequence set forth as SEQ ID NO:181 that encodes the anti-hTfR antibody light chain having the amino acid sequence set forth as SEQ ID NO:180, and then culturing the host cells. The fusion protein thus produced can be used as a therapeutic agent for Hunter disease, in particular, a therapeutic agent for central nervous system disorders accompanying Hunter disease.

Besides, in the case where the anti-hTfR antibody or human I2S is mutated by adding one or more amino acids to them on the C-terminus or the N-terminus thereof, if the added amino acids are positioned between the anti-hTfR antibody and human I2S, the added amino acids constitute part of a linker.

Though examples of fusion proteins between the anti-hTfR antibody and human I2S are described above, there is no particular limitation as to the amino acid sequence of the CDRs of the anti-hTfR antibody heavy chain and light chain in preferred embodiments of fusion protein of the anti-hTfR antibody and human I2S insofar as the antibody has a specific affinity to hTfR. However, the anti-hTfR antibody of the present invention exhibits a dissociation constant (K_{D}) with hTfR, as measured by the method described in Example 7, that is preferably not greater than 1×10⁻⁸M, more preferably not greater than 1×10⁻⁹ M, still more preferably not greater than 1×10⁻¹⁰. For example, one which exhibits a dissociation constant of 1×10⁻¹³ M to 1×10⁻⁹M or 1×10⁻¹³ M to 1×10⁻¹⁰ M is preferred. The same also applies when the antibody is a single-chain antibody. Further, in the case where the anti-hTfR antibody of the present invention has also an affinity to monkey TfR, the dissociation constant of the anti-hTfR antibody with monkey TfR, as measured by the method described in Example 7, is preferably not greater than 5×10⁻⁸ M, more preferably not greater than 2×10⁻⁸ M, still more preferably not greater than 1×10⁻⁸ M. For example, one which exhibits a dissociation constant of 1×10⁻¹³ M to 2×10⁻⁸ M is preferred. The same also applies when the antibody is a single-chain antibody.

It is also possible to link a relatively short peptide chain to the anti-hTfR antibody, in the same manner as in linking a different protein (A) to the anti-hTfR antibody. There is no particular limitation as to a peptide chain to be linked to the anti-hTfR antibody, insofar as the peptide chain has a desired physiological activity. For example, there are peptide chains comprising the amino acid sequence of such a region of various proteins that exhibits a physiological activity. Though there is no particular limitation as to the length of the peptide chain, they are composed of preferably 2 to 200 amino acids, for example of 5 to 50 amino acids.

In linking a low-molecular-weight compound to the anti-hTfR antibody, there is no particular limitation as to candidate low-molecular-weight compounds, but they are such low-molecular-weight compound that though needed to get inside the brain and function there, due to their inability to pass through the blood-brain barrier as it is, cannot be expected to function in the brain if simply administered intravenously. Examples of such low-molecular-weight compounds include anticancer drug such as cyclophosphamide, ifosfamide, melphalan, busulfan, thioTEPA, nimustine, ranimustine, dacarbazine, procarbazine, temozolomide, carmustine, streptozocin, bendamustine, cisplatin, carboplatin, oxaliplatin, nedaplatin, 5-fluorouracil, sulfadiazine, sulfamethoxazole, methotrexate, trimethoprim, pyrimethamine, fluorouracil, flucytosine, azathioprine, pentostatin, hydroxyurea, fludarabine, cytarabine, gemcitabine, irinotecan, doxorubicin, etoposide, levofloxacin, ciprofloxacin, vinblastine, vincristine, paclitaxel, docetaxel, Mitomycin C, doxorubicin, epirubicin. Further examples of low-molecular-weight compound to be linked to the anti-hTfR antibody include siRNAs, antisense DNAs, and short peptides.

In linking between the anti-hTfR antibody and a low-molecular-weight compound, either a low-molecular-weight compound may be linked only to either the light chain or the heavy chain, or it may be linked to both the light chain and the heavy chain, respectively. Further, insofar as it has an affinity to hTfR, the anti-hTfR antibody may comprise an amino acid sequence comprising the whole of part of the light chain variable region and/or an amino acid sequence comprising the whole of part of the heavy chain variable region.

Candidates for low-molecular-weight compounds to be fused with the anti-hTfR antibody can generally be those therapeutic agents for diseases such as neurodegenerative diseases such as Alzheimer's disease, Parkinson's disease, Huntington's disease; mental disorders such as schizophrenia, depression; multiple sclerosis; amyotrophic lateral sclerosis; tumor of the central nervous system including brain tumor; lysosomal storage diseases with accompanying encephalopathy; glycogenosis; muscular dystrophy; cerebral ischemia; encephalitis; prion diseases; traumatic disorders of the central nervous system. Further, therapeutic agents for viral and bacterial central nervous system diseases can also be candidates, in general, for low-molecular-weight compounds to be fused with the anti-hTfR antibody. Still further, those pharmaceutical agents which can be used for recovery after brain surgery or spinal surgery can also be candidates, in general, for low-molecular-weight compounds to be fused.

If an anti-hTfR antibody originates from a non-human animal, its administration to human could entail a substantial risk of causing an antigen-antibody interaction, thereby provoking adverse side-effects. By converting them to humanized antibodies, the antigenicity of non-human animal antibodies can be reduced and therefore the provocation of side-effects due to antigen-antibody interaction can be suppressed when administered to a human. Further, it has been reported that according to experiments using monkeys, humanized antibodies are more stable than mouse antibodies in the blood, and it is expected that their therapeutic effect can therefore become longer-lasting accordingly. Provocation of side-effects due to an antigen-antibody interaction can be suppressed also by employing a human antibody as the anti-hTfR antibody.

A detailed explanation will be given below regarding the case where the anti-hTfR antibody is a humanized antibody or human antibody. In human antibody light chain, there are λ and κ chains. The light chain constituting the human antibody may either be λ and κ chain. And in human heavy chain, there are γ, μ, α, σ, and ε chains, which correspond to IgG, IgM, IgA, IgD and IgE, respectively. Though the heavy chain constituting the anti-hTfR antibody may be any one of γ, μ, α, σ, and ε chains, preferred is a γ chain. Further, in γ chain of human heavy chain, there are γ1, γ2, γ3 and γ4 chains, which correspond to IgG1, IgG2, IgG3 and IgG4, respectively. Where the heavy chain constituting the anti-hTfR antibody is a γ chain, though the γ chain may be any of γ1, γ2, γ3 and γ4 chains, preferred is a γ1 or γ4 chain. In the case where the anti-hTfR antibody is a humanized antibody or human antibody and IgG, the human antibody light chain may either be λ chain or κ chain, and though the human antibody heavy chain may either be γ1, γ2, γ3 and γ4 chains, preferred is a γ1 or γ4 chain. For example, a preferable embodiment of anti-hTfR antibody includes one whose light chain is a λ chain and heavy chain is a γ1 chain.

In the case where the anti-hTfR antibody is a humanized antibody or a human antibody, the anti-hTfR antibody and a different protein (A) may be bound together by linking the anti-hTfR antibody, at the N-terminus (or the C-terminus) of the heavy chain or light chain, via a linker sequence or directly, to the C-terminus (or the N-terminus), respectively, of the different protein (A), by peptide bonds. When linking the different protein (A) to the anti-hTfR antibody heavy chain on the N-terminal side (or to the C-terminal side) thereof, the C-terminus (or the N-terminus), respectively, of the different protein (A) is linked to the N-terminus (or the C-terminus) of the γ, μ, α, σ or ε chain of anti-hTfR antibody, via a linker sequence or directly, by peptide bonds. When linking the different protein (A) to the anti-hTfR antibody light chain on the N-terminal side (or the C-terminal side) thereof, the C-terminus (or the N-terminus), respectively, of the different protein (A) in linked to the N-terminus (or the C-terminus) of the Δ chain and κ chain of anti-hTfR antibody, via a linker sequence or directly, by peptide bonds. However, in the case where the anti-hTfR antibody consists of the Fab region, or of the Fab region and the whole or part of the hinge region (Fab, F(ab′)₂, and F(ab′)), the different protein (A) may be linked at the C-terminus (or the N-terminus) thereof and via a linker sequence or directly, to the N-terminus (or the C-terminus), respectively, of the heavy chain or light chain that constitutes the Fab, F(ab′)₂ and F(ab′), by peptide bonds.

In a fusion protein produced by linking the different protein (A) to the light chain of the anti-hTfR antibody which is a humanized antibody, or a human antibody, on the C-terminal side thereof, the anti-human transferrin receptor antibody comprises an amino acid sequence comprising the whole or part of the light chain variable region and the amino acid sequence comprising the whole or part of the heavy chain variable region. The anti-hTfR antibody light chain and the different protein (A) here may be linked directly or via a linker.

In a fusion protein produced by linking the different protein (A) to the heavy chain of the anti-hTfR antibody which is a humanized antibody, or human antibody, on the C-terminal side thereof, the anti-human transferrin receptor antibody comprises an amino acid sequence comprising the whole or part of the light chain variable region and the amino acid sequence comprising the whole or part of the heavy chain variable region. The anti-hTfR antibody heavy chain and the different protein (A) here may be linked directly or via a linker.

In a fusion protein produced by linking the different protein (A) to the light chain of the anti-hTfR antibody which is a humanized antibody, or a human antibody, on the N-terminal side thereof, the anti-human transferrin receptor antibody comprises an amino acid sequence comprising the whole or part of the light chain variable region and the amino acid sequence comprising the whole or part of the heavy chain variable region. The anti-hTfR antibody light chain and the different protein (A) here may be linked directly or via a linker.

In a fusion protein produced by linking the different protein (A) to the heavy chain of the anti-hTfR antibody which is a humanized antibody, or human antibody, on the N-terminal side thereof, the anti-human transferrin receptor antibody comprises an amino acid sequence comprising the whole or part of the light chain variable region and the amino acid sequence comprising the whole or part of the heavy chain variable region. The anti-hTfR antibody heavy chain and the different protein (A) here may be linked directly or via a linker.

When placing a linker sequence between the anti-hTfR antibody and a different protein (A), the linker sequence is preferably a peptide chain consisting of 1 to 50 amino acids, though the number of the amino acids constituting such a linker sequence may be adjusted as desired in accordance with the different protein (A) to be linked to the anti-hTfR antibody, like 1 to 17, 1 to 10, 10 to 40, 20 to 34, 23 to 31, 25 to 29, and so on. While there is no particular limitation as to the specific amino acid sequence of such a linker sequence insofar as the anti-hTfR antibody and the different protein (A) linked by the linker sequence retain their respective functions (affinity to hTfR, and activity or function under a physiological condition), it is preferably composed of glycine or serine, for example, one consisting of a single amino acid either glycine or serine, the amino acid sequence Gly-Ser, the amino acid sequence Gly-Gly-Ser, the amino acid sequence Gly-Gly-Gly-Gly-Ser (SEQ ID NO:3), the amino acid sequence Gly-Gly-Gly-Gly-Gly-Ser (SEQ ID NO:4), the amino acid sequence Ser-Gly-Gly-Gly-Gly-Gly (SEQ ID NO:5), or a sequence which includes 1 to 10 or 2 to 5 of any of those amino acid sequences consecutively linked. For example, a linker sequence comprising 27 amino acids is preferably used that is composed of the amino acid sequence Gly-Ser followed by consecutively linked five copies of the amino acid sequence Gly-Gly-Gly-Gly-Ser (SEQ ID NO:3).

Besides, when stated here that a different protein (A) fused with the anti-hTfR antibody retains its activity or function under a physiological condition, or simply, it “retains the activity”, it means that in comparison with the intrinsic activity of the natural-type of the different protein (A), not lower than 3% of the activity or function is retained. However, such an activity or function is preferably not lower than 10%, more preferably not lower than 20%, still more preferably not lower than 50%, and even more preferably not lower than 80%, in comparison with the intrinsic activity of the natural-type of the different protein (A). The same also applies where the different protein (A) fused with the anti-hTfR antibody is a mutated one.

A further example of specific embodiments of the fusion protein between a humanized anti-hTfR antibody and a different protein (A) of the present invention is one produced by fusing the anti-hTfR antibody heavy chain, on the C-terminal side thereof, with a different protein (A), via a linker sequence consisting of 27 amino acids that is composed of the amino acid sequence Gly-Ser followed by consecutively linked five copies of the amino acid sequence Gly-Gly-Gly-Gly-Ser (SEQ ID NO:3).

In the case where the anti-hTfR antibody is Fab, an example of specific embodiments of fusion proteins between a humanized anti-hTfR antibody of the present invention and a different protein (A) is one produced by fusing a different protein (A), on the C-terminal side thereof via a linker sequences, with the region consisting of anti-hTfR antibody heavy chain variable region and its accompanying CHI region, wherein the linker sequence consists of 27 amino acids that is composed of the amino acid sequence Gly-Ser followed by consecutively linked five copies of the amino acid sequence Gly-Gly-Gly-Gly-Ser (SEQ ID NO:3). Though it is also allowed here that part of the hinge region is also besides the CHI region, the hinge region does not contain a cysteine residue that would form a disulfide bond between heavy chains.

The specific affinity of the anti-hTfR antibody to hTfR resides mainly in the amino acid sequences of CDRs of the heavy chain and light chain of the anti-hTfR antibody. There is no particular limitation as to the amino acid sequences of those CDRs insofar as the anti-hTfR antibody has a specific affinity to hTfR. However, the anti-hTfR antibody of the present invention is one whose dissociation constant (K_{D}) with hTfR as measured by the method described in Example 7 is preferably not greater than 1×10⁻⁸M, more preferably not greater than 1×10⁻⁹ M, still more preferably not greater than 1×10⁻¹⁰ M, and even more preferably not greater than 1×10⁻¹¹ M. For example, one having a dissociation constant of 1×10⁻¹³ M to 1×10⁻⁹M, or 1×10⁻¹³ M to 1×10⁻¹⁰ M is preferable. The same also applies when the antibody is a single-chain antibody. Further, where the anti-hTfR antibody of the present invention has affinity also to monkey TfR, the dissociation constant of the anti-hTfR antibody with monkey TfR, as measured by the method described in Example 7, is preferably not greater than 5×10⁻⁸ M, more preferably not greater than 2×10⁻⁸M, and still more preferably not greater than 1×10⁻⁸ M. For example, one which exhibits a dissociation constant of 1×10⁻¹³ M to 2×10⁻⁸M is preferred. The same also applies if the antibody is a single-chain antibody.

Examples of preferable embodiments of the antibody having affinity to hTfR include those whose light chain CDRs have amino acid sequences according to one of (1) to (14) below:
(1) the amino acid sequence set forth as SEQ ID NO:6 or SEQ ID NO:7 as CDR1; the amino acid sequence set forth as SEQ ID NO:8 or SEQ ID NO:9 or the amino acid sequence Trp-Thr-Ser as CDR2; and the amino acid sequence set forth as SEQ ID NO:10 as CDR3;
(2) the amino acid sequence set forth as SEQ ID NO:11 or SEQ ID NO:12 as CDR1; the amino acid sequence set forth as SEQ ID NO:13 or SEQ ID NO:14 or the amino acid sequence Tyr-Ala-Ser as CDR2; and the amino acid sequence set forth as SEQ ID NO:15 as CDR3;
(3) the amino acid sequence set forth as SEQ ID NO:16 or SEQ ID NO:17 as CDR1; the amino acid sequence set forth as SEQ ID NO:18 or SEQ ID NO:19 or the amino acid sequence Lys-Val-Ser as CDR2; and the amino acid sequence set forth as SEQ ID NO:20 as CDR3;
(4) the amino acid sequence set forth as SEQ ID NO:21 or SEQ ID NO:22 as CDR1; the amino acid sequence set forth as SEQ ID NO:23 or SEQ ID NO:24 or the amino acid sequence Asp-Thr-Ser as CDR2; and the amino acid sequence set forth as SEQ ID NO:25 as CDR3;
(5) the amino acid sequence set forth as SEQ ID NO:26 or SEQ ID NO:27 as CDR1; the amino acid sequence set forth as SEQ ID NO:28 or SEQ ID NO:29 or the amino acid sequence Asp-Thr-Ser as CDR2; and the amino acid sequence set forth as SEQ ID NO:30 as CDR3;
(6) the amino acid sequence set forth as SEQ ID NO:31 or SEQ ID NO:32 as CDR1; the amino acid sequence set forth as SEQ ID NO:33 or SEQ ID NO:34 or the amino acid sequence Ala-Ala-Ser as CDR2; and the amino acid sequence set forth as SEQ ID NO:35 as CDR3;
(7) the amino acid sequence set forth as SEQ ID NO:36 or SEQ ID NO:37 as CDR1; the amino acid sequence set forth as SEQ ID NO:38 or SEQ ID NO:39 or the amino acid sequence Gln-Th-Ser as CDR2; and the amino acid sequence set forth as SEQ ID NO:40 as CDR3;
(8) the amino acid sequence set forth as SEQ ID NO:41 or SEQ ID NO:42 as CDR1; the amino acid sequence set forth as SEQ ID NO:43 or SEQ ID NO:44 or the amino acid sequence Gly-Thr-Ser as CDR2; and the amino acid sequence set forth as SEQ ID NO:45 as CDR3;
(9) the amino acid sequence set forth as SEQ ID NO:46 or SEQ ID NO:47 as CDR1; the amino acid sequence set forth as SEQ ID NO:48 or SEQ ID NO:49 or the amino acid sequence Phe-Thr-Ser as CDR2; and the amino acid sequence set forth as SEQ ID NO:50 as CDR3;
(10) the amino acid sequence set forth as SEQ ID NO:51 or SEQ ID NO:52 as CDR1; the amino acid sequence set forth as SEQ ID NO:53 or SEQ ID NO:54 or the amino acid sequence Ala-Ala-Ser as CDR2; and the amino acid sequence set forth as SEQ ID NO:55 as CDR3;
(11) the amino acid sequence set forth as SEQ ID NO:56 or SEQ ID NO:57 as CDR1; the amino acid sequence set forth as SEQ ID NO:58 or SEQ ID NO:59 or the amino acid sequence Tyr-Ala-Ser as CDR2; and the amino acid sequence set forth as SEQ ID NO:60 as CDR3;
(12) the amino acid sequence set forth as SEQ ID NO:61 or SEQ ID NO:62 as CDR1; the amino acid sequence set forth as SEQ ID NO:63 or SEQ ID NO:64 or the amino acid sequence Trp-Ser-Ser as CDR2; and the amino acid sequence set forth as SEQ ID NO:65 as CDR3;
(13) the amino acid sequence set forth as SEQ ID NO:66 or SEQ ID NO:67 as CDR1; the amino acid sequence set forth as SEQ ID NO:68 or SEQ ID NO:69 or the amino acid sequence Tyr-Ala-Ser as CDR2; and the amino acid sequence set forth as SEQ ID NO:70 as CDR3; and
(14) the amino acid sequence set forth as SEQ ID NO:71 or SEQ ID NO:72 as CDR1; the amino acid sequence set forth as SEQ ID NO:73 or SEQ ID NO:74 or the amino acid sequence Asp-Thr-Ser as CDR2; and the amino acid sequence set forth as SEQ ID NO:75 as CDR3.

Examples of more specific embodiments of the antibody having affinity to hTfR include those whose light chain CDRs have amino acid sequences according to one of (1) to (14) below:
(1) the amino acid sequence set forth as SEQ ID NO:6 as CDR1; SEQ ID NO:8 as CDR2; and SEQ ID NO:10 as CDR3;
(2) the amino acid sequence set forth as SEQ ID NO:11 as CDR1; SEQ ID NO:13 as CDR2; and SEQ ID NO:15 as CDR3;
(3) the amino acid sequence set forth as SEQ ID NO:16 as CDR1; SEQ ID NO:18 as CDR2; and SEQ ID NO:20 as CDR3;
(4) the amino acid sequence set forth as SEQ ID NO:21 as CDR1; SEQ ID NO:23 as CDR2; and SEQ ID NO:25 as CDR3;
(5) the amino acid sequence set forth as SEQ ID NO:26 as CDR1; SEQ ID NO:28 as CDR2; and SEQ ID NO:30 as CDR3;
(6) the amino acid sequence set forth as SEQ ID NO:31 as CDR1; SEQ ID NO:33 as CDR2; and SEQ ID NO:35 as CDR3;
(7) the amino acid sequence set forth as SEQ ID NO:36 as CDR1; SEQ ID NO:38 as CDR2; and SEQ ID NO:40 as CDR3;
(8) the amino acid sequence set forth as SEQ ID NO:41 as CDR1; SEQ ID NO:43 as CDR2; and SEQ ID NO:45 as CDR3;
(9) the amino acid sequence set forth as SEQ ID NO:46 as CDR1; SEQ ID NO:48 as CDR2; and SEQ ID NO:50 as CDR3;
(10) the amino acid sequence set forth as SEQ ID NO:51 as CDR1; SEQ ID NO:53 as CDR2; and SEQ ID NO:55 as CDR3;
(11) the amino acid sequence set forth as SEQ ID NO:56 as CDR1; SEQ ID NO:58 as CDR2; and SEQ ID NO:60 as CDR3;
(12) the amino acid sequence set forth as SEQ ID NO:61 as CDR1; SEQ ID NO:63 as CDR2; and SEQ ID NO:65 as CDR3;
(13) the amino acid sequence set forth as SEQ ID NO:66 as CDR1; SEQ ID NO:68 as CDR2; and SEQ ID NO:70 as CDR3; and
(14) the amino acid sequence set forth as SEQ ID NO:71 as CDR1; SEQ ID NO:73 as CDR2; and SEQ ID NO:75 as CDR3;

Examples of preferable embodiments of the antibody having affinity to hTfR include those whose heavy chain CDRs have amino acid sequences according to one of (1) to (14) below:
(1) the amino acid sequence set forth as SEQ ID NO:76 or SEQ ID NO:77 as CDR1; the amino acid sequence set forth as SEQ ID NO:78 or SEQ ID NO:79 as CDR2; and the amino acid sequence set forth as SEQ ID NO:80 or SEQ ID NO:81 as CDR3;
(2) the amino acid sequence set forth as SEQ ID NO:82 or SEQ ID NO:83 as CDR1; the amino acid sequence set forth as SEQ ID NO:84 or SEQ ID NO:85 as CDR2; and the amino acid sequence set forth as SEQ ID NO:86 or SEQ ID NO:87 as CDR3;
(3) the amino acid sequence set forth as SEQ ID NO:88 or SEQ ID NO:89 as CDR1; the amino acid sequence set forth as SEQ ID NO:90 or SEQ ID NO:91 as CDR2; and the amino acid sequence set forth as SEQ ID NO:92 or SEQ ID NO:93 as CDR3;
(4) the amino acid sequence set forth as SEQ ID NO:94 or SEQ ID NO:95 as CDR1; the amino acid sequence set forth as SEQ ID NO:96 or SEQ ID NO:97 as CDR2; and the amino acid sequence set forth as SEQ ID NO:98 or SEQ ID NO:99 as CDR3;
(5) the amino acid sequence set forth as SEQ ID NO:100 or SEQ ID NO:101 as CDR1; the amino acid sequence set forth as SEQ ID NO:102 or SEQ ID NO:103 as CDR2; and the amino acid sequence set forth as SEQ ID NO:104 or SEQ ID NO:105 as CDR3;
(6) the amino acid sequence set forth as SEQ ID NO:106 or SEQ ID NO:107 as CDR1; the amino acid sequence set forth as SEQ ID NO:108 or SEQ ID NO:278 as CDR2; and the amino acid sequence set forth as SEQ ID NO:109 or SEQ ID NO:110 as CDR3;
(7) the amino acid sequence set forth as SEQ ID NO:111 or SEQ ID NO:112 as CDR1; the amino acid sequence set forth as SEQ ID NO:113 or SEQ ID NO:114 as CDR2; and the amino acid sequence set forth as SEQ ID NO:115 or SEQ ID NO:116 as CDR3;
(8) the amino acid sequence set forth as SEQ ID NO:117 or SEQ ID NO:118 as CDR1; the amino acid sequence set forth as SEQ ID NO:119 or SEQ ID NO:279 as CDR2; and the amino acid sequence set forth as SEQ ID NO:120 or SEQ ID NO:121 as CDR3;
(9) the amino acid sequence set forth as SEQ ID NO:122 or SEQ ID NO:123 as CDR1; the amino acid sequence set forth as SEQ ID NO:124 or SEQ ID NO:125 as CDR2; and the amino acid sequence set forth as SEQ ID NO:126 or SEQ ID NO:127 as CDR3;
(10) the amino acid sequence set forth as SEQ ID NO:128 or SEQ ID NO:129 as CDR1; the amino acid sequence set forth as SEQ ID NO:130 or SEQ ID NO:131 as CDR2; and SEQ ID NO:132 or SEQ ID NO:133 as CDR3;
(11) the amino acid sequence set forth as SEQ ID NO:134 or SEQ ID NO:135 as CDR1; the amino acid sequence set forth as SEQ ID NO:136 or SEQ ID NO:137 as CDR2, and the amino acid sequence set forth as SEQ ID NO:138 or SEQ ID NO:139 as CDR3;
(12) the amino acid sequence set forth as SEQ ID NO:140 or SEQ ID NO:141 as CDR1; the amino acid sequence set forth as SEQ ID NO:142 or SEQ ID NO:143 as CDR2; and the amino acid sequence set forth as SEQ ID NO:144 or SEQ ID NO:145 as CDR3;
(13) the amino acid sequence set forth as SEQ ID NO:146 or SEQ ID NO:147 as CDR1; the amino acid sequence set forth as SEQ ID NO:148 or SEQ ID NO:149 as CDR2; and the amino acid sequence set forth as SEQ ID NO:150 or SEQ ID NO:151 as CDR3; and
(14) the amino acid sequence set forth as SEQ ID NO:152 or SEQ ID NO:153 as CDR1; the amino acid sequence set forth as SEQ ID NO:154 or SEQ ID NO:155 as CDR2; and the amino acid sequence set forth as SEQ ID NO:156 or SEQ ID NO:157 as CDR3.

Examples of more specific embodiments of the antibody having affinity to hTfR include those whose heavy chain CDRs have amino acid sequences according to one of (1) to (14) below:
(1) the amino acid sequence set forth as SEQ ID NO:76 as CDR1; SEQ ID NO:78 as CDR2; and SEQ ID NO:80 as CDR3;
(2) the amino acid sequence set forth as SEQ ID NO:82 as CDR1; SEQ ID NO:84 as CDR2; and SEQ ID NO:86 as CDR3;
(3) the amino acid sequence set forth as SEQ ID NO:88 as CDR1; SEQ ID NO:90 as CDR2; and SEQ ID NO:92 as CDR3;
(4) the amino acid sequence set forth as SEQ ID NO:94 as CDR1; SEQ ID NO:96 as CDR2; and SEQ ID NO:98 as CDR3;
(5) the amino acid sequence set forth as SEQ ID NO:100 as CDR1; SEQ ID NO:102 as CDR2; and SEQ ID NO:104 as CDR3;
(6) the amino acid sequence set forth as SEQ ID NO:106 as CDR1; SEQ ID NO:108 as CDR2; and SEQ ID NO:109 as CDR3;
(7) the amino acid sequence set forth as SEQ ID NO:111 as CDR1; SEQ ID NO:113 as CDR2; and SEQ ID NO:115 as CDR3;
(8) the amino acid sequence set forth as SEQ ID NO:117 as CDR1; SEQ ID NO:119 as CDR2; and SEQ ID NO:120 as CDR3;
(9) the amino acid sequence set forth as SEQ ID NO:122 as CDR1; SEQ ID NO:124 as CDR2; and SEQ ID NO:126 as CDR3;
(10) the amino acid sequence set forth as SEQ ID NO:128 as CDR1; SEQ ID NO:130 as CDR2; and SEQ ID NO:132 as CDR3;
(11) the amino acid sequence set forth as SEQ ID NO:134 as CDR1; SEQ ID NO:136 as CDR2; and SEQ ID NO:138 as CDR3;
(12) the amino acid sequence set forth as SEQ ID NO:140 as CDR1; SEQ ID NO:142 as CDR2; and SEQ ID NO:144 as CDR3;
(13) the amino acid sequence set forth as SEQ ID NO:146 as CDR1; SEQ ID NO:148 as CDR2; and SEQ ID NO:150 as CDR3; and
(14) the amino acid sequence set forth as SEQ ID NO:152 as CDR1; SEQ ID NO:154 as CDR2; and SEQ ID NO:156 as CDR3.

Examples of preferable combinations of the light chain and heavy chain of the antibody having affinity to hTfR include those having the amino acid sequences as CDRs according to (1) to (14) below:
(1) a combination of
the light chain having the amino acid sequence set forth as SEQ ID NO:6 or SEQ ID NO:7 as CDR1; the amino acid sequence set forth as SEQ ID NO:8 or SEQ ID NO:9 or the amino acid sequence Trp-Thr-Ser as CDR2; and the amino acid sequence set forth as SEQ ID NO:10 as CDR3; and
the heavy chain having the amino acid sequence set forth as SEQ ID NO:76 or SEQ ID NO:77 as CDR1; the amino acid sequence set forth as SEQ ID NO:78 or SEQ ID NO:79 as CDR2; and the amino acid sequence set forth as SEQ ID NO:80 or SEQ ID NO:81 as CDR3;
(2) a combination of
the light chain having the amino acid sequence set forth as SEQ ID NO:11 or SEQ ID NO:12 as CDR1; the amino acid sequence set forth as SEQ ID NO:13 or SEQ ID NO:14 or the amino acid sequence Tyr Ala Ser as CDR2; and the amino acid sequence set forth as SEQ ID NO:15 as CDR3; and
the heavy chain having the amino acid sequence set forth as SEQ ID NO:82 or SEQ ID NO:83 as CDR1; the amino acid sequence set forth as SEQ ID NO:84 or SEQ ID NO:85 as CDR2; and the amino acid sequence set forth as SEQ ID NO:86 or SEQ ID NO:87 as CDR3;
(3) a combination of
the light chain having the amino acid sequence set forth as SEQ ID NO:16 or SEQ ID NO:17 as CDR1; the amino acid sequence set forth as SEQ ID NO:18 or SEQ ID NO:19 or the amino acid sequence Lys-Val-Ser as CDR2; and the amino acid sequence set forth as SEQ ID NO:20 as CDR3; and
the heavy chain having the amino acid sequence set forth as SEQ ID NO:88 or SEQ ID NO:89 as CDR1; the amino acid sequence set forth as SEQ ID NO:90 or SEQ ID NO:91 as CDR2; and the amino acid sequence set forth as SEQ ID NO:92 or SEQ ID NO:93 as CDR3;
(4) a combination of
the light chain having the amino acid sequence set forth as SEQ ID NO:21 or SEQ ID NO:22 as CDR1; the amino acid sequence set forth as SEQ ID NO:23 or SEQ ID NO:24 or the amino acid sequence Asp-Thr-Ser as CDR2; and the amino acid sequence set forth as SEQ ID NO:25 as CDR3; and
the heavy chain having the amino acid sequence set forth as SEQ ID NO:94 or SEQ ID NO:95 as CDR1; the amino acid sequence set forth as SEQ ID NO:96 or SEQ ID NO:97 as CDR2; and the amino acid sequence set forth as SEQ ID NO:98 or SEQ ID NO:99 as CDR3;
(5) a combination of
the light chain having the amino acid sequence set forth as SEQ ID NO:26 or SEQ ID NO:27 as CDR1; the amino acid sequence set forth as SEQ ID NO:28 or SEQ ID NO:29 or the amino acid sequence Asp-Thr-Ser as CDR2; and the amino acid sequence set forth as SEQ ID NO:30 as CDR3;
the heavy chain having the amino acid sequence set forth as SEQ ID NO:100 or SEQ ID NO:101 as CDR1; the amino acid sequence set forth as SEQ ID NO:102 or SEQ ID NO:103 as CDR2; and the amino acid sequence set forth as SEQ ID NO:104 or SEQ ID NO:105 as CDR3;
(6) a combination of
the light chain having the amino acid sequence set forth as SEQ ID NO:31 or SEQ ID NO:32 as CDR1; the amino acid sequence set forth as SEQ ID NO:33 or SEQ ID NO:34 or the amino acid sequence Ala-Ala-Ser as CDR2; and the amino acid sequence set forth as SEQ ID NO:35 as CDR3; and
the heavy chain having the amino acid sequence set forth as SEQ ID NO:106 or SEQ ID NO:107 as CDR1; the amino acid sequence set forth as SEQ ID NO:108 or SEQ ID NO:278 as CDR2; and the amino acid sequence set forth as SEQ ID NO:109 or SEQ ID NO:110 as CDR3;
(7) a combination of
the light chain having the amino acid sequence set forth as SEQ ID NO:36 or SEQ ID NO:37 as CDR1; the amino acid sequence set forth as SEQ ID NO:38 or SEQ ID NO:39 or the amino acid sequence Gln-Thr-Ser as CDR2; and the amino acid sequence set forth as SEQ ID NO:40 as CDR3; and
the heavy chain having the amino acid sequence set forth as SEQ ID NO:111 or SEQ ID NO:112 as CDR1; the amino acid sequence set forth as SEQ ID NO:113 or SEQ ID NO:114 as CDR2; and the amino acid sequence set forth as SEQ ID NO:115 or SEQ ID NO:116 as CDR3;
(8) a combination of
the light chain having the amino acid sequence set forth as SEQ ID NO:41 or SEQ ID NO:42 as CDR1; the amino acid sequence set forth as SEQ ID NO:43 or SEQ ID NO:44 or the amino acid sequence Gly-Thr-Ser as CDR2; and the amino acid sequence set forth as SEQ ID NO:45 as CDR3; and
the heavy chain having the amino acid sequence set forth as SEQ ID NO:117 or SEQ ID NO:118 as CDR1; the amino acid sequence set forth as SEQ ID NO:119 or SEQ ID NO:279 as CDR2; and the amino acid sequence set forth as SEQ ID NO:120 or SEQ ID NO:121 as CDR3;
(9) a combination of
the light chain having the amino acid sequence set forth as SEQ ID NO:46 or SEQ ID NO:47 as CDR1; the amino acid sequence set forth as SEQ ID NO:48 or SEQ ID NO:49 or the amino acid sequence Phe-Thr-Ser as CDR2; and the amino acid sequence set forth as SEQ ID NO:50 as CDR3; and
the heavy chain having the amino acid sequence set forth as SEQ ID NO:122 or SEQ ID NO:123 as CDR1; the amino acid sequence set forth as SEQ ID NO:124 or SEQ ID NO:125 as CDR2; and the amino acid sequence set forth as SEQ ID NO:126 or SEQ ID NO:127 as CDR3;
(10) a combination of
the light chain having the amino acid sequence set forth as SEQ ID NO:51 or SEQ ID NO:52 as CDR1; the amino acid sequence set forth as SEQ ID NO:53 or SEQ ID NO:54 or the amino acid sequence Ala-Ala-Ser as CDR2; and the amino acid sequence set forth as SEQ ID NO:55 as CDR3; and
the heavy chain having the amino acid sequence set forth as SEQ ID NO:128 or SEQ ID NO:129 as CDR1; the amino acid sequence set forth as SEQ ID NO:130 or SEQ ID NO:131 as CDR2; and the amino acid sequence set forth as SEQ ID NO:132 or SEQ ID NO:133 as CDR3;
(11) a combination of
the light chain having the amino acid sequence set forth as SEQ ID NO:56 or SEQ ID NO:57 as CDR1; the amino acid sequence set forth as SEQ ID NO:58 or SEQ ID NO:59 or the amino acid sequence Tyr-Ala-Ser as CDR2; and the amino acid sequence set forth as SEQ ID NO:60 as CDR3; and
the heavy chain having the amino acid sequence set forth as SEQ ID NO:134 or SEQ ID NO:135 as CDR1; the amino acid sequence set forth as SEQ ID NO:136 or SEQ ID NO:137 as CDR2; and the amino acid sequence set forth as SEQ ID NO:138 or SEQ ID NO:139 as CDR3;
(12) a combination of
the light chain having the amino acid sequence set forth as SEQ ID NO:61 or SEQ ID NO:62 as CDR1; the amino acid sequence set forth as SEQ ID NO:63 or SEQ ID NO:64 or the amino acid sequence Trp-Ser-Ser as CDR2; and the amino acid sequence set forth as SEQ ID NO:65 as CDR3; and
the heavy chain having the amino acid sequence set forth as SEQ ID NO:140 or SEQ ID NO:141 as CDR1; the amino acid sequence set forth as SEQ ID NO:142 or SEQ ID NO:143 as CDR2; and the amino acid sequence set forth as SEQ ID NO:144 or SEQ ID NO:145 as CDR3;
(13) a combination of
the light chain having the amino acid sequence set forth as SEQ ID NO:66 or SEQ ID NO:67 as CDR1; the amino acid sequence set forth as SEQ ID NO:68 or SEQ ID NO:69 or the amino acid sequence Tyr-Ala-Ser as CDR2; and the amino acid sequence set forth as SEQ ID NO:70 as CDR3; and
the heavy chain having the amino acid sequence set forth as SEQ ID NO:146 or SEQ ID NO:147 as CDR1; the amino acid sequence set forth as SEQ ID NO:148 or SEQ ID NO:149 as CDR2; and the amino acid sequence set forth as SEQ ID NO:150 or SEQ ID NO:151 as CDR3;
(14) a combination of
the light chain having the amino acid sequence set forth as SEQ ID NO:71 or SEQ ID NO:72 as CDR1; the amino acid sequence set forth as SEQ ID NO:73 or SEQ ID NO:74 or the amino acid sequence Asp-Thr-Ser as CDR2; and the amino acid sequence set forth as SEQ ID NO:75 as CDR3; and
the heavy chain having the amino acid sequence set forth as SEQ ID NO:152 or SEQ ID NO:153 as CDR1; the amino acid sequence set forth as SEQ ID NO:154 or SEQ ID NO:155 as CDR2; and he amino acid sequence set forth as SEQ ID NO:156 or SEQ ID NO:157 as CDR3.

Examples of specific embodiments of combinations of the light chain and heavy chain of the antibody having affinity to hTfR include those having the amino acid sequences as CDRs according to one of (1) to (14) below:
(1) a combination of
the light chain having the amino acid sequence set forth as SEQ ID NO:6 as CDR1; SEQ ID NO:8 as CDR2; and SEQ ID NO:10 as CDR3; and
the heavy chain having the amino acid sequence set forth as SEQ ID NO:76 as CDR1; SEQ ID NO:78 as CDR2; and SEQ ID NO:80 as CDR3;
(2) a combination of
the light chain having the amino acid sequence set forth as SEQ ID NO:11 as CDR1; SEQ ID NO:13 as CDR2; and SEQ ID NO:15 as CDR3; and
the heavy chain having the amino acid sequence set forth as SEQ ID NO:82 as CDR1; SEQ ID NO:84 as CDR2; and SEQ ID NO:86 as CDR3;
(3) a combination of
the light chain having the amino acid sequence set forth as SEQ ID NO:16 as CDR1; SEQ ID NO:18 as CDR2; and SEQ ID NO:20 as CDR3; and
the heavy chain having the amino acid sequence set forth as SEQ ID NO:88 as CDR1; SEQ ID NO:90 as CDR2; and SEQ ID NO:92 as CDR3;
(4) a combination of
the light chain having the amino acid sequence set forth as SEQ ID NO:21 as CDR1; SEQ ID NO:23 as CDR2; and SEQ ID NO:25 as CDR3; and
the heavy chain having the amino acid sequence set forth as SEQ ID NO:94 as CDR1; SEQ ID NO:96 as CDR2; and SEQ ID NO:98 as CDR3;
(5) a combination of
the light chain having the amino acid sequence set forth as SEQ ID NO:26 as CDR1; SEQ ID NO:28 as CDR2; and SEQ ID NO:30 as CDR3; and
the heavy chain having the amino acid sequence set forth as SEQ ID NO:100 as CDR1; SEQ ID NO:102 as CDR2; and SEQ ID NO:104 as CDR3;
(6) a combination of
the light chain having the amino acid sequence set forth as SEQ ID NO:31 as CDR1; SEQ ID NO:33 as CDR2; and SEQ ID NO:35 as CDR3; and
the heavy chain having the amino acid sequence set forth as SEQ ID NO:106 as CDR1; SEQ ID NO:108 as CDR2; and SEQ ID NO:109 as CDR3;
(7) a combination of
the light chain having the amino acid sequence set forth as SEQ ID NO:36 as CDR1; SEQ ID NO:38 as CDR2; and SEQ ID NO:40 as CDR3; and
the heavy chain having the amino acid sequence set forth as SEQ ID NO:111 as CDR1; SEQ ID NO:113 as CDR2; and SEQ ID NO:115 as CDR3;
(8) a combination of
the light chain having the amino acid sequence set forth as SEQ ID NO:41 as CDR1; SEQ ID NO:43 as CDR2; and SEQ ID NO:45 as CDR3; and
the heavy chain having the amino acid sequence set forth as SEQ ID NO:117 as CDR1; SEQ ID NO:119 as CDR2; and SEQ ID NO:120 as CDR3;
(9) a combination of
the light chain having the amino acid sequence set forth as SEQ ID NO:46 as CDR1; SEQ ID NO:48 as CDR2; and SEQ ID NO:50 as CDR3; and
the heavy chain having the amino acid sequence set forth as SEQ ID NO:122 as CDR1; SEQ ID NO:124 as CDR2; and SEQ ID NO:126 as CDR3;
(10) a combination of
the light chain having the amino acid sequence set forth as SEQ ID NO:51 as CDR1; SEQ ID NO:53 as CDR2; and SEQ ID NO:55 as CDR3; and
the heavy chain having the amino acid sequence set forth as SEQ ID NO:128 as CDR1; SEQ ID NO:130 as CDR2; and SEQ ID NO:132 as CDR3;
(11) a combination of
the light chain having the amino acid sequence set forth as SEQ ID NO:56 as CDR1; SEQ ID NO:58 as CDR2; and SEQ ID NO:60 as CDR3; and
the heavy chain having the amino acid sequence set forth as SEQ ID NO:134 as CDR1; SEQ ID NO:136 as CDR2; and SEQ ID NO:138 as CDR3;
(12) a combination of
the light chain having the amino acid sequence set forth as SEQ ID NO:61 as CDR1; SEQ ID NO:63 as CDR2; and SEQ ID NO:65 as CDR3; and
the heavy chain having the amino acid sequence set forth as SEQ ID NO:140 as CDR1; SEQ ID NO:142 as CDR2; and SEQ ID NO:144 as CDR3;
(13) a combination of
the light chain having the amino acid sequence set forth as SEQ ID NO:66 as CDR1; SEQ ID NO:68 as CDR2; and SEQ ID NO:70 as CDR3; and
the heavy chain having the amino acid sequence set forth as SEQ ID NO:146 as CDR1; SEQ ID NO:148 as CDR2; and SEQ ID NO:150 as CDR3; and
(14) a combination of
the light chain having the amino acid sequence set forth as SEQ ID NO:71 as CDR1; SEQ ID NO:73 as CDR2; and SEQ ID NO:75 as CDR3; and
the heavy chain having the amino acid sequence set forth as SEQ ID NO:152 as CDR1; SEQ ID NO:154 as CDR2; and SEQ ID NO:156 as CDR3.

As preferred embodiments of humanized antibodies having affinity to hTfR, there are humanized antibodies produced using the amino acid sequences of the light chain variable region and the heavy chain variable region of the mouse anti-human TfR antibody set forth as SEQ ID NO:218 to SEQ ID NO:245 as CDRs. The humanized antibodies are produced by replacing proper positions of a human antibody with the amino acid sequences of CDRs of the light chain variable region and the heavy chain variable region of mouse anti-human TfR antibody.

For example, the light chain of a humanized antibody can be made by replacing the amino acid sequences of corresponding to CDRs of the light chain of a human antibody with an amino acid sequence consisting of not less than 3, or not less than 6, consecutive amino acids at the positions 24th to 34th of the amino acid sequence set forth as SEQ ID NO:218 as CDR1; with an amino acid sequence consisting of not less than 3, or not less than 6, consecutive amino acids at the positions 50th to 56th of the amino acid sequence set forth as SEQ ID NO:218 as CDR2; and with an amino acid sequence consisting of not less than 3, or not less than 8, consecutive amino acids at the positions 89th to 97th of the amino acid sequence set forth as SEQ ID NO:218 as CDR3, and
the heavy chain of a humanized antibody can be made by replacing the amino acid sequences of corresponding to CDRs of the heavy chain of a human antibody with an amino acid sequence consisting of not less than 3 consecutive amino acids at the positions 26th to 35th of the amino acid sequence set forth as SEQ ID NO:219 as CDR1; with an amino acid sequence consisting of not less than 3, or not less than 8, consecutive amino acids at the positions 50th to 66th of the amino acid sequence set forth as SEQ ID NO:219 as CDR2; with an amino acid sequence consisting of not less than 3, or not less than 7, consecutive amino acids at the positions 97th to 105th of the amino acid sequence set forth as SEQ ID NO:219 as CDR3.

By combining the light chain and the heavy chain of the humanized antibody thus obtained, the humanized antibody can be prepared.

Further, for example, the light chain of a humanized antibody can be made by replacing the amino acid sequences of corresponding CDRs of the light chain of a human antibody with an amino acid sequence consisting of not less than 3, or not less than 6, consecutive amino acids at the positions 24th to 34th of the amino acid sequence set forth as SEQ ID NO:220 as CDR1; with an amino acid sequence consisting of not less than 3, or not less than 6, consecutive amino acids at the positions 50th to 56th of the amino acid sequence set forth as SEQ ID NO:220 as CDR2; and with an amino acid sequence consisting of not less than 3 or not less than 8, consecutive amino acids at the positions 89th to 97th of the amino acid sequence set forth as SEQ ID NO:220 as CDR3; and
the heavy chain of a humanized antibody can be made by replacing the amino acid sequences of corresponding to CDRs of the heavy chain of a human antibody with an amino acid sequence consisting of not less than 3 consecutive amino acids at the positions 26th to 35th of the amino acid sequence set forth as SEQ ID NO:221 as CDR1; with an amino acid sequence consisting of not less than 3, or not less than 8, consecutive amino acids at the positions 50th to 66th of the amino acid sequence set forth as SEQ ID NO:221 as CDR2; and with an amino acid sequence consisting of not less than 3, or not less than 14, consecutive amino acids at the positions 97th to 112nd of the amino acid sequence set forth as SEQ ID NO:221 as CDR3.

By combining the light chain and the heavy chain of the humanized antibody thus obtained, the humanized antibody can be prepared.

Further, for example, the light chain of a humanized antibody can be made by replacing the amino acid sequences of corresponding CDRs of the light chain of a human antibody with an amino acid sequence consisting of not less than 3, or not less than 11, consecutive amino acids at the positions 24th to 39th of the amino acid sequence set forth as SEQ ID NO:222 as CDR1; with an amino acid sequence consisting of not less than 3, not less than 6, consecutive amino acids at the positions 55th to 61st of the amino acid sequence set forth as SEQ ID NO:222 as CDR2; and with an amino acid sequence consisting of not less than 3, or not less than 8, consecutive amino acids at the positions 94th to 102nd of the amino acid sequence set forth as SEQ ID NO:222 as CDR3; and
the heavy chain of a humanized antibody can be made by replacing the amino acid sequences of corresponding to CDRs of the heavy chain of a human antibody with an amino acid sequence consisting of not less than 3 consecutive amino acids at the positions 26th to 35th of the amino acid sequence set forth as SEQ ID NO:223 as CDR1; with an amino acid sequence consisting of not less than 3, or not less than 8, consecutive amino acids at the positions 50th to 66th of the amino acid sequence set forth as SEQ ID NO:223 as CDR2; and with an amino acid sequence consisting of not less than 3, or not less than 9 consecutive amino acids at the positions 97th to 107th of the amino acid sequence set forth as SEQ ID NO:223 as CDR3.

By combining the light chain and the heavy chain of the humanized antibody thus obtained, the humanized antibody can be prepared.

Further, for example, the light chain of a humanized antibody can be made by replacing the amino acid sequences of corresponding CDRs of the light chain of a human antibody with an amino acid sequence consisting of not less than 3, or not less than 5, consecutive amino acids at the positions 24th to 33rd of the amino acid sequence set forth as SEQ ID NO:224 as CDR1; with an amino acid sequence consisting of not less than 3, or not less than 6, consecutive amino acids at the positions 49th to 55th of the amino acid sequence set forth as SEQ ID NO:224 as CDR2; and with an amino acid sequence consisting of not less than 3, or not less than 8, consecutive amino acids at the positions 88th to 96th of the amino acid sequence set forth as SEQ ID NO:224 as CDR3; and
the heavy chain of a humanized antibody can be made by replacing the amino acid sequences of corresponding to CDRs of the heavy chain of a human antibody with an amino acid sequence consisting of not less than 3 consecutive amino acids at the positions 26th to 35th of the amino acid sequence set forth as SEQ ID NO:225 as CDR1; with an amino acid sequence consisting of not less than 3, or not less than 8, consecutive amino acids at the positions 51st to 66th of the amino acid sequence set forth as SEQ ID NO:225 as CDR2; and with an amino acid sequence consisting of not less than 3, or not less than 13, consecutive amino acids at the positions 97th to 111st of the amino acid sequence set forth as SEQ ID NO:225 as CDR3.

By combining the light chain and the heavy chain of the humanized antibody thus obtained, the humanized antibody can be prepared.

Further, for example, the light chain of a humanized antibody can be made by replacing the amino acid sequences of corresponding CDRs of the light chain of a human antibody with an amino acid sequence consisting of not less than 3, or not less than 5, consecutive amino acids at the positions 24th to 33rd of the amino acid sequence set forth as SEQ ID NO:226 as CDR1; with an amino acid sequence consisting of not less than 3, or not less than 6, consecutive amino acids at the positions 49th to 55th of the amino acid sequence set forth as SEQ ID NO:226 as CDR2; and with an amino acid sequence consisting of not less than 3, or not less than 7, consecutive amino acids at the positions 88th to 95th of the amino acid sequence set forth as SEQ ID NO:226 as CDR3; and
the heavy chain of a humanized antibody can be made by replacing the amino acid sequences of corresponding to CDRs of the heavy chain of a human antibody with an amino acid sequence consisting of not less than 3 consecutive amino acids at the positions 26th to 35th of the amino acid sequence set forth as SEQ ID NO:227 as CDR1; with an amino acid sequence consisting of not less than 3, or not less than 8, consecutive amino acids at the positions 51st to 66th of the amino acid sequence set forth as SEQ ID NO:227 as CDR2; and with an amino acid sequence consisting of not less than 3, or not less than 9, consecutive amino acids at the positions 97th to 107th of the amino acid sequence set forth as SEQ ID NO:227 as CDR3.

By combining the light chain and the heavy chain of the humanized antibody thus obtained, the humanized antibody can be prepared.

Further, for example, the light chain of a humanized antibody can be made by replacing the amino acid sequences of corresponding CDRs of the light chain of a human antibody with an amino acid sequence consisting of not less than 3, or not less than 6, consecutive amino acids at the positions 24th to 34th of the amino acid sequence set forth as SEQ ID NO:228 as CDR1; with an amino acid sequence consisting of not less than 3, or not less than 6, consecutive amino acids at the positions 50th to 56th of the amino acid sequence set forth as SEQ ID NO:228 as CDR2; and with an amino acid sequence consisting of not less than 3, or not less than 8, consecutive amino acids at the positions 89th to 97th of the amino acid sequence set forth as SEQ ID NO:228 as CDR3; and
the heavy chain of a humanized antibody can be made by replacing the amino acid sequences of corresponding to CDRs of the heavy chain of a human antibody with an amino acid sequence consisting of not less than 3 consecutive amino acids at the positions 26th to 35th of the amino acid sequence set forth as SEQ ID NO:229 as CDR1; with an amino acid sequence consisting of not less than 3, or not less than 7, consecutive amino acids at the positions 50th to 65th of the amino acid sequence set forth as SEQ ID NO:229 as CDR2; and with an amino acid sequence consisting of not less than 3, or not less than 4, consecutive amino acids at the positions 96th to 101st of the amino acid sequence set forth as SEQ ID NO:229 as CDR3.

By combining the light chain and the heavy chain of the humanized antibody thus obtained, the humanized antibody can be prepared.

Further, for example, the light chain of a humanized antibody can be made by replacing the amino acid sequences of corresponding CDRs of the light chain of a human antibody with an amino acid sequence consisting of not less than 3, or not less than 5, consecutive amino acids at the positions 24th to 33rd of the amino acid sequence set forth as SEQ ID NO:230 as CDR1; with an amino acid sequence consisting of not less than 3, or not less than 6, consecutive amino acids at the positions 49th to 55th of the amino acid sequence set forth as SEQ ID NO:230 as CDR2; and with an amino acid sequence consisting of not less than 3, or not less than 8, consecutive amino acids at the positions 88th to 96th of the amino acid sequence set forth as SEQ ID NO:230 as CDR3; and
the heavy chain of a humanized antibody can be made by replacing the amino acid sequences of corresponding to CDRs of the heavy chain of a human antibody with an amino acid sequence consisting of not less than 3 consecutive amino acids at the positions 26th to 35th of the amino acid sequence set forth as SEQ ID NO:231 as CDR1; with an amino acid sequence consisting of not less than 3, or not less than 8, consecutive amino acids at the positions 51st to 66th of the amino acid sequence set forth as SEQ ID NO:231 as CDR2; and with an amino acid sequence consisting of not less than 3, or not less than 11, consecutive amino acids at the positions 97th to 109th of the amino acid sequence set forth as SEQ ID NO:231 as CDR3.

By combining the light chain and the heavy chain of the humanized antibody thus obtained, the humanized antibody can be prepared.

Further, for example, the light chain of a humanized antibody can be made by replacing the amino acid sequences of corresponding CDRs of the light chain of a human antibody with an amino acid sequence consisting of not less than 3, or not less than 5, consecutive amino acids at the positions 24th to 33rd of the amino acid sequence set forth as SEQ ID NO:232 as CDR1; with an amino acid sequence consisting of not less than 3, or not less than 6, consecutive amino acids at the positions 49th to 55th of the amino acid sequence set forth as SEQ ID NO:232 as CDR2; and with an amino acid sequence consisting of not less than 3, or not less than 8, consecutive amino acids at the positions 88th to 96th of the amino acid sequence set forth as SEQ ID NO:232 as CDR3; and
the heavy chain of a humanized antibody can be made by replacing the amino acid sequences of corresponding to CDRs of the heavy chain of a human antibody with an amino acid sequence consisting of not less than 3 consecutive amino acids at the positions 26th to 35th of the amino acid sequence set forth as SEQ ID NO:233 as CDR1; with an amino acid sequence consisting of not less than 3, or not less than 7, consecutive amino acids at the positions 50th to 65th of the amino acid sequence set forth as SEQ ID NO:233 as CDR2; and with an amino acid sequence consisting of not less than 3, or not less than 4, consecutive amino acids at the positions 96th to 101st of the amino acid sequence set forth as SEQ ID NO:233 as CDR3.

By combining the light chain and the heavy chain of the humanized antibody thus obtained, the humanized antibody can be prepared.

Further, for example, the light chain of a humanized antibody can be made by replacing the amino acid sequences of corresponding CDRs of the light chain of a human antibody with an amino acid sequence consisting of not less than 3, or not less than 5, consecutive amino acids at the positions 24th to 33rd of the amino acid sequence set forth as SEQ ID NO:234 as CDR1; with an amino acid sequence consisting of not less than 3, or not less than 6, consecutive amino acids at the positions 49th to 55th of the amino acid sequence set forth as SEQ ID NO:234 as CDR2; and with an amino acid sequence consisting of not less than 3, or not less than 8, consecutive amino acids at the positions 88th to 96th of the amino acid sequence set forth as SEQ ID NO:234 as CDR3, and
the heavy chain of a humanized antibody can be made by replacing the amino acid sequences of corresponding to CDRs of the heavy chain of a human antibody with an amino acid sequence consisting of not less than 3 consecutive amino acids at the positions 26th to 35th of the amino acid sequence set forth as SEQ ID NO:235 as CDR1; with an amino acid sequence consisting of not less than 3, or not less than 8, consecutive amino acids at the positions 51st to 66th of the amino acid sequence set forth as SEQ ID NO:235 as CDR2, and with an amino acid sequence consisting of not less than 3, or not less than 8, consecutive amino acids at the positions 97th to 106th of the amino acid sequence set forth as SEQ ID NO:235 as CDR3.

By combining the light chain and the heavy chain of the humanized antibody thus obtained, the humanized antibody can be prepared.

Further, for example, the light chain of a humanized antibody can be made by replacing the amino acid sequences of corresponding CDRs of the light chain of a human antibody with an amino acid sequence consisting of not less than 3, or not less than 6, consecutive amino acids at the positions 24th to 34th of the amino acid sequence set forth as SEQ ID NO:236 as CDR1; with an amino acid sequence consisting of not less than 3, or not less than 6 consecutive amino acids at the positions 50th to 56th of the amino acid sequence set forth as SEQ ID NO:236 as CDR2; and with an amino acid sequence consisting of not less than 3, or not less than 8, consecutive amino acids at the positions 89th to 97th of the amino acid sequence set forth as SEQ ID NO:236 as CDR3; and
the heavy chain of a humanized antibody can be made by replacing the amino acid sequences of corresponding to CDRs of the heavy chain of a human antibody with an amino acid sequence consisting of not less than 3 consecutive amino acids at the positions 26th to 35th of the amino acid sequence set forth as SEQ ID NO:237 as CDR1; with an amino acid sequence consisting of not less than 3, or not less than 8, consecutive amino acids at the positions 51st to 66th of the amino acid sequence set forth as SEQ ID NO:237 as CDR2; and with an amino acid sequence consisting of not less than 3, or not less than 11, consecutive amino acids at the positions 97th to 109th of the amino acid sequence set forth as SEQ ID NO:237 as CDR3.

By combining the light chain and the heavy chain of the humanized antibody thus obtained, the humanized antibody can be prepared.

Further, for example, the light chain of a humanized antibody can be made by replacing the amino acid sequences of corresponding CDRs of the light chain of a human antibody with an amino acid sequence consisting of not less than 3, or not less than 6, consecutive amino acids at the positions 24th to 34th of the amino acid sequence set forth as SEQ ID NO:238 as CDR1; with an amino acid sequence consisting of not less than 3, or not less than 6, consecutive amino acids at the positions 50th to 56th of the amino acid sequence set forth as SEQ ID NO:238 as CDR2; and with an amino acid sequence consisting of not less than 3, or not less than 8, consecutive amino acids at the positions 89th to 97th of the amino acid sequence set forth as SEQ ID NO:238 as CDR3; and
the heavy chain of a humanized antibody can be made by replacing the amino acid sequences of corresponding to CDRs of the heavy chain of a human antibody with an amino acid sequence consisting of not less than 3 consecutive amino acids at the positions 26th to 35th of the amino acid sequence set forth as SEQ ID NO:239 as CDR1; with an amino acid sequence consisting of not less than 3 or not less than 8, consecutive amino acids at the positions 51st to 66th of the amino acid sequence set forth as SEQ ID NO:239 as CDR2; and with an amino acid sequence consisting of not less than 3 or not less than 9, consecutive amino acids at the positions 97th to 107th of the amino acid sequence set forth as SEQ ID NO:239 as CDR3.

By combining the light chain and the heavy chain of the humanized antibody thus obtained, the humanized antibody can be prepared.

Further, for example, the light chain of a humanized antibody can be made by replacing the amino acid sequences of corresponding CDRs of the light chain of a human antibody with an amino acid sequence consisting of not less than 3 or not less than 6, consecutive amino acids at the positions 24th to 34th of the amino acid sequence set forth as SEQ ID NO:240 as CDR1; with an amino acid sequence consisting of not less than 3 or not less than 6, consecutive amino acids at the positions 50th to 56th of the amino acid sequence set forth as SEQ ID NO:240 as CDR2; and with an amino acid sequence consisting of not less than 3 or not less than 8, consecutive amino acids at the positions 89th to 97th of the amino acid sequence set forth as SEQ ID NO:240 as CDR3, and
the heavy chain of a humanized antibody can be made by replacing the amino acid sequences of corresponding to CDRs of the heavy chain of a human antibody with an amino acid sequence consisting of not less than 3 consecutive amino acids at the positions 26th to 35th of the amino acid sequence set forth as SEQ ID NO:241 as CDR1; with an amino acid sequence consisting of not less than 3 or not less than 8, consecutive amino acids at the positions 51st to 66th of the amino acid sequence set forth as SEQ ID NO:241 as CDR2; and with an amino acid sequence consisting of not less than 3 or not less than 10, consecutive amino acids at the positions 97th to 108th of the amino acid sequence set forth as SEQ ID NO:241 as CDR3.

By combining the light chain and the heavy chain of the humanized antibody thus obtained, the humanized antibody can be prepared.

Further, for example, the light chain of a humanized antibody can be made by replacing the amino acid sequences of corresponding CDRs of the light chain of a human antibody with an amino acid sequence consisting of not less than 3 or not less than 6, consecutive amino acids at the positions 24th to 34th of the amino acid sequence set forth as SEQ ID NO:242 as CDR1; with an amino acid sequence consisting of not less than 3 or not less than 6, consecutive amino acids at the positions 50th to 56th of the amino acid sequence set forth as SEQ ID NO:242 as CDR2; and with an amino acid sequence consisting of not less than 3 or not less than 8, consecutive amino acids at the positions 89th to 97th of the amino acid sequence set forth as SEQ ID NO:242 as CDR3; and
the heavy chain of a humanized antibody can be made by replacing the amino acid sequences of corresponding to CDRs of the heavy chain of a human antibody with an amino acid sequence consisting of not less than 3 consecutive amino acids at the positions 26th to 35th of the amino acid sequence set forth as SEQ ID NO:243 as CDR1; with an amino acid sequence consisting of not less than 3 or not less than 8, consecutive amino acids at the positions 51st to 66th of the amino acid sequence set forth as SEQ ID NO:243 as CDR2; and with an amino acid sequence consisting of not less than 3 or not less than 9, consecutive amino acids at the positions 97th to 107th of the amino acid sequence set forth as SEQ ID NO:243 as CDR3.

By combining the light chain and the heavy chain of the humanized antibody thus obtained, the humanized antibody can be prepared.

Further, for example, the light chain of a humanized antibody can be made by replacing the amino acid sequences of corresponding CDRs of the light chain of a human antibody with an amino acid sequence consisting of not less than 3 or not less than 5, consecutive amino acids at the positions 24th to 33rd of the amino acid sequence set forth as SEQ ID NO:244 as CDR1; with an amino acid sequence consisting of not less than 3 or not less than 6, consecutive amino acids at the positions 49th to 55th of the amino acid sequence set forth as SEQ ID NO:244 as CDR2; and with an amino acid sequence consisting of not less than 3 or not less than 9, consecutive amino acids at the positions 88th to 96th of the amino acid sequence set forth as SEQ ID NO:244 as CDR3; and
the heavy chain of a humanized antibody can be made by replacing the amino acid sequences of corresponding to CDRs of the heavy chain of a human antibody with an amino acid sequence consisting of not less than 3 consecutive amino acids at the positions 26th to 35th of the amino acid sequence set forth as SEQ ID NO:245 as CDR1; with an amino acid sequence consisting of not less than 3 or not less than 8, consecutive amino acids at the positions 51st to 66th of the amino acid sequence set forth as SEQ ID NO:245 as CDR2; and with an amino acid sequence consisting of not less than 3, or not less than 9, consecutive amino acids at the positions 97th to 107th of the amino acid sequence set forth as SEQ ID NO:245 as CDR3.

By combining the light chain and the heavy chain of the humanized antibody thus obtained, the humanized antibody can be prepared.

Examples of preferred embodiments of the humanized antibody having affinity to hTfR include those having an amino acid sequence according to one of (1) to (3) below:
(1) An anti-hTfR antibody,
wherein the light chain variable region thereof comprises an amino acid sequence selected from the group consisting of the amino acid sequences set forth as SEQ ID NO:158, SEQ ID NO:159, SEQ ID NO:160, SEQ ID NO:161, SEQ ID NO:162, and SEQ ID NO:163, and
wherein the heavy chain variable region thereof comprises an amino acid sequence selected from the group consisting of the amino acid sequences set forth as SEQ ID NO:166, SEQ ID NO:167, SEQ ID NO:168, SEQ ID NO:169, SEQ ID NO:170, and SEQ ID NO:171.
(2) an anti-hTfR antibody,
wherein the light chain variable region thereof comprises any amino acid sequence of SEQ ID NO:174, SEQ ID NO:175, SEQ ID NO:176, SEQ ID NO:177, SEQ ID NO:178, and SEQ ID NO:179, and
wherein the heavy chain variable region thereof comprises any amino acid sequence of SEQ ID NO:182, SEQ ID NO:183, SEQ ID NO:184, SEQ ID NO:185, SEQ ID NO:186, and SEQ ID NO:187.
(3) anti-hTfR antibody,
wherein the light chain variable region thereof comprises an amino acid sequence selected from the group consisting of the amino acid sequences set forth as SEQ ID NO:190, SEQ ID NO:191, SEQ ID NO:192, SEQ ID NO:193, SEQ ID NO:194, and SEQ ID NO:195, and
wherein the heavy chain variable region thereof comprises an amino acid sequence selected from the group consisting of the amino acid sequences set forth as SEQ ID NO:204, SEQ ID NO:205, SEQ ID NO:206, SEQ ID NO:207, SEQ ID NO:208 and SEQ ID NO:209.

The amino acid sequences of the light chain variable region set forth as SEQ ID NO:158, SEQ ID NO:159, SEQ ID NO:160, SEQ ID NO:161, SEQ ID NO:162 and SEQ ID NO:163 comprise the amino acid sequence set forth as SEQ ID NO:6 or 7 in CDR1; SEQ ID NO:8 or 9 in CDR2; and SEQ ID NO:10 in CDR3. However, the term CDRs as used above in regard to the amino acid sequences of the light chain variable region set forth as SEQ ID NOs: 158 to 162 is not limited to those specific sequences but may also include a region containing the amino acid sequences of one of the CDRs or include an amino acid sequence comprising not less than 3 consecutive amino acids of one of the above CDRs.

The amino acid sequences of the heavy chain variable region set forth as SEQ ID NO:166, SEQ ID NO:167, SEQ ID NO:168, SEQ ID NO:169, SEQ ID NO:170, and SEQ ID NO:171 comprise the amino acid sequence set forth as SEQ ID NO:76 or 77 in CDR1; SEQ ID NO:78 or 79 in CDR2; and SEQ ID NO:80 or 81 in CDR3. However, the term CDRs used above in regard to the amino acid sequences of the heavy chain variable region set forth as SEQ ID NOs: 166 to 171 is not limited to those specific sequences but may also include a region containing the amino acid sequences of one of the CDRs or include an amino acid sequence comprising not less than 3 consecutive amino acids of one of the above CDRs.

The amino acid sequences of the light chain variable region set forth as SEQ ID NO:174, SEQ ID NO:175, SEQ ID NO:176, SEQ ID NO:177, SEQ ID NO:178, and SEQ ID NO:179 comprise the amino acid sequence set forth as SEQ ID NO:11 or 12 in CDR1; SEQ ID NO:13 or 14 in CDR2; and SEQ ID NO:15 in CDR3. However, the term CDRs as used above in regard to the amino acid sequences of the light chain variable region set forth as SEQ ID NOs: 174 to 179 is not limited to those specific sequences but may also include a region containing the amino acid sequences of one of the CDRs or include an amino acid sequence comprising not less than 3 consecutive amino acids of one of the above CDRs.

The amino acid sequences of the heavy chain variable region set forth as SEQ ID NO:182, SEQ ID NO:183, SEQ ID NO:184, SEQ ID NO:185, SEQ ID NO:186, and SEQ ID NO:187 comprise the amino acid sequence set forth as SEQ ID NO:82 or 83 in CDR1; SEQ ID NO:84 or 85 in CDR2; and SEQ ID NO:86 or 87 in CDR3. However, the term CDRs used above in regard to the amino acid sequences of the heavy chain variable region set forth as SEQ ID NOs: 182 to 187 is not limited to those specific sequences but may also include a region containing the amino acid sequences of one of the CDRs or include an amino acid sequence comprising not less than 3 consecutive amino acids of one of the above CDRs.

The amino acid sequences of the light chain variable region set forth as SEQ ID NO:190, SEQ ID NO:191, SEQ ID NO:192, SEQ ID NO:193, SEQ ID NO:194, and SEQ ID NO:195 comprise the amino acid sequence set forth as SEQ ID NO:16 or 17 in CDR1, SEQ ID NO:18 or 19 in CDR2, and SEQ ID NO:20 in CDR3. However, the term CDRs as used above in regard to the amino acid sequences of the light chain variable region set forth as SEQ ID NOs: 190 to 195 is not limited to those specific sequences but may also include a region containing the amino acid sequences of one of the CDRs or include an amino acid sequence comprising not less than 3 consecutive amino acids of one of the above CDRs.

The amino acid sequences of the heavy chain variable region set forth as SEQ ID NO:204, SEQ ID NO:205, SEQ ID NO:206, SEQ ID NO:207, SEQ ID NO:208 and SEQ ID NO:209 comprise the amino acid sequence set forth as SEQ ID NO:88 or 89 in CDR1; SEQ ID NO:90 or 91 in CDR2, and SEQ ID NO:92 or 93 in CDR3. However, the term CDRs used above in regard to the amino acid sequences of the heavy chain variable region set forth as SEQ ID NOs: 204 to 209 is not limited to those specific sequences but may also include a region containing the amino acid sequences of one of the CDRs or include an amino acid sequence comprising not less than 3 consecutive amino acids of one of the above CDRs.

Examples of more specific embodiments of the humanized antibody having affinity to hTfR include:
the one that comprises the amino acid sequence set forth as SEQ ID NO:163 in the light chain variable region and comprises the amino acid sequence set forth as SEQ ID NO:171 in the heavy chain variable region,
the one that comprises the amino acid sequence set forth as SEQ ID NO:179 in the light chain variable region and comprises the amino acid sequence set forth as SEQ ID NO:187 in the heavy chain variable region,
the one that comprises the amino acid sequence set forth as SEQ ID NO:191 in the light chain variable region and comprises the amino acid sequence set forth as SEQ ID NO:205 in the heavy chain variable region,
the one that comprises the amino acid sequence set forth as SEQ ID NO:193 in the light chain variable region and comprises the amino acid sequence set forth as SEQ ID NO:205 in the heavy chain variable region,
the one that comprises the amino acid sequence set forth as SEQ ID NO:194 in the light chain variable region and comprises the amino acid sequence set forth as SEQ ID NO:205 in the heavy chain variable region, and
the one that comprises the amino acid sequence set forth as SEQ ID NO:195 in the light chain variable region and comprises the amino acid sequence set forth as SEQ ID NO:205 in the heavy chain variable region.

Examples of more specific embodiments of the humanized antibody having affinity to hTfR include:
the one that comprises the amino acid sequence set forth as SEQ ID NO:164 in the light chain and the amino acid sequence set forth as SEQ ID NO:172 in the heavy chain,
the one that comprises the amino acid sequence set forth as SEQ ID NO:180 in the light chain and the amino acid sequence set forth as SEQ ID NO:188, in the heavy chain,
the one that comprises the amino acid sequence set forth as SEQ ID NO:196 in the light chain and the amino acid sequence set forth as SEQ ID NO:210 in the heavy chain,
the one that comprises the amino acid sequence set forth as SEQ ID NO:198 in the light chain and the amino acid sequence set forth as SEQ ID NO:210 in the heavy chain,
the one that comprises the amino acid sequence set forth as SEQ ID NO:200 in the light chain and the amino acid sequence set forth as SEQ ID NO:210 in the heavy chain,
the one that comprises the amino acid sequence set forth as SEQ ID NO:202 in the light chain and the amino acid sequence set forth as SEQ ID NO:210 in the heavy chain,
the one that comprises the amino acid sequence set forth as SEQ ID NO:196 in the light chain and the amino acid sequence set forth as SEQ ID NO:212 in the heavy chain,
the one that comprises the amino acid sequence set forth as SEQ ID NO:198 in the light chain and the amino acid sequence set forth as SEQ ID NO:212 in the heavy chain,
the one that comprises the amino acid sequence set forth as SEQ ID NO:200 in the light chain and the amino acid sequence set forth as SEQ ID NO:212 in the heavy chain, and
the one that comprises the amino acid sequence set forth as SEQ ID NO:202 in the light chain and the amino acid sequence set forth as SEQ ID NO:212 in the heavy chain.

Preferred embodiments of the antibody having affinity to hTfR have been exemplified above. The light chain and heavy chain of those anti-hTfR antibodies may be mutated as desired, by substitution, deletion, addition and the like, in their variable-region amino acid sequences in order to adjust the affinity of the anti-hTfR antibody to hTfR to a suitable level.

When replacing on or more amino acids of the light chain variable-region amino acid sequence with other amino acids, the number of amino acids to be replaced is preferably 1 to 10, more preferably 1 to 5, still more preferably 1 to 3, and even more preferably 1 or 2. When deleting one or more amino acids of the light chain variable-region amino acid sequence, the number of amino acids to be deleted is preferably 1 to 10, more preferably 1 to 5, still more preferably 1 to 3, and even more preferably 1 or 2. Introduction of a combined mutation of such substitution and deletion of amino acids is also allowed.

When adding one or more amino acids to the light chain variable region, they may be added inside, or on the N-terminal side or the C-terminal side of, the light chain variable-region amino acid sequence, and preferably 1 to 10, more preferably 1 to 5, still more preferably 1 to 3, and even more preferably 1 or 2, in number. Introduction of a combined mutation of such addition, substitution, and deletion of amino acids is also allowed. Such a mutated light chain variable-region amino acid sequence has a homology preferably not lower than 80%, more preferably not lower than 90%, still more preferably not lower than 95%, to the amino acid sequence of the original light chain variable-region.

In particular, when replacing one or more amino acids of the amino acid sequence of respective CDRs in the light chain with other amino acids, the number of amino acids to be replaced is preferably 1 to 5, more preferably 1 to 3, still more preferably 1 or 2, and even more preferably 1. When deleting one or more amino acid of the amino acid sequence of the respective CDRs, the number of amino acids to be deleted is preferably 1 to 5, more preferably 1 to 3, still more preferably 1 or 2, and even more preferably 1. Introduction of a combined mutation of such substitution and deletion of amino acids is also allowed.

When adding one or more amino acids to the amino acid sequence of respective CDRs in the light chain, they are added inside, or on the N-terminal side or the C-terminal side of, the amino acid sequence, and preferably 1 to 5, more preferably 1 to 3, still more preferably 1 or 2, in number. Introduction of a combined mutation of such addition, substitution, and deletion of amino acids is also allowed. The amino acid sequence of each of such mutated CDRs has a homology preferably not lower than 80%, more preferably not lower than 90%, and still more preferably not lower than 95%, to the amino acid sequence of the respective original CDRs.

When replacing one or more amino acids of the heavy chain variable-region amino acid sequence with other amino acids, the number of amino acids to be replaced is preferably 1 to 10, more preferably 1 to 5, still more preferably 1 to 3, and even more preferably 1 or 2. When deleting one or more amino acids of the heavy chain variable-region amino acid sequence, the number of amino acids to be deleted is preferably 1 to 10, more preferably 1 to 5, still more preferably 1 to 3, and even more preferably 1 or 2. Introduction of a combined mutation of such substitution and deletion of amino acids is also allowed.

When adding one or more amino acids to the heavy chain variable region, they may be added inside, or on the N-terminal side or the C-terminal side of, the heavy chain variable-region amino acid sequence, and preferably 1 to 10, more preferably 1 to 5, still more preferably 1 to 3, and even more preferably 1 or 2, in number. Introduction of a combined mutation of such addition, substitution, and deletion of amino acids is also allowed. Such a mutated heavy chain variable-region amino acid sequence has a homology preferably not lower than 80%, more preferably not lower than 90%, still more preferably not lower than 95%, to the amino acid sequence of the original heavy chain variable-region.

In particular, when replacing one or more amino acids of the amino acid sequence of respective CDRs in the heavy chain with other amino acids, the number of amino acids to be replaced is preferably 1 to 5, more preferably 1 to 3, still more preferably 1 or 2, and even more preferably 1. When deleting one or more amino acid of the amino acid sequence of the respective CDRs, the number of amino acids to be deleted is preferably 1 to 5, more preferably 1 to 3, still more preferably 1 or 2, and even more preferably 1. Introduction of a combined mutation of such substitution and deletion of amino acids is also allowed.

When adding one or more amino acids to the amino acid sequence of respective CDRs in the heavy chain, they are added inside, or on the N-terminal side or the C-terminal side of, the amino acid sequence, and preferably 1 to 5, more preferably 1 to 3, still more preferably 1 or 2, and even more preferably 1 in number. Introduction of a combined mutation of such addition, substitution, and deletion of amino acids is also allowed. The amino acid sequence of each of such mutated CDRs has a homology preferably not lower than 80%, more preferably not lower than 90%, and still more preferably not lower than 95%, to the amino acid sequence of the respective original CDRs.

In the above, replacement of one or more amino acids of the above anti-hTfR antibody variable-region amino acid sequence with other amino acids is exemplified by interchange between acidic amino acids, i.e., aspartic acid and glutamic acid; interchange between amide-type amino acids, i.e., asparagine and glutamine; interchange between basic amino acids, i.e., lysine and arginine, interchange between branched amino acids, i.e., valine, leucine and isoleucine; interchange between aliphatic amino acids, i.e., glycine and alanine, interchange between hydroxyamino acids, i.e., serine and threonine, and interchange between aromatic amino acids, i.e., phenylalanine and tyrosine.

Besides, in the case where introducing a mutation into the anti-hTfR antibody by adding one or more amino acids on its C-terminus or the N-terminus, if the added amino acids are positioned between the anti-hTfR antibody and a different protein (A) when they are fused, the added amino acids constitute part of a linker.

In the above preferred embodiments of the antibody, including humanized antibody, having affinity to hTfR, there is no particular limitation as to the amino acid sequence of the anti-hTfR antibody heavy chain and light chain CDRs, insofar as the antibody has a specific affinity to hTfR. However, the anti-hTfR antibody of the present invention exhibits a dissociation constant (K_{D}) with hTfR, as measured by the method described in Example 7, which is preferably not greater than 1×10⁻⁸ M, more preferably not greater than 1×10⁻⁹ M, still more preferably not greater than 1×10⁻⁹ M, and even more preferably not greater than 1×10⁻⁹ M. For example, one which exhibits a dissociation constant of 1×10⁻¹³ M to 1×10⁻⁹ M or 1×10⁻¹³ M to 1×10⁻¹⁰ M is preferred. The same applies when the antibody is a single-chain antibody. Further, in the case where the anti-hTfR antibody of the present invention has an affinity also to a monkey TfR, the dissociation constant of the anti-hTfR antibody to the monkey TfR, as measured by the method described in Example 7, is preferably not greater than 5×10⁻⁸ M, more preferably not greater than 2×10⁻⁸M, still more preferably not greater than 1×10⁻⁸M. For example, one which exhibits a dissociation constant of 1×10⁻¹³ M to 2×10⁻⁸ M is preferred. The same applies when the antibody is a single-chain antibody.

Specific embodiments of the above fusion protein between the humanized antibody, which has affinity to hTfR, and a different protein (A) described above include: those in which the different protein (A) is human iduronate 2-sulfatase (hI2S), human erythropoietin (hEPO), human arylsulfatase A (hARSA), human PPT-1 (hPPT-1), human TPP-1 (hTPP-1), human α-L-iduronidase (hIDUA), human TNFα receptor (hTNFαR), and human heparan N-sulfatase (hSGSH).

Specific examples of the fusion protein where the different protein (A) is hI2S include;
(1) the one that is composed of: the portion consisting of hTfR heavy chain linked, on the C-terminal side thereof and via a linker sequence Gly-Ser, to hI2S, and the other portion consisting of the hTfR light chain, wherein the amino acid sequence of the former is set forth as SEQ ID NO:247, and the amino acid sequence of the latter is set forth as SEQ ID NO:164,
(2) the one that is composed of: the portion consisting of hTfR heavy chain linked, on the C-terminal side thereof and via a linker sequence Gly-Ser, to hI2S, and the other portion consisting of the hTfR light chain wherein the amino acid sequence of the former is set forth as SEQ ID NO:249, and the amino acid sequence of the latter is set forth as SEQ ID NO:180,
(3) the one that is composed of: the portion consisting of hTfR heavy chain linked, on the C-terminal side thereof and via a linker sequence Gly-Ser, to hI2S, and the other portion consisting of the hTfR light chain wherein the amino acid sequence of the former is set forth as SEQ ID NO:251, and the amino acid sequence of the latter is set forth as SEQ ID NO:196.
In (1) above, the amino acid sequence of the hTfR heavy chain, which is included in SEQ ID NO:247, is the one set forth as SEQ ID NO:172. Namely, the fusion protein according to (1) above, includes, as a humanized antibody, the amino acid sequence of the light chain set forth as SEQ ID NO:164 and the amino acid sequence of the heavy chain set forth as SEQ ID NO:172.
In (2) above, the amino acid sequence of the hTfR heavy chain, which is included in SEQ ID NO:249, is the one set forth as SEQ ID NO:188. Namely, the fusion protein according to (2) above, includes, as a humanized antibody, the amino acid sequence of the light chain set forth as SEQ ID NO:180 and the amino acid sequence of the heavy chain set forth as SEQ ID NO:188.
In (3) above, the amino acid sequence of the hTfR heavy chain, which is included in SEQ ID NO:251, is the one set forth as SEQ ID NO:210. Namely, the fusion protein according to (3) above, includes, as a humanized antibody, the amino acid sequence of the light chain set forth as SEQ ID NO:196 and the amino acid sequence of the heavy chain set forth as SEQ ID NO:210.

Specific examples of the fusion protein where the different protein (A) is human erythropoietin (hEPO) include;
(1) the one that is composed of: the portion consisting of hTfR heavy chain linked, on the C-terminal side thereof and via a peptide bond, to hEPO, and the other portion consisting of the hTfR light chain, wherein the amino acid sequence of hTfR heavy chain of the former is set forth as SEQ ID NO:172, and the amino acid sequence of the latter is set forth as SEQ ID NO:164,
(2) the one that is composed of: the portion consisting of hTfR heavy chain linked, on the C-terminal side thereof and via a peptide bond, to hEPO, and the other portion consisting of the hTfR light chain, wherein the amino acid sequence of hTfR heavy chain of the former is set forth as SEQ ID NO:188, and the amino acid sequence of the latter is set forth as SEQ ID NO:180, and
(3) the one that is composed of: the portion consisting of hTfR heavy chain linked, on the C-terminal side thereof and via a peptide bond, to hEPO, and the other portion consisting of the hTfR light chain, wherein the amino acid sequence of hTfR heavy chain of the former is set forth as SEQ ID NO:210, and the amino acid sequence of the latter is set forth as SEQ ID NO:196.

In any of (1) to (3) above, hEPO and the hTfR heavy chain may be linked by peptide bond, either directly or via a linker sequence. Namely, in any of (1) to (3) above, the meaning of the phrase “via a peptide bond” and that of the phrase “directly or via a linker sequence” are the same. The linker sequence employed here consists of 1 to 50 amino acid residues. Though there is no particular limitation as to the amino acid sequence of the linker, it is preferably made of glycine and serine: for example, a single amino acid either glycine or serine, the amino acid sequence Gly-Ser, the amino acid sequence Gly-Gly-Ser, the amino acid sequence Gly-Gly-Gly-Gly-Ser (SEQ ID NO:3), the amino acid sequence Gly-Gly-Gly-Gly-Gly-Ser (SEQ ID NO:4), the amino acid sequence Ser-Gly-Gly-Gly-Gly-Gly (SEQ ID NO:5), or a sequence consisting 1 to 50, or 2 to 17, 2 to 10, 10 to 40, 20 to 34, 23 to 31, 25 to 29, or 27 amino acids which are composed of 1 to 10 or 2 to 5 of any of those amino acid sequences consecutively linked. For example, an amino acid sequence comprising the amino acid sequence Gly-Ser may be preferably used as a linker sequence. Further, a linker sequence comprising 27 amino acids is preferably used that is composed of the amino acid sequence Gly-Ser followed by consecutively linked five copies of the amino acid sequence Gly-Gly-Gly-Gly-Ser (SEQ ID NO:3).

More specific embodiments of humanized antibody plus hEPO include the one that is composed of the part consisting of hEPO linked, via the linker sequence Gly-Ser, to the C-terminal side of the hTfR heavy chain, and the other part consisting of the hTfR light chain, wherein the amino acid sequence of the former is set forth as SEQ ID NO:257, and the amino acid sequence of the latter is set forth as SEQ ID NO:196.

Besides, in the present invention, though the term “human EPO” or “hEPO” refers, in particular to the hEPO having the same amino acid sequence as the natural-type hEPO set forth as SEQ ID NO:256, it also includes those amino acid sequences produced by introducing a mutation such as substitution, deletion, addition and the like into the amino acid sequence of the natural-type hEPO, insofar as they have the EPO activity. When replacing one or more of the amino acids of the amino acid sequence of hEPO with other amino acids, the number of amino acids to be replaced is preferably 1 to 10, more preferably 1 to 5, still more preferably 1 to 3, even more preferably 1 or 2. When deleting one or more amino acids of the amino acid sequence of hEPO, the number of amino acids to be deleted is preferably 1 to 10, more preferably 1 to 5, still more preferably 1 to 3, and even more preferably 1 or 2. It is also possible to introduce a combined mutation of such substitution and deletion of amino acids. When adding one or more amino acids to hEPO, they may be added, inside, or on the N-terminal side or the C-terminal side of, the amino acid sequence of hEPO, and the number of amino acids to be added is preferably 1 to 10, more preferably 1 to 5, still more preferably 1 to 3, even more preferably 1 or 2. It is also possible to introduce a combined mutation of such addition, substitution, and deletion of the amino acid. The amino acid sequence of each of such mutated hEPO has a homology preferably not lower than 80%, more preferably not lower than 90%, still more preferably not lower than 95%, to the amino acid sequence of the original hEPO. Darbepoetin is an example obtained by mutating natural-type hEPO.

Specific examples of the fusion protein where the different protein (A) is human arylsulfatase A (hARSA) include;
(1) the one that is composed of: the portion consisting of hTfR heavy chain linked, on the C-terminal side thereof and via a peptide bond, to hARSA, and the other portion consisting of the hTfR light chain, wherein the amino acid sequence of hTfR heavy chain of the former is set forth as SEQ ID NO:172, and the amino acid sequence of the latter is set forth as SEQ ID NO:164,
(2) the one that is composed of: the portion consisting of hTfR heavy chain linked, on the C-terminal side thereof and via a peptide bond, to hARSA, and the other portion consisting of the hTfR light chain, wherein the amino acid sequence of hTfR heavy chain of the former is set forth as SEQ ID NO:188, and the amino acid sequence of the latter is set forth as SEQ ID NO:180, and
(3) the one that is composed of: the portion consisting of hTfR heavy chain linked, on the C-terminal side thereof via a peptide bond, to hARSA, and the other portion consisting of the hTfR light chain, wherein the amino acid sequence of hTfR heavy chain of the former is set forth as SEQ ID NO:210, and the amino acid sequence of the latter is set forth as SEQ ID NO:196.

In any of (1) to (3) above, hARSA and the hTfR heavy chain may be linked by peptide bond, either directly or via a linker sequence. Namely, in any of (1) to (3) above, the meaning of the phrase “via a peptide bond” and that of the phrase “directly or via a linker sequence” are the same. The linker sequence employed here consists of 1 to 50 amino acid residues. Though there is no particular limitation as to the amino acid sequence of the linker, it is preferably made of glycine and serine: for example, a single amino acid either glycine or serine, the amino acid sequence Gly-Ser, the amino acid sequence Gly-Gly-Ser, the amino acid sequence Gly-Gly-Gly-Gly-Ser (SEQ ID NO:3), the amino acid sequence Gly-Gly-Gly-Gly-Gly-Ser (SEQ ID NO:4), the amino acid sequence Ser-Gly-Gly-Gly-Gly-Gly (SEQ ID NO:5), or a sequence consisting 1 to 50, or 2 to 17, 2 to 10, 10 to 40, 20 to 34, 23 to 31, 25 to 29, or 27 amino acids which are composed of 1 to 10 or 2 to 5 of any of those amino acid sequences consecutively linked. For example, an amino acid sequence comprising the amino acid sequence Gly-Ser may be preferably used as a linker sequence. Further, a linker sequence comprising 27 amino acids is preferably used that is composed of the amino acid sequence Gly-Ser followed by consecutively linked five copies of the amino acid sequence Gly-Gly-Gly-Gly-Ser (SEQ ID NO:3).

More specific embodiments of humanized antibody plus hARSA include the one that is composed of the part consisting of hARSA linked, via the linker sequence Gly-Ser, to the C-terminal side of the hTfR heavy chain, and the other part consisting of the hTfR light chain, wherein the amino acid sequence of the former is set forth as SEQ ID NO:260, and the amino acid sequence of the latter is set forth as SEQ ID NO:196.

Besides, in the present invention, though the term “human ARSA” or “hARSA” refers, in particular to the hARSA having the same amino acid sequence as the natural-type hARSA set forth as SEQ ID NO:259, it also includes those amino acid sequences produced by introducing a mutation such as substitution, deletion, addition and the like into the amino acid sequence of the natural-type hARSA, insofar as they have the ARSA activity. When replacing one or more of the amino acids of the amino acid sequence of hARSA with other amino acids, the number of amino acids to be replaced is preferably 1 to 10, more preferably 1 to 5, still more preferably 1 to 3, even more preferably 1 or 2. When deleting one or more amino acids of the amino acid sequence of hARSA, the number of amino acids to be deleted is preferably 1 to 10, more preferably 1 to 5, still more preferably 1 to 3, and even more preferably 1 or 2. It is also possible to introduce a combined mutation of such substitution and deletion of amino acids. When adding one or more amino acids to hARSA, they may be added, inside, or on the N-terminal side or the C-terminal side of, the amino acid sequence of hARSA, and the number of amino acids to be added is preferably 1 to 10, more preferably 1 to 5, still more preferably 1 to 3, even more preferably 1 or 2. It is also possible to introduce a combined mutation of such addition, substitution, and deletion of the amino acid. The amino acid sequence of each of such mutated hARSA has a homology preferably not lower than 80%, more preferably not lower than 90%, and still more preferably not lower than 95%, to the amino acid sequence of the original hARSA.

Specific examples of the fusion protein where the different protein (A) is human PPT-1 (hPPT-1) include;
(1) the one that is composed of: the portion consisting of hTfR heavy chain linked, on the C-terminal side thereof and via a peptide bond, to hPPT-1, and the other portion consisting of the hTfR light chain, wherein the amino acid sequence of hTfR heavy chain of the former is set forth as SEQ ID NO:172, and the amino acid sequence of the latter is set forth as SEQ ID NO:164,
(2) the one that is composed of: the portion consisting of hTfR heavy chain linked, on the C-terminal side thereof and via a peptide bond, to hPPT-1, and the other portion consisting of the hTfR light chain, wherein the amino acid sequence of hTfR heavy chain of the former is set forth as SEQ ID NO:188, and the amino acid sequence of the latter is set forth as SEQ ID NO:180, and
(3) the one that is composed of: the portion consisting of hTfR heavy chain linked, on the C-terminal side thereof and via a peptide bond, to hPPT-1, and the other portion consisting of the hTfR light chain, wherein the amino acid sequence of hTfR heavy chain of the former is set forth as SEQ ID NO:210, and the amino acid sequence of the latter is set forth as SEQ ID NO:196.

In any of (1) to (3) above, hPPT-1 and the hTfR heavy chain may be linked by peptide bond, either directly or via a linker sequence. Namely, in any of (1) to (3) above, the meaning of the phrase “via a peptide bond” and that of the phrase “directly or via a linker sequence” are the same. The linker sequence employed here consists of 1 to 50 amino acid residues. Though there is no particular limitation as to the amino acid sequence of the linker, it is preferably made of glycine and serine: for example, a single amino acid either glycine or serine, the amino acid sequence Gly-Ser, the amino acid sequence Gly-Gly-Ser, the amino acid sequence Gly-Gly-Gly-Gly-Ser (SEQ ID NO:3), the amino acid sequence Gly-Gly-Gly-Gly-Gly-Ser (SEQ ID NO:4), the amino acid sequence Ser-Gly-Gly-Gly-Gly-Gly (SEQ ID NO:5), or a sequence consisting 1 to 50, or 2 to 17, 2 to 10, 10 to 40, 20 to 34, 23 to 31, 25 to 29, or 27 amino acids which are composed of 1 to 10 or 2 to 5 of any of those amino acid sequences consecutively linked. For example, an amino acid sequence comprising the amino acid sequence Gly-Ser may be preferably used as a linker sequence. Further, a linker sequence comprising 27 amino acids is preferably used that is composed of the amino acid sequence Gly-Ser followed by consecutively linked five copies of the amino acid sequence Gly-Gly-Gly-Gly-Ser (SEQ ID NO:3).

More specific embodiments of humanized antibody plus hPPT-1 include the one that is composed of the part consisting of hPPT-1 linked, via the linker sequence Gly-Ser, to the C-terminal side of the hTfR heavy chain, and the other part consisting of the hTfR light chain, wherein the amino acid sequence of the former is set forth as SEQ ID NO:263, and the amino acid sequence of the latter is set forth as SEQ ID NO:196.

Besides, in the present invention, though the term “human PPT-1” or “hPPT-1” refers, in particular to the hPPT-1 having the same amino acid sequence as the natural-type hPPT-1 set forth as SEQ ID NO:262, it also includes those amino acid sequences produced by introducing a mutation such as substitution, deletion, addition and the like into the amino acid sequence of the natural-type hPPT-1, insofar as they have the PPT-1 activity. When replacing one or more of the amino acids of the amino acid sequence of hPPT-1 with other amino acids, the number of amino acids to be replaced is preferably 1 to 10, more preferably 1 to 5, still more preferably 1 to 3, even more preferably 1 or 2. When deleting one or more amino acids of the amino acid sequence of hPPT-1, the number of amino acids to be deleted is preferably 1 to 10, more preferably 1 to 5, still more preferably 1 to 3, and even more preferably 1 or 2. It is also possible to introduce a combined mutation of such substitution and deletion of amino acids. When adding one or more amino acids to hPPT-1, they may be added, inside, or on the N-terminal side or the C-terminal side of, the amino acid sequence of hPPT-1, and the number of amino acids to be added is preferably 1 to 10, more preferably 1 to 5, still more preferably 1 to 3, even more preferably 1 or 2. It is also possible to introduce a combined mutation of such addition, substitution, and deletion of the amino acid. The amino acid sequence of each of such mutated hPPT-1 has a homology preferably not lower than 80%, more preferably not lower than 90%, and still more preferably not lower than 95%, to the amino acid sequence of the original hPPT-1.

Specific examples of the fusion protein where the different protein (A) is human PPT-1 (hPPT-1) include;
(1) the one that is composed of: the portion consisting of hTfR heavy chain linked, on the C-terminal side thereof via a peptide bond, to hTPP-1, and the other portion consisting of the hTfR light chain, wherein the amino acid sequence of hTfR heavy chain of the former is set forth as SEQ ID NO:172, and the amino acid sequence of the latter is set forth as SEQ ID NO:164,
(2) the one that is composed of: the portion consisting of hTfR heavy chain linked, on the C-terminal side thereof via a peptide bond, to hTPP-1, and the other portion consisting of the hTfR light chain, wherein the amino acid sequence of hTfR heavy chain of the former is set forth as SEQ ID NO:188, and the amino acid sequence of the latter is set forth as SEQ ID NO:180, and
(3) the one that is composed of: the portion consisting of hTfR heavy chain linked, on the C-terminal side thereof via a peptide bond, to hTPP-1, and the other portion consisting of the hTfR light chain, wherein the amino acid sequence of hTfR heavy chain of the former is set forth as SEQ ID NO:210, and the amino acid sequence of the latter is set forth as SEQ ID NO:196.

In any of (1) to (3) above, hTPP-1 and the hTfR heavy chain may be linked by peptide bond, either directly or via a linker sequence. Namely, in any of (1) to (3) above, the meaning of the phrase “via a peptide bond” and that of the phrase “directly or via a linker sequence” are the same. The linker sequence employed here consists of 1 to 50 amino acid residues. Though there is no particular limitation as to the amino acid sequence of the linker, it is preferably made of glycine and serine: for example, a single amino acid either glycine or serine, the amino acid sequence Gly-Ser, the amino acid sequence Gly-Gly-Ser, the amino acid sequence Gly-Gly-Gly-Gly-Ser (SEQ ID NO:3), the amino acid sequence Gly-Gly-Gly-Gly-Gly-Ser (SEQ ID NO:4), the amino acid sequence Ser-Gly-Gly-Gly-Gly-Gly (SEQ ID NO:5), or a sequence consisting 1 to 50, or 2 to 17, 2 to 10, 10 to 40, 20 to 34, 23 to 31, 25 to 29, or 27 amino acids which are composed of 1 to 10 or 2 to 5 of any of those amino acid sequences consecutively linked. For example, an amino acid sequence comprising the amino acid sequence Gly-Ser may be preferably used as a linker sequence. Further, a linker sequence comprising 27 amino acids is preferably used that is composed of the amino acid sequence Gly-Ser followed by consecutively linked five copies of the amino acid sequence Gly-Gly-Gly-Gly-Ser (SEQ ID NO:3).

More specific embodiments of humanized antibody plus hTPP-1 include the one that is composed of the part consisting of hTPP-1 linked, via the linker sequence Gly-Ser, to the C-terminal side of the hTfR heavy chain, and the other part consisting of the hTfR light chain, wherein the amino acid sequence of the former is set forth as SEQ ID NO:266, and the amino acid sequence of the latter is set forth as SEQ ID NO:196.

Besides, in the present invention, though the term “human TPP-1” or “hTPP-1” refers, in particular to the hTPP-1 having the same amino acid sequence as the natural-type hTPP-1 set forth as SEQ ID NO:265, it also includes those amino acid sequences produced by introducing a mutation such as substitution, deletion, addition and the like into the amino acid sequence of the natural-type hTPP-1, insofar as they have the TPP-1 activity. When replacing one or more of the amino acids of the amino acid sequence of hTPP-1 with other amino acids, the number of amino acids to be replaced is preferably 1 to 10, more preferably 1 to 5, still more preferably 1 to 3, even more preferably 1 or 2. When deleting one or more amino acids of the amino acid sequence of hTPP-1, the number of amino acids to be deleted is preferably 1 to 10, more preferably 1 to 5, still more preferably 1 to 3, and even more preferably 1 or 2. It is also possible to introduce a combined mutation of such substitution and deletion of amino acids. When adding one or more amino acids to hTPP-1, they may be added, inside, or on the N-terminal side or the C-terminal side of, the amino acid sequence of hTPP-1, and the number of amino acids to be added is preferably 1 to 10, more preferably 1 to 5, still more preferably 1 to 3, even more preferably 1 or 2. It is also possible to introduce a combined mutation of such addition, substitution, and deletion of the amino acid. The amino acid sequence of each of such mutated hTPP-1 has a homology preferably not lower than 80%, more preferably not lower than 90%, and still more preferably not lower than 95%, to the amino acid sequence of the original hTPP-1.

Specific examples of the fusion protein where the different protein (A) is humanα-L-iduronidase (hIDUA) include;
(1) the one that is composed of: the portion consisting of hTfR heavy chain linked, on the C-terminal side thereof and via a peptide bond, to hIDUA, and the other portion consisting of the hTfR light chain, wherein the amino acid sequence of hTfR heavy chain of the former is set forth as SEQ ID NO:172, and the amino acid sequence of the latter is set forth as SEQ ID NO:164,
(2) the one that is composed of: the portion consisting of hTfR heavy chain linked, on the C-terminal side thereof and via a peptide bond, to hIDUA, and the other portion consisting of the hTfR light chain, wherein the amino acid sequence of hTfR heavy chain of the former is set forth as SEQ ID NO:188, and the amino acid sequence of the latter is set forth as SEQ ID NO:180, and
(3) the one that is composed of: the portion consisting of hTfR heavy chain linked, on the C-terminal side thereof and via a peptide bond, to hIDUA, and the other portion consisting of the hTfR light chain, wherein the amino acid sequence of hTfR heavy chain of the former is set forth as SEQ ID NO:210, and the amino acid sequence of the latter is set forth as SEQ ID NO:196.

In any of (1) to (3) above, hIDUA and the hTfR heavy chain may be linked by peptide bond, either directly or via a linker sequence. Namely, in any of (1) to (3) above, the meaning of the phrase “via a peptide bond” and that of the phrase “directly or via a linker sequence” are the same. The linker sequence employed here consists of 1 to 50 amino acid residues. Though there is no particular limitation as to the amino acid sequence of the linker, it is preferably made of glycine and serine: for example, a single amino acid either glycine or serine, the amino acid sequence Gly-Ser, the amino acid sequence Gly-Gly-Ser, the amino acid sequence Gly-Gly-Gly-Gly-Ser (SEQ ID NO:3), the amino acid sequence Gly-Gly-Gly-Gly-Gly-Ser (SEQ ID NO:4), the amino acid sequence Ser-Gly-Gly-Gly-Gly-Gly (SEQ ID NO:5), or a sequence consisting 1 to 50, or 2 to 17, 2 to 10, 10 to 40, 20 to 34, 23 to 31, 25 to 29, or 27 amino acids which are composed of 1 to 10 or 2 to 5 of any of those amino acid sequences consecutively linked. For example, an amino acid sequence comprising the amino acid sequence Gly-Ser may be preferably used as a linker sequence. Further, a linker sequence comprising 27 amino acids is preferably used that is composed of the amino acid sequence Gly-Ser followed by consecutively linked five copies of the amino acid sequence Gly-Gly-Gly-Gly-Ser (SEQ ID NO:3).

More specific embodiments of humanized antibody plus hIDUA include the one that is composed of the part consisting of hIDUA linked, via the linker sequence Gly-Ser, to the C-terminal side of the hTfR heavy chain, and the other part consisting of the hTfR light chain, wherein the amino acid sequence of the former is set forth as SEQ ID NO:269, and the amino acid sequence of the latter is set forth as SEQ ID NO:196.

In the present invention, though the term “human IDUA” or “hIDUA” refers, in particular, to the hIDUA having the same amino acid sequence as the natural-type hIDUA set forth as SEQ ID NO:268, it also includes those amino acid sequences produced by introducing a mutation such as substitution, deletion, addition and the like into the amino acid sequence of the natural-type hIDUA, insofar as they have the hIDUA activity. When replacing one or more of the amino acids of the amino acid sequence of hIDUA with other amino acids, the number of amino acids to be replaced is preferably 1 to 10, more preferably 1 to 5, still more preferably 1 to 3, even more preferably 1 or 2. When deleting one or more amino acids of the amino acid sequence of hIDUA, the number of amino acids to be deleted is preferably 1 to 10, more preferably 1 to 5, still more preferably 1 to 3, and even more preferably 1 or 2. It is also possible to introduce a combined mutation of such substitution and deletion of amino acids. When adding one or more amino acids to hIDUA, they may be added, inside, or on the N-terminal side or the C-terminal side of, the amino acid sequence of hIDUA, and the number of amino acids to be added is preferably 1 to 10, more preferably 1 to 5, still more preferably 1 to 3, even more preferably 1 or 2. It is also possible to introduce a combined mutation of such addition, substitution, and deletion of the amino acid. The amino acid sequence of the mutated hIDUA has a homology of preferably not lower than 80%, more preferably not lower than 90%, still more preferably not lower than 95% to the amino acid sequence of the original hIDUA.

Specific examples of the fusion protein where the different protein (A) is human TNF-α receptor (hTNFαR) include;
(1) the one that is composed of: the portion consisting of hTfR heavy chain linked, on the C-terminal side thereof and via a peptide bond, to hTNFαR, and the other portion consisting of the hTfR light chain, wherein the amino acid sequence of hTfR heavy chain of the former is set forth as SEQ ID NO:172, and the amino acid sequence of the latter is set forth as SEQ ID NO:164,
(2) the one that is composed of: the portion consisting of hTfR heavy chain linked, on the C-terminal side thereof and via a peptide bond, to hTNFαR, and the other portion consisting of the hTfR light chain, wherein the amino acid sequence of hTfR heavy chain of the former is set forth as SEQ ID NO:188, and the amino acid sequence of the latter is set forth as SEQ ID NO:180, and
(3) the one that is composed of: the portion consisting of hTfR heavy chain linked, on the C-terminal side thereof and via a peptide bond, to hTNFαR, and the other portion consisting of the hTfR light chain, wherein the amino acid sequence of hTfR heavy chain of the former is set forth as SEQ ID NO:210, and the amino acid sequence of the latter is set forth as SEQ ID NO:196.

In any of (1) to (3) above, hTNFαR and the hTfR heavy chain may be linked by peptide bond, either directly or via a linker sequence. Namely, in any of (1) to (3) above, the meaning of the phrase “via a peptide bond” and that of the phrase “directly or via a linker sequence” are the same. The linker sequence employed here consists of 1 to 50 amino acid residues. Though there is no particular limitation as to the amino acid sequence of the linker, it is preferably made of glycine and serine: for example, a single amino acid either glycine or serine, the amino acid sequence Gly-Ser, the amino acid sequence Gly-Gly-Ser, the amino acid sequence Gly-Gly-Gly-Gly-Ser (SEQ ID NO:3), the amino acid sequence Gly-Gly-Gly-Gly-Gly-Ser (SEQ ID NO:4), the amino acid sequence Ser-Gly-Gly-Gly-Gly-Gly (SEQ ID NO:5), or a sequence consisting 1 to 50, or 2 to 17, 2 to 10, 10 to 40, 20 to 34, 23 to 31, 25 to 29, or 27 amino acids which are composed of 1 to 10 or 2 to 5 of any of those amino acid sequences consecutively linked. For example, an amino acid sequence comprising the amino acid sequence Gly-Ser may be preferably used as a linker sequence. Further, a linker sequence comprising 27 amino acids is preferably used that is composed of the amino acid sequence Gly-Ser followed by consecutively linked five copies of the amino acid sequence Gly-Gly-Gly-Gly-Ser (SEQ ID NO:3).

More specific embodiments of humanized antibody plus hTNFαR include the one that is composed of the part consisting of hTNFαR linked, via the linker sequence Gly-Ser, to the C-terminal side of the hTfR heavy chain, and the other part consisting of the hTfR light chain, wherein the amino acid sequence of the former is set forth as SEQ ID NO:272, and the amino acid sequence of the latter is set forth as SEQ ID NO:196.

Besides, in the present invention, though the term “human TNFαR” or “hTNFαR” refers, in particular to the hTNFαR having the same amino acid sequence as the natural-type hTNFαR set forth as SEQ ID NO:271, it also includes those amino acid sequences produced by introducing a mutation such as substitution, deletion, addition and the like into the amino acid sequence of the natural-type hTNFαR, insofar as they have the activity or function as TNFαR. When replacing one or more of the amino acids of the amino acid sequence of hTNFαR with other amino acids, the number of amino acids to be replaced is preferably 1 to 10, more preferably 1 to 5, still more preferably 1 to 3, even more preferably 1 or 2. When deleting one or more amino acids of the amino acid sequence of hTNFαR, the number of amino acids to be deleted is preferably 1 to 10, more preferably 1 to 5, still more preferably 1 to 3, and even more preferably 1 or 2. It is also possible to introduce a combined mutation of such substitution and deletion of amino acids. When adding one or more amino acids to hTNFαR, they may be added, inside, or on the N-terminal side or the C-terminal side of, the amino acid sequence of hTNFαR, and the number of amino acids to be added is preferably 1 to 10, more preferably 1 to 5, still more preferably 1 to 3, even more preferably 1 or 2. It is also possible to introduce a combined mutation of such addition, substitution, and deletion of the amino acid. The amino acid sequence of each of such mutated hTNFαR has a homology preferably not lower than 80%, more preferably not lower than 90%, and still more preferably not lower than 95%, to the amino acid sequence of the original hTNFαR.

Specific examples of the fusion protein where the different protein (A) is human heparan N-sulfatase (hSGSH) include:
(1) the one that is composed of: the portion consisting of hTfR heavy chain linked, on the C-terminal side thereof and via a peptide bond, to hSGSH, and the other portion consisting of the hTfR light chain, wherein the amino acid sequence of hTfR heavy chain of the former is set forth as SEQ ID NO:172, and the amino acid sequence of the latter is set forth as SEQ ID NO:164,
(2) the one that is composed of: the portion consisting of hTfR heavy chain linked, on the C-terminal side thereof and via a peptide bond, to hSGSH, and the other portion consisting of the hTfR light chain, wherein the amino acid sequence of hTfR heavy chain of the former is set forth as SEQ ID NO:188, and the amino acid sequence of the latter is set forth as SEQ ID NO:180, and
(3) the one that is composed of: the portion consisting of hTfR heavy chain linked, on the C-terminal side thereof and via a peptide bond, to hSGSH, and the other portion consisting of the hTfR light chain, wherein the amino acid sequence of hTfR heavy chain of the former is set forth as SEQ ID NO:210, and the amino acid sequence of the latter is set forth as SEQ ID NO:196.

In any of (1) to (3) above, hSGSH and the hTfR heavy chain may be linked by peptide bond, either directly or via a linker sequence. Namely, in any of (1) to (3) above, the meaning of the phrase “via a peptide bond” and that of the phrase “directly or via a linker sequence” are the same. The linker sequence employed here consists of 1 to 50 amino acid residues. Though there is no particular limitation as to the amino acid sequence of the linker, it is preferably made of glycine and serine: for example, a single amino acid either glycine or serine, the amino acid sequence Gly-Ser, the amino acid sequence Gly-Gly-Ser, the amino acid sequence Gly-Gly-Gly-Gly-Ser (SEQ ID NO:3), the amino acid sequence Gly-Gly-Gly-Gly-Gly-Ser (SEQ ID NO:4), the amino acid sequence Ser-Gly-Gly-Gly-Gly-Gly (SEQ ID NO:5), or a sequence consisting 1 to 50, or 2 to 17, 2 to 10, 10 to 40, 20 to 34, 23 to 31, 25 to 29, or 27 amino acids which are composed of 1 to 10 or 2 to 5 of any of those amino acid sequences consecutively linked. For example, an amino acid sequence comprising the amino acid sequence Gly-Ser may be preferably used as a linker sequence. Further, a linker sequence comprising 27 amino acids is preferably used that is composed of the amino acid sequence Gly-Ser followed by consecutively linked five copies of the amino acid sequence Gly-Gly-Gly-Gly-Ser (SEQ ID NO:3).

More specific embodiments of humanized antibody plus hSGSH include the one that is composed of the part consisting of hSGSH linked, via the linker sequence Gly-Ser, to the C-terminal side of the hTfR heavy chain, and the other part consisting of the hTfR light chain, wherein the amino acid sequence of the former is set forth as SEQ ID NO:275, and the amino acid sequence of the latter is set forth as SEQ ID NO:196.

Besides, in the present invention, though the term “human SGSH” or “hSGSH” refers, in particular to the hSGSH having the same amino acid sequence as the natural-type hSGSH set forth as SEQ ID NO:274, it also includes those amino acid sequences produced by introducing a mutation such as substitution, deletion, addition and the like into the amino acid sequence of the natural-type hSGSH, insofar as they have the SGSH activity. When replacing one or more of the amino acids of the amino acid sequence of hSGSH with other amino acids, the number of amino acids to be replaced is preferably 1 to 10, more preferably 1 to 5, still more preferably 1 to 3, even more preferably 1 or 2. When deleting one or more amino acids of the amino acid sequence of hSGSH, the number of amino acids to be deleted is preferably 1 to 10, more preferably 1 to 5, still more preferably 1 to 3, and even more preferably 1 or 2. It is also possible to introduce a combined mutation of such substitution and deletion of amino acids. When adding one or more amino acids to hSGSH, they may be added, inside, or on the N-terminal side or the C-terminal side of, the amino acid sequence of hSGSH, and the number of amino acids to be added is preferably 1 to 10, more preferably 1 to 5, still more preferably 1 to 3, even more preferably 1 or 2. It is also possible to introduce a combined mutation of such addition, substitution, and deletion of the amino acid. The amino acid sequence of each of such mutated hSGSH has a homology preferably not lower than 80%, more preferably not lower than 90%, and still more preferably not lower than 95%, to the amino acid sequence of the original hSGSH.

In the case where the different protein (A) fused with the anti-hTfR antibody is human iduronate 2-sulfatase (hI2S), human erythropoietin (hEPO), human arylsulfatase A (hARSA), human PPT-1 (hPPT-1), human TPP-1 (hTPP-1), human α-L-iduronidase (hIDUA), human TNF-α receptor (hTNFαR), or human heparan N-sulfatase (hSGSH), the expression the different protein (A) fused with the anti-hTfR antibody retains the activity or function that the different protein (A) exhibits under a physiological condition, (or simply, it retains the activity or function), means that the not less than 3% of the activity or function is retained as compared with the activity or function that the corresponding natural-type proteins intrinsically have. However, their activity or function is preferably not less than 10%, more preferably not less than 20%, still more preferably not less than 50%, and even more preferably not less than 80%, as compared with the activity or function that the corresponding natural-type different protein (A) intrinsically have. The same applies where the different protein (A) fused with the anti-hTfR antibody in mutated.

The anti-hTfR antibody according to the present invention can be used for the production of a pharmaceutical agent for parenteral administration for the treatment of a disease condition of the central nervous system by binding it to the molecule of a physiologically active protein or a pharmacologically active low-molecular-weight compound. And the anti-hTfR antibody conjugated with the molecule of a physiologically active protein or a pharmacologically active low-molecular-weight compound, can be used in the method of treatment of a patient with a disease condition of the central nervous system, in which a therapeutically effective amount of a physiologically active protein or pharmacologically active low-molecular-weight compound is administered to the patient with a disease of the central nervous system parenterally (including intravenous injection such as intravenous infusion). The anti-hTfR antibody conjugated with the molecule of a physiologically active protein or a pharmacologically active low-molecular-weight compound, after parenterally administered, can not only get inside the brain but also reach other organs where hTfR is expressed.

In particular, as the anti-hTfR antibody of the present invention can, as a conjugate with human iduronate 2-sulfatase (hI2S), enable hI2S to pass through the blood-brain barrier and function in the brain, the antibody can be used for the production of a pharmaceutical agent for parenteral administration for the treatment of a disease condition of the central nervous system accompanying Hunter syndrome. Further, the anti-hTfR antibody conjugated with hI2S can be used in the method of treatment of a patient with a disease condition of the central nervous system disorder accompanying Hunter syndrome, in which a therapeutically effective amount of the antibody is administered to the patient with Hunter syndrome parenterally (including intravenous injection such as intravenous infusion). The anti-hTfR antibody conjugated with hI2S, after parenterally administered, can not only get inside the brain but also reach other organs where hTfR is expressed.

Further, as the anti-hTfR antibody of the present invention can, as a conjugate with human erythropoietin (hEPO), enable hEPO to pass through the blood-brain barrier and function in the brain, the antibody can be used for the production of a pharmaceutical agent for parenteral administration for the treatment of a disease condition of the central nervous system accompanying cerebral ischemia. Further, the anti-hTfR antibody conjugated with hEPO can be used in the method of treatment of a patient with a disease condition of the central nervous system accompanying cerebral ischemia, in which a therapeutically effective amount of the conjugated antibody is administered to the patient with cerebral ischemia parenterally (including intravenous injection such as intravenous infusion). The anti-hTfR antibody conjugated with hEPO, after parenterally administered, can not only get inside the brain but also reach other organs where hTfR is expressed. The same applies if human erythropoietin is replaced with human darbepoetin.

Further, as the anti-hTfR antibody of the present invention can, as a conjugate with human arylsulfatase A (hARSA), enable hARSA to pass through the blood-brain barrier and function in the brain, the antibody can be used for the production of a pharmaceutical agent for parenteral administration for the treatment of a disease condition of the central nervous system accompanying metachromatic white matter degeneration (metachromatic leukodystrophy). Further, the anti-hTfR antibody conjugated with hARSA can be used in the method of treatment of a patient with a disease condition of the central nervous system accompanying metachromatic white matter degeneration (metachromatic leukodystrophy), in which a therapeutically effective amount of the conjugated antibody is administered to the patient with the disease parenterally (including intravenous injection such as intravenous infusion). The anti-hTfR antibody conjugated with hARSA, after parenterally administered, can not only get inside the brain but also reach other organs where hTfR is expressed.

Further, as the anti-hTfR antibody of the present invention can, as a conjugate with human PPT-1 (hPPT-1), enable hPPT-1 to pass through the blood-brain barrier and function in the brain, the antibody can be used for the production of a pharmaceutical agent for parenteral administration for the treatment of a disease condition of the central nervous system accompanying neuronal ceroid lipofuscinosis or Santavuori-Haltia disease. Further, the anti-hTfR antibody conjugated with hPPT-1 can be used in the method of treatment of a patient with a disease condition of the central nervous system accompanying neuronal ceroid lipofuscinosis or Santavuori-Haltia disease, in which a therapeutically effective amount of the conjugated antibody is administered to the patient with any of these diseases parenterally (including intravenous injection such as intravenous infusion). The anti-hTfR antibody conjugated with hPPT-1, after parenterally administered, can not only get inside the brain but also reach other organs where hTfR is expressed.

Further, as the anti-hTfR antibody of the present invention can, as a conjugate with human TPP-1 (hTPP-1), enable hTPP-1 to pass through the blood-brain barrier and function in the brain, the antibody can be used for the production of a pharmaceutical agent for parenteral administration for the treatment of a disease condition of the central nervous system accompanying neuronal ceroid lipofuscinosis or Jansky-Bielschowsky disease. Further, the anti-hTfR antibody conjugated with hPPT-1 can be used in the method of treatment of a patient with a disease condition of the central nervous system accompanying neuronal ceroid lipofuscinosis or Jansky-Bielschowsky disease, in which a therapeutically effective amount of the conjugated antibody is administered to the patient with any of these diseases parenterally (including intravenous injection such as intravenous infusion). The anti-hTfR antibody conjugated with hTPP-1, after parenterally administered, can not only get inside the brain but also reach other organs where hTfR is expressed.

Further, as the anti-hTfR antibody of the present invention can, as a conjugate with human α-L-iduronidase (hIDUA), enable hIDUA to pass through the blood-brain barrier and function in the brain, the antibody can be used for the production of a pharmaceutical agent for parenteral administration for the treatment of a disease condition of the central nervous system accompanying Hurler syndrome or Hurler-Scheie syndrome. Further, the anti-hTfR antibody conjugated with hIDUA can be used in the method of treatment of a patient with a disease condition of the central nervous system accompanying Hurler syndrome or Hurler-Scheie syndrome, in which a therapeutically effective amount of the conjugated antibody is administered to the patient with any of these diseases parenterally (including intravenous injection such as intravenous infusion). The anti-hTfR antibody conjugated with hIDUA, after parenterally administered, can not only get inside the brain but also reach other organs where hTfR is expressed.

Further, as the anti-hTfR antibody of the present invention can, as a conjugate with human TNF-α receptor (hTNFαR), enable hTNFαR to pass through the blood-brain barrier and function in the brain, the antibody can be used for the production of a pharmaceutical agent for parenteral administration for the treatment of a disease condition of the central nervous system accompanying cerebral ischemia or encephalitis. Further, the anti-hTfR antibody conjugated with hTNFαR can be used in the method of treatment of a patient with a disease condition of the central nervous system accompanying cerebral ischemia or encephalitis, in which a therapeutically effective amount of the conjugated antibody is administered to the patient with any of these diseases parenterally (including intravenous injection such as intravenous infusion). The anti-hTfR antibody conjugated with hTNFαR, after parenterally administered, can not only get inside the brain but also reach other organs where hTfR is expressed.

Further, as the anti-hTfR antibody of the present invention can, as a conjugate with human heparan N-sulfatase (hSGSH), enable hSGSH to pass through the blood-brain barrier and function in the brain, the antibody can be used for the production of a pharmaceutical agent for parenteral administration for the treatment of a disease condition of the central nervous system accompanying Sanfilippo syndrome. Further, the anti-hTfR antibody conjugated with hSGSH can be used in the method of treatment of a patient with a disease condition of the central nervous system accompanying Sanfilippo syndrome, in which a therapeutically effective amount of the conjugated antibody is administered to the patient with any of these diseases parenterally (including intravenous injection such as intravenous infusion). The anti-hTfR antibody conjugated with hSGSH, after parenterally administered, can not only get inside the brain but also reach other organs where hTfR is expressed.

The proteins, low-molecular-weight compound and the like that are conjugated with the anti-hTfR antibody of the present invention can be used as pharmaceutical agents which are to exhibit their functions in the central nervous system (CNS) after parenterally administered. Such pharmaceutical agents may be administered to patients generally by intravenous injection such as intravenous injection, subcutaneous injection, intramuscular injection and the like, though there is no particular limitation as to the route of their administration.

The proteins, the low-molecular-weight compounds and the like that are conjugated with the anti-hTfR antibody of the present invention can be provided to medical facilities as pharmaceutical agents in such a form as a lyophilized product or an aqueous preparation. In the case of an aqueous preparation, it can be provided in the form of preparations in which one of the pharmaceutical agents is dissolved in a solution containing a stabilizer, buffer, and an isotonizer in advance, and sealed in vials or syringes. A type of preparations sealed in a syringe is generally called a prefilled syringe-type preparation. Taking the form of a prefilled syringe-type preparation facilitates patients' self-administration of the pharmaceutical agent.

Where an aqueous preparation is provided, the concentration of the protein, the low-molecular-weight compound or the like conjugated with the anti-hTfR antibody in the aqueous preparation is, e.g., 1 to 4 mg/mL, though it is to be adjusted as desired in accordance with the dosage. Where there is no particular limitation as to stabilizers to be contained in the aqueous preparation insofar as they are pharmaceutically available, nonionic surfactants may preferably be used. Examples of such nonionic surfactants include polysorbate and poloxamer, either of which may be used alone or in combination. Among polysorbates, polysorbate 20 and polysorbate 80 are preferably used. As poloxamer, poloxamer 188 (polyoxyethylene (160) polyoxypropylene (30) glycol) is particularly preferred. Further, the concentration of nonionic surfactant contained in the aqueous preparation is preferably 0.01 to 1 mg/mL, more preferably, 0.01 to 0.5 mg/mL, and still more preferably 0.1 to 0.5 mg/mL. As stabilizers, amino acids such as histidine, arginine, methionine, and glycine may also be used. Where employed as a stabilizer, the concentration of an amino acid in the aqueous preparation is preferably 0.1 to 40 mg/mL, more preferably 0.2 to 5 mg/mL, and still more preferably 0.5 to 4 mg/mL. While there is no particular limitation as to a buffer to be contained in the aqueous preparation insofar as it is pharmaceutically available, phosphate buffer is preferred, and more preferred is sodium phosphate buffer. Where used as a buffer, the concentration of sodium phosphate is preferably 0.01 to 0.04 M. The pH of the aqueous preparation adjusted with a buffer is preferably 5.5 to 7.2. While there is no particular limitation as to an isotonizer to be contained in the aqueous preparation insofar as it is pharmaceutically available, sodium chloride or mannitol may be preferably used alone or in combination as an isotonizer.

### EXAMPLES

Though the present invention is described in further detail below with reference to examples, it is not intended that the present invention be limited to those examples.

### [Example 1] Construction of hTfR Expression Vector

Employing human spleen Quick Clone CDNA (Clontech Inc.) as a template and using primer hTfR5′ (SEQ ID NO:214) and primer hTfR3′ (SEQ ID NO:215), PCR was performed to amplify the gene fragment encoding human transferrin receptor (hTfR). The amplified fragment encoding hTfR was digested with MluI and NotI, and then inserted between MluI and NotI sites of vector pCI-neo (Promega Corp.). The vector thus prepared was designated pCI-neo (hTfR). This vector then was digested with MluI and NotI to cut out the gene fragment encoding hTfR, and this fragment was inserted between MluI and NotI sites of pE-mIRES-GS-puro, an expression vector disclosed in an international publication WO 2012/063799 to construct an hTfR expression vector, pE-mIRES-GS-puro (hTfR).

### [Example 2] Preparation of Recombinant hTfR

Into CHO-K1 cells was introduced pE-mIRES-GS-puro (hTfR) by electroporation, and the cells then were subjected to selection culture in a CD OptiCHO™ medium (Invitrogen Inc.) containing methionine sulfoximine (MSX) and puromycin to prepare recombinant hTfR expressing cells. The recombinant hTfR expressing cells were cultured, and recombinant hTfR was prepared.

### [Example 3] Immunization of Mouse with Recombinant hTfR

Mice were immunized with recombinant hTfR prepared in Example 2 as antigen. Immunization was carried out by intravenously or intraperitoneally injecting the mice with the antigen.

### [Example 4] Preparation of Hybridoma Cells

About one week after the last injection, the spleens of the mice were excised and homogenized to isolate spleen cells. The spleen cells thus obtained were fused with cells of mouse myeloma cell line (P3.X63.Ag8.653) by the polyethylene glycol method. After cell fusion, the cells were suspended in a RPMI 1640 medium containing (1×) HAT supplement (Life Technologies Inc.) and 10% Ultra low IgG fetal bovine serum (Life Technologies Inc.), and the cell suspension was dispensed to twenty 96-well plates, each at 200 μL/well. After the cells were cultured for 10 days in a carbon dioxide gas incubator (37° C., 5% CO₂), each well was examined under a microscope, and the wells that contain a single colony were selected.

When the cells in each well reached near confluence, the culture supernatant was collected as a culture supernatant of hybridoma, and subjected to the following screening process.

### [Example 5] Screening of High Affinity Antibody Producing Cell Line

The recombinant hTfR solution (Sino Biologics Inc.) was diluted with 50 mM sodium phosphate buffer (pH 9.5 to 9.6) to 5 μg/mL to prepare a solid phase solution. After 50 μL of the solid phase solution was added to each well of a Nunc MaxiSorp™ flat-bottom 96-well plate (substrate: polystyrene, mfd. by Nunc Inc.), the plate was left to stand for one hour at room temperature to let the recombinant hTfR adhere to the plate and become immobilized. The solid phase solution was discarded, each well was washed three times with 250 μL of washing solution (PBS containing 0.05% Tween20), 200 μL of a blocking solution (PBS containing 1% BSA) then was added to each well, and the plate was left to stand for one hour at room temperature.

The blocking solution was discarded, and each well was washed three times with 250 μL washing solution (PBS containing 0.05% Tween20). To each well was added 50 μL of the hybridoma culture supernatant, and the plate was left to stand for one hour at room temperature to let the mouse anti-hTfR antibody contained in the culture supernatant bind to the recombinant hTfR. At the same time, to some wells was added 50 μL of culture supernatant of a hybridoma that did not produce mouse anti-hTfR antibody, as a control. In addition, 50 μL of the medium for hybridoma culture was added to the wells, as mock wells, beside the wells to which the culture supernatant was added. Measurement was conducted in an n=2 fashion. Then, the solution was discarded, and each well was washed three times with 250 μL of washing solution (PBS containing 0.05% Tween20).

To each of the above wells was added 100 μL of HRP-labelled goat anti-mouse immunoglobulin antibody solution (Promega Inc.), and the plate was left to stand for one minute at room temperature. The solution then was discarded, and each well was washed three times with 250 μL of washing solution (PBS containing 0.05% Tween20). To each well as added 50 μL of a chromogenic substrate solution, TMB Stabilized Substrate for Horseradish Peroxidase (Promega Inc.), and the wells were left to stand for 10 to 20 minutes at room temperature. Then, following addition of 100 μL of a stop solution (2N sulfuric acid), the absorbance of each well was measured on a plate reader at 450 nm. Of the two wells for each of the culture supernatant and control, the mean values were taken, respectively, and from each of the mean values, the respective mean value for the two mock wells placed corresponding to each of the culture supernatant and the control, was subtracted, giving the measurement.

Fourteen types of hybridoma cells corresponding to culture supernatants added to the wells which exhibited the higher measurements were selected as the cell lines (high affinity antibody producing cell line) that produce antibodies exhibiting high affinities to hTfR (high affinity anti-hTfR antibody). These fourteen types of cell lines were designated as Clone 1 line to Clone 14 line. Further, the anti-hTfR antibodies produced by Clone 1 line to Clone 14 line were designated as anti-hTfR antibodies Nos. 1 to 14, respectively.

### [Example 6] Analysis of the Variable-Region Amino Acid Sequence of the High Affinity Anti-hTfR Antibodies

From each of the Clone 1 line to Clone 14 line selected in Example 5, cDNA were prepared, using which as a template the genes encoding the light chain and the heavy chain of the antibody were amplified. By translating the nucleotide sequence of the amplified genes, the respective amino acid sequences of the light chain and heavy chain variable regions were determined for the anti-hTfR antibodies Nos. 1 to 14 produced by the cell lines.

The anti-hTfR antibody No. 1 was found to include the amino acid sequence set forth as SEQ ID NO:218 as the light chain variable region, and the amino acid sequence set forth as SEQ ID NO:219 as the heavy chain variable region. The light chain variable region was found to include the amino acid sequence set forth as SEQ ID NO:6 or 7 as CDR1; SEQ ID NO:8 or 9 as CDR2, and SEQ ID NO:10 as CDR3; and the heavy chain variable region to include the amino acid sequence set forth as SEQ ID NO:76 or 77 as CDR1, SEQ ID NO:78 or 79 as CDR2, and SEQ ID NO:80 or 81 as CDR3. However, it was also considered that CDRs are not limited to those which consist of these amino acid sequences, but they can also either be regions of amino acid sequences that include any of the above sequences, or amino acid sequences consisting of not less than three consecutive amino acids containing part of the above sequences.

The anti-hTfR antibody No. 2 was found to include the amino acid sequence set forth as SEQ ID NO:220 as the light chain variable region, and the amino acid sequence set forth as SEQ ID NO:221 as the heavy chain variable region. The light chain variable region was found to include the amino acid sequence set forth as SEQ ID NO:11 or 12 as CDR1; SEQ ID NO:13 or 14 as CDR2, and SEQ ID NO:15 as CDR3; and the heavy chain variable region to include the amino acid sequence set forth as SEQ ID NO:82 or 83 as CDR1, SEQ ID NO:84 or 85 as CDR2, and SEQ ID NO:86 or 87 as CDR3. However, it was also considered that CDRs are not limited to those which consist of these amino acid sequences, but they can also either be regions of amino acid sequences that include any of the above sequences, or amino acid sequences consisting of not less than three consecutive amino acids containing part of the above sequences.

The anti-hTfR antibody No. 3 was found to include the amino acid sequence set forth as SEQ ID NO:222 as the light chain variable region, and the amino acid sequence set forth as SEQ ID NO:223 as the heavy chain variable region. The light chain variable region was found to include the amino acid sequence set forth as SEQ ID NO:16 or 17 as CDR1; SEQ ID NO:18 or 19 as CDR2, and SEQ ID NO:20 as CDR3; and the heavy chain variable region to include the amino acid sequence set forth as SEQ ID NO:88 or 89 as CDR1, SEQ ID NO:90 or 91 as CDR2, and SEQ ID NO:92 or 93 as CDR3. However, it was also considered that CDRs are not limited to those which consist of these amino acid sequences, but they can also either be regions of amino acid sequences that include any of the above sequences, or amino acid sequences consisting of not less than three consecutive amino acids containing part of the above sequences.

The anti-hTfR antibody No. 4 was found to include the amino acid sequence set forth as SEQ ID NO:224 as the light chain variable region, and the amino acid sequence set forth as SEQ ID NO:225 as the heavy chain variable region. The light chain variable region was found to include the amino acid sequence set forth as SEQ ID NO:21 or 22 as CDR1; SEQ ID NO:23 or 24 as CDR2, and SEQ ID NO:25 as CDR3; and the heavy chain variable region to include the amino acid sequence set forth as SEQ ID NO:94 or 95 as CDR1, SEQ ID NO:96 or 97 as CDR2, and SEQ ID NO:98 or 99 as CDR3. However, it was also considered that CDRs are not limited to those which consist of these amino acid sequences, but they can also either be regions of amino acid sequences that include any of the above sequences, or amino acid sequences consisting of not less than three consecutive amino acids containing part of the above sequences.

The anti-hTfR antibody No. 5 was found to include the amino acid sequence set forth as SEQ ID NO:226 as the light chain variable region, and the amino acid sequence set forth as SEQ ID NO:227 as the heavy chain variable region. The light chain variable region was found to include the amino acid sequence set forth as SEQ ID NO:26 or 27 as CDR1; SEQ ID NO:28 or 29 as CDR2, and SEQ ID NO:30 as CDR3; and the heavy chain variable region to include the amino acid sequence set forth as SEQ ID NO:100 or 101 as CDR1, SEQ ID NO:102 or 103 as CDR2, and SEQ ID NO:104 or 105 as CDR3. However, it was also considered that CDRs are not limited to those which consist of these amino acid sequences, but they can also either be regions of amino acid sequences that include any of the above sequences, or amino acid sequences consisting of not less than three consecutive amino acids containing part of the above sequences.

The anti-hTfR antibody No. 6 was found to include the amino acid sequence set forth as SEQ ID NO:228 as the light chain variable region, and the amino acid sequence set forth as SEQ ID NO:229 as the heavy chain variable region. The light chain variable region was found to include the amino acid sequence set forth as SEQ ID NO:31 or 32 as CDR1; SEQ ID NO:33 or 34 as CDR2, and SEQ ID NO:35 as CDR3; and the heavy chain variable region to include the amino acid sequence set forth as SEQ ID NO:106 or 107 as CDR1, SEQ ID NO:108 or 278 as CDR2, and SEQ ID NO:109 or 110 as CDR3. However, it was also considered that CDRs are not limited to those which consist of these amino acid sequences, but they can also either be regions of amino acid sequences that include any of the above sequences, or amino acid sequences consisting of not less than three consecutive amino acids containing part of the above sequences.

The anti-hTfR antibody No. 7 was found to include the amino acid sequence set forth as SEQ ID NO:230 as the light chain variable region, and the amino acid sequence set forth as SEQ ID NO:231 as the heavy chain variable region. The light chain variable region was found to include the amino acid sequence set forth as SEQ ID NO:36 or 37 as CDR1; SEQ ID NO:38 or 39 as CDR2, and SEQ ID NO:40 as CDR3; and the heavy chain variable region to include the amino acid sequence set forth as SEQ ID NO:111 or 112 as CDR1, SEQ ID NO:113 or 114 as CDR2, and SEQ ID NO:115 or 116 as CDR3. However, it was also considered that CDRs are not limited to those which consist of these amino acid sequences, but they can also either be regions of amino acid sequences that include any of the above sequences, or amino acid sequences consisting of not less than three consecutive amino acids containing part of the above sequences.

The anti-hTfR antibody No. 8 was found to include the amino acid sequence set forth as SEQ ID NO:232 as the light chain variable region, and the amino acid sequence set forth as SEQ ID NO:233 as the heavy chain variable region. The light chain variable region was found to include the amino acid sequence set forth as SEQ ID NO:41 or 42 as CDR1; SEQ ID NO:43 or 44 as CDR2, and SEQ ID NO:45 as CDR3; and the heavy chain variable region to include the amino acid sequence set forth as SEQ ID NO:117 or 118 as CDR1, SEQ ID NO:119 or 279 as CDR2, and SEQ ID NO:120 or 121 as CDR3. However, it was also considered that CDRs are not limited to those which consist of these amino acid sequences, but they can also either be regions of amino acid sequences that include any of the above sequences, or amino acid sequences consisting of not less than three consecutive amino acids containing part of the above sequences.

The anti-hTfR antibody No. 9 was found to include the amino acid sequence set forth as SEQ ID NO:234 as the light chain variable region, and the amino acid sequence set forth as SEQ ID NO:235 as the heavy chain variable region. The light chain variable region was found to include the amino acid sequence set forth as SEQ ID NO:46 or 47 as CDR1; SEQ ID NO:48 or 49 as CDR2, and SEQ ID NO:50 as CDR3; and the heavy chain variable region to include the amino acid sequence set forth as SEQ ID NO:122 or 123 as CDR1, SEQ ID NO:124 or 125 as CDR2, and SEQ ID NO:126 or 127 as CDR3. However, it was also considered that CDRs are not limited to those which consist of these amino acid sequences, but they can also either be regions of amino acid sequences that include any of the above sequences, or amino acid sequences consisting of not less than three consecutive amino acids containing part of the above sequences.

The anti-hTfR antibody No. 10 was found to include the amino acid sequence set forth as SEQ ID NO:236 as the light chain variable region, and the amino acid sequence set forth as SEQ ID NO:237 as the heavy chain variable region. The light chain variable region was found to include the amino acid sequence set forth as SEQ ID NO:51 or 52 as CDR1; SEQ ID NO:53 or 54 as CDR2, and SEQ ID NO:55 as CDR3; and the heavy chain variable region to include the amino acid sequence set forth as SEQ ID NO:128 or 129 as CDR1, SEQ ID NO:130 or 131 as CDR2, and SEQ ID NO:132 or 133 as CDR3. However, it was also considered that CDRs are not limited to those which consist of these amino acid sequences, but they can also either be regions of amino acid sequences that include any of the above sequences, or amino acid sequences consisting of not less than three consecutive amino acids containing part of the above sequences.

The anti-hTfR antibody No. 11 was found to include the amino acid sequence set forth as SEQ ID NO:238 as the light chain variable region, and the amino acid sequence set forth as SEQ ID NO:239 as the heavy chain variable region. The light chain variable region was found to include the amino acid sequence set forth as SEQ ID NO:56 or 57 as CDR1; SEQ ID NO:58 or 59 as CDR2, and SEQ ID NO:60 as CDR3; and the heavy chain variable region to include the amino acid sequence set forth as SEQ ID NO:134 or 135 as CDR1, SEQ ID NO:136 or 137, as CDR2, and SEQ ID NO:138 or 139 as CDR3. However, it was also considered that CDRs are not limited to those which consist of these amino acid sequences, but they can also either be regions of amino acid sequences that include any of the above sequences, or amino acid sequences consisting of not less than three consecutive amino acids containing part of the above sequences.

The anti-hTfR antibody No. 12 was found to include the amino acid sequence set forth as SEQ ID NO:240 as the light chain variable region, and the amino acid sequence set forth as SEQ ID NO:241 as the heavy chain variable region. The light chain variable region was found to include the amino acid sequence set forth as SEQ ID NO:61 or 62 as CDR1; SEQ ID NO:63 or 64 as CDR2, and SEQ ID NO:65 as CDR3; and the heavy chain variable region to include the amino acid sequence set forth as SEQ ID NO:140 or 141 as CDR1, SEQ ID NO:142 or 143 as CDR2, and SEQ ID NO:144 or 145 as CDR3. However, it was also considered that CDRs are not limited to those which consist of these amino acid sequences, but they can also either be regions of amino acid sequences that include any of the above sequences, or amino acid sequences consisting of not less than three consecutive amino acids containing part of the above sequences.

The anti-hTfR antibody No. 13 was found to include the amino acid sequence set forth as SEQ ID NO:242 as the light chain variable region, and the amino acid sequence set forth as SEQ ID NO:243 as the heavy chain variable region. The light chain variable region was found to include the amino acid sequence set forth as SEQ ID NO:66 or 67 as CDR1; SEQ ID NO:68 or 69 as CDR2, and SEQ ID NO:70 as CDR3; and the heavy chain variable region to include the amino acid sequence set forth as SEQ ID NO:146 or 147 as CDR1, SEQ ID NO:148 or 149 as CDR2, and SEQ ID NO:150 or 151 as CDR3. However, it was also considered that CDRs are not limited to those which consist of these amino acid sequences, but they can also either be regions of amino acid sequences that include any of the above sequences, or amino acid sequences consisting of not less than three consecutive amino acids containing part of the above sequences.

The anti-hTfR antibody No. 14 was found to include the amino acid sequence set forth as SEQ ID NO:244 as the light chain variable region, and the amino acid sequence set forth as SEQ ID NO:245 as the heavy chain variable region. The light chain variable region was found to include the amino acid sequence set forth as SEQ ID NO:71 or 72 as CDR1; SEQ ID NO:73 or 74 as CDR2, and SEQ ID NO:75 as CDR3; and the heavy chain variable region to include the amino acid sequence set forth as SEQ ID NO:152 or 153 as CDR1, SEQ ID NO:154 or 155 as CDR2, and SEQ ID NO:156 or 157 as CDR3. However, it was also considered that CDRs are not limited to those which consist of these amino acid sequences, but they can also either be regions of amino acid sequences that include any of the above sequences, or amino acid sequences consisting of not less than three consecutive amino acids containing part of the above sequences.

Table 1 shows collectively the SEQ ID NOs of the respective amino acid sequences included in the light chain and the heavy chain variable regions of the anti-hTfR antibody Nos. 1 to 14.

TABLE 1
Sequence numbers of respective amino acid sequences
included in the light and the heavy chain variable
regions of the anti-hTfR antibodies Nos. 1 to 14

| Antibody | light chain | heavy chain |
|---|---|---|
| No. | variable region | variable region |
| 1 | 218 | 219 |
| 2 | 220 | 221 |
| 3 | 222 | 223 |
| 4 | 224 | 225 |
| 5 | 226 | 227 |
| 6 | 228 | 229 |
| 7 | 230 | 231 |
| 8 | 232 | 233 |
| 9 | 234 | 235 |
| 10 | 236 | 237 |
| 11 | 238 | 239 |
| 12 | 240 | 241 |
| 13 | 242 | 243 |
| 14 | 244 | 245 |

Table 2 shows collectively the SEQ ID NOs of the respective amino acid sequences contained in CDR1 to CDR3 of the light chain variable region and CDR1 to CDR3 of the heavy chain variable region of anti-hTfR antibodies Nos. 1 to 14. However, Table 2 shows those amino acid sequence only as examples and does not limit the amino acid sequence of each CDR to those in Table 2, but it was considered that CDRs are not limited to those which consist of these amino acid sequences, but they can also either be regions of amino acid sequences that include any of the above sequences, or amino acid sequences consisting of not less than three consecutive amino acids containing part of the above sequences.

TABLE 2
Sequence numbers of respective amino acid sequences contained
in CDR1 to CDR3 of the light chain and the heavy chain variable
regions of anti-hTfR antibodies Nos. 1 to 14

| Anti- | light chain | | | heavy chain | | |
|---|---|---|---|---|---|---|
| body | variable region | | | variable region | | |
| No. | CDR1 | CDR2 | CDR3 | CDR1 | CDR2 | CDR3 |
| 1 | 6, 7 | 8, 9 | 10 | 76, 77 | 78, 79 | 80, 81 |
| 2 | 11, 12 | 13, 14 | 15 | 82, 83 | 84, 85 | 86, 87 |
| 3 | 16, 17 | 18, 19 | 20 | 88, 89 | 90, 91 | 92, 93 |
| 4 | 21, 22 | 23, 24 | 25 | 94, 95 | 96, 97 | 98, 99 |
| 5 | 26, 27 | 28, 29 | 30 | 100, 101 | 102, 103 | 104, 105 |
| 6 | 31, 32 | 33, 34 | 35 | 106, 107 | 108, 278 | 109, 110 |
| 7 | 36, 37 | 38, 29 | 40 | 111, 112 | 113, 114 | 115, 116 |
| 8 | 41, 42 | 43, 44 | 45 | 117, 118 | 119, 279 | 120, 121 |
| 9 | 46, 47 | 48, 49 | 50 | 122, 123 | 124, 125 | 126, 127 |
| 10 | 51, 52 | 53, 54 | 55 | 128, 129 | 130, 131 | 132, 133 |
| 11 | 56, 57 | 58, 59 | 60 | 134, 135 | 136, 137 | 138, 139 |
| 12 | 61, 62 | 63, 64 | 65 | 140, 141 | 142, 143 | 144, 145 |
| 13 | 66, 67 | 68, 69 | 70 | 146, 147 | 148, 149 | 150, 151 |
| 14 | 71, 72 | 73, 74 | 75 | 152, 153 | 154, 155 | 156, 157 |

### [Example 7] Measurement of the Affinity of Anti-hTfR Antibody to Human and Monkey TfRs

The affinity of the anti-hTfR antibody to human and monkey TfRs were measured on Octet RED96 (ForteBio Inc., a division of Pall Corporation), a system for analysis of interactions between biomolecules utilizing bio-layer interferometry (BLI). The basic principles of bio-layer interferometry are briefly explained below. When a layer of a biomolecule immobilized on the surface of a sensor tip is irradiated with light of a certain wavelength, the light is reflected from two of the surfaces, the one of the biomolecule and the other of inner, reference layer, producing interfering light waves. A molecule in the sample being measured binds to the biomolecule on the surface of the sensor tip and thus increases the thickness of the layers on the sensor tip, which results in a shift between the interfering waves. By measuring the variations of this shift between the interfering waves, determination of the number of the molecules bound to the layer of the biomolecules immobilized to the sensor tip surface and kinetic analysis of it can be performed in real time. The measurement was performed according generally to the operating manual attached to Octet RED96. As a human TfR, a recombinant human TfR (r human TfR: Sino Biological Inc.) was used, which had the amino acid sequence of the hTfR extracellular region, i.e., the cysteine residue at the position 89th from the N-terminal side to the phenylalanine at the C-terminus, of the amino acid sequence set forth as SEQ ID NO:1, with a histidine tag attached to the N-terminus. As a monkey TfR, a recombinant monkey TfR (r monkey TfR: Sino Biological Inc.) was used, which had the amino acid sequence of the cynomolgus monkey TfR extracellular region, i.e., the cysteine residue at the position 89th from the N-terminal side to the phenylalanine at the C-terminus, of the amino acid sequence set forth as SEQ ID NO:2, with a histidine tag attached to the N-terminus.

Clone 1 line to Clone 14 line selected in Example 5 were respectively diluted with a RPMI 1640 medium containing (1×) HAT Supplement (Life Technologies Inc.) and 10% Ultra low IgG fetal bovine serum (Life Technologies Inc.) so as to adjust the cell density to approximately 2×10⁵ cells/mL. To a 1-L conical flask were added 200 mL of each cell suspension, and the culture was performed for 6 to 7 days in a humid environment at 37° C., 5% CO₂ and 95% air, with stirring at a rate of about 70 rpm. The culture supernatant was collected by centrifugation, and filtered through a 0.22 μm filter (Millipore Inc.) to prepare the culture supernatant. The culture supernatant thus collected was loaded onto a Protein G column (column volume: 1 mL, GE Healthcare Inc.) that had been equilibrated in advance with three column volumes of 20 mM Tris buffer (pH 8.0) containing 150 mM NaCl. After the column was washed with 5 column volumes of the same buffer, adsorbed antibody was eluted with 4 column volumes of 50 mM glycine buffer (pH 2.8) containing 150 mM NaCl, and eluted fractions were collected. The eluted fractions were adjusted to pH 7.0 by addition of 1 M Tris buffer (pH 8.0). These were used as purified products of anti-hTfR antibodies Nos. 1 to 14 in the experiments described below.

Each of the antibodies (anti-hTfR antibody Nos. 1 to 14) purified above was subjected to 2-fold dilution steps with HBS-P+ (10 mM HEPES containing 150 mM NaCl, 50 μM EDTA and 0.05% Surfactant P20) to prepare antibody solutions of 7 different concentrations, 0.78125 to 50 nM (0.117 to 7.5 μg/mL). These antibody solutions were used as the sample solutions. The r human and r monkey TfRs were respectively diluted with HBS-P+ to prepare 25 μg/mL solutions, which were used as r human TfR-ECD (Histag) solution and r monkey TfR-ECD (Histag) solution, respectively.

Each of the sample solutions prepared above by 2-fold dilution steps was added, 200 μL/well, to a 96-well plate, black (Greiner Bio-One Inc.). Each of the r human TfR-ECD (Histag) solution and the r monkey TfR-ECD (Histag) solutions prepared above was added, 200 μL/well, to predetermined wells. To respective wells for baseline, dissociation and washing were added HBS-P+, 200 μL/well. To wells for regeneration were added 10 mM Glycine-HCl, pH 1.7, 200 μL/well. To wells for activation was added 0.5 mM NiCl₂ solution, 200 μL/well. The plate and biosensor (Biosensor/Ni-NTA: ForteBio Inc., a division of Pall Corporation) were set in the prescribed positions of Octet RED96. Octet RED96 was run under the conditions shown in Table 3 below to collect data, on which then, using the analyzing software attached to Octet RED96, and fitting the binding reaction curve to 1:1 binding model or 2:1 binding model, the association rate constant (kₒₙ) and dissociation rate constant (k_{off}) of anti-hTfR antibody to r human TfR and r monkey TfR were measured and the dissociation constant (Kd) was calculated. The measurement was performed at 25 to 30° C.

TABLE 3
Operating conditions of Octet RED96

| | | Contact time | | |
|---|---|---|---|---|
| | Step | (sec) | Rate (rpm) | Threshold |
| 1 | Baseline 1 | 60 | 1000 | — |
| 2 | Load | 600 | 1000 | 1.5-2.0 |
| 3 | Baseline 2 | 60 | 1000 | |
| 4 | Association | 180 | 1000 | |
| 5 | Dissociation | 540 | 1000 | |
| 6 | Regeneration | 5 | 1000 | |
| 7 | Washing | 5 | 1000 | |
| Steps 6-7 repeated 6 to 7 times | | | | |
| 8 | Activation | 60 | 1000 | — |

Steps 1-8 repeated until all the samples measured

Table 4 shows the results of measurement of association rate constant (kₒₙ), dissociation rate constant (k_{off}) of anti-hTfR antibody Nos. 1 to 14 (corresponding to antibody Nos. 1 to 14, respectively, in the table), and dissociation constant (k_{D}) to human TfR.

TABLE 4
Affinity of anti-hTfR antibodies to human TfR

| Antibody | kon | koff | K_{D} |
|---|---|---|---|
| No. | (M⁻¹s⁻¹) | (s⁻¹) | (M) |
| 1 | 5.00 × 10⁵ | 2.55 × 10⁻⁶ | 5.09 × 10⁻¹² |
| 2 | 1.11 × 10⁶ | 1.23 × 10⁻⁵ | 1.12 × 10⁻¹¹ |
| 3 | 6.53 × 10⁵ | <1.0 × 10⁻⁷ | <1.0 × 10⁻¹² |
| 4 | 1.91 × 10⁶ | 2.29 × 10⁻⁴ | 1.20 × 10⁻¹⁰ |
| 5 | 6.71 × 10⁵ | 2.44 × 10⁻⁵ | 3.64 × 10⁻¹¹ |
| 6 | 7.54 × 10⁵ | 7.23 × 10⁻⁴ | 9.58 × 10⁻¹⁰ |
| 7 | 3.69 × 10⁵ | 3.03 × 10⁻⁵ | 8.22 × 10⁻¹¹ |
| 8 | 6.96 × 10⁵ | <1.0 × 10⁻⁷ | <1.0 × 10⁻¹² |
| 9 | 7.82 × 10⁵ | 9.46 × 10⁻⁵ | 1.21 × 10⁻¹⁰ |
| 10 | 6.79 × 10⁵ | 7.66 × 10⁻⁴ | 1.13 × 10⁻⁹ |
| 11 | 2.72 × 10⁵ | 2.28 × 10⁻⁵ | 8.37 × 10⁻¹¹ |
| 12 | 7.54 × 10⁵ | 7.23 × 10⁻⁴ | 4.32 × 10⁻¹⁰ |
| 13 | 8.35 × 10⁵ | <1.0 × 10⁻⁷ | <1.0 × 10⁻¹² |
| 14 | 9.61 × 10⁵ | <1.0 × 10⁻⁷ | <1.0 × 10⁻¹² |

Table 5 shows the results of measurement of association rate constant (kₒₙ), dissociation rate constant (k_{off}) of anti-hTfR antibody Nos. 1 to 14 (corresponding to antibody Nos. 1 to 14, respectively, in the table), and dissociation constant (k_{D}) to monkey TfR.

TABLE 5
Affinity of anti-hTfR antibodies to monkey TfR

| Antibody | kon | koff | K_{D} |
|---|---|---|---|
| No. | (M⁻¹s⁻¹) | (s⁻¹) | (M) |
| 1 | 2.80 × 10⁵ | <1.0 × 10⁻⁷ | <1.0 × 10⁻¹² |
| 2 | 4.18 × 10⁵ | 1.75 × 10⁻⁶ | 4.18 × 10⁻¹¹ |
| 3 | 3.89 × 10⁵ | <1.0 × 10⁻⁷ | <1.0 × 10⁻¹² |
| 4 | 7.54 × 10⁵ | 1.21 × 10⁻⁴ | 1.61 × 10⁻¹⁰ |
| 5 | 5.19 × 10⁵ | 7.58 × 10⁻⁴ | 1.46 × 10⁻⁹ |
| 6 | 4.95 × 10⁵ | 2.36 × 10⁻⁴ | 1.23 × 10⁻¹⁰ |
| 7 | 2.66 × 10⁵ | 4.54 × 10⁻⁶ | 1.71 × 10⁻¹¹ |
| 8 | 5.52 × 10⁵ | 5.07 × 10⁻³ | 9.18 × 10⁻⁹ |
| 9 | 6.99 × 10⁵ | 1.47 × 10⁻⁴ | 2.10 × 10⁻⁹ |
| 10 | 3.87 × 10⁵ | 1.22 × 10⁻² | 3.16 × 10⁻⁸ |
| 11 | 1.24 × 10⁵ | 4.21 × 10⁻⁴ | 3.38 × 10⁻⁹ |
| 12 | 5.05 × 10⁵ | 1.26 × 10⁻⁴ | 2.49 × 10⁻¹⁰ |
| 13 | 5.91 × 10⁵ | 7.29 × 10⁻⁵ | 1.23 × 10⁻¹⁰ |
| 14 | 7.00 × 10⁵ | 3.61 × 10⁻⁵ | 5.16 × 10⁻¹¹ |

As a result of the affinity measurement of those anti-hTfR antibodies to human TfR, the dissociation constant with human TfR was not more than 1×10⁻⁸ M for all the antibodies; and for 13 antibodies except antibody No. 10, the dissociation constant with human TfR was not more than 1×10⁻⁹ M; and for antibodies Nos. 3, 8, 13 and 14, in particular, the dissociation constant was not more than 1×10⁻¹²M (Table 4). The result demonstrates that all of the 14 antibodies are antibodies having a high-affinity antibody to human TfR. Then looking to the result of the measurement of the affinity of the anti-hTfR antibodies to monkey TfR, the dissociation constant with monkey TfR was not more than 5×10⁻⁸ M for all the antibodies, and for antibodies Nos. 1 and 3, in particular, the dissociation constant with monkey TfR was not more than 1×10⁻¹² M (Table 5). The result shows that all the 14 antibodies are antibodies having a high-affinity antibody not only to human TfR but also to monkey TfR.

### [Example 7-2] Evaluation of Brain Uptake of the Anti-hTfR Antibodies Using Mice

Then, for 13 antibodies, anti-hTfR antibodies Nos. 1 to 9 and 11 to 14, evaluation was performed about their transfer into the brain through the BBB, by using hTfR knock-in mice (hTfR-KI mice) in which the gene encoding the extracellular region of mouse transferrin receptor has been replaced with a gene encoding the extracellular region of human transferrin receptor. The hTfR-KI mice were produced by the method described below as a whole. Besides, the purified antibodies of Example 7 were used as the anti-hTfR antibodies.

A DNA fragment having a nucleotide sequence set forth as SEQ ID NO:253 was chemically synthesized, in which a neomycin resistance gene flanked by loxP sequences was placed on the 3′-side of a cDNA encoding a chimeric hTfR whose intracellular region consisted of the amino acid sequence of mouse hTfR and the extracellular region consisted of the amino acid sequence of human hTfR sequence. This DNA fragment was inserted by a conventional method into a targeting vector having as the 5′-arm sequence a nucleotide sequence set forth as SEQ ID NO:254 and as the 3′-arm sequence a nucleotide sequence set forth as SEQ ID NO:255, and the construct was introduced into mouse ES cells by electroporation. The mouse ES cells to which the gene had been introduced were subjected to selection culture in a medium in the presence of neomycin to select those mouse ES cells in which the targeting vector had been incorporated into the chromosome through homologous recombination. The recombinant mouse ES cells thus obtained were injected into 8-cell stage embryos (host embryos) of ICR mice, and the embryos thus prepared were implanted into pseudo pregnant mice (recipient mice) which had been obtained through mating with mice having undergone vasoligation. The offspring (chimeric mice) obtained were examined by their hair color, and those mice which had the higher proportion of white hairs in their total body hairs were selected, i.e., those mice in which the ES cells had contributed at the higher rates in the development of the individual organisms. Each of these chimeric mice was mated with ICR mice to generate F1 mice. F1 mice with white hair were selected, the DNAs extracted from their tail tissue were analyzed, and those mice whose mouse transferrin receptor gene on their chromosomes had been replaced with chimeric hTfR, were regarded as hTfR-KI mice.

The above 13 anti-hTfR antibodies were fluorescently labeled with fluorescein isothiocyanate (FITC) using Fluorescein Labeling Kit-NH₂ (Dojindo Laboratories) according to the attached manual. PBS solutions were prepared each containing one of the FITC fluorescent labeled 13 anti-hTfR antibodies. Each of these PBS antibody solutions was intravenously injected to an hTfR-KI mouse (male, 10 to 12-week old), at the anti-hTfR antibody dosage of 3 mg/kg. As a control, a PBS solution containing mouse IgG1 (Sigma Inc.), fluorescently labeled with FITC in the same manner as above, was intravenously injected to an hTfR-KI mouse (male, 10 to 12-week old), at the dose of 3 mg/kg. About eight hours after the intravenous injection, the whole body was perfused with saline, and the brain (part including the cerebrum and the cerebellum) was obtained. The brain thus excised was weighed (wet weight), and then the brain tissues were homogenized with T-PER (Thermo Fisher Scientific Inc.) containing Protease Inhibitor Cocktail (Sigma Inc.). The homogenate was centrifuged, the supernatant was collected, and the amount of the FITC fluorescent labeled antibody contained in the supernatant was measured in the following manner. First, 10 μL of anti-FITC Antibody (Bethyl Inc.) was added to each well of a High Bind Plate (Meso Scale Diagnostics Inc.) and left to stand for one hour so as to immobilize it to the plate. Then, the plate was blocked by addition of 150 μL of SuperBlock Blocking buffer in PBS (Thermo Fisher Scientific Inc.) to each well and shaking of the plate for one hour. Then, 25 μL of the supernatant of a brain tissue homogenate was added to each well, and the plate was shaken for one hour. Then, 25 μL of SULFO-TAG Anti-Mouse Antibody (Goat) (Meso Scale Diagnostics Inc.) were added to each well, and shaking was continued for one hour. Then, to each well was added 150 μL of Read buffer T (Meso Scale Diagnostics Inc.), and the amount of luminescence from each well was read on a Sector™ Imager 6000 reader. The amount of the antibody contained per one gram brain (wet weight) (the concentration of the anti-hTfR antibody in the brain tissues) was calculated, by producing a standard curve based on measurements of standard samples containing known concentrations of fluorescently FITC-labeled anti-hTfR antibody, and then interpolating the measurement of each of the samples with reference to the standard. The results are shown in Table 5-2.

The concentration of any of the antibodies designated anti-hTfR antibodies Nos. 1 to 9 and 11 to 14 in brain tissues was over 25 times greater than that of the control. The concentration of anti-hTfR antibodies Nos. 5 and 6 was both over 100 times greater than that of the control, with anti-hTfR antibody No. 6, in particular, the value reached approximately 160 times as high as that of the control. The results indicate that the antibodies designated anti-hTfR antibody Nos. 1 to 9 and 11 to 14 transfers into the brain, actively passing through the BBB.

TABLE 5-2
Concentration of anti-hTfR antibodies in brain tissues

| | | Relative |
|---|---|---|
| Antibody | Brain tissues | value to the |
| No. | (μg/g wet weight) | control |
| Control | 0.003 | 1 |
| 1 | 0.141 | 47.0 |
| 2 | 0.126 | 42.0 |
| 3 | 0.0833 | 27.8 |
| 4 | 0.221 | 73.7 |
| 5 | 0.335 | 112 |
| 6 | 0.492 | 164 |
| 7 | 0.0855 | 28.5 |
| 8 | 0.133 | 44.3 |
| 9 | 0.112 | 37.3 |
| 11 | 0.103 | 34.3 |
| 12 | 0.215 | 71.7 |
| 13 | 0.127 | 42.3 |
| 14 | 0.213 | 71.0 |

### [Example 8] Pharmacokinetic Analysis of Anti-hTfR Antibodies in Monkey

Each of anti-hTfR antibodies Nos. 1 to 3 was intravenously administered once to a male cynomolgus monkey at a dosage of 5.0 mg/kg, and 8 hours after the administration, whole body irrigation was carried out with physiological saline. As a negative control, a monkey which had not received anti-hTfR antibody was subjected to whole body irrigation in the same manner. After the irrigation, brain tissues including the medulla oblongata were excised. Using the brain tissues, the concentration of the anti-hTfR antibody was measured, and immunohistochemical staining was performed. Besides, the anti-hTfR antibodies employed were purification products of those described in Example 7.

Measurement of the concentration of anti-hTfR antibodies in brain tissues were carried out largely following the procedure described below. Collected brain tissues were divided into the cerebrum, the cerebellum, the hippocampus, and the medulla oblongata, and they were respectively homogenized with RIPA Buffer (Wako Pure Chemical Industries Inc.) containing Protease Inhibitor Cocktail (Sigma-Aldrich Inc.), and centrifuged to collect the supernatant. Affinipure Goat Anti mouse IgG Fcγ pAb (Jackson ImmunoResearch Inc.) was added, 10 μL each, to the wells of a High Bind Plate (Meso Scale Diagnostics Inc.), and the plate was left to stand for one hour to immobilize the antibody. Then, the plate was blocked by addition of 150 μL of SuperBlock Blocking buffer in PBS (Thermo Fisher Scientific Inc.) to each well and shaken for one hour. Then, 25 μL of the supernatant of a brain tissue homogenate was added to each well, and the plate was shaken for one hour. Then, 25 μL of Affinipure Goat Anti mouse IgG Fab-Biotin (Jackson ImmunoResearch Inc.) was added to each well, and shaking was continued for one hour. Then, 25 μL or SULFO-Tag-Streptavidin (Meso Scale Diagnostics Inc.) was added to each well, and shaking was continued for half an hour. To each well was added 150 μL of Read buffer T (Meso Scale Diagnostics Inc.), and the amount of luminescence from each well was read on a Sector™ Imager 6000 reader (Meso Scale Diagnostics). The amount of the antibody contained per one gram of brain (wet weight) (the concentration of the anti-hTfR antibody in brain tissues) was calculated, by producing a standard curve based on measurements of standard samples containing known concentrations of the anti-hTfR antibody, and then interpolating the measurement of each of the samples with reference to the standard.

The result of the measurement of the concentration of the anti-hTfR antibodies in brain tissues is shown in Table 6. Though any of anti-hTfR antibodies Nos. 1 to 3 was observed to accumulate in the cerebrum, the cerebellum, the hippocampus and the medulla oblongata, in their amount there was a relation of anti-hTfR antibody No. 1<anti-hTfR antibody No. 3<anti-hTfR antibody No. 2, showing the lowest with anti-hTfR antibody No. 1 and highest in anti-hTfR antibody No. 2. In comparison with anti-hTfR antibody No. 1, the accumulation of anti-hTfR antibody No. 2 was approximately 4.3 times in the cerebrum, approximately 6.6 times in the cerebellum, approximately 4.6 times in the hippocampus, and approximately 2 times in the medulla oblongata. These results demonstrate that these 3 antibodies had a property to pass through the blood-brain barrier and accumulate in the brain tissues, and show that by binding these antibodies to a pharmaceutical agent which needs to be brought into function in the brain tissues, it is possible to let those pharmaceutical agents efficiently accumulate in the brain tissues.

TABLE 6
Concentration of anti-hTfR antibodies
in brain tissues (μg/g wet weight)

| Antibody | | | | Medulla |
|---|---|---|---|---|
| No. | Cerebrum | Cerebellum | Hippocampus | oblongata |
| 1 | 0.18 | 0.15 | 0.12 | 0.22 |
| 2 | 0.78 | 0.99 | 0.56 | 0.43 |
| 3 | 0.82 | 0.6 | 0.33 | 0.31 |

Immunohistochemical staining of the anti-hTfR antibodies in these brain tissues was carried out using the following procedures described below basically. The collected tissues were rapidly frozen to −80° C. in a Tissue-Tek Cryo 3DM (Sakura Finetek Inc.) to prepare frozen blocks of tissues. The frozen blocks were sliced into 4-μm sections, and which were affixed to MAS coated glass slides (Matsunami Glass Inc.). The tissue sections were reacted with 4% paraformaldehyde (Wako Pure Chemical Industries Inc.) for 5 minutes at 4° C. and fixed to glass slides. Then, the tissue sections were reacted with methanol solution containing 0.3% hydrogen peroxide (Wako Pure Chemical Industries Inc.) for 30 min to inactivate endogenous peroxidases. Then, the glass slides were blocked by reacting SuperBlock blocking buffer in PBS for 30 min at room temperature. Then, the tissue sections were reacted with Mouse IgG-heavy and light chain Antibody (Bethyl Laboratories Inc.) for one hour at room temperature. The tissue sections were allowed to visualize with DAB substrate (3,3′-diaminobenzidine, Vector Laboratories Inc.), counterstained with Mayer's hematoxylin solution (Merck Inc.), embedded after dehydration and clearing, and observed under an optical microscope.

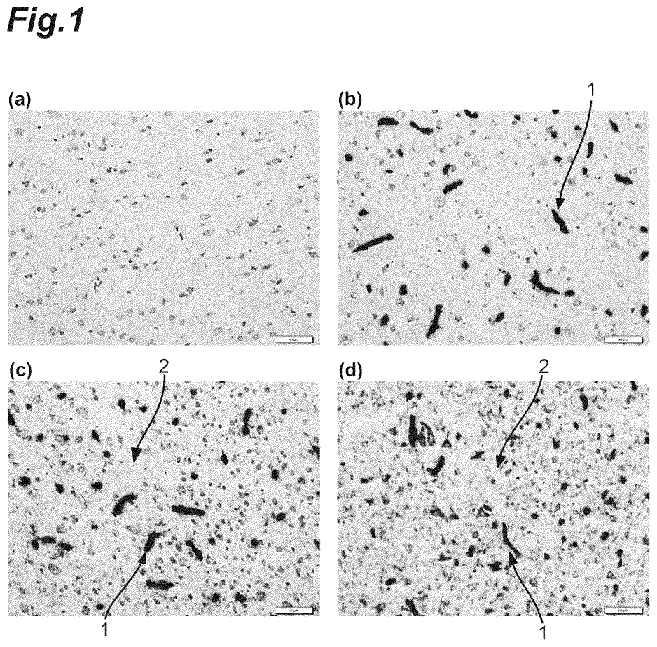

FIG. 1 shows the result of the immunohistochemical staining of the anti-hTfR antibodies in the cerebral cortex. In the cerebral cortex of monkeys administered anti-hTfR antibodies Nos. 1 to 3, specific staining in the blood vessels were observed (FIG. 1b to d, respectively). In particular, in the cerebral cortex of the monkeys administered anti-hTfR antibodies No. 2 or 3, specific staining was also observed extensively in the brain parenchyma region, outside the blood vessels (FIGS. 1c and 1d, respectively). Besides, no staining was observed in the cerebral cortex of the control monkey non-administered anti-hTfR antibody, indicating that there was almost no background staining (FIG. 1a).

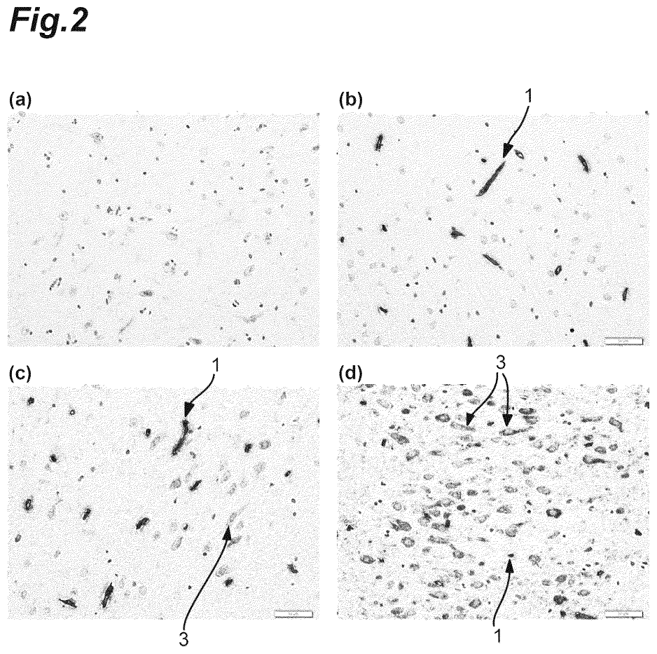

FIG. 2 shows the result of immunohistochemical staining of anti-hTfR antibodies in the hippocampus. In the cerebrum of monkeys administered anti-hTfR antibodies Nos. 1 to 3, specific staining of blood vessels were observed (FIG. 2b to d, respectively). In particular, in the hippocampus of the monkeys administered anti-hTfR antibodies No. 2 or 3, specific staining of neuron-like cells was also observed (FIGS. 2c and 2d, respectively), and specific and extensive staining of the brain parenchyma region, outside the blood vessels, was also observed. Besides, no staining was observed in the hippocampus of the control monkey non-administered anti-hTfR antibody, indicating that there was almost no background staining (FIG. 2a).

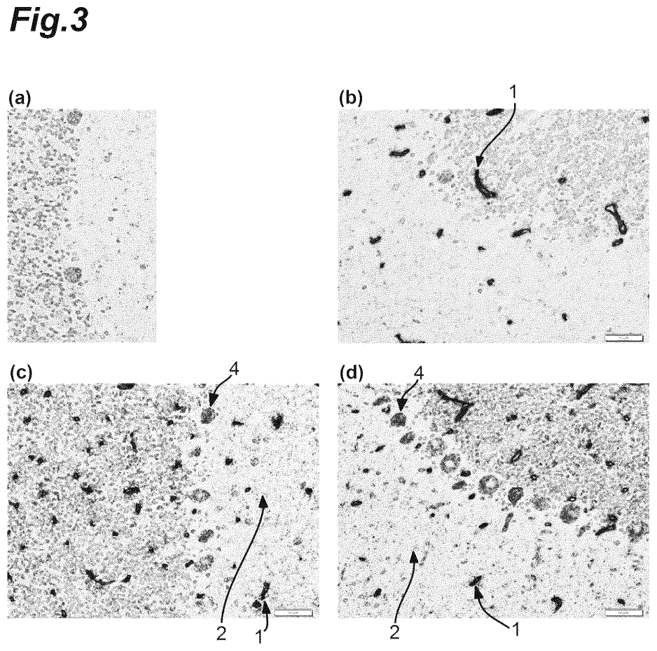

FIG. 3 shows the result of immunohistochemical staining of the anti-hTfR antibodies in the cerebellum. In the cerebellum of monkeys administered anti-hTfR antibodies Nos. 1 to 3, specific staining of blood vessels were observed (FIG. 3b to d, respectively). In particular, in the cerebellum of the monkeys administered anti-hTfR antibodies No. 2 or 3, specific staining of Purkinje cells was also observed (FIGS. 3c and 3d, respectively). Besides, no staining was observed in the cerebellum of the control with no anti-hTfR antibody administered, indicating that there was almost no background staining (FIG. 3a).

From the above results of immunohistochemical staining in the cerebrum, the hippocampus, and the cerebellum, it was considered that while anti-hTfR antibody No. 1 can bind to hTfR existing on the endothelium of blood vessels in the brain, relatively small amount of it transfers to the brain parenchyma compared with anti-hTfR antibodies Nos. 2 and 3. On the other hand, it was found that anti-hTfR antibodies Nos. 2 and 3 can bind to hTfR existing on the endothelium of blood vessels of the brain, and after binding to hTfR, they pass through the blood-brain barrier and transfer into the brain parenchyma, and further, are taken up into the brain parenchyma and neuron-like cells in the hippocampus, and are taken up by Purkinje cells in the cerebellum.

### [Example 9] Preparation of Humanized Anti-hTfR Antibodies

Humanization was tried of the amino acid sequence included in the light chain and the heavy chain variable regions of anti-hTfR antibodies Nos. 1 to 3 shown in Table 1. From anti-hTfR antibody No. 1 were obtained a humanized light chain variable region having one of the amino acid sequences set forth as SEQ ID NO:158 to SEQ ID NO:163, and a humanized heavy chain variable region having one of the amino acid sequences set forth as SEQ ID NO:166 to SEQ ID NO:171.

From anti-hTfR antibody No. 2 were obtained a humanized light chain variable region having one of the amino acid sequences set forth as SEQ ID NO:174 to SEQ ID NO:179, and a humanized heavy chain variable region having one of the amino acid sequences set forth as SEQ ID NO:182 to SEQ ID NO:187.

From anti-hTfR antibody No. 3 were obtained a humanized light chain variable region having one of the amino acid sequences set forth as SEQ ID NO:190 to SEQ ID NO:195, and a humanized heavy chain variable region having one of the amino acid sequences set forth as SEQ ID NO:204 to SEQ ID NO:209.

### [Example 10] Construction of Genes Encoding Humanized Anti-hTfR Antibodies

For each of anti-hTfR antibodies Nos. 1 to 3 above, DNA fragments were artificially synthesized which contained a gene encoding the full length of the light chain, and of the heavy chain, having humanized anti-hTfR antibody light chain and heavy chain variable regions, respectively. In doing this, a MluI sequences and a sequence encoding a leader peptide was added, in this order from the 5′ end, on the 5′ side of the gene encoding the full length of the light chain, and on the 3′ side was added a NotI sequence. And, a MluI sequences and a sequence encoding a leader peptide was added, in this order from the 5′ end, on the 5′ side of the gene encoding the full length of the heavy chain, and on the 3′ side was added a NotI sequence. The leader peptide introduced above is to function as secretion signal when the light chain and heavy chain of the humanized antibody is expressed in mammalian cells as host cells so that the light chain and the heavy chain are secreted out of the cells.

For the light chain of anti-hTfR antibody No. 1, a DNA fragment (SEQ ID NO:165) was synthesized, which included a gene encoding the full length of the light chain (the light chain of humanized anti-hTfR antibody No. 1) consisting of the amino acid sequence set forth as SEQ ID NO:164, which had in the variable region the amino acid sequence set forth as SEQ ID NO:163.

For the heavy chain of anti-hTfR antibody No. 1, a DNA fragment (SEQ ID NO:173) was synthesized, which included a gene encoding the full length of the heavy chain (the heavy chain of humanized anti-hTfR antibody No. 1) consisting of the amino acid sequence set forth as SEQ ID NO:172, which had in the variable region the amino acid sequence set forth as SEQ ID NO:171.

The heavy chain of the humanized anti-hTfR antibody encoded by the DNA fragment set forth as SEQ ID NO:173 is IgG1.

For the light chain of anti-hTfR antibody No. 2, a DNA fragment (SEQ ID NO:181) was synthesized, which included a gene encoding the full length of the light chain (the light chain of humanized anti-hTfR antibody No. 2) consisting of the amino acid sequence set forth as SEQ ID NO:180, which had in the variable region the amino acid sequence set forth as SEQ ID NO:179.

For the heavy chain of anti-hTfR antibody No. 2, a DNA fragment (SEQ ID NO:189) was synthesized, which included a gene encoding the full length of the heavy chain (the heavy chain of humanized anti-hTfR antibody No. 2) consisting of the amino acid sequence set forth as SEQ ID NO:188, which had in the variable region the amino acid sequence set forth as SEQ ID NO:187.

The heavy chain of the humanized anti-hTfR antibody encoded by the DNA fragment set forth as SEQ ID NO:189 is IgG1.

For the light chain of anti-hTfR antibody No. 3, a DNA fragment (SEQ ID NO:197) was synthesized, which included a gene encoding the full length of the light chain (the light chain of humanized anti-hTfR antibody No. 3) consisting of the amino acid sequence set forth as SEQ ID NO:196, which had in the variable region the amino acid sequence set forth as SEQ ID NO:191.

For the heavy chain of anti-hTfR antibody No. 3, a DNA fragment (SEQ ID NO:211) was synthesized, which included a gene encoding the full length of the heavy chain (the heavy chain of humanized anti-hTfR antibody No. 3) consisting of the amino acid sequence set forth as SEQ ID NO:210, which had in the variable region the amino acid sequence set forth as SEQ ID NO:205.

The heavy chain of the humanized anti-hTfR antibody encoded by the DNA fragment set forth as SEQ ID NO:211 is IgG1.

As to the light chain of anti-hTfR antibody No. 3, also synthesized were,
a DNA fragment (SEQ ID NO:199) encoding the full length amino acid sequence of the light chain (the light chain of humanized anti-hTfR antibody No. 3-2) consisting of the amino acid sequence set forth as SEQ ID NO:198, which had in the variable region the amino acid sequence set forth as SEQ ID NO:193;
a DNA fragment (SEQ ID NO:201) encoding the full length amino acid sequence of the light chain (the light chain of humanized anti-hTfR antibody No. 3-3) consisting of the amino acid sequence set forth as SEQ ID NO:200, which had in the variable region the amino acid sequence set forth as SEQ ID NO:194;
a DNA fragment (SEQ ID NO:203) encoding the full length amino acid sequence of the light chain (the light chain of humanized anti-hTfR antibody No. 3-4) consisting of the amino acid sequence set forth as SEQ ID NO:202, which had in the variable region the amino acid sequence set forth as SEQ ID NO:195;

Further, for the heavy chain of anti-hTfR antibody No. 3, also synthesized was a DNA fragment (SEQ ID NO:213) encoding the full length amino acid sequence of the heavy chain (the heavy chain IgG4 of humanized anti-hTfR antibody No. 3) consisting of the amino acid sequence set forth as SEQ NO:212, which had in the variable region the amino acid sequence set forth as SEQ ID NO:205;

The heavy chain of the humanized anti-hTfR antibody encoded by the DNA fragment set forth as SEQ ID NO:213 is IgG4.

### [Example 11] Construction of Humanized Anti-hTfR Antibody Expression Vector

Vector pEF/myc/nuc (Invitrogen Inc.) was digested with KpnI and NcoI to cut out a region including EF-1α promoter and its first intron, and this was blunt-ended with T4 DNA polymerase. A region including the CMV enhancer/promoter and intron was removed from pCI-neo (Invitrogen Inc.) by digesting it with BgIII and EcoRI, and the remaining fragment thus left was blunt-ended with T4 DNA polymerase. To this was inserted the above-mentioned region including EF-1α promoter and its first intron to construct pE-neo vector. This vector, pE-neo, was digested with SfiI and BstXI to remove a region of approximately 1 kb including a neomycin resistance gene. PCR was performed employing pcDNA3.1/Hygro (+) (Invitrogen) as a template and using primer Hyg-Sfi5′ (SEQ ID NO:216) and primer Hyg-BstX3′ (SEQ ID NO:217) to amplify hygromycin gene. The hygromycin gene thus amplified was digested with SfiI and BstXI and inserted into the above pE-neo vector from which neomycin resistance gene had been removed to construct a vector pE-hygr.

Vectors pE-hygr and pE-neo were both digested with MluI and NotI. The DNA fragment (SEQ ID NO:165) encoding the light chain of humanized anti-hTfR antibody No. 1 and the DNA fragment (SEQ ID NO:173) encoding the heavy chain of the antibody, both synthesized in Example 10, were digested with MluI and NotI, and the fragments thus obtained were inserted into vector pE-hygr and vector pE-neo, respectively, between their MluI and NotI sites. The vectors thus obtained were used as an expression vector for the light chain of humanized anti-hTfR antibody No. 1, pE-hygr(LC1), and as an expression vector for the heavy chain of humanized anti-hTfR antibody No. 1, pE-neo(HC1), in the experiments described below.

In an analogous manner, the DNA fragment (SEQ ID NO:181) encoding the light chain of humanized anti-hTfR antibody No. 2 and the DNA fragment (SEQ ID NO:189) encoding the heavy chain of the antibody, both synthesized in Example 10, were both digested with MluI and NotI, and the fragments thus obtained were inserted into vector pE-hygr and vector pE-neo, respectively, between their MluI and NotI sites. The vectors thus obtained were used as an expression vector for the light chain of humanized anti-hTfR antibody No. 2, pE-hygr(LC2), and as an expression vector for the heavy chain of the antibody of humanized anti-hTfR antibody No. 2, pE-neo(HC2), in the experiments described below.

Further, in the same manner as above, the DNA fragment (SEQ ID NO:197) encoding the light chain of humanized anti-hTfR antibody No. 3 and the DNA fragment (SEQ ID NO:211) encoding the heavy chain of the antibody, both synthesized in Example 10, were both digested with MluI and NotI, and the fragments thus obtained were inserted into vector pE-hygr and vector pE-neo, respectively, between their MluI and NotI sites. The vectors thus obtained were used as an expression vector for the light chain of humanized anti-hTfR antibody No. 3, pE-hygr(LC3), and as an expression vector for the heavy chain of the antibody, pE-neo(HC3), in the experiments described below.

Further, as to the light chain of anti-hTfR antibody No. 3, the following fragments synthesized in Example 10, namely:
the DNA fragment (SEQ ID NO:199) encoding the light chain of humanized anti-hTfR antibody No. 3-2,
the DNA fragment (SEQ ID NO:201) encoding the light chain of humanized anti-hTfR antibody No. 3-3, and
the DNA fragment (SEQ ID NO:203) encoding the light chain of humanized anti-hTfR antibody No. 3-4, were digested with MluI and NotI, and inserted into the vector pE-hygr between the MluI and NotI sites thereof to construct
pE-hygr(LC3-2), an expression vector for the light chain of humanized anti-hTfR antibody No. 3-2,
pE-hygr(LC3-3), an expression vector for the light chain of humanized anti-hTfR antibody No. 3-3, and
pE-hygr(LC3-4), an expression vector for the light chain of humanized anti-hTfR antibody No. 3-4, respectively.

Further, in the same manner as above, as to the heavy chain of anti-hTfR antibody No. 3, the DNA fragment (SEQ ID NO:213) encoding the heavy chain IgG4 of humanized anti-hTfR antibody No. 3 synthesized in Example 10 was digested with MluI and NotI, and inserted into the vector pE-neo between the MluI and NotI sites thereof to construct pE-neo(HC3-IgG4), an expression vector for the heavy chain IgG4 of humanized anti-hTfR antibody No. 3.

### [Example 12] Construction of Cells for Expression of Humanized Anti-hTfR Antibody

CHO cells (CHO-K1: obtained from American Type Culture Collection) were transformed with pE-hygr(LC1), the vector for light chain expression, and pE-neo(HC1), the vector for heavy chain expression, both constructed in Example 11, as follows, using GenePulser (Bio-Rad Inc.). Transformation of the cells was performed in the following manner as a whole. 5×10⁵ of CHO-K1 cells were seeded in a 3.5-cm culture dish containing CD OptiCHO™ medium (Life Technologies Inc.) and cultured overnight at 37° C., 5% CO₂. The medium was replaced with Opti-MEM™ I medium (Life Technologies Inc.), and the cells were suspended at the density of 5×10⁶ cells/mL. 100 μL of the cell suspension were taken, to which was added 5 μL each of a pE-hygr(LC1) and a pE-neo(HC1) plasmid DNA solution both having been diluted with Opti-MEM™ I medium to 100 μg/mL. These plasmids were introduced into the cells by electroporation using GenePulser (Bio-Rad Inc.). The cells then were cultured overnight under the condition of 37° C., 5% CO₂, and subjected to selection culture in CD OptiCHO™ medium supplemented with 0.5 mg/ml of hygromycin and 0.8 mg/mL of G418.

Then, the cells selected above through the selection culture were seeded on 96-well plates so that not more than one cell might be seeded per well by limiting dilution. The cells then were cultured for about 10 days so that monoclonal colonies were formed. Respective culture supernatants of the wells in which monoclonal colony was formed were collected, the amount of the humanized antibody contained in culture supernatants was determined by ELISA, and humanized antibody high-expressing cell lines were selected.

The ELISA above was conducted as follows in general. To each well of 96-well microtiter plates (Nunc Inc.) were added 100 μL of a goat anti-human IgG polyclonal antibody solution diluted with 0.05 M sodium bicarbonate buffer (pH 9.6) to 4 μg/mL, and the plate was left to stand for at least one hour at room temperature so as to allow the antibody to be adsorbed by the plates. Then, after each well was washed three times with a phosphate-buffered saline (pH 7.4) supplemented with 0.05% Tween20 (PBS-T), 200 μL of Starting Block (PBS) Blocking Buffer (Thermo Fisher Scientific Inc.) was added to each well, and the plates were left to stand for 30 minutes at room temperature. After each well was washed with PBS-T three times, the culture supernatant or the human IgG reference standard product which had been diluted with a PBS supplemented with 0.5% BSA and 0.05% Tween20 (PBS-BT) to appropriate concentrations, was added to each well, in the amount of 100 μL, and the plates were left to stand for at least one hour at room temperature. After the plates were washed three times with PBS-T, 100 μL of HRP-labeled anti-human IgG polyclonal antibody solution which had been diluted with PBS-BT, was added to each well, and the plates were left to stand for at least one hour at room temperature. After the wells were washed three times with PBS-T, 0.4 mg/mL o-phenylenediamine in citrate-phosphate buffer (pH 5.0) was added to each well, in the amount of 100 μL, and the wells were left to stand for 8 to 20 minutes at room temperature. Then, 1 mol/L sulfuric acid was added to each well, in the amount of 100 μL to terminate the reaction, and the absorbance for each well was measured at 490 nm using a 96-well plate reader. The cells corresponding to the wells which exhibited the higher measurements were regarded as a high-expressing cell line for humanized anti-hTfR antibody No. 1. This was designated antibody No. 1 expressing cell line.

In the same manner, CHO cells were transformed with the light chain expression vector pE-hygr(LC2) and the heavy chain expression vector pE-neo(HC2), both constructed in Example 11, and a high-expressing cell line for humanized anti-hTfR antibody No. 2 was obtained. This was designated antibody No. 2 expressing cell line.

Further, in the same manner, CHO cells were transformed with the light chain expression vector pE-hygr(LC3) and the heavy chain expression vector pE-neo(HC3), both constructed in Example 11, and a high-expressing cell line for humanized anti-hTfR antibody No. 3 was obtained. This was designated antibody No. 3 expressing cell line.

Further, in the same manner, CHO cells were transformed with the light chain expression vector pE-hygr(LC3-2) and the heavy chain expression vector pE-neo(HC3), both constructed in Example 11, and a high-expressing cell line for humanized anti-hTfR antibody No. 3-2 was obtained. This was designated antibody No. 3-2 expressing cell line.

Further, in the same manner, CHO cells were transformed with the light chain expression vector pE-hygr(LC3-3) and the heavy chain expression vector pE-neo(HC3), both constructed in Example 11, and a high-expressing cell line for humanized anti-hTfR antibody No. 3-3 was obtained. This was designated antibody No. 3-3 expressing cell line.

Further, in the same manner, CHO cells were transformed with the light chain expression vector pE-hygr(LC3-4) and the heavy chain expression vector pE-neo(HC3) both constructed in Example 11, and a high-expressing cell line for humanized anti-hTfR antibody No. 3-4 was obtained. This was designated antibody No. 3-4 expressing cell line.

Further, in the same manner, CHO cells were transformed with the light chain expression vector pE-hygr(LC3) and the heavy chain expression vector pE-neo(HC3-IgG4) both constructed in Example 11, and a high-expressing cell line for humanized anti-hTfR antibody No. 3 (IgG4) was obtained. This was designated antibody No. 3 (IgG4) expressing cell line.

Further, in the same manner, CHO cells were transformed with the light chain expression vector pE-hygr(LC3-2) and the heavy chain expression vector pE-neo(HC3-IgG4) both constructed in Example 11, and a high-expressing cell line for humanized anti-hTfR antibody No. 3-2 (IgG4) was obtained. This was designated antibody No. 3-2 (IgG4) expressing cell line.

### [Example 13] Purification of Humanized Anti-hTfR Antibodies

Antibody No. 1 expressing cell line, antibody No. 2 expressing cell line, antibody No. 3 expressing cell line, antibody No. 3-2 expressing cell line, antibody No. 3-3 expressing cell line and antibody No. 3-4 expressing cell line obtained in Example 12 were respectively diluted with CD OptiCHO™ medium to the density of approximately 2×10⁵ cells/mL. The cell suspensions, 200 mL, was added to a 1 L-conical flask, and cultured for 6 to 7 days in a humid environment at 37° C., 5% CO₂, 95% air, with stirring at a rate of about 70 rpm. Each culture supernatant was collected by centrifugation, and filtered through a 0.22 μm filter (Millipore Inc.) to prepare the culture supernatant. To each culture supernatant thus obtained was added five volumes of 20 mM Tris buffer (pH 8.0) containing 150 mM NaCl, and loaded onto a Protein A column (column volume: 1 mL, Bio-Rad Inc.) which had been equilibrated in advance with three column volumes of 20 mM Tris buffer (pH 8.0) containing 150 mM NaCl. Then, the column was washed with five column volumes of the same buffer, and the adsorbed humanized antibody was eluted with four column volumes of 50 mM glycine buffer (pH 2.8) containing 150 mM NaCl, and the eluted fraction was collected. The eluted fractions was added and neutralized with 1 M Tris buffer (pH 8.0) and used as the purified antibody preparation.

In the above, the antibody purified from the culture supernatant of antibody No. 1 expressing cell line was designated humanized anti-hTfR antibody No. 1. The antibody purified from the culture supernatant of antibody No. 2 expressing cell line was designated humanized anti-hTfR antibody No. 2. The antibody purified from the culture supernatant of antibody No. 3 expressing cell line was designated humanized anti-hTfR antibody No. 3. The antibody purified from the culture supernatant of antibody No. 3-2 expressing cell line was designated humanized anti-hTfR antibody No. 3-2. The antibody purified from the culture supernatant of antibody No. 3-3 expressing cell line was designated humanized anti-hTfR antibody No. 3-3. The antibody purified from the culture supernatant of antibody No. 3-4 expressing cell line was designated humanized anti-hTfR antibody No. 3-4.

Further, antibody No. 3 (IgG4) expressing cell line and antibody No. 3-2 (IgG4) expressing cell line obtained in Example 12 also were cultured in the same manner as above, and from their culture supernatants were obtained purified humanized anti-hTfR antibody No. 3 (IgG4) and humanized anti-hTfR antibody No. 3-2 (IgG4), respectively. These two antibodies were employed in the pharmacokinetic analysis using monkeys described in Example 15.

### [Example 14] Measurement of Affinity of Humanized Anti-hTfR Antibodies to Human TfR and Monkey TfR

The affinity of the humanized anti-hTfR antibodies obtained in Example 13 to human and monkey TfRs was measured by the method described in Example 7. Table 7 shows the result of the measurement of the association rate constant (kₒₙ), dissociation rate constant (k_{off}), and dissociation constant (k_{D}) of humanized anti-hTfR antibodies Nos. 1 to 3-4 (corresponding to Nos. 1 to 3-4, respectively, in the table) to human TfR.

TABLE 7
Affinity of humanized anti-hTfR antibodies to human TfR

| Antibody | kon | koff | K_{D} |
|---|---|---|---|
| No. | (M⁻¹s⁻¹) | (s⁻¹) | (M) |
| 1 | 3.93 × 10⁵ | <1.0 × 10⁻⁷ | <1.0 × 10⁻¹² |
| 2 | 1.97 × 10⁵ | <1.0 × 10⁻⁷ | <1.0 × 10⁻¹² |
| 3 | 1.19 × 10⁶ | <1.0 × 10⁻⁷ | <1.0 × 10⁻¹² |
| 3-2 | 6.06 × 10⁵ | 1.45 × 10⁻⁵ | 2.39 × 10⁻¹¹ |
| 3-3 | 6.00 × 10⁵ | 1.25 × 10⁻⁵ | 2.09 × 10⁻¹¹ |
| 3-4 | 1.01 × 10⁶ | <1.0 × 10⁻⁷ | <1.0 × 10⁻¹² |

Table 8 shows the result of the measurement of the association rate constant (kₒₙ), dissociation rate constant (k_{off}), and dissociation constant (k_{D}) of humanized anti-hTfR antibodies Nos. 1 to 3-4 (corresponding to Nos. 1 to 3-4, respectively, in the table) to monkey TfR.

TABLE 8
Affinity of humanized anti-hTfR antibodies to monkey TfR

| Antibody | kon | koff | K_{D} |
|---|---|---|---|
| No. | (M⁻¹s⁻¹) | (s⁻¹) | (M) |
| 1 | 2.53 × 10⁵ | <1.0 × 10⁻⁷ | <1.0 × 10⁻¹² |
| 2 | 4.87 × 10⁵ | 3.67 × 10⁻⁵ | 7.55 × 10⁻¹¹ |
| 3 | 6.03 × 10⁵ | 6.76 × 10⁻⁴ | 1.12 × 10⁻⁹ |
| 3-2 | 4.95 × 10⁵ | 8.76 × 10⁻⁴ | 1.77 × 10⁻⁹ |
| 3-3 | 4.88 × 10⁵ | 9.32 × 10⁻⁴ | 1.91 × 10⁻⁹ |
| 3-4 | 5.19 × 10⁵ | 1.35 × 10⁻⁴ | 2.60 × 10⁻¹⁰ |

The result of the measurement of the affinity of humanized anti-hTfR antibody Nos. 1 to 3-4 to human TfR showed that the dissociation constant between humanized anti-hTfR antibodies Nos. 1, 2, 3, and 3-4 and human TfR was less than 1×10⁻¹² M (Table 7). And the dissociation constant between humanized anti-hTfR antibodies Nos. 3-2 and 3-3 and human TfR was 2.39×10⁻¹¹ M and 2.09×10⁻¹¹ M, respectively. At the same time, the dissociation constant between the pre-humanized anti-hTfR antibodies corresponding to those antibodies and human TfR was: 5.09×10⁻¹² M for antibody No. 1, 1.12×10⁻¹¹ M for antibody No. 2, and less than 1×10⁻¹² M for antibody No. 3 (Table 4). These results demonstrate that the high affinity of those pre-humanized anti-hTfR antibodies to human TfR was maintained after humanization of the antibodies, and indicate that anti-hTfR antibodies Nos. 4 to 14 would also maintain their affinity to human TfR after their humanization.

Then, looking to the result of measurement of the affinity of humanized anti-hTfR antibodies to monkey TfR, it is seen that the dissociation constant of humanized anti-hTfR antibody No. 1 was less than 1×10⁻¹² M, indicating that the pre-humanized affinity was maintained after humanization, and also with regard to humanized anti-hTfR antibody No. 2, the dissociation constant was 4.18×10⁻¹¹ M before humanization and 7.55×10⁻¹¹ M after humanization, indicating the affinity was maintained (Table 5, Table 8). On the other hand, regarding to humanized anti-hTfR antibodies Nos. 3 to 3-4, while the dissociation constant of anti-hTfR antibody No. 3, the pre-humanized antibody corresponding to them, to monkey TfR was less than 1×10⁻¹² M, their dissociation constant after humanization was 2.60×10⁻¹⁰ M to 1.91×10⁻⁹M, showing a lowering of the affinity to monkey TfR. As to the humanized anti-hTfR antibody No. 3, although a lowering of affinity to monkey TfR was observed, the result indicates that the pre-humanized high affinity of anti-hTfR antibody to monkey TfR was not lost after its humanization but was maintained as a whole. It indicates that as to humanized anti-hTfR antibodies Nos. 4 to 14, too, the pre-humanized affinity to monkey TfR could be maintained after their humanization.

### [Example 15] Pharmacokinetic Analysis of Humanized Anti-hTfR Antibody in Monkey

Using monkeys, pharmacokinetic analysis was performed with four antibodies: humanized anti-hTfR antibody No. 3, humanized anti-hTfR antibody No. 3-2, humanized anti-hTfR antibody No. 3 (IgG4), and humanized anti-hTfR antibody No. 3-2 (IgG4). Besides, the heavy chain of humanized anti-hTfR antibody No. 3 was IgG1, while in humanized anti-hTfR antibody No. 3 (IgG4), the heavy chain of humanized anti-hTfR antibody No. 3 had been converted into IgG4, with its variable region kept intact. Further, the heavy chain of humanized anti-hTfR antibody No. 3-2 was IgG1, while in humanized anti-hTfR antibody No. 3-2 (IgG4), the heavy chain of humanized anti-hTfR antibody No. 3-2 had been converted into IgG4 with its variable region kept intact. These four antibodies were respectively intravenously administered once to male cynomolgus monkeys, at a dosage of 5.0 mg/kg, and their peripheral blood was sampled before the administration, 2 minutes, 30 minutes, 2 hours, 4 hours and 8 hours after the administration, and then they were subjected to whole body irrigation. As a negative control, trastuzumab (Herceptin™, Chugai Pharmaceutical Co., Ltd.), a humanized antibody to HER2 protein, was intravenously administered once to a single monkey in the same manner, and its peripheral blood was sampled before the administration, 2 minutes, 30 minutes, 2 hours, 4 hours and 8 hours after the administration, and then it was subjected to the whole body irrigation. After the irrigation, the brain and spine tissues including the medulla oblongata and other tissues (liver, heart, spleen and bone marrow) were excised. Using these brain and spinal tissues and other tissues, the concentration of the humanized anti-hTfR antibodies was measured and immunohistochemical staining was carried out.

Measurement of the concentration of humanized anti-hTfR antibodies in tissues and peripheral blood was carried out largely following the procedure described below. Besides, as to the brain, the collected tissues were separated into the cerebral cortex, the cerebellum, the hippocampus and the medulla oblongata, and then the concentration of the humanized anti-hTfR antibodies were measured. The respective tissues thus obtained were homogenized with RIPA Buffer (Wako Pure Chemical Industries Inc.) containing Protease Inhibitor Cocktail (Sigma-Aldrich Inc.), centrifuged, and the supernatant collected. From the above peripheral blood, serum was separated. To each well of High Bind Plate (Meso Scale Diagnostics) was added 10 μL of Affinipure Goat Anti mouse IgG Fcγ pAb (Jackson ImmunoResearch Inc.), and the plate was left to stand for one hour to provide a solid phase. Then, 150 μL of SuperBlock blocking buffer in PBS (Thermo Fisher Scientific Inc.) was added to each well, and the plate was blocked by one-hour shaking. Then, 25 μL of the supernatant of the homogenate or the serum was added to each well, and the wells were shaken for one hour. Then, 25 μL of Affinipure Goat Anti mouse IgG Fab-Biotin (Jackson ImmunoResearch Inc.) was added to each well, and shaking was continued for one hour. Then, 25 μL of SULFO-Tag-Streptavidin (Meso Scale Diagnostics Inc.) was added to each well, followed by shaking for half an hour. To each well was added 150 μL of Read buffer T (Meso Scale Diagnostics Inc.), and the amount of luminescence from each well was read on a Sector™ Imager 6000 reader. The amount of the antibody contained in each tissue and the peripheral blood was calculated by producing a standard curve based on measurements of standard samples containing known concentrations of the anti-hTfR antibody, and then interpolating the measurement of each of the samples with reference to the standard. Measurement of concentration was repeated three times for each sample.

The result of measurement of the concentration of humanized anti-hTfR antibodies in the brain and spinal tissues is shown in Table 9.

TABLE 9
Concentration of humanized anti-hTfR antibodies in brain tissues (μg/g wet weight)

| Antibody | Cerebral | | | Medulla | |
|---|---|---|---|---|---|
| No. | cortex | Cerebellum | Hippocampus | oblongata | Spinal cord |
| 3 | 0.67 ± 0.12 | 0.61 ± 0.02 | 0.49 ± 0.02 | 0.59 ± 0.10 | 0.46 ± 0.17 |
| 3-2 | 1.05 ± 0.07 | 0.72 ± 0.04 | 0.72 ± 0.07 | 0.69 ± 0.03 | 0.46 ± 0.02 |
| 3 (IgG4) | 0.65 ± 0.05 | 0.59 ± 0.03 | 0.56 ± 0.02 | 0.59 ± 0.02 | 0.46 ± 0.07 |
| 3-2 (IgG4) | 0.76 ± 0.02 | 0.57 ± 0.07 | 0.62 ± 0.05 | 0.73 ± 0.16 | 0.48 ± 0.03 |
| Negative | 0.0082 ± 0.0032 | 0.0090 ± 0.0067 | 0.0053 ± 0.0009 | 0.011 ± 0.003 | 0.15 ± 0.04 |
| control | | | | | |

All the antibodies, i.e., humanized anti-hTfR antibody No. 3, humanized anti-hTfR antibody No. 3-2, humanized anti-hTfR antibody No. 3 (IgG4) and humanized anti-hTfR antibody No. 3-2 (IgG4), were observed to accumulate in the cerebral cortex, cerebellum, hippocampus, medulla oblongata and spinal cord (Table 9). The respective amount accumulated was as follow:
with humanized anti-hTfR antibody No. 3, approximately 82 times in the cerebral cortex, approximately 68 times in the cerebellum, approximately 92 times in the hippocampus, approximately 54 times in the medulla oblongata, and approximately 3.1 times in the spinal cord, in comparison with the negative control, trastuzumab (Herceptin™),
with humanized anti-hTfR antibody No. 3-2, approximately 128 times in the cerebral cortex, approximately 80 times in the cerebellum, approximately 136 times in the hippocampus, approximately 63 times in the medulla oblongata, approximately 3.1 times in the spinal cord, in comparison with the negative control, trastuzumab,
with humanized anti-hTfR antibody No. 3 (IgG4), approximately 79 times in the cerebral cortex, approximately 66 times in the cerebellum, approximately 106 times in the hippocampus, approximately 54 times in the medulla oblongata, approximately 3.1 times in the spinal cord, in comparison with the negative control, trastuzumab, and
with humanized anti-hTfR antibody No. 3-2 (IgG4), approximately 93 times in the cerebral cortex, approximately 63 times in the cerebellum, approximately 117 times in the hippocampus, approximately 66 times in the medulla oblongata, approximately 3.2 times in the spinal cord, in comparison with the negative control, trastuzumab (Table 10).

These results indicate that these four humanized anti-hTfR antibodies have a property that allows them to pass through the blood-brain barrier and accumulate in the brain tissues, and that it is now possible to let pharmaceutical agents which need to be brought into function in the brain tissues efficiently accumulate there, by binding such pharmaceutical agents to one of these antibodies.

TABLE 10
Amount of humanized anti-hTfR antibodies
accumulated in brain tissues
(factors in comparison with negative control)

| Antibody | Cerebral | | Hip- | Medulla | Spinal |
|---|---|---|---|---|---|
| No. | cortex | Cerebellum | pocampus | oblongata | cord |
| 3 | 82 | 68 | 92 | 54 | 3.1 |
| 3-2 | 128 | 80 | 136 | 63 | 3.1 |
| 3 (IgG4) | 79 | 66 | 106 | 54 | 3.1 |
| 3-2 (IgG4) | 93 | 63 | 117 | 66 | 3.2 |
| Negative | 1 | 1 | 1 | 1 | 1 |
| control | | | | | |

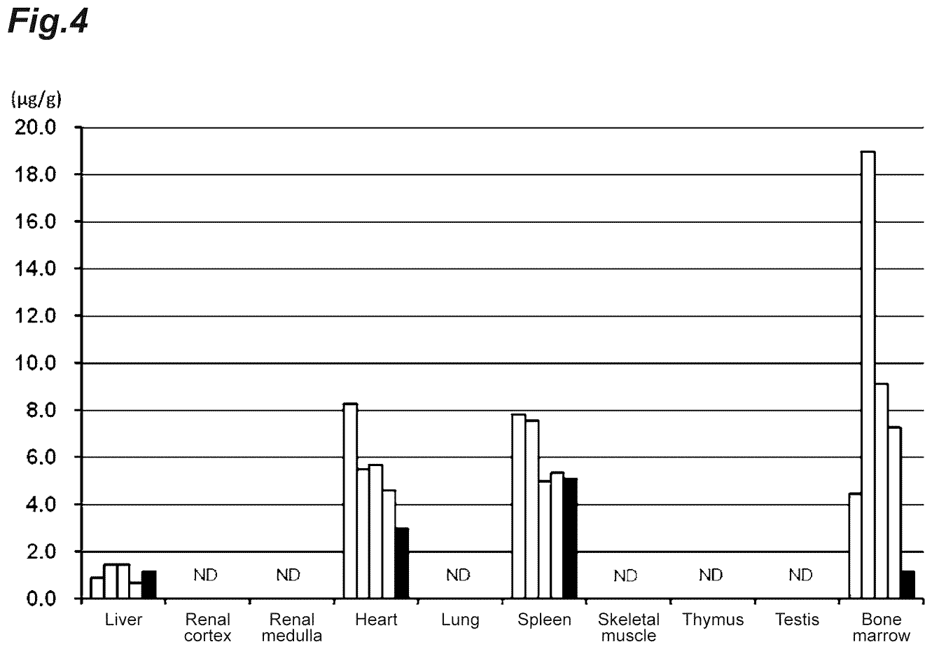

Then, FIG. 4 shows the result of measurement of the concentration of the humanized anti-hTfR antibodies in the tissues of the liver, heart, spleen and bone marrow. The four humanized anti-hTfR antibodies, as well as the negative control, trastuzumab, were observed to accumulate in the liver and spleen, and their amount accumulated was equal between the four humanized anti-hTfR antibodies and trastuzumab. In the heart, the humanized anti-hTfR antibodies tended to accumulate more than trastuzumab, the negative control, but the amount was only about 1.5 to 2.8 times that of the negative control. In bone marrow, the humanized anti-hTfR antibodies tended to accumulate markedly more than trastuzumab, the negative control, and the amount was 3.5 to 16 times that of the negative control. The cause of this accumulation of the humanized anti-hTfR antibodies in bone marrow is thought to be that TfR is expressed at high levels in bone marrow, hematopoietic organ, and more humanized anti-hTfR antibodies, therefore, accumulate through binding to TfR, than the negative control. These data indicate that the four humanized anti-hTfR antibodies has a property that allows them to specifically accumulate the cerebrum, cerebellum, hippocampus and medulla oblongata, which constitute the central nervous system, and that it is now possible to let pharmaceutical agents which need to be brought into function in the brain tissues efficiently accumulate there, by binding such pharmaceutical agents to one of these antibodies.

Then, Table 11 shows the result of pharmacokinetic measurement of the humanized anti-hTfR antibodies in the blood. As that of the negative control, trastuzumab, the blood concentration of the four humanized anti-hTfR antibodies was maintained at high levels, higher than 60 μg/mL, even eight hours after administration, indicating that they are stable in the blood.

TABLE 11
Pharmacokinetics of humanized anti-hTfR
antibodies in blood
(μg/mL blood( )

| Antibody | Time after administration | | | | |
|---|---|---|---|---|---|
| No. | 2 min | 30 min | 2 hr | 4 hr | 8 hr |
| 3 | 173 | 147 | 128 | 117 | 97.5 |
| 3-2 | 124 | 99.5 | 78.5 | 76.5 | 61 |
| 3 (IgG4) | 141 | 113 | 99 | 95 | 83 |
| 3-2 (IgG4) | 132 | 111 | 98.5 | 99 | 95.5 |
| Negative | 124 | 92.5 | 96 | 75.5 | 60.5 |
| control | | | | | |

Immunohistochemical staining of the humanized anti-hTfR antibodies in brain tissues was performed in the following manner. The collected tissues were rapidly frozen to −80° C. in a Tissue-Tek Cryo 3DM (Sakura Finetek Inc.) to prepare frozen blocks of tissues. The frozen blocks were sliced into 4 μm sections, which were affixed to MAS coated glass slides (Matsunami Glass Inc.). The tissue sections were reacted with 4% paraformaldehyde (Wako Pure Chemical Industries Inc.) for 5 minutes at 4° C. and fixed to glass slides. Then, the tissue sections were reacted with methanol solution containing 0.3% hydrogen peroxide (Wako Pure Chemical Industries Inc.) for 30 min to inactivate endogenous peroxidases. Then, the glass slides were blocked by reacting SuperBlock blocking buffer in PBS for 30 min at room temperature. Then, the tissue sections were reacted with Mouse IgG-heavy and light chain Antibody (Bethyl Laboratories) for one hour at room temperature. The tissue sections were allowed to visualize with DAB substrate (3,3′-diaminobenzidine, Vector Laboratories Inc.), counterstained with Mayer's hematoxylin solution (Merck Inc.), embedded after dehydration and cleaning, and observed under a optical microscope.

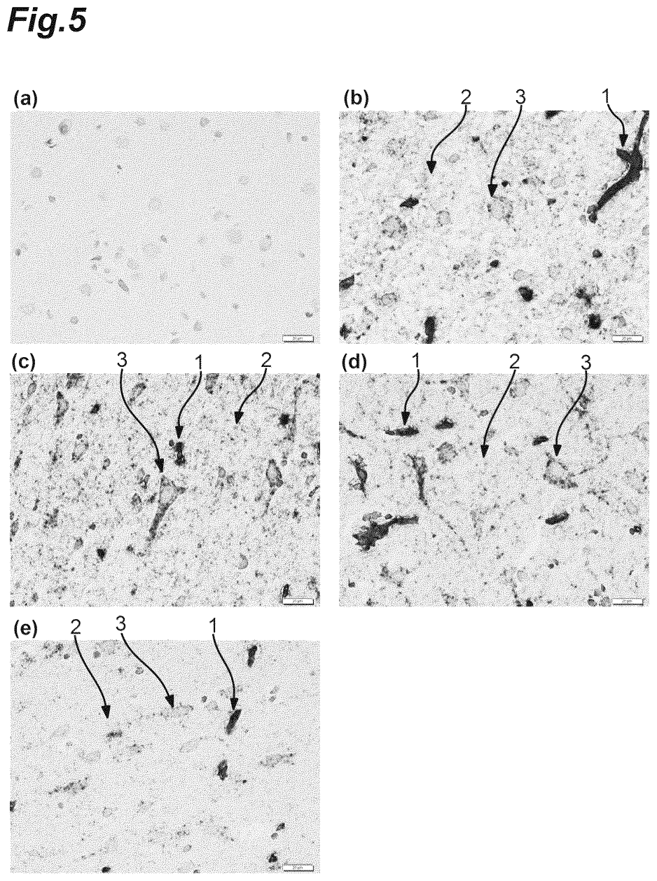

FIG. 5 shows the result of immunohistochemical staining of the humanized anti-hTfR antibodies in the cerebral cortex. Specific staining of blood vessels and neuron-like cells were observed in the cerebral cortex of the monkeys administered humanized anti-hTfR antibody No. 3, humanized anti-hTfR antibody No. 3-2, humanized anti-hTfR antibody No. 3 (IgG4), and humanized anti-hTfR antibody No. 3-2 (IgG4) (FIGS. 5b to 5e, respectively). In the cerebral cortex of the monkey administered humanized anti-hTfR antibody No. 3-2, in particular, (FIG. 5c), the brain parenchyma region, outside the blood vessels, was also observed specifically stained extensively. Besides, no staining was observed in the cerebral cortex of the monkey administered Herceptin as a control, indicating that the tissue staining observed in FIGS. 5b to 5e was specific for the humanized anti-hTfR antibodies (FIG. 5a).

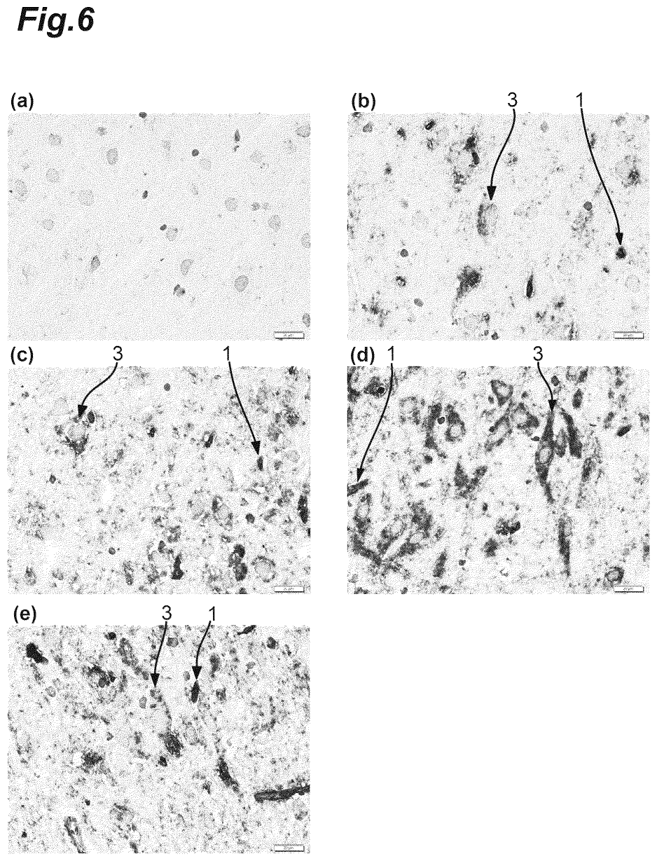

FIG. 6 shows the result of immunohistochemical staining of the humanized anti-hTfR antibodies in the hippocampus. Specific staining of blood vessels and neuron-like cells were observed in the hippocampus of the monkeys administered humanized anti-hTfR antibody No. 3, humanized anti-hTfR antibody No. 3-2, humanized anti-hTfR antibody No. 3 (IgG4), and humanized anti-hTfR antibody No. 3-2 (IgG4) (FIGS. 6b to 6e, respectively). Besides, no staining was observed in the hippocampus of the monkey administered Herceptin as a control, indicating that the tissue staining observed in FIGS. 6b to 6e was specific for the humanized anti-hTfR antibodies (FIG. 6a).

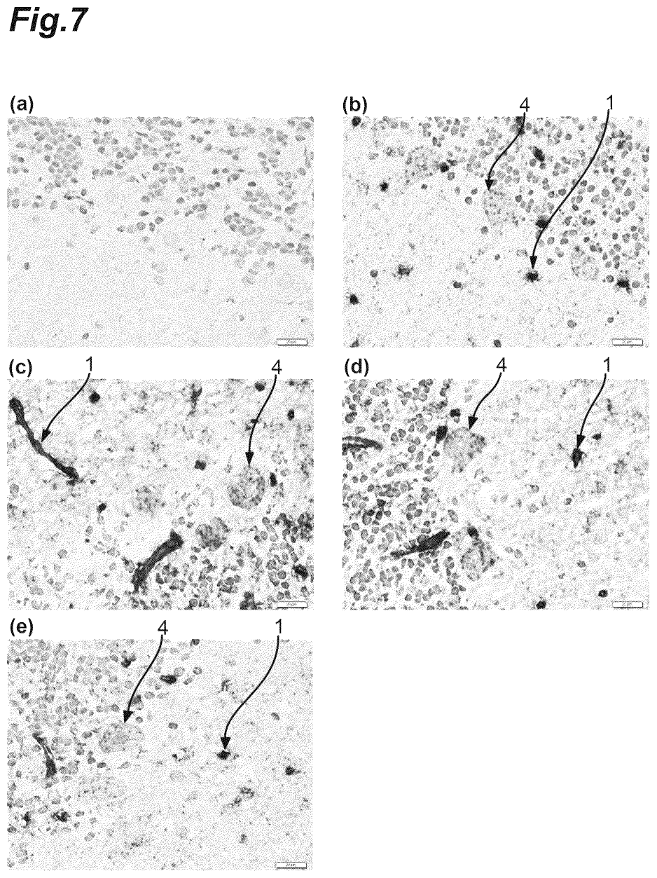

FIG. 7 shows the result of immunohistochemical staining of the humanized anti-hTfR antibodies in the cerebellum. Specific staining of blood vessels and Purkinje cells were observed in the cerebellum of the monkeys administered humanized anti-hTfR antibody No. 3, humanized anti-hTfR antibody No. 3-2, humanized anti-hTfR antibody No. 3 (IgG4), and humanized anti-hTfR antibody No. 3-2 (IgG4) (FIGS. 7b to 7e, respectively). Besides, no staining was observed in the cerebellum of the monkey administered Herceptin as a control, indicating that the tissue staining observed in FIGS. 7b to 7e was specific for the humanized anti-hTfR antibodies (FIG. 7a).

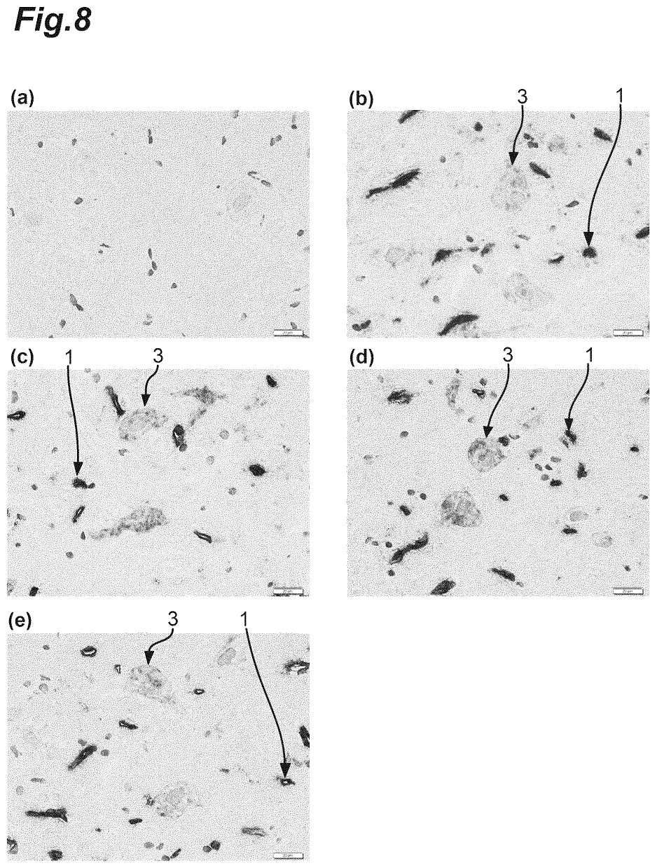

FIG. 8 shows the result of immunohistochemical staining of the humanized anti-hTfR antibodies in the medulla oblongata. Specific staining of blood vessels and neuron-like cells were observed in the medulla oblongata of the monkeys administered humanized anti-hTfR antibody No. 3, humanized anti-hTfR antibody No. 3-2, humanized anti-hTfR antibody No. 3 (IgG4), and humanized anti-hTfR antibody No. 3-2 (IgG4) (FIGS. 8b to 8e, respectively). Besides, no staining was observed in the medulla oblongata of the monkey administered Herceptin as a control, indicating that the tissue staining observed in FIGS. 8b to 8e was specific for the humanized anti-hTfR antibodies (FIG. 8a).

From the result of immunohistochemical staining of the cerebrum and cerebellum in Example 8, it had been anticipated that though the anti-hTfR antibody No. 1, a pre-humanized mouse antibody, can bind to hTfR existing on the endothelium of blood vessel in the brain, the amount transferring to the brain parenchyma would be small. On the other hand, it was shown that anti-hTfR antibodies Nos. 2 and 3, pre-humanized mouse antibodies, can bind to hTfR existing on the endothelium of blood vessel in the brain, and after binding to hTfR, pass through the blood-brain barrier into the brain parenchyma, and further be taken up into the brain parenchyma and neuron-like cells in the hippocampus, and into Purkinje cells in the cerebellum.

From the result of immunohistochemical staining in the cerebrum, hippocampus, cerebellum, and medulla oblongata in Example 15, it was revealed that the tested four humanized anti-hTfR antibodies obtained by humanizing anti-hTfR antibody No. 3 subjected to the experiment can bind to hTfR existing on the endothelium of blood vessels of the brain, and after binding to hTfR, pass through the blood-brain barrier and transfer into the brain parenchyma, and further, be taken up into neuron-like cells in the cerebral cortex; into the brain parenchyma and the neuron-like cells in the hippocampus; into Purkinje cells in the cerebellum; and into neuron-like cells in the medulla oblongata.

### [Example 16] Preparation of hI2S-Humanized Anti-hTfR Antibody Fusion Protein Expression Cells

By digesting pEF/myc/nucvector (Invitrogen Inc.) with KpnI and NcoI, a region including EF-1α promoter and its first intron was cut out, which then was blunt-ended with T4 DNA polymerase. After digesting pCI-neo (Invitrogen Inc.) with BgIII and EcoRI to remove a region including the enhancer/promoter and intron of CMV, the vector was blunt-ended with T4 DNA polymerase, and into which the above mentioned region including EF-1α promoter and its first intron was inserted to construct a vector pE-neo. The vector pE-neo was digested with SfiI and BstXI to cut out a region of approximately 1 kbp including a neomycin resistance gene. Employing pcDNA3.1/Hygro(+) (Invitrogen Inc.) as a template and using primer Hyg-Sfi5′ (SEQ ID NO:216) and primer Hyg-BstX3′ (SEQ ID NO:217), PCR was performed to amplify the hygromycin gene. The hygromycin gene thus amplified was digested with SfiI and BstXI and inserted into the above vector pE-neo, of which the neomycin resistance gene had been removed, to construct vector pE-hygr.

A DNA fragment was artificially synthesized having the nucleotide sequence set forth as SEQ ID NO:248, which included a gene encoding a protein in which the humanized anti-hTfR antibody heavy chain having the amino acid sequence set forth as SEQ ID NO:172 was linked, on the C-terminal side thereof and via a linker sequence (Gly Ser), to hI2S having the amino acid sequence set forth as SEQ ID NO:246. This DNA fragment encoded a protein in which humanized anti-hTfR antibody heavy chain having the amino acid sequence set forth as SEQ ID NO:247 was linked, via a linker sequence (Gly Ser), to hI2S. This DNA fragment had, on its 5′ side, a MluI sequence, and a sequence encoding a leader peptide acting as a secretion signal in this order from the 5′ end, and a NotI sequence on its 3′ side. The DNA fragment was digested with MluI and NotI, and inserted into the vector pE-neo, between the MluI and NotI thereof, to construct pE-neo(HC-I2S-1).

A DNA fragment was artificially synthesized having the nucleotide sequence set forth as SEQ ID NO:250, which included a gene encoding a protein in which the humanized anti-hTfR antibody heavy chain having the amino acid sequence set forth as SEQ ID NO:188 was linked, on the C-terminal side thereof and via a linker sequence (Gly Ser), to hI2S having the amino acid sequence set forth as SEQ ID NO:246. This DNA fragment encoded a protein in which humanized anti-hTfR antibody heavy chain having the amino acid sequence set forth as SEQ ID NO:249 was linked, via a linker sequence (Gly Ser), to hI2S. This DNA fragment had, on its 5′ side, a MluI sequence and a sequence encoding a leader peptide acting as a secretion signal in this order from the 5′ end, and a NotI sequence on its 3′ side. The DNA fragment was digested with MluI and NotI, and inserted into the vector pE-neo, between the MluI and NotI thereof, to construct pE-neo(HC-12S-2).

A DNA fragment was artificially synthesized having the nucleotide sequence set forth as SEQ ID NO:252, which included a gene encoding a protein in which the humanized anti-hTfR antibody heavy chain having the amino acid sequence set forth as SEQ ID NO:210 was linked, on the C-terminal side thereof and via a linker sequence (Gly Ser), to hI2S having the amino acid sequence set forth as SEQ ID NO:246. This DNA fragment encoded a protein having the amino acid sequence set forth as SEQ ID NO:251 in which humanized anti-hTfR antibody heavy chain was linked, via a linker sequence (Gly Ser), to hI2S. This DNA fragment had, on its 5′ side, a MluI sequence and a sequence encoding a leader peptide acting as a secretion signal in this order from the 5′ end, and a NotI sequence on its 3′ side. The DNA fragment was digested with MluI and NotI, and inserted into the vector pE-neo, between the MluI and NotI thereof, to construct pE-neo(HC-I2S-3).

CHO cells (CHO-K1: obtained from American Type Culture Collection) were transformed according to the method described in Example 12 with pE-neo(HC-I2S-1) and pE-hygr(LC1) which had been prepared in Example 11, to obtain a cell line expressing a fusion protein between hI2S and a humanized anti-hTfR antibody. This cell line was designated hI2S-anti-hTfR antibody expressing cell line 1. The fusion protein between hI2S and a humanized anti-hTfR antibody expressed by the cell lines was designated I2S-anti-hTfR antibody 1.

In the same manner, CHO cells were transformed with pE-neo(HC-12S-2) and pE-hygr(LC2) which had been prepared in Example 11, to obtain a cell line expressing a fusion protein between hI2S and a humanized anti-hTfR antibody. This cell line was designated hI2S-anti-hTfR antibody expressing cell line 2. The fusion protein between hI2S and a humanized anti-hTfR antibody expressed by the cell lines was designated I2S-anti-hTfR antibody 2.

Further, in the same manner, CHO cells were transformed with pE-neo(HC-12S-3) and pE-hygr(LC3) which had been prepared in Example 11, to obtain a cell line expressing a fusion protein between hI2S and a humanized anti-hTfR antibody. This cell line was designated hI2S-anti-hTfR antibody expressing cell line 3. The fusion protein between hI2S and a humanized anti-hTfR antibody expressed by the cell lines was designated I2S-anti-hTfR antibody 3.

### [Example 17] Production of I2S-Anti-hTfR Antibodies

I2S-anti-hTfR antibodies were produced by the following method. With CD OptiCHO™ medium, hI2S-anti-hTfR antibody expressing cell lines 1, 2 and 3 obtained in Example 16 were diluted to the density of approximately 2×10⁵ cells/mL, respectively. The cell suspensions, 200 mL, were added to corresponding 1 L-conical flasks, and cultured for 6 to 7 days in a humid environment at 37° C., 5% CO₂, 95% air, with stirring at a rate of about 70 rpm. Each culture supernatant was collected by centrifugation, and filtered through a 0.22 μm filter (Millipore Inc.) to prepare the culture supernatant. To each culture supernatant thus obtained was added five column volumes of 20 mM Tris buffer (pH 8.0) containing 150 mM NaCl, and loaded onto a Protein A column (column volume: 1 mL, Bio-Rad Inc.) which had been equilibrated in advance with three column volumes of 20 mM Tris buffer (pH 8.0) containing 150 mM NaCl. Then, the column was washed with five column volumes of the same buffer, and the adsorbed I2S-anti-hTfR antibody was eluted with four column volumes of 50 mM glycine buffer (pH 2.8) containing 150 mM NaCl. The pH of the eluate containing I2S-anti-hTfR antibody was adjusted to pH 7.0 with 1 M Tris buffer (pH 8.0), and then the buffer was replaced with PBS buffer using Amicon Ultra 30 kDa membrane (Millipore Inc.) to obtain a I2S-anti-hTfR antibody purified product.

The affinity of the I2S-anti-hTfR antibodies thus produced to human and monkey TfRs can be measured by, e.g., the method described in Example 7. Pharmacokinetic analysis of the I2S-anti-hTfR antibodies after their intravenous administration can be conducted by, e.g., the method described in Example 8. The pharmacological effects of the I2S-anti-hTfR antibodies can be evaluated by, e.g., intravenously injecting the I2S-anti-hTfR antibodies to I2S-KO/hTfR KI mice, which can be obtained through mating of iduronate 2-sulfatasegene knockout mice (12S-KO mice), model mice for Hunter syndrome, with hTfR-KI mice described in Example 7-2, and measuring the reduction of glycosaminoglycans accumulated in the brain.

### [Example 18] Evaluation of Transfer of I2S-Anti-hTfR Antibodies into the Brain-1

Each of the purification products of the I2S-anti-hTfR antibody 3 prepared in Example 17 was intravenously injected, at the dose of 1 mg/kg, to hTfR-KI mice generated by the method described in Example 7-2 (I2S-anti-hTfR antibody administered group). As a control, recombinant hI2S (rhI2S) was intravenously injected, at the dose of 1 mg/kg, to hTfR-KI mice (control group). Fifteen hTfR-KI mice were administered in I2S-anti-hTfR antibody administered group, and three in the control group (male, 15 to 18-week old). Besides, the rhI2S used in the above had been prepared in accordance with a conventional method, which was described in an international patent publication (WO 2012/102998). It is also possible to use Elaprase®, a medicinal product available on the market as the rhI2S.

In an I2S-anti-hTfR antibody administered group, three mice were subjected whole body irrigation with physiological saline after 15 min, 1 hr, 4 hr, 8 hr, and 24 hr, respectively, of the administration of I2S-anti-hTfR antibody 3, and the brains (cerebrum and cerebellum) were taken. As for the control group, whole body irrigation with physiological saline was carried out one hour after the rhI2S administration, and the brains (cerebrum and cerebellum) were taken. The weight (wet weight) of the cerebrum and cerebellum excised was weighed, and then the cerebrum and cerebellum were homogenized with T-PER (Thermo Fisher Scientific Inc.) containing Protease Inhibitor Cocktail (Sigma Inc.), and their supernatants were collected after centrifugation. The amount of I2S-anti-hTfR antibody contained in the supernatant of homogenate was measured for I2S-anti-hTfR antibody administered group, and the amount of rhI2S contained in the supernatant of homogenate for the control group by ECL method described in Examples 20 and 21, respectively, and the amount of I2S-anti-hTfR antibody (concentration of I2S-anti-hTfR antibody in brain tissues) as well as the amount of rhI2S (concentration of rhI2S in brain tissues) contained in 1 g of the brain (wet weight) were calculated. The result is shown in Table 12.

The concentration of I2S-anti-hTfR antibody and rhI2S in the brain tissues one hour after administration was 0.368±0.019 μg/g wet weight and 0.00134±0.00232 μg/g wet weight, respectively, showing that the concentration of I2S-anti-hTfR antibody reached approximately 270 times that of rhI2S. The result indicates that rhI2S, which scarcely passes through the blood-brain barrier, in general, and does not transfer to the brain, can be made to pass through the blood-brain barrier and transfer into the brain tissues, by combining it with the anti-hTfR antibody. Further, the concentration of the I2S-anti-hTfR antibody in the brain tissues reached 0.263±0.038 μg/g wet weight in only 15 min after administration, which was approximately 200 times that of rhI2S in the brain tissues one hour after administration. The result also indicates that by combining with the anti-hTfR antibody, it becomes possible to let hI2S rapidly transfer into the brain tissues.

TABLE 12
Concentration of I2S-anti-hTfR
antibody and rhI2S in brain tissues
(μg/g wet weight)

| Time after | | |
|---|---|---|
| administration | I2S-anti-hTfR | |
| (hr) | antibody | rhI2S |
| 0.25 | 0.263 ± 0.038 | — |
| 1 | 0.368 ± 0.019 | 0.00134 ± 0.00232 |
| 4 | 0.440 ± 0.033 | — |
| 8 | 0.382 ± 0.011 | — |
| 24 | 0.245 ± 0.012 | — |

### [Example 19] Evaluation of Transfer of 12S-Anti-hTfR Antibodies into the Brain-2

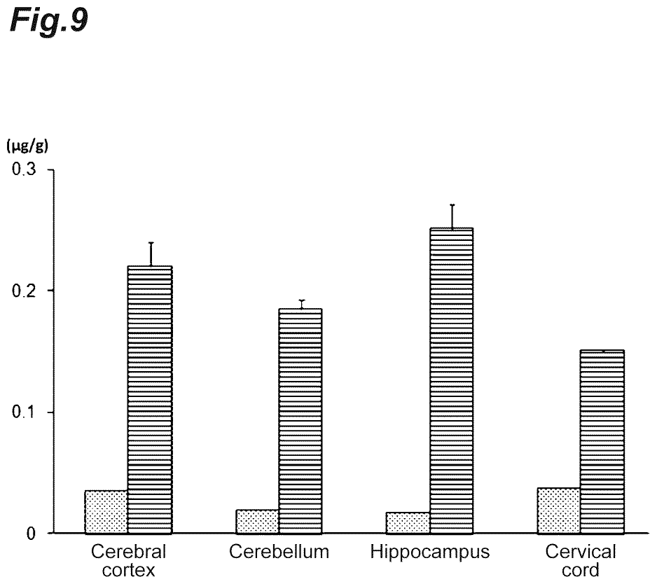

The purification product of I2S-anti-hTfR antibody 3 prepared in Example 17 was intravenously administered once to male cynomolgus monkeys, at a dosage of 5 mg/kg (I2S-anti-hTfR antibody administered group). In the same manner, rhI2S was administered once to male cynomolgus monkeys, at a dosage of 5 mg/kg (rhI2S group). The rhI2S employed here had been prepared in accordance with a conventional method, which was described in an international patent publication (WO 2012/102998). It is also possible to use Elaprase®, a medicinal product available on the market as the rhI2S. Two monkeys were administered in each group. Eight hours after administration, whole body irrigation was carried out. After the irrigation, the brain tissues including the cervical cord were excised. The brain tissues thus excised were separated into the cerebral cortex, the cerebellum, the hippocampus and the cervical cord, and each of them was homogenized with T-PER (Thermo Fisher Scientific Inc.) containing Protease Inhibitor Cocktail (Sigma Inc.), centrifuged, and the supernatant was collected. The amount of the I2S-anti-hTfR antibody contained in the supernatant of homogenate was measured in I2S-anti-hTfR antibody administered group, and the amount of rhI2S contained in the supernatant of homogenate in the control group by ECL method described in Examples 20 and 21, respectively. From the measurement thus obtained, the amount of I2S-anti-hTfR antibody contained in 1 g (g wet weight) of the cerebral cortex, cerebellum, hippocampus, and cervical cord (concentration of I2S-anti-hTfR antibody in those brain tissues), as well as the amount of rhI2S (concentration of rhI2S in those brain tissues), were calculated. The result is shown in FIG. 9.

The concentration of I2S-anti-hTfR antibody in the cerebral cortex was approximately 0.22 μg/g, whereas that of rhI2S was 0.035 μg/g. The concentration of I2S-anti-hTfR antibody in the cerebellum was approximately 0.18 μg/g, whereas that of rhI2S was 0.02 μg/g. The concentration of I2S-anti-hTfR antibody in the hippocampus was approximately 0.25 μg/g, whereas that of rhI2S was 0.017 μg/g. And the concentration of I2S-anti-hTfR antibody in the cervical cord was approximately 0.15 μg/g, whereas that of rhI2S was 0.039 μg/g. Thus, the concentration of I2S-anti-hTfR antibody in the cerebral cortex, cerebellum, hippocampus, and cervical cord was shown to be approximately 6.3 times, approximately 9.0 times, approximately 14.7 times, and approximately 3.8 times that of rhI2S, respectively. The result indicates that by binding hI2S to the anti-hTfR antibody, it becomes possible to let hI2S actively pass through the blood-brain barrier and get into the brain efficiently. In particular, that the concentration of the I2S-anti-hTfR antibody in the hippocampus was approximately as high as approximately 12.5 times that of rhI2S indicates that administration of I2S-anti-hTfR antibody could let I2S exhibit its activity in the hippocampus among others. Encephalopathy in Hunter syndrome patients cannot be ameliorated by enzyme replacement therapy with conventional rhI2S because little rhI2S can pass through the blood-brain barrier. In contrast, as I2S-anti-hTfR antibody can pass through the blood-brain barrier, administration of I2S-anti-hTfR antibody could supplement the activity of I2S in the brain tissues such as the cerebral cortex, hippocampus, and cerebellum. Therefore, the I2S-anti-hTfR antibody (especially I2S-anti-hTfR antibody 3) can be used as therapeutic agents to supplement the I2S activity in the brain of Hunter syndrome patients. Thus, by administering I2S-anti-hTfR antibody (especially I2S-anti-hTfR antibody 3), prophylaxis and treatment of encephalopathy in Hunter syndrome patients are possible, which is difficult by enzyme replacement therapy with conventional rhI2S. It is especially promising as a therapeutic agent for patients with Hunter syndrome accompanied disorders of the hippocampus.

### [Example 20] Quantitative Determination of I2S-Anti-hTfR Antibody by ECL Method

To each well of a 96-well Streptavidin Gold Plate (Meso Scale Diagnostics Inc.), a streptavidin-coated plate, was added 150 μL of SuperBlock blocking buffer in PBS (Thermo Fisher Scientific Inc.), and the plate was left to stand for one hour for blocking. Anti-Human Kappa Light chain Goat IgG Biotin (Monkey Absorbed) (IBL Inc.), a biotin-labeled antibody, was diluted with SuperBlock blocking buffer in PBS to 0.5 μg/mL. SULFO-labeled anti-human I2S antibody was diluted with SuperBlock blocking buffer in PBS to 1.0 μg/mL. The diluted solutions of the biotin-labeled antibody and the SULFO-labeled antibody, 25 μL each, were mixed with 25 μL of each sample, incubated for one hour to prepare an antibody reaction sample.

Each well of the plate after blocking was washed with 200 μL of PBS-T (Sigma Inc.), and 25 μL of the antibody reaction sample was added, and incubated for one hour. After incubation, each well of the plate was washed with 200 μL of PBS-T, Read buffer T (Meso scale Diagnostics Inc.) was added to each well, and the amount of luminescence from each well was measured on Sector™ Imager 6000 (Meso scale Diagnostics Inc.). A standard curve was produced on measurements of standard samples containing known concentrations of I2S-anti-hTfR antibody, and the amount of I2S-anti-hTfR antibody was determined by interpolating the measurement of each of the samples with reference to the standard.

Besides, the anti-human I2S antibody used in the above was a monoclonal antibody obtained by immunizing mice with rhI2S, as antigen, which had been prepared accordance with a conventional method described in an international patent publication (WO 2012/102998). Multiple monoclonal antibodies had been obtained. As SULFO-labeled anti-human 12S antibody, an anti-human I2S antibody was labelled with SULFO using MSD SULFO-TAG NHS-Ester (Meso scale Diagnostics Inc.) in accordance with the attached manual.

### [Example 21] Quantitative Determination of hI2S by ECL Method

To each well of a 96-well Streptavidin Gold Plate (Meso Scale Diagnostics Inc.), a streptavidin-coated plate, was added 150 μL of SuperBlock blocking buffer in PBS (Thermo Fisher Scientific Inc.), and the plate was left to stand for one hour for blocking. Biotin-labeled anti-human I2S antibody was diluted with SuperBlock blocking buffer in PBS to 0.5 μg/mL. SULFO-labeled anti-human I2S antibody was diluted with SuperBlock blocking buffer in PBS to 1.0 μg/mL. The diluted solutions of the biotin-labeled antibody and the SULFO-labeled antibody, 25 μL each, were mixed with 25 μL of each sample, incubated for one hour to prepare an antibody reaction sample.

Each well of the plate after blocking was washed with 200 μL of PBS-T (Sigma Inc.), and 25 μL of the antibody reaction sample was added, and incubated for one hour. After incubation, each well of the plate was washed with 200 μL of PBS-T, Read buffer T (Meso scale Diagnostics Inc.) was added to each well, and the amount of luminescence from each well was measured on Sector™ Imager 6000 (Meso scale Diagnostics Inc.). A standard curve was produced on measurements of standard samples containing known concentrations of hI2S, and the amount of rhI2S was determined by interpolating the measurement of each of the samples with reference to the standard.

Besides, the anti-human I2S antibody used in the above was a monoclonal antibody obtained by immunizing mice with rhI2S, as antigen, which had been prepared accordance with a conventional method described in an international patent publication (WO 2012/102998). As SULFO-labeled anti-human I2S antibody, an anti-human I2S antibody was labelled with SULFO using MSD SULFO-TAG NHS-Ester (Meso scale Diagnostics Inc.) in accordance with the attached manual. Further, as biotin-labeled anti-human I2S antibody, another anti-human I2S antibody, except the one that was employed for labeling with SULFO, was labelled with biotin using Biotin Labelling Kit-NH₂ (Dojindo Laboratories Inc.) in accordance with the attached manual.

### [Example 22] Assessment of Pharmacological Effect of I2S-Anti-hTfR Antibody

The pharmacological effect of I2S-anti-hTfR antibody was evaluated by measuring the concentration of the glycosaminoglycans (GAG) that was known to accumulate in the organs of Hunter syndrome patients, who genetically lacked hI2S activity. The purification product of I2S-anti-hTfR antibody 3 prepared in Example 17 was intravenously injected to 12S-KO/hTfR-KI mice at a dosage of 0.5 mg/kg, 1.0 mg/kg, and 2.0 mg/kg (0.5 mg/kg-administered group, 1.0 mg/kg-administered group, and 2.0 mg/kg-administered group). Administration was made at a frequency of once a week for four weeks, the mice were euthanized by exsanguination under anesthesia four weeks after the first administration, and the brain, liver, lung, and heart were excised. The excised organs then were lyophilized in a lyophilizer (EYELA Inc.), pulverized, and their dry weight was measured. To 100 mg (dry weight) of each of the dried organ preparations was added 1 mL of 0.5 M Tris buffer (pH 7.5), and heated for 10 min at about 100° C. Then, 50 mg/mL Actinase E solution (Kaken Pharmaceutical Inc.) was added to the dry preparation so that 1 mg of actinase E was added to 50 mg (dry weight) the mixture was incubated for 16 hr at about 60° C. to decompose the protein, and heating was continued for 10 min at about 100° C. After centrifugation for 10 min at 15,000 rpm, the supernatant was collected. The amount of GAG contained in the supernatant was measured using Wieslab™ sGAG quantitative kit (Euro-Diagnostica Inc.), and the amount of GAG contained in 1 g (g dry weight) of each organ was calculated. Besides, as the control group, 12S-KO/hTfR-KI mice non-administered I2S-anti-hTfR antibody was employed. At the same time, the amount of GAG in the organs of wild-type mice was measured. This experiment was conducted with three I2S-KO/hTfR-KI mice (male and female, 19 to 25-week old) in each measurement group. Besides, three wild-type mice, male and female, 19 to 25-week old, were employed.

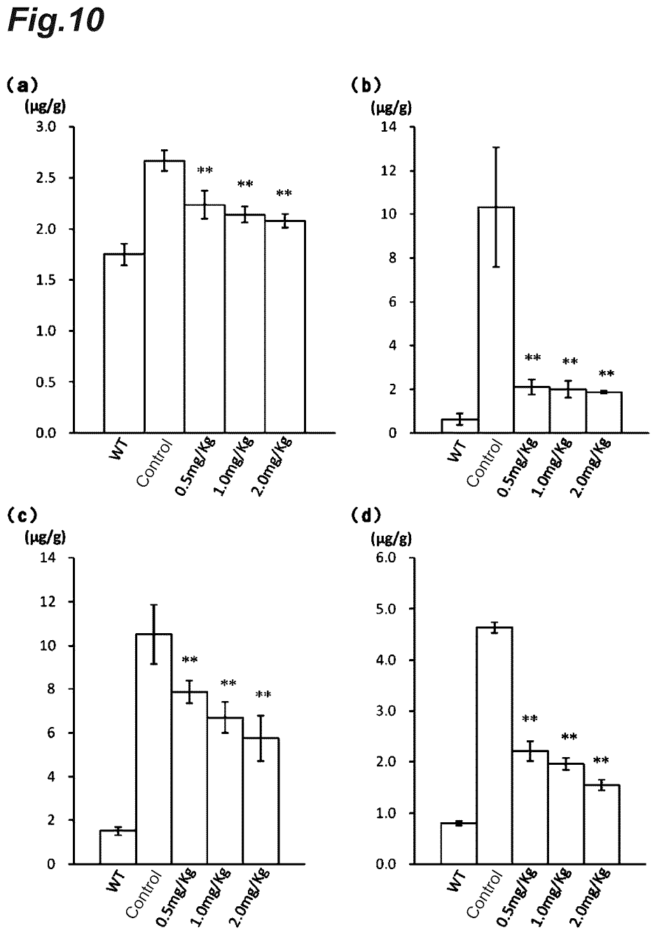

The result is shown in FIG. 10. It was observed that in all the brain, liver, lung and heart, the concentration of GAG significantly decreased dose dependently by I2S-anti-hTfR antibody administration (FIGS. 10a to 10d).

In the brain, the concentration of GAG was approximately 2.66 μg/g in the brain tissues of the control group, whereas the concentration of GAG in the brain tissues of 0.5 mg/kg, 1.0 mg/kg, and 2.0 mg/kg administered groups was approximately 2.23 μg/g, approximately 2.15 μg/g, approximately 2.10 μg/g, respectively, showing a dose dependent decrease (FIG. 10a). Abnormal amount of GAG in the brain tissues of I2S-KO/hTfR-KI mice can be determined as being approximately 0.90 μg/g, i.e., the amount that remains after the concentration of GAG in the brain tissues of the wile-type mice (approximately 1.76 μg/g) is subtracted from the concentration of GAG in the brain tissues of the control group (approximately 2.66 μg/g). Thus, it can be concluded that approximately 48%, approximately 57%, and approximately 62% of GAG abnormally accumulated in the brain tissues of I2S-KO/hTfR-KI mice were decomposed by the I2S-anti-hTfR antibody administered at the doses of 0.5 mg/kg, 1.0 mg/kg, and 2.0 mg/kg, respectively. The result indicates that it is possible to decompose and remove GAG abnormally accumulated in the brain tissues of a Hunter syndrome patient by administering the I2S-anti-hTfR antibody to the patient, and that the I2S-anti-hTfR antibody (I2S-anti-hTfR antibody 3, in particular) could prevent and treat brain lesions caused by accumulation and the like of GAG or its fragments observed in Hunter syndrome patients.

In the liver, the concentration of GAG was approximately 10.3 μg/g in the liver tissue of the control group, whereas the concentration of GAG in the liver tissue of 0.5 mg/kg, 1.0 mg/kg, and 2.0 mg/kg administered groups was approximately 2.2 μg/g, approximately 2.0 μg/g, approximately 1.9 μg/g, respectively, showing a dose dependent decrease (FIG. 10b). Abnormal portion of the amount of GAG in the liver tissue of I2S-KO/hTfR-KI mice can be determined as being approximately 10 μg/g, i.e., the amount that remains after the concentration of GAG in the liver tissue of the wild-type mice (approximately 0.3 μg/g) is subtracted from the concentration of GAG in the liver tissue of the control group (approximately 10.3 μg/g). Thus, it can be concluded that not less than 80% of GAG abnormally accumulated in the liver tissue of I2S-KO/hTfR-KI mice was decomposed by the I2S-anti-hTfR antibody administered at the doses of 0.5 to 2.0 mg/kg.

In the lung, the concentration of GAG was approximately 10.5 μg/g in the lung tissue of the control group, whereas the concentration of GAG in the lung tissue of 0.5 mg/kg, 1.0 mg/kg, and 2.0 mg/kg administered groups was approximately 7.8 μg/g, approximately 6.7 μg/g, approximately 5.7 μg/g, respectively, showing a dose dependent decrease (FIG. 10c). Abnormal portion of the amount of GAG in the lung tissue of I2S-KO/hTfR-KI mice can be determined as being approximately 9.0 μg/g, i.e., the amount that remains after the concentration of GAG in the lung tissue of the wild-type mice (approximately 1.5 μg/g) is subtracted from the concentration of GAG in the lung tissue of the control group (approximately 10.5 μg/g). Thus, it can be concluded that approximately 30%, approximately 42%, and approximately 53% of GAG abnormally accumulated in the lung tissue of I2S-KO/hTfR-KI mice was decomposed by the I2S-anti-hTfR antibody administered at the doses of 0.5 mg/kg, 1.0 mg/kg, and 2.0 mg/kg, respectively.

In the heart, the concentration of GAG was approximately 4.6 μg/g in the heart tissue of the control group, whereas the concentration of GAG in the heart tissue of 0.5 mg/kg, 1.0 mg/kg, and 2.0 mg/kg administered group was approximately 2.2 μg/g, approximately 2.0 μg/g, and approximately 1.5 μg/g, respectively, showing a dose dependent decrease (FIG. 10d). Abnormal portion of the amount of GAG in the heat tissue of 12S-KO/hTfR-KI mice can be determined as being approximately 3.8 μg/g, i.e., the amount that remains after the concentration of GAG in the heart tissue of the wild-type mice (approximately 0.8 μg/g) is subtracted from the concentration of GAG in the heart tissue of the control group (approximately 4.6 μg/g). Thus, it can be concluded that approximately 63%, approximately 70%, and approximately 81% of GAG abnormally accumulated in the heart tissue of I2S-KO/hTfR-KI mice was decomposed by the I2S-anti-hTfR antibody administered at the doses of 0.5 mg/kg, 1.0 mg/kg, and 2.0 mg/kg, respectively.

The above results in the liver, lung and heart demonstrate that the I2S-anti-hTfR antibody decomposes accumulated GAG not only in the brain but also in other organs. This indicates that the I2S-anti-hTfR antibody (especially I2S-anti-hTfR antibody 3), when administered to patients with Hunter syndrome as a pharmaceutical agent in enzyme replacement therapy, could supplement all the patients' organs including the brain with the enzyme. Also indicated is that the I2S-anti-hTfR antibody can supplement the all the patients' organs including the brain with the enzyme through its intravenous administration.

### [Example 23] Method for Measurement of GAG

Measurement of GAG was conducted as follows in general, using Wieslab™ sGAG quantitative kit (EURO-DIAGNOSTICA Inc.) in accordance with the attached operating manual. A sample or a standard solution (blank or water), 50 μL each, was added to a 1.5-mL tube. To each tube was added 50 μL of a GuHCl solution, and a reaction was allowed to take place for 15 min at room temperature. A SAT solution, 50 μL, was added to each tube, and a reaction was allowed to proceed for 15 min at room temperature. A mixture solution of water/SAT solution/Alcian Blue stock solution (9/5/1) was prepared and added to the tubes, 750 μL each, and a reaction was allowed to proceed for 15 min at room temperature. The reaction mixture liquid thus obtained was centrifuged at 12500×g for 15 min to remove the supernatant. To each tube was added 500 μL of DMSO, and following 15-min shaking for mixing at room temperature, the mixture was centrifuged to remove the supernatant. Gu-Prop solution was added to the tubes, 500 μL each, and mixed by shaking for 15 min at room temperature. The mixture solution thus prepared was dispensed, 200 μL each, to the wells of a 96-well plate, and the absorbance of each well was measured at 600 nm using a plate reader. A standard curve was produced on measurements of solutions containing known concentrations of GAG, and the GAG concentration was determined by interpolating the measurement of each sample with reference to the standard.

### [Example 24] Preparation of Fusion Proteins Between Humanized Anti-hTfR Antibody and Various Physiologically Active Peptides

The experiments carried out in Examples 1 to 22 above have shown that human I2S linked to the humanized anti-hTfR antibody passes through the blood-brain barrier, gets into the brain tissues, and exhibits I2S activity in the brain. Then, fusion proteins were prepared between the humanized anti-hTfR antibody and various physiologically active peptides to examine whether those fusion proteins pass through the BBB and get into the brain tissues. Here, human erythropoietin, human arylsulfatase A, human PPT-1, human TPP-1, human α-L-iduronidase, human TNFα receptor, and human N-sulphoglucosamine sulphohydrolase (heparan N-sulfatase) were selected as physiologically active peptides to be fused with the humanized anti-hTfR antibody. The expression vectors used to express fusion proteins between the humanized anti-hTfR antibody and physiologically active peptides, as well as the resulting fusion proteins, were prepared according to Examples 25 to 31 below.

### [Example 25] Method for Preparation of Fusion Protein Between Humanized Anti-hTfR Antibody and Human Erythropoietin

A DNA fragment was artificially synthesized comprising the nucleotide sequence set forth as SEQ ID NO:258, which included the cDNA encoding a protein consisting of the humanized anti-hTfR antibody having the amino acid sequence set forth as SEQ ID NO:210 and human erythropoietin (hEPO) having the amino acid sequence set forth as SEQ ID NO:256 and linked, via a linker sequence Gly-Ser, on the C-terminal side of the antibody's heavy chain. This DNA fragment encodes a protein having the amino acid sequence set forth as SEQ ID NO:257, which consists of the humanized anti-hTfR antibody and hEPO, which is linked, via a linker sequence Gly-Ser, to the antibody's heavy chain on the C-terminal side thereof. The DNA fragment has in the 5′ side a MluI sequence and a sequence encoding a leader peptide acting as a secretion signal, in this order from the 5′ end, and a NotI sequence in the 3′ side. The DNA fragment was digested with MluI and NotI, and inserted into the vector pE-neo between MluI and NotI sites to construct pE-neo(HC-hEPO).

By the method described in Example 12, CHO cells were transformed with pE-neo(HC-hEPO) and pE-hygr(LC3), which was constructed in Example 11, a cell line was obtained which expresses a fusion protein between hEPO and the humanized anti-hTfR antibody. The cell line was designated hEPO-anti-hTfR antibody expressing cell line. The fusion protein between hEPO and the humanized anti-hTfR antibody, expressed by this cell line, were designated EPO-anti-hTfR antibody.

Cells of hEPO-anti-hTfR antibody expressing cell line were diluted to the density of approximately 2×10⁵ cells/mL with CD OptiCHO™ medium, and 200 mL of the cell suspension was added to a 1-L conical flask and cultured for 6 to 7 days in a humid environment at 37° C., 5% CO₂, 95% air, with stirring at a rate of about 70 rpm. The culture medium was collected by centrifugation and filtered through a 0.22 μm filter (Millipore Inc.) to prepare a culture supernatant. To the culture supernatant thus collected was added five column volumes of 20 mM Tris buffer (pH 8.0) containing 150 mM NaCl, and loaded onto a Protein A column (column volume 1: mL, Bio-Rad Inc.) that had been equilibrated in advance with three column volumes of 20 mM Tris buffer (pH 8.0) containing 150 mM NaCl. Then, five column volumes of the same buffer was supplied to wash the column, the adsorbed EPO-anti-hTfR was eluted with four column volumes of 50 mM glycine buffer (pH 2.8) containing 150 mM NaCl. The eluate containing EPO-anti-hTfR antibody was adjusted to pH 7.0 with 1 M Tris buffer (pH 8.0), and then the buffer exchanged for PBS using Amicon Ultra 30 kDa membrane (Millipore Inc.). The resulting solution was used as the purification product of EPO-anti-hTfR antibody.

### [Example 26] Method for Preparation of Fusion Protein Between Humanized Anti-hTfR Antibody and Human Arylsulfatase A

A DNA fragment was artificially synthesized comprising the nucleotide sequence set forth as SEQ ID NO:261, which included the cDNA encoding a protein consisting of the humanized anti-hTfR antibody having the amino acid sequence set forth as SEQ ID NO:210 and human arylsulfatase A (hARSA) having the amino acid sequence set forth as SEQ ID NO:259 and linked, via a linker sequence Gly-Ser, to the antibody's heavy chain on the C-terminal side thereof. This DNA fragment encodes a protein having the amino acid sequence set forth as SEQ ID NO:260, which consists of the humanized anti-hTfR antibody and hARSA, which is linked, via a linker sequence Gly-Ser, to the antibody's heavy chain on the C-terminal side thereof. The DNA fragment has in the 5′ side a MluI sequence and a sequence encoding a leader peptide acting as a secretion signal, in this order from the 5′ end, and a NotI sequence in the 3′ side. The DNA fragment was digested with MluI and NotI, and inserted into the vector pE-neo between MluI and NotI sites to construct pE-neo(HC-hARSA).

By the method described in Example 12, CHO cells were transformed with pE-neo(HC-hARSA) and pE-hygr(LC3), which was constructed in Example 11, a cell line was obtained which expresses a fusion protein between hARSA and the humanized anti-hTfR antibody. The cell line was designated hARSA-anti-hTfR antibody expressing cell line. The fusion protein between hARSA and the humanized anti-hTfR antibody, expressed by this cell line, were designated ARSA-anti-hTfR antibody.

Cells of hARSA-anti-hTfR antibody expressing cell line were diluted to the density of approximately 2×10⁵ cells/mL with CD OptiCHO™ medium, and 200 mL of the cell suspension was added to a 1-L conical flask and cultured for 6 to 7 days in a humid environment at 37° C., 5% CO₂, 95% air, with stirring at a rate of about 70 rpm. The culture medium was collected by centrifugation and filtered through a 0.22 μm filter (Millipore Inc.) to prepare a culture supernatant. To the culture supernatant thus collected was added five column volumes of 20 mM Tris buffer (pH 8.0) containing 150 mM NaCl, and loaded onto a Protein A column (column volume: 1 mL, Bio-Rad Inc.) that had been equilibrated in advance with three column volumes of 20 mM Tris buffer (pH 8.0) containing 150 mM NaCl. Then, five column volumes of the same buffer was supplied to wash the column, the adsorbed ARSA-anti-hTfR was eluted with four column volumes of 50 mM glycine buffer (pH 2.8) containing 150 mM NaCl. The eluate containing ARSA-anti-hTfR antibody was adjusted to pH 7.0 with 1 M Tris buffer (pH 8.0), and then the buffer exchanged for PBS using Amicon Ultra 30 kDa membrane (Millipore Inc.). The resulting solution was used as the purification product of ARSA-anti-hTfR antibody.

### [Example 27] Method for Preparation of Fusion Protein Between Humanized Anti-hTfR Antibody and Human PPT-1

A DNA fragment was artificially synthesized comprising the nucleotide sequence set forth as SEQ ID NO:264, which included the cDNA encoding a protein consisting of the humanized anti-hTfR antibody having the amino acid sequence set forth as SEQ ID NO:210 and human PPT-1 (hPPT-1) having the amino acid sequence set forth as SEQ ID NO:262 and linked, via a linker sequence Gly-Ser, to the antibody's heavy chain on the C-terminal side thereof. This DNA fragment encodes a protein having the amino acid sequence set forth as SEQ ID NO:263, which consists of the humanized anti-hTfR antibody and hPPT-1, which is linked, via a linker sequence Gly-Ser, to the antibody's heavy chain on the C-terminal side thereof. The DNA fragment has in the 5′ side a MluI sequence and a sequence encoding a leader peptide acting as a secretion signal, in this order from the 5′ end, and a NotI sequence in the 3′ side. The DNA fragment was digested with MluI and NotI, and inserted into the vector pE-neo between MluI and NotI sites to construct pE-neo(HC-hPPT-1).

By the method described in Example 12, CHO cells were transformed with pE-neo(HC-hPPT-1) and pE-hygr(LC3), which was constructed in Example 11, a cell line was obtained which expresses a fusion protein between hPPT-1 and the humanized anti-hTfR antibody. The cell line was designated hPPT-1-anti-hTfR antibody expressing cell line. The fusion protein between hPPT-1 and the humanized anti-hTfR antibody, expressed by this cell line, were designated PPT-1-anti-hTfR antibody.

Cells of hPPT-1-anti-hTfR antibody expressing cell line were diluted to the density of approximately 2×10⁵ cells/mL with CD OptiCHO™ medium, and 200 mL of the cell suspension was added to a 1-L conical flask and cultured for 6 to 7 days in a humid environment at 37° C., 5% CO₂, 95% air, with stirring at a rate of about 70 rpm. The culture medium was collected by centrifugation and filtered through a 0.22 μm filter (Millipore Inc.) to prepare a culture supernatant. To the culture supernatant thus collected was added five column volumes of 20 mM Tris buffer (pH 8.0) containing 150 mM NaCl, and loaded onto a Protein A column (column volume: 1 mL, Bio-Rad Inc.) that had been equilibrated in advance with three column volumes of 20 mM Tris buffer (pH 8.0) containing 150 mM NaCl. Then, five column volumes of the same buffer was supplied to wash the column, the adsorbed PPT-1-anti-hTfR was eluted with four column volumes of 50 mM glycine buffer (pH 2.8) containing 150 mM NaCl. The eluate containing PPT-1-anti-hTfR antibody was adjusted to pH 7.0 with 1 M Tris buffer (pH 8.0), and then the buffer exchanged for PBS using Amicon Ultra 30 kDa membrane (Millipore Inc.). The resulting solution was used as the purification product of PPT-1-anti-hTfR antibody.

### [Example 28] Method for Preparation of Fusion Protein Between Humanized Anti-hTfR Antibody and Human TPP-1

A DNA fragment was artificially synthesized comprising the nucleotide sequence set forth as SEQ ID NO:267, which included the cDNA encoding a protein consisting of the humanized anti-hTfR antibody having the amino acid sequence set forth as SEQ ID NO:210 and human TPP-1 (hTPP-1) having the amino acid sequence set forth as SEQ ID NO:265 and linked, via a linker sequence Gly-Ser, to the antibody's heavy chain on the C-terminal side thereof. This DNA fragment encodes a fusion protein having the amino acid sequence set forth as SEQ ID NO:266, which consists of the humanized anti-hTfR antibody and hTPP-1 which is linked, via a linker sequence Gly-Ser, to the antibody's heavy chain on the C-terminal side thereof. The DNA fragment has in the 5′ side a MluI sequence and a sequence encoding a leader peptide acting as a secretion signal, in this order from the 5′ end, and a NotI sequence in the 3′ side. The DNA fragment was digested with MluI and NotI, and inserted into the vector pE-neo between MluI and NotI sites to construct pE-neo(HC-hTPP-1).

By the method described in Example 12, CHO cells were transformed with pE-neo(HC-hTPP-1) and pE-hygr(LC3), which was constructed in Example 11, a cell line was obtained which expresses a fusion protein between hTPP-1 and the humanized anti-hTfR antibody. The cell line was designated hTPP-1-anti-hTfR antibody expressing cell line. The fusion protein between hTPP-1 and the humanized anti-hTfR antibody, expressed by this cell line, were designated TPP-1-anti-hTfR antibody.

Cells of hTPP-1-anti-hTfR antibody expressing cell line were diluted to the density of approximately 2×10⁵ cells/mL with CD OptiCHO™ medium, and 200 mL of the cell suspension was added to a 1-L conical flask and cultured for 6 to 7 days in a humid environment at 37° C., 5% CO₂, 95% air, with stirring at a rate of about 70 rpm. The culture medium was collected by centrifugation and filtered through a 0.22 μm filter (Millipore Inc.) to prepare a culture supernatant. To the culture supernatant thus collected was added five column volumes of 20 mM Tris buffer (pH 8.0) containing 150 mM NaCl, and loaded onto a Protein A column (column volume: 1 mL, Bio-Rad Inc.) that had been equilibrated in advance with three column volumes of 20 mM Tris buffer (pH 8.0) containing 150 mM NaCl. Then, five column volumes of the same buffer was supplied to wash the column, the adsorbed TPP-1-anti-hTfR was eluted with four column volumes of 50 mM glycine buffer (pH 2.8) containing 150 mM NaCl. The eluate containing TPP-1-anti-hTfR antibody was adjusted to pH 7.0 with 1 M Tris buffer (pH 8.0), and then the buffer exchanged for PBS using Amicon Ultra 30 kDa membrane (Millipore Inc.). The resulting solution was used as the purification product of TPP-1-anti-hTfR antibody.

### [Example 29] Method for Preparation of Fusion Protein Between Humanized Anti-hTfR Antibody and Human α-L-Iduronidase

A DNA fragment was artificially synthesized comprising the nucleotide sequence set forth as SEQ ID NO:270, which included the cDNA encoding a protein consisting of the humanized anti-hTfR antibody having the amino acid sequence set forth as SEQ ID NO:210 and human α-L-iduronidase (hIDUA) having the amino acid sequence set forth as SEQ ID NO:268 and linked, via a linker sequence Gly-Ser, to the antibody's heavy chain on the C-terminal side thereof. This DNA fragment encodes a fusion protein having the amino acid sequence set forth as SEQ ID NO:269, which consists of the humanized anti-hTfR antibody and hIDUA which is linked, via a linker sequence Gly-Ser, to the antibody's heavy chain on the C-terminal side thereof. The DNA fragment has in the 5′ side a MluI sequence and a sequence encoding a leader peptide acting as a secretion signal, in this order from the 5′ end, and a NotI sequence in the 3′ side. The DNA fragment was digested with MluI and NotI, and inserted into the vector pE-neo between MluI and NotI sites to construct pE-neo(HC-hIDUA).

By the method described in Example 12, CHO cells were transformed with pE-neo(HC-hIDUA) and pE-hygr(LC3), which was constructed in Example 11, a cell line was obtained which expresses a fusion protein between hIDUA and the humanized anti-hTfR antibody. The cell line was designated hIDUA-anti-hTfR antibody expressing cell line. The fusion protein between hIDUA and the humanized anti-hTfR antibody, expressed by this cell line, were designated IDUA-anti-hTfR antibody.

Cells of hIDUA-anti-hTfR antibody expressing cell line were diluted to the density of approximately 2×10⁵ cells/mL with CD OptiCHO™ medium, and 200 mL of the cell suspension was added to a 1-L conical flask and cultured for 6 to 7 days in a humid environment at 37° C., 5% CO₂, 95% air, with stirring at a rate of about 70 rpm. The culture medium was collected by centrifugation and filtered through a 0.22 μm filter (Millipore Inc.) to prepare a culture supernatant. To the culture supernatant thus collected was added five column volumes of 20 mM Tris buffer (pH 8.0) containing 150 mM NaCl, and loaded onto a Protein A column (column volume: 1 mL, Bio-Rad Inc.) that had been equilibrated in advance with three column volumes of 20 mM Tris buffer (pH 8.0) containing 150 mM NaCl. Then, five column volumes of the same buffer was supplied to wash the column, the adsorbed IDUA-anti-hTfR was eluted with four column volumes of 50 mM glycine buffer (pH 2.8) containing 150 mM NaCl. The eluate containing IDUA-anti-hTfR antibody was adjusted to pH 7.0 with 1 M Tris buffer (pH 8.0), and then the buffer exchanged for PBS using Amicon Ultra 30 kDa membrane (Millipore Inc.). The resulting solution was used as the purification product of IDUA-anti-hTfR antibody.

### [Example 30] Method for Preparation of Fusion Protein Between Humanized Anti-hTfR Antibody and Human TNFα Receptor

A DNA fragment was artificially synthesized comprising the nucleotide sequence set forth as SEQ ID NO:273, which included the cDNA encoding a protein consisting of the humanized anti-hTfR antibody having the amino acid sequence set forth as SEQ ID NO:210 and human TNFα receptor (hTNFαR) having the amino acid sequence set forth as SEQ ID NO:271 and linked, via a linker sequence Gly-Ser, to the antibody's heavy chain on the C-terminal side thereof. This DNA fragment encodes a fusion protein having the amino acid sequence set forth as SEQ ID NO:272, which consists of the humanized anti-hTfR antibody and hTNFαR which is linked, via a linker sequence Gly-Ser, to the antibody's heavy chain on the C-terminal side thereof. The DNA fragment has in the 5′ side a MluI sequence and a sequence encoding a leader peptide acting as a secretion signal, in this order from the 5′ end, and a NotI sequence in the 3′ side. The DNA fragment was digested with MluI and NotI, and inserted into the vector pE-neo between MluI and NotI sites to construct pE-neo(HC-hTNFαR).

By the method described in Example 12, CHO cells were transformed with pE-neo(HC-hTNFαR) and pE-hygr(LC3), which was constructed in Example 11, a cell line was obtained which expresses a fusion protein between hTNFαR and the humanized anti-hTfR antibody. The cell line was designated hTNFαR-anti-hTfR antibody expressing cell line. The fusion protein between hTNFαR and the humanized anti-hTfR antibody, expressed by this cell line, were designated TNFαR-anti-hTfR antibody.

Cells of hTNFαR-anti-hTfR antibody expressing cell line were diluted to the density of approximately 2×10⁵ cells/mL with CD OptiCHO™ medium, and 200 mL of the cell suspension was added to a 1-L conical flask and cultured for 6 to 7 days in a humid environment at 37° C., 5% CO₂, 95% air, with stirring at a rate of about 70 rpm. The culture medium was collected by centrifugation and filtered through a 0.22 μm filter (Millipore Inc.) to prepare a culture supernatant. To the culture supernatant thus collected was added five column volumes of 20 mM Tris buffer (pH 8.0) containing 150 mM NaCl, and loaded onto a Protein A column (column volume: 1 mL, Bio-Rad Inc.) that had been equilibrated in advance with three column volumes of 20 mM Tris buffer (pH 8.0) containing 150 mM NaCl. Then, five column volumes of the same buffer was supplied to wash the column, the adsorbed hTNFαR-anti-hTfR was eluted with four column volumes of 50 mM glycine buffer (pH 2.8) containing 150 mM NaCl. The eluate containing TNFαR-anti-hTfR antibody was adjusted to pH 7.0 with 1 M Tris buffer (pH 8.0), and then the buffer exchanged for PBS using Amicon Ultra 30 kDa membrane (Millipore Inc.). The resulting solution was used as the purification product of TNFαR-anti-hTfR antibody.

### [Example 31] Method for Preparation of Fusion Protein Between Humanized Anti-hTfR Antibody and Human Heparan N-Sulfatase

A DNA fragment was artificially synthesized comprising the nucleotide sequence set forth as SEQ ID NO:276, which included the cDNA encoding a protein consisting of the humanized anti-hTfR antibody having the amino acid sequence set forth as SEQ ID NO:210 and human heparan N-sulfatase (hSGSH) having the amino acid sequence set forth as SEQ ID NO:274 and linked, via a linker sequence Gly-Ser, to the antibody's heavy chain on the C-terminal side thereof. This DNA fragment encodes a fusion protein having the amino acid sequence set forth as SEQ ID NO:275, which consists of the humanized anti-hTfR antibody and hSGSH which is linked, via a linker sequence Gly-Ser, to the antibody's heavy chain on the C-terminal side thereof. The DNA fragment has in the 5′ side a MluI sequence and a sequence encoding a leader peptide acting as a secretion signal, in this order from the 5′ end, and a NotI sequence in the 3′ side. The DNA fragment was digested with MluI and NotI, and inserted into the vector pE-neo between MluI and NotI sites to construct pE-neo(HC-hSGSH).

By the method described in Example 12, CHO cells were transformed with pE-neo(HC-hSGSH) and pE-hygr(LC3), which was constructed in Example 11, a cell line was obtained which expresses a fusion protein between hSGSH and the humanized anti-hTfR antibody. The cell line was designated hSGSH-anti-hTfR antibody expressing cell line. The fusion protein between hSGSH and the humanized anti-hTfR antibody, expressed by this cell line, were designated SGSH-anti-hTfR antibody.

Cells of hSGSH-anti-hTfR antibody expressing cell line were diluted to the density of approximately 2×10⁵ cells/mL with CD OptiCHO™ medium, and 200 mL of the cell suspension was added to a 1-L conical flask and cultured for 6 to 7 days in a humid environment at 37° C., 5% CO₂, 95% air, with stirring at a rate of about 70 rpm. The culture medium was collected by centrifugation and filtered through a 0.22 μm filter (Millipore Inc.) to prepare a culture supernatant. To the culture supernatant thus collected was added five column volumes of 20 mM Tris buffer (pH 8.0) containing 150 mM NaCl, and loaded onto a Protein A column (column volume: 1 mL, Bio-Rad Inc.) that had been equilibrated in advance with three column volumes of 20 mM Tris buffer (pH 8.0) containing 150 mM NaCl. Then, five column volumes of the same buffer was supplied to wash the column, the adsorbed SGSH-anti-hTfR was eluted with four column volumes of 50 mM glycine buffer (pH 2.8) containing 150 mM NaCl. The eluate containing SGSH-anti-hTfR antibody was adjusted to pH 7.0 with 1 M Tris buffer (pH 8.0), and then the buffer exchanged for PBS using Amicon Ultra 30 kDa membrane (Millipore Inc.). The resulting solution was used as the purification product of SGSH-anti-hTfR antibody.

### [Example 32] Evaluation of Transfer of Fusion Proteins Between Humanized Anti-hTfR Antibody and Various Physiologically Active Peptides into the Brain

Each purification product of EPO-anti-hTfR antibody, ARSA-anti-hTfR antibody, PPT-1-anti-hTfR antibody, TPP-1-anti-hTfR antibody, IDUA-anti-hTfR antibody, TNFαR-anti-hTfR antibody and SGSH-anti-hTfR antibody prepared in Examples 25 to 31, respectively, was intravenously administered once to an hTfR-KI mouse at a dose of 3 mg/kg. As a control, a human immunoglobulin preparation (Benesis: human immunoglobulin for intramuscular injection, Mitsubishi Tanabe Pharma Inc.) was intravenously injected once to an hTfR-KI mouse (17 to 28 week-old) at a dose of 3 mg/kg. A single hTfR-KI mouse (male, 17 to 28 week-old) was used for the administration of each of the fusion proteins and the control.

Eight hours after the intravenous injection, each mouse was subjected to whole body irrigation with physiological saline, and its brain tissues then were excised after the irrigation. The excised brain tissues then were homogenized with T-PER (Thermo Fisher Scientific Inc.) containing Protease Inhibitor Cocktail (Sigma Inc.), and the supernatant was collected after centrifugation. The concentration of the fusion protein contained in the collected supernatant of the homogenate was measured by the following method. Besides the biotin-labeled goat anti-human IgG Fc polyclonal antibody used was prepared by biotin-labeling goat anti-human IgG Fc polyclonal antibody (Bethyl Inc.) with Biotin Labelling Kit-NH₂ (Dojindo Laboratories Inc.) in accordance with the attached manual. Further, the SULFO-labeled goat anti-human IgG Fc polyclonal antibody used was prepared by SULFO-labeling goat anti-human IgG Fc polyclonal antibody (Bethyl Inc.) with MSD SULFO-TAG NHS-Ester (Meso scale Diagnostics Inc.) according to the attached manual.

To each well of a Streptavidin Gold Plate 96-well (Meso scale Diagnostics Inc.) was added 150 μL of SuperBlock blocking buffer in PBS (Thermo Fisher Scientific Inc.) and left to stand for one hour at room temperature to block the plate. The biotin-labeled goat anti-human IgG Fc polyclonal antibody was diluted to 0.5 μg/mL with SuperBlock blocking buffer in PBS. The SULFO-labeled goat anti-human IgG Fc polyclonal antibody was diluted to 1.0 μg/mL with SuperBlock blocking buffer in PBS. The diluted solutions of biotin-labeled antibody and SULFO-labeled antibody, 25 μL each, were mixed with 25 μL of each sample, and incubated for one hour to prepare samples for antibody reaction.

After each well of the blocked plate was washed with 200 μL of PBS-T (Sigma Inc.), 25 μL of a sample for antibody reaction was added to the well, and incubated for one hour. Following the incubation, each well of the plate was washed with 200 μL of PBS-T, and Read buffer T (Meso scale Diagnostics Inc.) was added and the amount of luminescence from each well was measured on Sector™ Imager 6000 (Meso scale Diagnostics Inc.). The amount of the fusion protein contained per one gram of brain tissues (g wet weight) (the concentration of the fusion protein in the brain tissues) was calculated by producing a standard curve on measurements of standard samples containing known concentrations of the sample, and then interpolating the measurement of each of the samples with reference to the standard. The result is shown in Table 13.

When the concentration of the human immunoglobulin, the control, in the brain tissues is taken as a unit value, the relative values of concentration of EPO-anti-hTfR antibody, ARSA-anti-hTfR antibody, PPT-1-anti-hTfR antibody, TPP-1-anti-hTfR antibody, IDUA-anti-hTfR antibody, TNFαR-anti-hTfR antibody and SGSH-anti-hTfR antibody in the brain tissues are 4.33, 3.39, 4.87, 6.48, 5.62, 7.44, and 2.24, respectively, demonstrating that these physiologically active peptides linked to the anti-hTfR antibody actively transfer into the brain tissues. The result thus indicates that these physiologically active proteins, which usually do not pass through the blood-brain barrier, can be made to pass through the blood-brain barrier and get into the brain tissues by fusing them to the anti-hTfR antibody.

Thus, the above results indicate that EPO-anti-hTfR antibody can be used as a therapeutic agent for cerebral ischemia, ARSA-anti-hTfR antibody, arylsulfatase A as a therapeutic agent for central nervous system disorders in metachromatic white matter degeneration (metachromatic leukodystrophy), PPT-1-anti-hTfR antibody as a therapeutic agent for central nervous system disorders in neuronal ceroid lipofuscinosis or Santavuori-Haltia disease, TPP-1-anti-hTfR antibody as a therapeutic agent for central nervous system disorders in neuronal ceroid lipofuscinosis or Jansky-Bielschowsky disease, IDUA-anti-hTfR antibody as a therapeutic agent for central nervous system disorders in Hurler syndrome or Hurler-Scheie syndrome, SGSH-anti-hTfR antibody as a therapeutic agent for central nervous system disorders in Sanfilippo syndrome, IDUA-anti-hTfR antibody as a therapeutic agent for central nervous system disorders in Hurler syndrome or Hurler-Scheie syndrome, and TNFαR-anti-hTfR antibody as a therapeutic agent for cerebral ischemia and encephalitis. Besides, the above results also indicate physiologically active proteins of interest which do not usually pass through the blood-brain barrier can be made to pass through the blood-brain barrier and get into the brain tissues, by fusing them with the anti-hTfR antibody.

TABLE 13
Concentration of fusion proteins in brain tissues
(μg/g wet weight)

| | | Relative |
|---|---|---|
| | | value to |
| Fusion protein | Concentration | control |
| Control | 0.0199 | 1 |
| EPO-anti-hTfR antibody | 0.0862 | 4.33 |
| ARSA-anti-hTfR antibody | 0.0675 | 3.39 |
| PPT-1-anti-hTfR antibody | 0.0970 | 4.87 |
| TPP-1-anti-hTfR antibody | 0.129 | 6.48 |
| IDUA-1-anti-hTfR antibody | 0.112 | 5.62 |
| TNFαR-anti-hTfR antibody | 0.148 | 7.44 |
| SGSH-anti-hTfR antibody | 0.0445 | 2.24 |

### INDUSTRIAL APPLICABILITY

The anti-hTfR antibody of the present invention, when fused with physiologically active proteins, low-molecular-weight compounds and the like of interest, can make them able to pass through the blood-brain barrier, and is, therefore, highly useful in providing a means to deliver physiologically active proteins to the brain, low-molecular-weight compounds and the like which are needed to act in the central nervous system.

### REFERENCE SIGNS LIST

1 Blood vessel
2 Brain parenchyma
3 Neuron-like cells
4 Purkinje cells

### Sequence Listing Free Text

SEQ ID NO:3: Amino acid sequence of exemplified linker 1
SEQ ID NO:4: Amino acid sequence of exemplified linker 2
SEQ ID NO:5: Amino acid sequence of exemplified linker 3
SEQ ID NO:6: Amino acid sequence 1 of CDR1 in the light chain of mouse anti-hTfR antibody No. 1
SEQ ID NO:7: Amino acid sequence 2 of CDR1 in the light chain of mouse anti-hTfR antibody No. 1
SEQ ID NO:8: Amino acid sequence 1 of CDR2 in the light chain of mouse anti-hTfR antibody No. 1
SEQ ID NO:9: Amino acid sequence 2 of CDR2 in the light chain of mouse anti-hTfR antibody No. 1
SEQ ID NO:10: Amino acid sequence of CDR3 in the light chain of mouse anti-hTfR antibody No. 1
SEQ ID NO:11: Amino acid sequence 1 of CDR1 in the light chain of mouse anti-hTfR antibody No. 2
SEQ ID NO:12: Amino acid sequence 2 of CDR1 in the light chain of mouse anti-hTfR antibody No. 2
SEQ ID NO:13: Amino acid sequence 1 of CDR2 in the light chain of mouse anti-hTfR antibody No. 2
SEQ ID NO:14: Amino acid sequence 2 of CDR2 in the light chain of mouse anti-hTfR antibody No. 2
SEQ ID NO:15: Amino acid sequence of CDR3 in the light chain of mouse anti-hTfR antibody No. 2
SEQ ID NO:16: Amino acid sequence 1 of CDR1 in the light chain of mouse anti-hTfR antibody No. 3
SEQ ID NO:17: Amino acid sequence 2 of CDR1 in the light chain of mouse anti-hTfR antibody No. 3
SEQ ID NO:18: Amino acid sequence 1 of CDR2 in the light chain of mouse anti-hTfR antibody No. 3
SEQ ID NO:19: Amino acid sequence 2 of CDR2 in the light chain of mouse anti-hTfR antibody No. 3
SEQ ID NO:20: Amino acid sequence of CDR3 in the light chain of mouse anti-hTfR antibody No. 3
SEQ ID NO:21: Amino acid sequence 1 of CDR1 in the light chain of mouse anti-hTfR antibody No. 4
SEQ ID NO:22: Amino acid sequence 2 of CDR1 in the light chain of mouse anti-hTfR antibody No. 4
SEQ ID NO:23: Amino acid sequence 1 of CDR2 in the light chain of mouse anti-hTfR antibody No. 4
SEQ ID NO:24: Amino acid sequence 2 of CDR2 in the light chain of mouse anti-hTfR antibody No. 4
SEQ ID NO:25: Amino acid sequence of CDR3 in the light chain of mouse anti-hTfR antibody No. 4
SEQ ID NO:26: Amino acid sequence 1 of CDR1 in the light chain of mouse anti-hTfR antibody No. 5
SEQ ID NO:27: Amino acid sequence 2 of CDR1 in the light chain of mouse anti-hTfR antibody No. 5
SEQ ID NO:28: Amino acid sequence 1 of CDR2 in the light chain of mouse anti-hTfR antibody No. 5
SEQ ID NO:29: Amino acid sequence 2 of CDR2 in the light chain of mouse anti-hTfR antibody No. 5
SEQ ID NO:30: Amino acid sequence of CDR3 in the light chain of mouse anti-hTfR antibody No. 5
SEQ ID NO:31: Amino acid sequence 1 of CDR1 in the light chain of mouse anti-hTfR antibody No. 6
SEQ ID NO:32: Amino acid sequence 2 of CDR1 in the light chain of mouse anti-hTfR antibody No. 6
SEQ ID NO:33: Amino acid sequence 1 of CDR2 in the light chain of mouse anti-hTfR antibody No. 6
SEQ ID NO:34: Amino acid sequence 2 of CDR2 in the light chain of mouse anti-hTfR antibody No. 6
SEQ ID NO:35: Amino acid sequence of CDR3 in the light chain of mouse anti-hTfR antibody No. 6
SEQ ID NO:36: Amino acid sequence 1 of CDR1 in the light chain of mouse anti-hTfR antibody No. 7
SEQ ID NO:37: Amino acid sequence 2 of CDR1 in the light chain of mouse anti-hTfR antibody No. 7
SEQ ID NO:38: Amino acid sequence 1 of CDR2 in the light chain of mouse anti-hTfR antibody No. 7
SEQ ID NO:39: Amino acid sequence 2 of CDR2 in the light chain of mouse anti-hTfR antibody No. 7
SEQ ID NO:40: Amino acid sequence of CDR3 in the light chain of mouse anti-hTfR antibody No. 7
SEQ ID NO:41: Amino acid sequence 1 of CDR1 in the light chain of mouse anti-hTfR antibody No. 8
SEQ ID NO:42: Amino acid sequence 2 of CDR1 in the light chain of mouse anti-hTfR antibody No. 8
SEQ ID NO:43: Amino acid sequence 1 of CDR2 in the light chain of mouse anti-hTfR antibody No. 8
SEQ ID NO:44: Amino acid sequence 2 of CDR2 in the light chain of mouse anti-hTfR antibody No. 8
SEQ ID NO:45: Amino acid sequence of CDR3 in the light chain of mouse anti-hTfR antibody No. 8
SEQ ID NO:46: Amino acid sequence 1 of CDR1 in the light chain of mouse anti-hTfR antibody No. 9
SEQ ID NO:47: Amino acid sequence 2 of CDR1 in the light chain of mouse anti-hTfR antibody No. 9
SEQ ID NO:48: Amino acid sequence 1 of CDR2 in the light chain of mouse anti-hTfR antibody No. 9
SEQ ID NO:49: Amino acid sequence 2 of CDR2 in the light chain of mouse anti-hTfR antibody No. 9
SEQ ID NO:50: Amino acid sequence of CDR3 in the light chain of mouse anti-hTfR antibody No. 9
SEQ ID NO:51: Amino acid sequence 1 of CDR1 in the light chain of mouse anti-hTfR antibody No. 10
SEQ ID NO:52: Amino acid sequence 2 of CDR1 in the light chain of mouse anti-hTfR antibody No. 10
SEQ ID NO:53: Amino acid sequence 1 of CDR2 in the light chain of mouse anti-hTfR antibody No. 10
SEQ ID NO:54: Amino acid sequence 2 of CDR2 in the light chain of mouse anti-hTfR antibody No. 10
SEQ ID NO:55: Amino acid sequence of CDR3 in the light chain of mouse anti-hTfR antibody No. 10
SEQ ID NO:56: Amino acid sequence 1 of CDR1 in the light chain of mouse anti-hTfR antibody No. 11
SEQ ID NO:57: Amino acid sequence 2 of CDR1 in the light chain of mouse anti-hTfR antibody No. 11
SEQ ID NO:58: Amino acid sequence 1 of CDR2 in the light chain of mouse anti-hTfR antibody No. 11
SEQ ID NO:59: Amino acid sequence 2 of CDR2 in the light chain of mouse anti-hTfR antibody No. 11
SEQ ID NO:60: Amino acid sequence of CDR3 in the light chain of mouse anti-hTfR antibody No. 11
SEQ ID NO:61: Amino acid sequence 1 of CDR1 in the light chain of mouse anti-hTfR antibody No. 12
SEQ ID NO:62: Amino acid sequence 2 of CDR1 in the light chain of mouse anti-hTfR antibody No. 12
SEQ ID NO:63: Amino acid sequence 1 of CDR2 in the light chain of mouse anti-hTfR antibody No. 12
SEQ ID NO:64: Amino acid sequence 2 of CDR2 in the light chain of mouse anti-hTfR antibody No. 12
SEQ ID NO:65: Amino acid sequence of CDR3 in the light chain of mouse anti-hTfR antibody No. 12
SEQ ID NO:66: Amino acid sequence 1 of CDR1 in the light chain of mouse anti-hTfR antibody No. 13
SEQ ID NO:67: Amino acid sequence 2 of CDR1 in the light chain of mouse anti-hTfR antibody No. 13
SEQ ID NO:68: Amino acid sequence 1 of CDR2 in the light chain of mouse anti-hTfR antibody No. 13
SEQ ID NO:69: Amino acid sequence 2 of CDR2 in the light chain of mouse anti-hTfR antibody No. 13
SEQ ID NO:70: Amino acid sequence of CDR3 in the light chain of mouse anti-hTfR antibody No. 13
SEQ ID NO:71: Amino acid sequence 1 of CDR1 in the light chain of mouse anti-hTfR antibody No. 14
SEQ ID NO:72: Amino acid sequence 2 of CDR1 in the light chain of mouse anti-hTfR antibody No. 14
SEQ ID NO:73: Amino acid sequence 1 of CDR2 in the light chain of mouse anti-hTfR antibody No. 14
SEQ ID NO:74: Amino acid sequence 2 of CDR2 in the light chain of mouse anti-hTfR antibody No. 14
SEQ ID NO:75: Amino acid sequence of CDR3 in the light chain of mouse anti-hTfR antibody No. 14
SEQ ID NO:76: Amino acid sequence 1 of CDR1 in the heavy chain of mouse anti-hTfR antibody No. 1
SEQ ID NO:77: Amino acid sequence 2 of CDR1 in the heavy chain of mouse anti-hTfR antibody No. 1
SEQ ID NO:78: Amino acid sequence 1 of CDR2 in the heavy chain of mouse anti-hTfR antibody No. 1
SEQ ID NO:79: Amino acid sequence 2 of CDR2 in the heavy chain of mouse anti-hTfR antibody No. 1
SEQ ID NO:80: Amino acid sequence 1 of CDR3 in the heavy chain of mouse anti-hTfR antibody No. 1
SEQ ID NO:81: Amino acid sequence 2 of CDR3 in the heavy chain of mouse anti-hTfR antibody No. 1
SEQ ID NO:82: Amino acid sequence 1 of CDR1 in the heavy chain of mouse anti-hTfR antibody No. 2
SEQ ID NO:83: Amino acid sequence 2 of CDR1 in the heavy chain of mouse anti-hTfR antibody No. 2
SEQ ID NO:84: Amino acid sequence 1 of CDR2 in the heavy chain of mouse anti-hTfR antibody No. 2
SEQ ID NO:85: Amino acid sequence 2 of CDR2 in the heavy chain of mouse anti-hTfR antibody No. 2
SEQ ID NO:86: Amino acid sequence 1 of CDR3 in the heavy chain of mouse anti-hTfR antibody No. 2
SEQ ID NO:87: Amino acid sequence 2 of CDR3 in the heavy chain of mouse anti-hTfR antibody No. 2
SEQ ID NO:88: Amino acid sequence 1 of CDR1 in the heavy chain of mouse anti-hTfR antibody No. 3
SEQ ID NO:89: Amino acid sequence 2 of CDR1 in the heavy chain of mouse anti-hTfR antibody No. 3
SEQ ID NO:90: Amino acid sequence 1 of CDR2 in the heavy chain of mouse anti-hTfR antibody No. 3
SEQ ID NO:91: Amino acid sequence 2 of CDR2 in the heavy chain of mouse anti-hTfR antibody No. 3
SEQ ID NO:92: Amino acid sequence 1 of CDR3 in the heavy chain of mouse anti-hTfR antibody No. 3
SEQ ID NO:93: Amino acid sequence 2 of CDR3 in the heavy chain of mouse anti-hTfR antibody No. 3
SEQ ID NO:94: Amino acid sequence 1 of CDR1 in the heavy chain of mouse anti-hTfR antibody No. 4
SEQ ID NO:95: Amino acid sequence 2 of CDR1 in the heavy chain of mouse anti-hTfR antibody No. 4
SEQ ID NO:96: Amino acid sequence 1 of CDR2 in the heavy chain of mouse anti-hTfR antibody No. 4
SEQ ID NO:97: Amino acid sequence 2 of CDR2 in the heavy chain of mouse anti-hTfR antibody No. 4
SEQ ID NO:98: Amino acid sequence 1 of CDR3 in the heavy chain of mouse anti-hTfR antibody No. 4
SEQ ID NO:99: Amino acid sequence 2 of CDR3 in the heavy chain of mouse anti-hTfR antibody No. 4
SEQ ID NO:100: Amino acid sequence 1 of CDR1 in the heavy chain of mouse anti-hTfR antibody No. 5
SEQ ID NO:101: Amino acid sequence 2 of CDR1 in the heavy chain of mouse anti-hTfR antibody No. 5
SEQ ID NO:102: Amino acid sequence 1 of CDR2 in the heavy chain of mouse anti-hTfR antibody No. 5
SEQ ID NO:103: Amino acid sequence 2 of CDR2 in the heavy chain of mouse anti-hTfR antibody No. 5
SEQ ID NO:104: Amino acid sequence 1 of CDR3 in the heavy chain of mouse anti-hTfR antibody No. 5
SEQ ID NO:105: Amino acid sequence 2 of CDR3 in the heavy chain of mouse anti-hTfR antibody No. 5
SEQ ID NO:106: Amino acid sequence 1 of CDR1 in the heavy chain of mouse anti-hTfR antibody No. 6
SEQ ID NO:107: Amino acid sequence 2 of CDR1 in the heavy chain of mouse anti-hTfR antibody No. 6
SEQ ID NO:108: Amino acid sequence of CDR2 in the heavy chain of mouse anti-hTfR antibody No. 6
SEQ ID NO:109: Amino acid sequence 1 of CDR3 in the heavy chain of mouse anti-hTfR antibody No. 6
SEQ ID NO:110: Amino acid sequence 2 of CDR3 in the heavy chain of mouse anti-hTfR antibody No. 6
SEQ ID NO:111: Amino acid sequence 1 of CDR1 in the heavy chain of mouse anti-hTfR antibody No. 7
SEQ ID NO:112: Amino acid sequence 2 of CDR1 in the heavy chain of mouse anti-hTfR antibody No. 7
SEQ ID NO:113: Amino acid sequence 1 of CDR2 in the heavy chain of mouse anti-hTfR antibody No. 7
SEQ ID NO:114: Amino acid sequence 2 of CDR2 in the heavy chain of mouse anti-hTfR antibody No. 7
SEQ ID NO:115: Amino acid sequence 1 of CDR3 in the heavy chain of mouse anti-hTfR antibody No. 7
SEQ ID NO:116: Amino acid sequence 2 of CDR3 in the heavy chain of mouse anti-hTfR antibody No. 7
SEQ ID NO:117: Amino acid sequence 1 of CDR1 in the heavy chain of mouse anti-hTfR antibody No. 8
SEQ ID NO:118: Amino acid sequence 2 of CDR1 in the heavy chain of mouse anti-hTfR antibody No. 8
SEQ ID NO:119: Amino acid sequence of CDR2 in the heavy chain of mouse anti-hTfR antibody No. 8
SEQ ID NO:120: Amino acid sequence 1 of CDR3 in the heavy chain of mouse anti-hTfR antibody No. 8
SEQ ID NO:121: Amino acid sequence 2 of CDR3 in the heavy chain of mouse anti-hTfR antibody No. 8
SEQ ID NO:122: Amino acid sequence 1 of CDR1 in the heavy chain of mouse anti-hTfR antibody No. 9
SEQ ID NO:123: Amino acid sequence 2 of CDR1 in the heavy chain of mouse anti-hTfR antibody No. 9
SEQ ID NO:124: Amino acid sequence 1 of CDR2 in the heavy chain of mouse anti-hTfR antibody No. 9
SEQ ID NO:125: Amino acid sequence 2 of CDR2 in the heavy chain of mouse anti-hTfR antibody No. 9
SEQ ID NO:126: Amino acid sequence 1 of CDR3 in the heavy chain of mouse anti-hTfR antibody No. 9
SEQ ID NO:127: Amino acid sequence 2 of CDR3 in the heavy chain of mouse anti-hTfR antibody No. 9
SEQ ID NO:128: Amino acid sequence 1 of CDR1 in the heavy chain of mouse anti-hTfR antibody No. 10
SEQ ID NO:129: Amino acid sequence 2 of CDR1 in the heavy chain of mouse anti-hTfR antibody No. 10
SEQ ID NO:130: Amino acid sequence 1 of CDR2 in the heavy chain of mouse anti-hTfR antibody No. 10
SEQ ID NO:131: Amino acid sequence 2 of CDR2 in the heavy chain of mouse anti-hTfR antibody No. 10
SEQ ID NO:132: Amino acid sequence 1 of CDR3 in the heavy chain of mouse anti-hTfR antibody No. 10
SEQ ID NO:133: Amino acid sequence 2 of CDR3 in the heavy chain of mouse anti-hTfR antibody No. 10
SEQ ID NO:134: Amino acid sequence 1 of CDR1 in the heavy chain of mouse anti-hTfR antibody No. 11
SEQ ID NO:135: Amino acid sequence 2 of CDR1 in the heavy chain of mouse anti-hTfR antibody No. 11
SEQ ID NO:136: Amino acid sequence 1 of CDR2 in the heavy chain of mouse anti-hTfR antibody No. 11
SEQ ID NO:137: Amino acid sequence 2 of CDR2 in the heavy chain of mouse anti-hTfR antibody No. 11
SEQ ID NO:138: Amino acid sequence 1 of CDR3 in the heavy chain of mouse anti-hTfR antibody No. 11
SEQ ID NO:139: Amino acid sequence 2 of CDR3 in the heavy chain of mouse anti-hTfR antibody No. 11
SEQ ID NO:140: Amino acid sequence 1 of CDR1 in the heavy chain of mouse anti-hTfR antibody No. 12
SEQ ID NO:141: Amino acid sequence 2 of CDR1 in the heavy chain of mouse anti-hTfR antibody No. 12
SEQ ID NO:142: Amino acid sequence 1 of CDR2 in the heavy chain of mouse anti-hTfR antibody No. 12
SEQ ID NO:143: Amino acid sequence 2 of CDR2 in the heavy chain of mouse anti-hTfR antibody No. 12
SEQ ID NO:144: Amino acid sequence 1 of CDR3 in the heavy chain of mouse anti-hTfR antibody No. 12
SEQ ID NO:145: Amino acid sequence 2 of CDR3 in the heavy chain of mouse anti-hTfR antibody No. 12
SEQ ID NO:146: Amino acid sequence 1 of CDR1 in the heavy chain of mouse anti-hTfR antibody No. 13
SEQ ID NO:147: Amino acid sequence 2 of CDR1 in the heavy chain of mouse anti-hTfR antibody No. 13
SEQ ID NO:148: Amino acid sequence 1 of CDR2 in the heavy chain of mouse anti-hTfR antibody No. 13
SEQ ID NO:149: Amino acid sequence 2 of CDR2 in the heavy chain of mouse anti-hTfR antibody No. 13
SEQ ID NO:150: Amino acid sequence 1 of CDR3 in the heavy chain of mouse anti-hTfR antibody No. 13
SEQ ID NO:151: Amino acid sequence 2 of CDR3 in the heavy chain of mouse anti-hTfR antibody No. 13
SEQ ID NO:152: Amino acid sequence 1 of CDR1 in the heavy chain of mouse anti-hTfR antibody No. 14
SEQ ID NO:153: Amino acid sequence 2 of CDR1 in the heavy chain of mouse anti-hTfR antibody No. 14
SEQ ID NO:154: Amino acid sequence 1 of CDR2 in the heavy chain of mouse anti-hTfR antibody No. 14
SEQ ID NO:155: Amino acid sequence 2 of CDR2 in the heavy chain of mouse anti-hTfR antibody No. 14
SEQ ID NO:156: Amino acid sequence 1 of CDR3 in the heavy chain of mouse anti-hTfR antibody No. 14
SEQ ID NO:157: Amino acid sequence 2 of CDR3 in the heavy chain of mouse anti-hTfR antibody No. 14
SEQ ID NO:158: Amino acid sequence 1 of the light chain variable region of humanized anti-hTfR antibody No. 1
SEQ ID NO:159: Amino acid sequence 2 of the light chain variable region of humanized anti-hTfR antibody No. 1
SEQ ID NO:160: Amino acid sequence 3 of the light chain variable region of humanized anti-hTfR antibody No. 1
SEQ ID NO:161: Amino acid sequence 4 of the light chain variable region of humanized anti-hTfR antibody No. 1
SEQ ID NO:162: Amino acid sequence 5 of the light chain variable region of humanized anti-hTfR antibody No. 1
SEQ ID NO:163: Amino acid sequence 6 of the light chain variable region of humanized anti-hTfR antibody No. 1
SEQ ID NO:164: Amino acid sequence of the light chain of humanized anti-hTfR antibody No. 1 containing amino acid sequence 6 as the variable region, synthetic sequence
SEQ ID NO:165: Nucleotide sequence encoding the amino acid sequence of the light chain of humanized anti-hTfR antibody No. 1 containing amino acid sequence 6 as the variable region, synthetic sequence
SEQ ID NO:166: Amino acid sequence 1 of the heavy chain variable region of humanized anti-hTfR antibody No. 1
SEQ ID NO:167: Amino acid sequence 2 of the heavy chain variable region of humanized anti-hTfR antibody No. 1
SEQ ID NO:168: Amino acid sequence 3 of the heavy chain variable region of humanized anti-hTfR antibody No. 1
SEQ ID NO:169: Amino acid sequence 4 of the heavy chain variable region of humanized anti-hTfR antibody No. 1
SEQ ID NO:170: Amino acid sequence 5 of the heavy chain variable region of humanized anti-hTfR antibody No. 1
SEQ ID NO:171: Amino acid sequence 6 of the heavy chain variable region of humanized anti-hTfR antibody No. 1
SEQ ID NO:172: Amino acid sequence of the heavy chain of humanized anti-hTfR antibody No. 1 containing amino acid sequence 6 as the variable region
SEQ ID NO:173: Nucleotide sequence encoding the amino acid sequence of the heavy chain of humanized anti-hTfR antibody No. 1 containing amino acid sequence 6 as the variable region, synthetic sequence
SEQ ID NO:174: Amino acid sequence 1 of the light chain variable region of humanized anti-hTfR antibody No. 2
SEQ ID NO:175: Amino acid sequence 2 of the light chain variable region of humanized anti-hTfR antibody No. 2
SEQ ID NO:176: Amino acid sequence 3 of the light chain variable region of humanized anti-hTfR antibody No. 2
SEQ ID NO:177: Amino acid sequence 4 of the light chain variable region of humanized anti-hTfR antibody No. 2
SEQ ID NO:178: Amino acid sequence 5 of the light chain variable region of humanized anti-hTfR antibody No. 2
SEQ ID NO:179: Amino acid sequence 6 of the light chain variable region of humanized anti-hTfR antibody No. 2
SEQ ID NO:180: Amino acid sequence of the light chain of humanized anti-hTfR antibody No. 2 containing amino acid sequence 6 as the variable region
SEQ ID NO:181: Nucleotide sequence comprising a nucleotide sequence encoding the amino acid sequence of the light chain of humanized anti-hTfR antibody No. 2 containing amino acid sequence 6 as the variable region, synthetic sequence
SEQ ID NO:182: Amino acid sequence 1 of the heavy chain variable region of humanized anti-hTfR antibody No. 2
SEQ ID NO:183: Amino acid sequence 2 of the heavy chain variable region of humanized anti-hTfR antibody No. 2
SEQ ID NO:184: Amino acid sequence 3 of the heavy chain variable region of humanized anti-hTfR antibody No. 2
SEQ ID NO:185: Amino acid sequence 4 of the heavy chain variable region of humanized anti-hTfR antibody No. 2
SEQ ID NO:186: Amino acid sequence 5 of the heavy chain variable region of humanized anti-hTfR antibody No. 2
SEQ ID NO:187: Amino acid sequence 6 of the heavy chain variable region of humanized anti-hTfR antibody No. 2
SEQ ID NO:188: Amino acid sequence of the heavy chain of humanized anti-hTfR antibody No. 2 containing amino acid sequence 6 as the variable region
SEQ ID NO:189: Nucleotide sequence comprising a nucleotide sequence encoding the amino acid sequence of the heavy chain of humanized anti-hTfR antibody No. 2 containing amino acid sequence 6 as the variable region, synthetic sequence
SEQ ID NO:190: Amino acid sequence 1 of the light chain variable region of humanized anti-hTfR antibody No. 3
SEQ ID NO:191: Amino acid sequence 2 of the light chain variable region of humanized anti-hTfR antibody No. 3
SEQ ID NO:192: Amino acid sequence 3 of the light chain variable region of humanized anti-hTfR antibody No. 3
SEQ ID NO:193: Amino acid sequence 4 of the light chain variable region of humanized anti-hTfR antibody No. 3
SEQ ID NO:194: Amino acid sequence 5 of the light chain variable region of humanized anti-hTfR antibody No. 3
SEQ ID NO:195: Amino acid sequence 6 of the light chain variable region of humanized anti-hTfR antibody No. 3
SEQ ID NO:196: Amino acid sequence of the light chain of humanized anti-hTfR antibody No. 3 containing amino acid sequence 2 as the variable region
SEQ ID NO:197: Nucleotide sequence encoding the amino acid sequence of the light chain of humanized anti-hTfR antibody No. 3 containing amino acid sequence 2 as the variable region, synthetic sequence
SEQ ID NO:198: Amino acid sequence of the light chain of humanized anti-hTfR antibody No. 3 containing amino acid sequence 4 as the variable region
SEQ ID NO:199: Nucleotide sequence encoding the amino acid sequence of the light chain of humanized anti-hTfR antibody No. 3 containing amino acid sequence 4 as the variable region, synthetic sequence
SEQ ID NO:200: Amino acid sequence of the light chain of humanized anti-hTfR antibody No. 3 containing amino acid sequence 5 as the variable region
SEQ ID NO:201: Nucleotide sequence encoding the amino acid sequence of the light chain of humanized anti-hTfR antibody No. 3 containing amino acid sequence 5 as the variable region, synthetic sequence
SEQ ID NO:202: Humanized anti-hTfR antibody No. 3 containing amino acid sequence 6 as the variable region
SEQ ID NO:203: Nucleotide sequence encoding the amino acid sequence of the light chain of humanized anti-hTfR antibody No. 3 containing amino acid sequence 6 as the variable region, synthetic sequence
SEQ ID NO:204: Amino acid sequence 1 of the heavy chain variable region of humanized anti-hTfR antibody No. 3
SEQ ID NO:205: Amino acid sequence 2 of the heavy chain variable region of humanized anti-hTfR antibody No. 3
SEQ ID NO:206: Amino acid sequence 3 of the heavy chain variable region of humanized anti-hTfR antibody No. 3
SEQ ID NO:207: Amino acid sequence 4 of the heavy chain variable region of humanized anti-hTfR antibody No. 3
SEQ ID NO:208: Amino acid sequence 5 of the heavy chain variable region of humanized anti-hTfR antibody No. 3
SEQ ID NO:209: Amino acid sequence 6 of the heavy chain variable region of humanized anti-hTfR antibody No. 3
SEQ ID NO:210: Amino acid sequence of the heavy chain of humanized anti-hTfR antibody No. 3 containing amino acid sequence 2 as the variable region
SEQ ID NO:211: Nucleotide sequence encoding the amino acid sequence of the heavy chain of humanized anti-hTfR antibody No. 3 containing amino acid sequence 2 as the variable region, synthetic sequence
SEQ ID NO:212: Amino acid sequence of the heavy chain (IgG4) of humanized anti-hTfR antibody No. 3 containing amino acid sequence 2 as the variable region
SEQ ID NO:213: Nucleotide sequence encoding the amino acid sequence of the heavy chain (IgG4) of humanized anti-hTfR antibody No. 3 containing amino acid sequence 2 as the variable region, synthetic sequence
SEQ ID NO:214: Primer hTfR5′, synthetic sequence
SEQ ID NO:215: Primer hTfR3′, synthetic sequence
SEQ ID NO:216: Primer Hyg-Sfi5′, synthetic sequence
SEQ ID NO:217: Primer Hyg-BstX3′, synthetic sequence
SEQ ID NO:218: Amino acid sequence of the light chain variable region of mouse anti-hTfR antibody No. 1
SEQ ID NO:219: Amino acid sequence of the heavy chain variable region of mouse anti-hTfR antibody No. 1
SEQ ID NO:220: Amino acid sequence of the light chain variable region of mouse anti-hTfR antibody No. 2
SEQ ID NO:221: Amino acid sequence of the heavy chain variable region of mouse anti-hTfR antibody No. 2
SEQ ID NO:222: Amino acid sequence of the light chain variable region of mouse anti-hTfR antibody No. 3
SEQ ID NO:223: Amino acid sequence of the heavy chain variable region of mouse anti-hTfR antibody No. 3
SEQ ID NO:224: Amino acid sequence of the light chain variable region of mouse anti-hTfR antibody No. 4
SEQ ID NO:225: Amino acid sequence of the heavy chain variable region of mouse anti-hTfR antibody No. 4
SEQ ID NO:226: Amino acid sequence of the light chain variable region of mouse anti-hTfR antibody No. 5
SEQ ID NO:227: Amino acid sequence of the heavy chain variable region of mouse anti-hTfR antibody No. 5
SEQ ID NO:228: Amino acid sequence of the light chain variable region of mouse anti-hTfR antibody No. 6
SEQ ID NO:229: Amino acid sequence of the heavy chain variable region of mouse anti-hTfR antibody No. 6
SEQ ID NO:230: Amino acid sequence of the light chain variable region of mouse anti-hTfR antibody No. 7
SEQ ID NO:231: Amino acid sequence of the heavy chain variable region of mouse anti-hTfR antibody No. 7
SEQ ID NO:232: Amino acid sequence of the light chain variable region of mouse anti-hTfR antibody No. 8
SEQ ID NO:233: Amino acid sequence of the heavy chain variable region of mouse anti-hTfR antibody No. 8
SEQ ID NO:234: Amino acid sequence of the light chain variable region of mouse anti-hTfR antibody No. 9
SEQ ID NO:235: Amino acid sequence of the heavy chain variable region of mouse anti-hTfR antibody No. 9
SEQ ID NO:236: Amino acid sequence of the light chain variable region of mouse anti-hTfR antibody No. 10
SEQ ID NO:237: Amino acid sequence of the heavy chain variable region of mouse anti-hTfR antibody No. 10
SEQ ID NO:238: Amino acid sequence of the light chain variable region of mouse anti-hTfR antibody No. 11
SEQ ID NO:239: Amino acid sequence of the heavy chain variable region of mouse anti-hTfR antibody No. 11
SEQ ID NO:240: Amino acid sequence of the light chain variable region of mouse anti-hTfR antibody No. 12
SEQ ID NO:241: Amino acid sequence of the heavy chain variable region of mouse anti-hTfR antibody No. 12
SEQ ID NO:242: Amino acid sequence of the light chain variable region of mouse anti-hTfR antibody No. 13
SEQ ID NO:243: Amino acid sequence of the heavy chain variable region of mouse anti-hTfR antibody No. 13
SEQ ID NO:244: Amino acid sequence of the light chain variable region of mouse anti-hTfR antibody No. 14
SEQ ID NO:245: Amino acid sequence of the heavy chain variable region of mouse anti-hTfR antibody No. 14
SEQ ID NO:247: Amino acid sequence of the fusion protein of heavy chain of anti-hTfR antibody No. 1 (humanized 6) and hI2S
SEQ ID NO:248: Nucleotide sequence encoding the amino acid sequence of fusion protein of heavy chain of anti-hTfR antibody No. 1 (humanized 6) and hI2S, synthetic sequence
SEQ ID NO:249: Amino acid sequence of the fusion protein of heavy chain of anti-hTfR antibody No. 2 (humanized 6) and hI2S
SEQ ID NO:250: Nucleotide sequence encoding the amino acid sequence of fusion protein of heavy chain of anti-hTfR antibody No. 2 (humanized 6) and hI2S, synthetic sequence
SEQ ID NO:251: Amino acid sequence of fusion protein of heavy chain of anti-hTfR antibody No. 3 (humanized 2) and hI2S, synthetic sequence
SEQ ID NO:252: Nucleotide sequence encoding the amino acid sequence of fusion protein of heavy chain of anti-hTfR antibody No. 3 (humanized 2) and hI2S, synthetic sequence
SEQ ID NO:253: Nucleotide sequence of the DNA in which a neomycin resistance gene flanked by loxP sequences was placed on the cDNA's 3′ side of a cDNA encoding chimeric hTfR, synthetic sequence
SEQ ID NO:254: Nucleotide sequence of the 5′-arm of targeting vector, synthetic sequence
SEQ ID NO:255: Nucleotide sequence of the 3′-arm of targeting vector, synthetic sequence
SEQ ID NO:257: Amino acid sequence of the fusion protein of heavy chain of anti-hTfR antibody No. 3 (humanized 2) and hEPO
SEQ ID NO:258: Nucleotide sequence encoding the amino acid sequence of fusion protein of heavy chain of anti-hTfR antibody No. 3 (humanized 2) and hEPO, synthetic sequence
SEQ ID NO:260: Amino acid sequence of the fusion protein of heavy chain of anti-hTfR antibody No. 3 (humanized 2) and hARSA
SEQ ID NO:261: Nucleotide sequence encoding the amino acid sequence of fusion protein of heavy chain of anti-hTfR antibody No. 3 (humanized 2) and hARSA, synthetic sequence
SEQ ID NO:263: Amino acid sequence of the fusion protein of heavy chain of anti-hTfR antibody No. 3 (humanized 2) and hPPT-1
SEQ ID NO:264: Nucleotide sequence encoding the amino acid sequence of fusion protein of heavy chain of anti-hTfR antibody No. 3 (humanized 2) and hPPT-1, synthetic sequence
SEQ ID NO:266: Amino acid sequence of the fusion protein of heavy chain of anti-hTfR antibody No. 3 (humanized 2) and hTPP-1
SEQ ID NO:267: Nucleotide sequence encoding the amino acid sequence of fusion protein of heavy chain of anti-hTfR antibody No. 3 (humanized 2) and hTPP-1, synthetic sequence
SEQ ID NO:269: Amino acid sequence of the fusion protein of heavy chain of anti-hTfR antibody No. 3 (humanized 2) and hIDUA
SEQ ID NO:270: Nucleotide sequence encoding the amino acid sequence of fusion protein of heavy chain of anti-hTfR antibody No. 3 (humanized 2) and hIDUA, synthetic sequence
SEQ ID NO:272: Amino acid sequence of the fusion protein of heavy chain of anti-hTfR antibody No. 3 (humanized 2) and hTNFαR
SEQ ID NO:273: Nucleotide sequence encoding the amino acid sequence of fusion protein of heavy chain of anti-hTfRα antibody No. 3 (humanized 2) and hTNFαR, synthetic sequence
SEQ ID NO:275: Amino acid sequence of the fusion protein of heavy chain of anti-hTfR antibody No. 3 (humanized 2) and hSGSH
SEQ ID NO:276: Nucleotide sequence encoding the amino acid sequence of fusion protein of heavy chain of anti-hTfR antibody No. 3 (humanized 2) and hSGSH, synthetic sequence
SEQ ID NO:277: Amino acid sequence of anti-hTfR single-chain antibody
SEQ ID NO:278: Amino acid sequence 2 of CDR2 in the heavy chain of mouse anti-hTfR antibody No. 6
SEQ ID NO:279: Amino acid sequence 2 of CDR2 in the heavy chain of mouse anti-hTfR antibody No. 8

1-69. (canceled)

70. An anti-human transferrin receptor antibody, wherein the light chain variable region of the antibody comprises:
a) the amino acid sequence set forth as SEQ ID NO:17 in CDR1,
b) the amino acid sequence set forth as SEQ ID NO:19 in CDR2, and
c) the amino acid sequence set forth as SEQ ID NO:20 in CDR3; and
the heavy chain variable region comprises:
a) the amino acid sequence set forth as SEQ ID NO:88 in CDR1,
b) the amino acid sequence set forth as SEQ ID NO:91 in CDR2, and
c) the amino acid sequence set forth as SEQ ID NO:92 in CDR3.

71. The anti-human transferrin receptor antibody according to claim 70, wherein the antibody has an affinity to both the extracellular region of human transferrin receptor and the extracellular region of monkey transferrin receptor.

72. The anti-human transferrin receptor antibody according to claim 71, wherein the dissociation constant of its complex with the extracellular region of human transferrin receptor is not greater than 1×10⁻⁸ M, and the dissociation constant of its complex with the extracellular region of monkey transferrin receptor is not greater than 5×10⁻⁸ M.

73. The anti-human transferrin receptor antibody according to claim 70, wherein the light chain variable region of the antibody has the amino acid sequence set forth as SEQ ID NO:191, and wherein the heavy chain variable region of the antibody has the amino acid sequence set forth as SEQ ID NO:205.

74. The anti-human transferrin receptor antibody according to claim 70, wherein the antibody is a Fab antibody, F(ab′)₂ antibody, or F(ab′) antibody.

75. The anti-human transferrin receptor antibody according to claim 70, wherein the light chain of the antibody has the amino acid sequence set forth as SEQ ID NO:196, and wherein the heavy chain of the antibody has the amino acid sequence set forth as SEQ ID NO:210.

76. A fusion protein comprising the anti-human transferrin receptor antibody according to claim 70 and the amino acid sequence of a different protein (A) linked to the light chain of the antibody on the C-terminal side or the N-terminal side thereof.

77. A fusion protein comprising the anti-human transferrin receptor antibody according to claim 70 and the amino acid sequence of a different protein (A) linked to the heavy chain of the antibody on the C-terminal side or the N-terminal side thereof.

78. The fusion protein according to claim 77, wherein the different protein (A) is linked, via a linker sequence, to the heavy chain on the C-terminal side or the N-terminal side thereof.

79. The fusion protein according to claim 78, wherein the linker sequence consists of 1 to 50 amino acid residues.

80. The fusion protein according to claim 78, wherein the linker sequence comprises an amino acid sequence selected from the group consisting of a single glycine, a single serine, the amino acid sequence Gly-Ser, the amino acid sequence Gly-Gly-Ser, the amino acid sequence set forth as SEQ ID NO:3, the amino acid sequence set forth as SEQ ID NO:4, the amino acid sequence set forth as SEQ ID NO:5, and the amino acid sequences consisting of 1 to 10 thereof that are consecutively linked.

81. The fusion protein according to claim 76, wherein the different protein (A) is a protein originating from human.

82. The fusion protein according to claim 76, wherein the different protein (A) is selected from the group consisting of nerve growth factor (NGF), lysosomal enzymes, ciliary neurotrophic factor (CNTF), glial cell line-derived neurotrophic factor (GDNF), neurotrophin-3, neurotrophin-4/5, neurotrophin-6, neuregulin-1, erythropoietin, darbepoetin, activin, basic fibroblast growth factor (bFGF), fibroblast growth factor 2 (FGF2), epidermal growth factor (EGF), vascular endothelial growth factor (VEGF), interferon α, interferon β, interferon γ, interleukin 6, granulocyte-macrophage colony stimulating factor (GM-CSF), granulocyte colony stimulating factor (G-CSF), macrophage colony stimulating factor (M-CSF), cytokines, tumor necrosis factor α receptor (TNF-α receptor), PD-1 ligands, enzymes having β-amyloid-degrading activity, anti-β-amyloid antibody, anti-BACE antibody, anti-EGFR antibody, anti-PD-1 antibody, anti-PD-L1 antibody, anti-HER2 antibody, anti-TNF-α antibody, and other antibody medicines.

83. The fusion protein according to claim 76, wherein the different protein (A) is a lysosomal enzyme, and wherein the lysosomal enzyme is selected from the group consisting of α-L-iduronidase, iduronate 2-sulfatase, glucocerebrosidase, β-galactosidase, GM2 activator protein, β-hexosaminidase A, β-hexosaminidase B, N-acetylglucosamine-1-phosphotransferase, α-mannosidase, β-mannosidase, galactosylceramidase, saposin C, arylsulfatase A, α-L-fucosidase, aspartylglucosaminidase, α-N-acetylgalactosaminidase, acidic sphingomyelinase, α-galactosidase A, β-glucuronidase, heparan N-sulfatase, α-N-acetylglucosaminidase, acetyl COA:α-glucosaminide N-acetyltransferase, N-Acetylglucosamine-6-sulfate sulfatase, acid ceramidase, amylo-1,6-glucosidase, sialidase, palmitoyl protein thioesterase 1, tripeptidyl-peptidase 1, hyaluronidase 1, CLN1 and CLN2.

84. A method for treatment of a disorder of the central nervous system, the method comprising parenterally administering to a patient with the disorder a therapeutically effective amount of a physiologically active protein, or pharmacologically active low-molecular-weight compound, for the disorder, in the form of a conjugate with the anti-human transferrin receptor antibody according to claim 70.
